SEQUENCE LISTING (120 sequences)

SEQ ID NO: 1 - protein, 345 aa, Saccharomyces cerevisiae
MSVADLKNNIHKLDTGYGLMSLTWRAEPIPQSQAFEAMHRVVELSRERGHKAFFNVGEFYGPDFINLSYVHDFFAKYPDLRKDVVISCKGGADNATLTPRGSHDDVVQSVKNSVSAIGGYIDIFEVARIDTSLCTKGEVYPYESFEALAEMISEGVIGGISLSEVNEEQIRAIHKDWGKFLTCVEVELSLFSNDILHNGIAKTCAELGLSIICYSPLGRGLLTGQLKSNADIPEGDFRKSLKRFSDESLKKNLTLVRFLQEEIVDKRPQNNSITLAQLALGWVKHWNKVPEYSGAKFIPIPSGSSISKVNENFDEQKTKLTDQEFNAINKYLTTFHTVGDRYEMA

SEQ ID NO: 2 - protein, 333 aa, Schizosaccharomyces pombe
MPIVSGFKVGPIGFGLMGLTWKPKQTPDEEAFEVMNYALSQGSNYWDAGEFYGVDPPTSNLDLLARYFEKYPENANKVFLSVKGGLDFKTLFLVGNRTSFPRSVENVIAHLRGTQKLDLFQCARVDPNVPIETTMKTLKGFVDSGKISCVGLSEVSAETIKRAHAVVPIAAVEVEYSLFSRDIETNGIMDICRKLSIPIIAYSPFCRGLLTGRIKTVEDLKEFAKSFPFLEYLDRFSPDVFAKNLPFLQAVEQLAKKFGMTMPEFSLLFIMASGNGLVIPIPGSTSVSRTKSNLNALNKSLSPEQFKEAKEVLSKYPIYGLRYNEQLAGTLSV

SEQ ID NO: 3 - protein, 393 aa, Mesorhizobium loti
MMRYPEHADPVITLTAGPVNAYPEVLRGLGRTVLYDYDPAFQLLYEKVVDKAQKAMRLSNKPVILHGEPVLGLEAAAASLISPDDVVLNLASGVYGKGFGYWAKRYSPHLLEIEVPYNEAIDPQAVADMLKAHPEITVVSVCHHDTPSGTINPIDAIGALVSAHGAYLIVDAVSSFGGMKTHPEDCKADIYVTGPNKCLGAPPGLTMMGVSERAWAKMKANPLAPRASMLSIVDWENAWSRDKPFPFTPSVSEINGLDVALDLYLNEGPEAVWARHALTAKAMRAGVTAMGLSVWAASDSIASPTTTAVRTPDGVDEKALRQAARARYGVVFSSGRGETLGKLTRIGHMGPTAQPIYAIAALTALGGAMNAAGRKLAIGKGIEAALAVIDADA

SEQ ID NO: 4 - protein, 396 aa, Escherichia coli
MFENITAAPADPILGLADLFRADERPGKINLGIGVYKDETGKTPVLTSVKKAEQYLLENETTKNYLGIDGIPEFGRCTQELLFGKGSALINDKRARTAQTPGGTGALRVAADFLAKNTSVKRVWVSNPSWPNHKSVFNSAGLEVREYAYYDAENHTLDFDALINSLNEAQAGDVVLFHGCCHNPTGIDPTLEQWQTLAQLSVEKGWLPLFDFAYQGFARGLEEDAEGLRAFAAMHKELIVASSYSKNFGLYNERVGACTLVAADSETVDRAFSQMKAAIRANYSNPPAHGASVVATILSNDALRAIWEQELTDMRQRIQRMRQLFVNTLQEKGANRDFSFIIKQNGMFSFSGLTKEQVLRLREEFGVYAVASGRVNVAGMTPDNMAPLCEAIVAVL

SEQ ID NO: 5 - protein, 371 aa, Artificial
  Modified Alanine Dehydrogenase
MIIGVPTEIKNHEYRVGMVPSSVRELTIKGHVVYVQSDAGVGIGFTDQDYIDAGASILATAAEVFAKSDMIVKVKEPQAVERAMLRHDQILFTYLHLAPDLPQTEELITSGAVCIAYETVTDDRGGLPLLAPMSEVAGRMSIQAGARALEKSLGGRGMLLGGVPGVEPAKVVIIGGGMVGTNAAQMAVGMGADVVVLARSIDALRRLNVQFGSAVKAIYSTADAIERHVLEADLVIGGVLVPGAAAPKLITRDMVKRMKPGSAIVDVAIDQGGCVETSHATTHQDPTYIVDDVVHYCVANMPGAVARTSTFALNNATLPYIIKLANQGYKQALLNDKHLLNGLNVMHGKVVCKEVAEALNLEFTEPKSLLA

SEQ ID NO: 6 - protein, 447 aa, Escherichia coli
MDQTYSLESFLNHVQKRDPNQTEFAQAVREVMTTLWPFLEQNPKYRQMSLLERLVEPERVIQFRVVWVDDRNQIQVNRAWRVQFSSAIGPYKGGMRFHPSVNLSILKFLGFEQTFKNALTTLPMGGGKGGSDFDPKGKSEGEVMRFCQALMTELYRHLGADTDVPAGDIGVGGREVGFMAGMMKKLSNNTACVFTGKGLSFGGSLIRPEATGYGLVYFTEAMLKRHGMGFEGMRVSVSGSGNVAQYAIEKAMEFGARVITASDSSGTVVDESGFTKEKLARLIEIKASRDGRVADYAKEFGLVYLEGQQPWSLPVDIALPCATQNELDVDAAHQLIANGVKAVAEGANMPTTIEATELFQQAGVLFAPGKAANAGGVATSGLEMAQNAARLGWKAEKVDARLHHIMLDIHHACVEHGGEGEQTNYVQGANIAGFVKVADAMLAQGVI

SEQ ID NO: 7 - DNA, 1038 bp, Saccharomyces cerevisiae
atgtctgtcgccgatttgaaaaacaacatccacaagttagatactggctatggtttaatgagtttgacttggagagccgagcctatccctcagtcgcaggctttcgaggccatgcacagagtggttgagttatccagagaacgtgggcacaaggcctttttcaacgttggtgaattctatggtcccgattttattaatttgtcgtatgttcacgacttctttgcgaaatacccagatttgagaaaggatgtggttatcagttgtaaaggtggtgcagacaatgctaccttaacccccagaggcagtcacgatgatgttgtacaaagcgtaaagaattcagttagtgctattggtggctacatcgacatcttcgaagtcgcaagaatcgacacttccctatgcacgaaaggagaggtctacccctacgaatcgttcgaagcgcttgctgagatgatctccgaaggcgttattggcggtatttcattaagtgaagttaatgaagagcaaattagagctattcacaaggattggggaaagtttttgacctgcgttgaagtggaactttctttgttcagtaatgacattttacacaacggaattgctaaaacatgtgctgaattggggttgtccatcatctgctactccccactgggcagaggattgttgacaggtcaattgaagtcaaacgctgatatccctgagggtgactttagaaagtcgttaaagagatttagcgacgagtctttgaaaaaaaacctgaccttggtcaggtttctacaggaagaaatagtcgacaagcgcccacaaaacaactccattactcttgcacaactggctttgggatgggttaagcactggaacaaagttccggaatacagtggcgccaaatttatcccaattccaagtggctcttctatttccaaggttaatgaaaactttgatgaacagaaaaccaaacttaccgatcaagagttcaatgccattaacaaatatttgactactttccatactgttggtgacagatacgaaatggcgtaa

SEQ ID NO: 8 - DNA, 1002 bp, Schizosaccharomyces pombe
atgcctatcgttagcggatttaaagtcggacctattggctttggtcttatgggtttaacctggaagcccaagcaaactccagatgaagaagcctttgaagtaatgaattatgccttatcccaaggctccaattattgggatgcgggtgaattttatggtgtcgaccctcccacctctaatttagatttattagctagatattttgaaaaatatcccgaaaacgctaataaggtgtttttatctgtcaaaggtggtttggattttaaaacactattcctggtgggaaatcggacttcgtttccaaggagtgttgaaaatgtgatagctcatttacgtgggacccaaaagctagatctcttccaatgtgctcgagtcgacccaaatgttcccattgagacaaccatgaagacattgaaggggtttgtggactctggaaagatttcatgtgtcggccttagcgaagtttctgctgaaactatcaaacgtgctcacgctgtggtccccattgctgcagtcgaagtcgaatactccttgttttctcgtgatatcgagacaaacggtattatggatatttgtaggaagttatctatccctattattgcatactctcctttctgtcgtggccttttgactggtcgcattaagactgtcgaagaccttaaagaatttgctaaatctttcccattcttagaatatttggacagatttagccctgatgtttttgctaaaaatcttccattcctccaagctgtcgagcaacttgctaaaaagtttggtatgactatgcccgagttttcacttctttttataatggcgtctggaaacggtctggtcattcctattcctggctccacatctgtttctcgtacaaagagcaatctcaatgcactgaataaatctttgtctcctgaacaatttaaagaggccaaagaggtcttgagtaaatacccaatctacgggttacgttataatgaacagcttgcgggcactctttccgtttaa

SEQ ID NO: 9 - DNA, 1182 bp, Mesorhizobium loti
atgatgcgctatcccgaacatgccgatccggtcatcaccctgaccgcggggccggtgaacgcctatccagaagtgttgcgcggcctcggccgtacggtgctttatgattacgatcctgcattccagctcctttacgagaaggtggtcgacaaggcgcagaaggcgatgcggctgtcgaacaagccggtcatcctgcatggcgagccggtgctcggcctcgaagcggcggcggcgtcgctgatctcgcccgacgacgttgtgctcaatcttgcctcgggcgtctacggcaagggctttggctattgggcgaagcggtactcgccgcatctgctcgagatcgaagtgccctataacgaggcgatcgatccgcaggcggtcgccgacatgctcaaggcgcatccggagatcaccgttgtgtcggtctgtcatcacgacacgccgtcgggcaccatcaatccgatcgacgccatcggcgcgctggtctcggcgcatggcgcctatctgatcgtcgacgcggtctcgtcctttggcggcatgaagacgcatcccgaggattgcaaagccgatatttatgtcaccggccccaacaaatgcctgggcgcccctcccggcctcaccatgatgggcgtcagcgagcgggcctgggccaagatgaaggccaaccccctggcgccgcgcgcatcgatgctgagcattgtcgactgggaaaatgcctggtcgagggacaagccgtttccgttcacgccgtcggtctcggagatcaacgggctcgacgtcgcgctcgatctttacctcaatgagggaccggaggcggtatgggcgcgccatgcgctcaccgccaaggccatgcgagcgggcgtcaccgcgatgggcctgtcggtctgggccgccagcgacagcatcgcgtcgccgaccaccaccgccgtccgcacccccgatggtgtcgacgagaaggcgcttcgccaggccgcgcgtgcccgttacggcgtggtgttttcgtcggggcggggcgagacgttggggaagctgacgcgcatcggccatatggggccgaccgcccagccgatctacgcgatcgcggcgctgacggcacttggcggcgccatgaacgcggccggccggaaactcgcaatcggcaaaggcatcgaggcggcgctggccgtaatcgacgccgacgcctga

SEQ ID NO: 10 - DNA, 1191 bp, Escherichia coli
atgtttgagaacattaccgccgctcctgccgacccgattctgggcctggccgatctgtttcgtgccgatgaacgtcccggcaaaattaacctcgggattggtgtctataaagatgagacgggcaaaaccccggtactgaccagcgtgaaaaaggctgaacagtatctgctcgaaaatgaaaccaccaaaaattacctcggcattgacggcatccctgaatttggtcgctgcactcaggaactgctgtttggtaaaggtagcgccctgatcaatgacaaacgtgctcgcacggcacagactccggggggcactggcgcactacgcgtggctgccgatttcctggcaaaaaataccagcgttaagcgtgtgtgggtgagcaacccaagctggccgaaccataagagcgtctttaactctgcaggtctggaagttcgtgaatacgcttattatgatgcggaaaatcacactcttgacttcgatgcactgattaacagcctgaatgaagctcaggctggcgacgtagtgctgttccatggctgctgccataacccaaccggtatcgaccctacgctggaacaatggcaaacactggcacaactctccgttgagaaaggctggttaccgctgtttgacttcgcttaccagggttttgcccgtggtctggaagaagatgctgaaggactgcgcgctttcgcggctatgcataaagagctgattgttgccagttcctactctaaaaactttggcctgtacaacgagcgtgttggcgcttgtactctggttgctgccgacagtgaaaccgttgatcgcgcattcagccaaatgaaagcggcgattcgcgctaactactctaacccaccagcacacggcgcttctgttgttgccaccatcctgagcaacgatgcgttacgtgcgatttgggaacaagagctgactgatatgcgccagcgtattcagcgtatgcgtcagttgttcgtcaatacgctgcaggaaaaaggcgcaaaccgcgacttcagctttatcatcaaacagaacggcatgttctccttcagtggcctgacaaaagaacaagtgctgcgtctgcgcgaagagtttggcgtatatgcggttgcttctggtcgcgtaaatgtggccgggatgacaccagataacatggctccgctgtgcgaagcgattgtggcagtgctgtaa

SEQ ID NO: 11 - DNA, 1116 bp, Artificial
  Modified Nucleotide Sequence for Alanine Dehydrogenase
atgattattggtgttccaacagaaatcaaaaaccacgaataccgtgtaggcatggtcccttcaagcgtacgtgaattaaccataaaaggtcacgttgtttatgttcaatcagatgcgggtgtgggtattggttttaccgatcaagactatattgatgcgggcgcctcgattttagctacagcagctgaagtgtttgctaaatcagacatgattgtaaaagttaaagagccacaagcagttgaacgtgcaatgttacgtcacgaccagattttattcacttacttgcaccttgcaccagatttgccacaaactgaagaattaatcaccagtggcgctgtttgtattgcttacgaaacagtcactgatgatcgcggcggattaccattacttgccccaatgtcagaagttgctggccgcatgtcaatccaagcaggcgcacgcgcacttgaaaaatcattaggcggccgtggtatgttgcttggtggtgttccgggtgttgagccagctaaagtggtcatcattggtggcggtatggttggtacaaacgcagcacaaatggctgttggtatgggtgctgatgttgttgttttggctcgcagtatcgatgcattacgtcgcctaaatgttcaatttggttcagcggttaaagctatttactcaacagctgatgctattgagcgtcacgtattagaagcagaccttgttattggcggtgtattagttccaggtgctgcagcaccaaagttaatcactcgtgacatggttaagcgcatgaaacctggcagtgcaattgttgacgttgcaattgaccaaggtggttgtgttgaaacttcgcacgctaccactcaccaagacccaacttacattgttgatgacgtagtgcattactgtgtggctaacatgccaggtgcagtagcgcgtacttctacgtttgcattaaacaatgcaacactaccttacattatcaagttagcgaaccaaggctacaaacaagcattattaaatgacaaacatttattaaatggcttaaacgtaatgcacggtaaagtcgtctgtaaagaagtggctgaagcgttgaaccttgagttcactgaaccaaaaagcctacttgcttaa

SEQ ID NO: 12 - DNA, 1344 bp, Escherichia coli
atggatcagacatattctctggagtcattcctcaaccatgtccaaaagcgcgacccgaatcaaaccgagttcgcgcaagccgttcgtgaagtaatgaccacactctggccttttcttgaacaaaatccaaaatatcgccagatgtcattactggagcgtctggttgaaccggagcgcgtgatccagtttcgcgtggtatgggttgatgatcgcaaccagatacaggtcaaccgtgcatggcgtgtgcagttcagctctgccatcggcccgtacaaaggcggtatgcgcttccatccgtcagttaacctttccattctcaaattcctcggctttgaacaaaccttcaaaaatgccctgactactctgccgatgggcggtggtaaaggcggcagcgatttcgatccgaaaggaaaaagcgaaggtgaagtgatgcgtttttgccaggcgctgatgactgaactgtatcgccacctgggcgcggataccgacgttccggcaggtgatatcggggttggtggtcgtgaagtcggctttatggcggggatgatgaaaaagctctccaacaataccgcctgcgtcttcaccggtaagggcctttcatttggcggcagtcttattcgcccggaagctaccggctacggtctggtttatttcacagaagcaatgctaaaacgccacggtatgggttttgaagggatgcgcgtttccgtttctggctccggcaacgtcgcccagtacgctatcgaaaaagcgatggaatttggtgctcgtgtgatcactgcgtcagactccagcggcactgtagttgatgaaagcggattcacgaaagagaaactggcacgtcttatcgaaatcaaagccagccgcgatggtcgagtggcagattacgccaaagaatttggtctggtctatctcgaaggccaacagccgtggtctctaccggttgatatcgccctgccttgcgccacccagaatgaactggatgttgacgccgcgcatcagcttatcgctaatggcgttaaagccgtcgccgaaggggcaaatatgccgaccaccatcgaagcgactgaactgttccagcaggcaggcgtactatttgcaccgggtaaagcggctaatgctggtggcgtcgctacatcgggcctggaaatggcacaaaacgctgcgcgcctgggctggaaagccgagaaagttgacgcacgtttgcatcacatcatgctggatatccaccatgcctgtgttgagcatggtggtgaaggtgagcaaaccaactacgtgcagggcgcgaacattgccggttttgtgaaggttgccgatgcgatgctggcgcagggtgtgatttaa

SEQ ID NO: 13 - DNA, 1055 bp, Artificial
  Artificial Fragment for Pyridoxine Dehydrogenase
acaaaaaggataaaacaatgtcagtagctgatctaaagaacaacatacacaaactagatacaggatatgggctaatgagcctgacctggcgtgcggaaccgatcccgcagagccaagcgttcgaggcgatgcaccgtgtggttgagctgagccgtgaacgtggtcacaaggcgttctttaacgtgggcgagttctacggcccggactttatcaacctgagctacgttcacgatttctttgcgaagtatccggacctgcgtaaagatgtggttattagctgcaagggtggcgcggacaacgcgaccctgaccccgcgtggcagccacgatgatgtggttcagagcgtgaaaaacagcgttagcgcgatcggtggctatatcgacattttcgaggtggcgcgtattgataccagcctgtgcaccaaaggtgaagtttacccgtatgagagctttgaagcgctggcggagatgatcagcgaaggcgtgatcggtggcattagcctgagcgaggttaacgaggaacaaatccgtgcgattcacaaggactggggtaaattcctgacctgcgttgaggtggaactgagcctgtttagcaacgatatcctgcacaacggtattgcgaagacctgcgcggaactgggcctgagcatcatttgctacagcccgctgggtcgtggcctgctgaccggtcagctgaaaagcaacgcggacatcccggagggcgatttccgtaagagcctgaaacgttttagcgacgaaagcctgaagaaaaacctgaccctggtgcgtttcctgcaagaggaaatcgttgataagcgtccgcagaacaacagcattaccctggcgcaactggcgctgggttgggtgaagcactggaacaaagttccggagtatagcggtgcgaagtttatcccgattccgagcggcagcagcattagcaaagtgaacgagaacttcgacgaacagaagaccaaactgaccgatcaagaatttaatgcgattaacaaatacctgaccaccttccacaccgttggcgaccgctatgagatggcgtaa

SEQ ID NO: 14 - DNA, 1199 bp, Artificial
  Artificial Fragment for Pyridoxamine Pyruvate Transaminase
acaaaaaggataaaacaatgatgaggtatcccgaacacgctgatccagtaataactttaacagcaggacccgtaaacgcgtatccggaagttctgcgtggtctgggccgtaccgttctgtacgactatgatccggcgttccagctgctgtacgagaaggtggttgataaggcgcaaaaagcgatgcgtctgagcaacaaaccggtgattctgcacggcgaaccggttctgggtctggaagcggcggcggcgagcctgatcagcccggacgatgtggttctgaacctggcgagcggcgtgtacggcaagggctttggttactgggcgaaacgttatagcccgcacctgctggagattgaagtgccgtataacgaggcgattgacccgcaggcggttgcggatatgctgaaggcgcacccggaaatcaccgtggttagcgtgtgccaccacgacaccccgagcggtaccatcaacccgattgacgcgattggtgcgctggttagcgcgcacggtgcgtacctgattgtggacgcggttagcagcttcggtggcatgaagacccacccggaggactgcaaagcggatatctatgtgaccggtccgaacaaatgcctgggtgctccgccgggtctgaccatgatgggtgttagcgaacgtgcgtgggcgaagatgaaagcgaacccgctggcgccgcgtgcgagcatgctgagcattgtggactgggagaacgcgtggagccgtgataagccgttcccgtttaccccgagcgtgagcgaaatcaacggcctggacgttgcgctggatctgtatctgaacgaaggtccggaagcggtttgggcgcgtcatgcgctgaccgcgaaggcgatgcgtgcgggcgtgaccgcgatgggtctgagcgtttgggcggcgagcgatagcattgcgagcccgaccaccaccgcggtgcgtaccccggatggtgttgatgaaaaggcgctgcgtcaggcggcgcgtgcgcgttacggcgtggtttttagcagcggccgtggtgaaaccctgggtaaactgacccgtattggccacatgggtccgaccgcgcaaccgatttatgcgattgcggcgctgaccgcgctgggtggcgcgatgaacgcggcgggtcgtaagctggcgattggcaaaggtatcgaagcggcgctggcggttatcgacgcggatgcgtga

SEQ ID NO: 15 - DNA, 1133 bp, Artificial
  Artificial Fragment for Alanine Dehydrogenase
acaaaaaggataaaacaatgataataggagtacccacagaaataaagaaccacgaatacagggttgggatggtaccgagcagcgtgcgtgaactgaccatcaagggccacgtggtttacgttcagagcgatgcgggcgtgggtattggtttcaccgaccaagattatatcgatgcgggtgcgagcattctggcgaccgcggcggaagtgtttgcgaaaagcgacatgatcgttaaggtgaaagaaccgcaggcggtggaacgtgcgatgctgcgtcacgaccaaattctgttcacctacctgcacctggcgccggatctgccgcagaccgaggaactgatcaccagcggtgcggtttgcattgcgtatgagaccgtgaccgatgatcgtggtggcctgccgctgctggcgccgatgagcgaggttgcgggtcgtatgagcatccaagcgggtgcgcgtgcgctggagaagagcctgggtggtcgtggtatgctgctgggtggcgttccgggcgtggaaccggcgaaggtggttatcattggtggcggtatggttggtaccaacgcggcgcagatggcggtgggtatgggtgcggatgtggttgtgctggcgcgtagcattgatgcgctgcgtcgtctgaacgttcaattcggcagcgcggtgaaagcgatctacagcaccgcggacgcgattgagcgtcacgttctggaagcggatctggtgatcggcggtgttctggttccgggtgcggcggcgccgaagctgattacccgtgacatggttaagcgtatgaaaccgggcagcgcgatcgttgacgtggcgattgatcagggcggttgcgttgaaaccagccacgcgaccacccaccaagatccgacctacatcgtggacgatgttgtgcactattgcgttgcgaacatgccgggtgcggtggcgcgtaccagcacctttgcgctgaacaacgcgaccctgccgtacatcattaagctggcgaaccagggctataaacaagcgctgctgaacgataagcacctgctgaacggtctgaacgtgatgcacggcaaggttgtgtgcaaggaagtggcggaggcgctgaacctggagtttaccgagccgaagagcctgctggcgtaa

SEQ ID NO: 16 - DNA, 34 bp, Artificial
  Primer Sequence
acgcgtcgacacaaaaaggataaaacaatgtcag

SEQ ID NO: 17 - DNA, 29 bp, Artificial
  Primer Sequence
cccaagcttttacgccatctcatagcggt

SEQ ID NO: 18 - DNA, 29 bp, Artificial
  Primer Sequence
ccggaattcacaaaaaggataaaacaatg

SEQ ID NO: 19 - DNA, 28 bp, Artificial
  Primer Sequence
cgcggatcctcacgcatccgcgtcgata

SEQ ID NO: 20 - DNA, 29 bp, Artificial
  Primer Sequence
cgcggatccacaaaaaggataaaacaatg

SEQ ID NO: 21 - DNA, 30 bp, Artificial
  Primer Sequence
acgcgtcgacttacgccagcaggctcttcg

SEQ ID NO: 22 - protein, 345 aa, Saccharomyces eubayanus
MSVTELRNDIHKLDTGYGLMSLTWRAEPIPQSQAFEAMHRAVELARERGHKAFFNVGEFYGPDFVNLIFVRDFFAKYPDLRKHVVISCKGGMEVSTLTPNGSHDAVIKSVKNSVSAIGGYIDIFEVARLDTSLCAKGEVYPYESFEALAEMISEGVIGGISLSEVTEEQIRAIYKDWGKFLTCVELEVSLFSTDIFHNGIAKTCAELGLTVICYSPLGRGLLTGQLKSNADIPEGDFRKALKRFSDESLKKNLDLVRFLQEEIVGKRSKDNSITLPQLALGWIKHWNYVPEYKGAKFIPIPSGSSISKVNENFDEKKTQLTDKEFKAINDFLATFHTVGDRYEFN

SEQ ID NO: 23 - protein, 349 aa, Torulaspora delbrueckii
MVEQSAINKLKQDLSAIDTGYGLMSLTWRAEPIPETQAHETMKRVVELAQSKGHKAFFNCGEFYGPNRINLTYIRDFFVKNPELRKYVIISCKGGCDCETLTPKGKHDDVIKSVEASVAAIGGFIDIFEVARLDLSLCTNGETYPYESFEALAEMVDNGAIGAISLSEVTAEQIRGISKDWHKYLVSVEVELSMFTKAILTNGVVKANNDLNLVTILYSPLGRGLLTGAVTSSKDIPAGDFRNLLKRFDDESLRQNLTLLDFLRDEIIAKRPANNEITLPQVALGWDKYWNKTGEYPNTHFLPIPSGSSVKKLEENFDEAKTQITAEEFRKINDFLKNFKTVGDAYEMR

SEQ ID NO: 24 - protein, 341 aa, Zygosaccharomyces bailii
MSKTGELKRQLNQLHTGYGLMSLTWRADPVPQEQAFEAMERVVSLAQASGNKAFFNVGEFYGPDYSNLELVKAFFEAKPELREHVLISCKGGVDNTKLLPKGKYADVLSSIEQCVKHLGTHLDIFEVARLDKSLGGEYPRESFEAMASMVDKGIIDGISLSEVTADEIRAIGHDWAKYLVCVEVEFSMFSPQILHNGVLDACNDLGLIVIAYSPLGRGLLTGTITKDSAFKDFRSSLKRFQKDSLQKNLALTEFLQDQIVDQRSSTNPISLPQVALGWVKSLNNGKYPHTHIVPIPSGSDKRKVEENFDESRAKLTVSEIEKINDFLKSFNVVGDRYEWVS

SEQ ID NO: 25 - protein, 320 aa, Aspergillus oryzae
MPSLVGRDVGHTGYGLMRMTWVPQPPPQEQCFEALNTALAHGSNFWNAGELYGTPEYNSLHLLHSYFAQHPENADKVVLSIKGGLKPGQLVPDGSEANIRRSVDECLRVLDGKKKIDIFECARQDPKTTVEQTVTVLAQLVKEGKIGGIGLSEVDAETIRRAHKVHPIAAVEVEMSLFDLTILQNDVAKVCAELNIPIVAYSPLGRGVLAGAFTTAADIPDGDFRKTLPKFQDEAMKQNIKLVNEVNDLAARKGVAPVQIALAWVLTLSGKPGMPTIIPIPGGTTSAKVAQNLQAPRLTDAEMAEIDAILKRNEIVGTRY

SEQ ID NO: 26 - protein, 343 aa, Kluyveromyces marxianus
MPSPSDYRKELLDVEVGYGLMSLTWRKDPLPAEKVFPTMKKVVEKAGNKKALFNVGEFYGPHLANYKLLQSFFDKYPEDRKKVIISAKGGVNVETLQPTGDADSISASIENALKVFGGYLDIYEPARIDLALAEKNGEKLFPRETFDTIVKYIKDGKVGGFSLSEVTNEQIRAIYKEYGEYLACVEVELSIASPEIISNGILDTCNEFGIPVVAYAPLGRGLLTGSLKSTADIPEGDFRGQLKRFQDDAIAHNLTLVKFLQDEIAAKRSDNPSLPQVAIGWVRGLRREYPKTNIIPIPSGSSVEKVSVNFDHIDLSDDEMSKIKDFLKSFKIAGDRYEFAQAH

SEQ ID NO: 27 - protein, 349 aa, Candida albicans
MSFKPVEISGKFGFGTMSMTWTPTPPPAQQSIDTLKFVTSHPKFGTKLINGGEFYGPDFANLKLLKQFLEENDPEENKQLIISIKGGADNETLKPNGTKEFVSKSIENIVSFFPKQKQNRPKLLFEMARVDPSVPYGETIGYISEYVKSGVIDGISLSEVGKESIQAALKVFPISCVELELSLFSQEVITTGILEELSKHNLPLIAYSPLCRGLLTDYAVENSDTFLASIPQGDIRHHLDKFQPDTFNKNLPALKELYKFAHEVKNTTLESLALSWIVTVSEARNFRGIEKVTRILPIPSGSTKKRVESNFGSLIELTDDDLQSIDEIFAKYPISGLRYNQFLEGTLFQ

SEQ ID NO: 28 - protein, 329 aa, Yarrowia lipolytica
MTFNKITPTSKGFGLMGFTWRETETPDEIAFPTMKRAIEKGILVWNSAEFYGTKDPLGNLKLIRRYFEKYPEDADKVCLMVKGGLGPNLVPNGSKEFVQKSIDTVLETLGGSVPVSIFQCARVDPNTPIEETVAAIKEYVDAGKIGALGLSEVGAATIEKVHSLSPIAAVENELSLWATDIFTNGVAEVCAKYNIPITAYAPLGSGVLTGRFKSTQDVPEGDARKHMVKYQEENLKQNLVLVDRLKEVATKAGIPLPQMAIAWVKAQSNHNGHGTIIPIPGNTTVARLEENHNDLTLSAETLKEIDDVLKSTKIVGDRGFTLGGEFMNG

SEQ ID NO: 29 - protein, 392 aa, Mesorhizobium sp. YR577
MRYPDHTEPVITLTAGPVNAYPDVLRGLGRTVLYDYDPAFQLFYEKVIGKAQKAMRLSNKPVVLHGEPVMGLEAAAASLISRDDVVLNLASGVYGKGFGYWAKRYSPNLLEIEVPYNEAIDPQSVADMLKSHPEITVVSVCHHDTPSGTINPIDEIGAIVSAHGGYLIVDAVSSFGGMKTHPEDCKADIYVTGPNKCLGAPPGLTLMSVSDRAWTRMKANSAAPRASVLSILDWEHAWSKEKPFPFTPSVAEMNGLDVALDLYLNEGPEAVWARHALTAKATRAGAQAMGLSIWAANEKIASPTTTAIRTPDGVDEKALRQAARERYGVVFSSGRGETLGKLTRIGHMGPTAQPIYAIAALTALGGALNAAGRKLQVGKGIEAALAVIDADA

SEQ ID NO: 30 - protein, 392 aa, Pseudaminobacter salicylatoxidans
MRYKDNADPVITLTSGPVNAYADVLRGLSRTVLYDYDPAFQLFYENVVAKAQKAMRLSTRPVLLQGEPVMGLEAAAASLISSEDVVLNLASGVYGKGFGYWAKRYSPNLLEIETPYNESIDPQAVADMLKKHPEITVVSVCHHDTPSGTINPIDEIGAIVSAHGAYFIVDAVSSFGGMKTHPEDCKADLYVTGPNKCLGAPPGLTMMGVSDRAWARMKANPSAPRASVLSILDWEDAWSHEKPFPFTPSVAEINGLDVALDLYLNEGPEVVWARHALTAKATRAGALAMGLSLWAASEKIASPTTTSIRTPQGVDEAALRQAVRERYGVVFSSGRGETLGKLTRIGHMGPTAQPIYAVAALTALGGALNAMGQKLAVGRGIDAALAVIDADA

SEQ ID NO: 31 - protein, 392 aa, Bauldia litoralis
MRYALDADPVITLTSGPVNAYPDVLRALARTVLYDYDPAFQLHYEQVIGKMQTAMRTSNRPVILHGEPVLGLEAAAASLITADDTVLNLASGVYGKGFGYWAARYSPNQLEIEVPYNQAIDPQQVADMLKANPAISVVAVCHHDTPSGTINPIDEIGAIVADHGAYLIVDAVSSFGGMKTHPEDCKADIYVTGPNKCLGAPPALTMLGVSERAWQKMKDNPAAPRASMLSILDWENAWSRDEAFPFTPSVAEVNGLDVALDLYLAEGPEAVWARHALTAAATRAGILAMGLDLWAADAAIASPTTTAVRTPEGIDEAKLRATVRERYGVVMSSGRGETLGKLTRIGHMGPTAQPIYAIAALTAFGGALQALGHPAPVGKAIDAALAVIDAAA

SEQ ID NO: 32 - protein, 391 aa, Skermanella stibiiresistens
MTVERIVEPAFTLSSGPVDAYPAVLRALSRTVLYDFDPVFQHFYEAVTDKAKVALRTAAPPVILHGEPVLGLEAAAASLIARDDVVLNLASGVYGKGFGFWAKRYCAELVEIEVPYDDAIDPAAVEAAFRERPDIRVVSVCHHDTPSGTLNPIDEIGRIVAAHGAYLIVDAVSSFGGMDVHPEACQADIFVTGPNKCLGCPPGLTLLAVSGRAWDKMKANPDAPRASILSILDWENAWRRDQPFPFTPSISEVNALDAALDLYLEEGPEAVWRRHALTSKACRDGIRAMGMTIWPAREAIASPTTTAVRVPDGIDADALLATARDGFGVSFSSGRGETKGKLIRIGHMGRTAQPLYAVVGLAALGGALRRLGANVDAGAGMEAALAVISNQ

SEQ ID NO: 33 - protein, 394 aa, Rhizobium sp. AC44/96
MNQTKIADPIFTLTTGPVDAYPAVLRALSRPVLYDYDPAFLAFYEAVNAKVQRAFHTKTPPVILQGEPVLGLEAAAASLIAKKDVVLNLVSGVYGKGFGFWAARYARELVELEVPYNDVLTADAVADMLKKRPDIAVVALCHHDTPSGTVNPVAEIGAVVRAAGKLFIVDSVSAFGGMDVLPETACADIFVTGPNKCLGCPPGLSLLHVSEAAWEKIAANPDAPTASMLSISDWKRAHEAGQPFPFTPSVSEINALDAAMDLYLEEGEQAVWARHALTAKACRAGVQAAGLKLWAAREDIASPTCTAVAIPDGIDEAKLRASIRDRYGVVFSSGRAETLGKLTRIGHMGPTARPTYSLVSVAAIVGGLKAAGVKDLDVEAGVAAAMKVIDSAEK

SEQ ID NO: 34 - protein, 390 aa, Erwinia toletana
MMRSAFAQPLFTLTTGPVDVYPAVSRALSMPVWYDYDPAFQNCFERVSLKAAQALESATPPLILQGEPVLALEAAAASLLSAEDMVLNLVSGVYAAGFSEWARRYSRQVEELRVEFNQVIDPVAVDAWLSRHPQITVVVVCHHDTPSGTLNPLDEIGRVVKKHGKLLLVDAVSSFAGVAVTAASCQADLFITGPNKCLGCPPGLSLVAVSDAAWRKIEANDAAPRASVLSLLDWRDAWSAEKPFPYTPSVAEINGLEAALDGYLQEGPAQVWARHRLTAQAFRAGIQAMGLSLWAADESIASPTATAIRVPDGVNEAQWRLRARECYGVMFSSGRGETLGKVLRVGHMGPTAQPMFAIIALTALAGALNQLREPQLDIGAGVAAAMAVIG

SEQ ID NO: 35 - protein, 386 aa, Herbiconiux ginsengi
MAHPDFTLSAGPVTVTARTLAGLGSPILYHYDPEFLATFRRTQGKVGQMFQTDNDIILMQGEAIVGLEGALRSLITPGMHVLNLVQGVFGKGTGYWIADFGAVLHEIEVGYDDAVSPAQVEEYLDAHPEIQMVCLVASETPSGTVTDVAAIGPMCRDRGILTYIDTVSGVLGMPWKTDEWGLDLCVAGAQKCLGGPPGVALISVSQKAWDVMYANPAAPRDSYLSLIDWKEKWLGEGRFPYTPSVSDMNGLEAALDQALEEGIDAVVARHEAAAAVTRAGARAMGLELWAKSEEIASACVTSIRLPDDIDNAVVRDHAREVYGVMLSHGQGAGNLVRLSHMGPTAGGLHSVVGLAALGRTLADLGMSVDIGAGLEAALALLSTQRR

SEQ ID NO: 36 - protein, 371 aa, Shewanella sp. AC10
MIIGVPTEIKNHEYRVGMVPSSVRELTIKGHVVYVQSDAGVGIGFTDQDYIDAGASILATAAEVFAKSDMIVKVKEPQAVERAMLRHDQILFTYLHLAPDLPQTEELITSGAVCIAYETVTDDRGGLPLLAPMSEVAGRMSIQAGARALEKSLGGRGMLLGGVPGVEPAKVVIIGGGMVGTNAAQMAVGMGADVVVLDRSIDALRRLNVQFGSAVKAIYSTADAIERHVLEADLVIGGVLVPGAAAPKLITRDMVKRMKPGSAIVDVAIDQGGCVETSHATTHQDPTYIVDDVVHYCVANMPGAVARTSTFALNNATLPYIIKLANQGYKQALLNDKHLLNGLNVMHGKVVCKEVAEALNLEFTEPKSLLA

SEQ ID NO: 37 - protein, 371 aa, Aeromonas hydrophila
MIIGVPKEIKNHEYRVGMTPAGVQELTARGHKVLVQQQGGEAIGLSDAEYQAAGAELVASAAEIFARAEMIVKVKEPQPEECELLRPGQLLFTYLHLAPDPVQTQLLVHSGAVAIAYETVTDDRGGLPLLAPMSEVAGRMAIQAGAHSLEKAQGGNGTLLGGVPAVAPAKVVVLGGGVVGVNSARMALGLGADVTILDRSLPRLRELDSLYGPALKTLYSTRTNIEACIAQADLVIGAVLIPGAAAPKLLTRDMLKLMRKGSVLVDVAIDQGGCFETSHPTTHEEPTYVVDGIIHYCVANMPGGVARTSTFALTNATLPFVLALADKGYVRALQEDRHLRAGLNVFKGKITQAAVAQALGYEYVSAEVVLD

SEQ ID NO: 38 - protein, 372 aa, Rhizobium sp. LPU83
MRVGCPKEIKNHEYRVGLTPASVREYVAHGHEVRVETKAGAGIGADDAAYVAAGAKIAASAKEIFEKCDMIVKVKEPQPSEWAQLRDGQILYTYLHLAPDPEQTKGLLASGVTAVAYETVTDDRGGLPLLAPMSEVAGRLSIQAGATALQKANGGLGILLGGVPGVLPAKVTVIGGGVVGLHAARMAAGLGADVSILDKSLPRLRQLDDIFGGRVHTRYSSIQALEEEVFSADLVIGAVLIPGAAAPKLVTREMLSGMKRGSVIVDVAIDQGGCFETSHATTHSEPTYVVDDVVHYCVANMPGAVPVTSAHALNNATLVYGLALADRGLRAIAEDRHLRNGLNVHKGRVTNKPVAEALGYESYTPESVLNVA

SEQ ID NO: 39 - protein, 373 aa, Pseudomonas mendocina
MRIGVPKEIKNHEYRVGLTPQSVAELTTLGHEVWIETLAGAAIGFADEDYRKAGAQIAPCAGEVFQQAQLIVKVKEPLAVERAKLREQHTLFTYLHLAPDRPQTDELMASGATCIAYETVTDVQGRLPLLAPMSEVAGRMSIQAGAGCLEKARGGRGVLLGGVPGVAPGKVVILGGGVVGSHALAMAVGLGADVTVLDKSVDALRRLDAQYGNRITTLYSTRAAVQEQVLAADLVIGGVLIPGAAAPKLISADMVRQMKAGAVLVDVAIDQGGCAETSRATTHAEPTYVVDDVVHYCVANMPGAVARTSTLALNNATLPFVVALAQKGTRRALEDDPHLLAGLNVARGAITCASVAEAHGLPFQPPASVLERL

SEQ ID NO: 40 - protein, 371 aa, Bradyrhizobium japonicum
MRVGVPKEIKMQEYRVGLTPGAVREYVAAGHQVTVETGAGSGIGASDEVYQRAGAAIAENARDIFDSSDMIVKVKEPQKSEWAQLRESQILFTYLHLAPDPEQAKGLLTSGCTAVAYETVTDAAGHLPLLAPMSEVAGRLAIEAAGAALKRSAGGRGVLLGGVPGVQPARVVVLGGGVVGTQAARMAAGLGAEVTVIDRSVPRLRELDDVFLGRVRTRFSTIEAVEDEVFAADVVIGAVLVPGASAPKLVTRGMLKSMRPGAVLVDVAIDQGGCFETSHPTTHADPTYQVDGVVHYCVANMPGAVPVTSSQGLNNATLPFGLMLANKGFAAVLENPHLRNGLNVHRGRITNKAVAESLGLEFTPVESGLAA

SEQ ID NO: 41 - protein, 370 aa, Bradyrhizobium japonicum
MKVGVPKEIKAHEYRVGLTPGAAREYVAAGHRVMIETNAGAGIGATDGDYRNAGATILTSAAEVFASSEMIVKVKEPQPAEWSQLREDQILFTYLHLAPDPEQAAGLLKSGCIAIAYETVTDAHGGLPLLAPMSEVAGRLSIEAAGSALKRSTGGRGLLIGGVPGVQPARIVVIGGGVVGTHAARMAAGLGAEVTIIDRSIIRLRELDELFEGRVRTRFSTIESVEEEVFAADVVIGAVLVPGASAPKLVRRSMLSSMRKRAVLVDVAIDQGGCFETSRPTTHADPTYEVDGIIHYCVANMPGAVPLTSSQALNNATLPFGLALANKGFSAVLENPHLRAGLNVHRGRLTYKAVAESLGLPFSPIEQAAA

SEQ ID NO: 42 - protein, 371 aa, Streptomyces aureofaciens
MKVGIPREVKNNEFRVAITPAGVHELVRHGHQVVIEHGAGVGSSIPDEEYVAAGARILDTADEVWATADLLLKVKEPIAQEYHRLRKDQTLFTYLHLAASKECTDALVESGTTAIAYETVELPGRALPLLAPMSEVAGRIAPQVGAYHLMAPNGGRGVLPGGVPGVTPAKAVVIGGGVSGWNATQIAVGMGFDVTLLDRDINKLREADRIFGTKVKAVMSNSFELEKAVVDADLVIGAVLIPGAKAPKLVTNELVSRMKPGSVLVDIAIDQGGCFEDSHPTTHAEPTFRVHNSVFYCVANMPGAVPNTSTNALTNATLPYIVELADRGWAEALRRDPALARGLNTHDGKVVYREVAEAHGLEYVDPATLLA

SEQ ID NO: 43 - protein, 363 aa, Anabaena cylindrica
MEIGVPKETKDQEFRVGLSPSSVRVLRENGHSIFVQTQAGTGAGFTDEDYISAGAEIVSTLEAAWNRELVIKVKEPLVSEYNLLQKGQLLFTYLHLAADRKLTEHLLDCGISAIAYETVEQAGVNRLPLLTPMSIIAGRLAVQFGARFLERQQGGKGVLLGGVPGVKAGKVVILGGGVVGTEAAKIAVGMGAIVQILDVNVERLSYLETLFGSRVELLYSNSAHIETAVKEANLLIGAVLIPGRRAPILVSRDLVKQMHPGSVIVDVAVDQGGCVETLHPTSHTSPVYIDEGVVHYGVPNMPGAVPWTSTQALNNSTLPYAVQLANLGIKALDVNPALAKGLNVQNHRLIHPAVQEVFPDLVS

SEQ ID NO: 44 - protein, 378 aa, Bacillus subtilis
MIIGVPKEIKNNENRVALTPGGVSQLISNGHRVLVETGAGLGSGFENEAYESAGAEIIADPKQVWDAEMVMKVKEPLPEEYVYFRKGLVLFTYLHLAAEPELAQALKDKGVTAIAYETVSEGRTLPLLTPMSEVAGRMAAQIGAQFLEKPKGGKGILLAGVPGVSRGKVTIIGGGVVGTNAAKMAVGLGADVTIIDLNADRLRQLDDIFGHQIKTLISNPVNIADAVAEADLLICAVLIPGAKAPTLVTEEMVKQMKPGSVIVDVAIDQGGIVETVDHITTHDQPTYEKHGVVHYAVANMPGAVPRTSTIALTNVTVPYALQIANKGAVKALADNTALRAGLNTANGHVTYEAVARDLGYEYVPAEKALQDESSVAGA

SEQ ID NO: 45 - protein, 371 aa, Artificial Sequence
  Modified Alanine Dehydrogenase
MIIGVPKEIKNHEYRVGMTPAGVQELTARGHKVLVQQQGGEAIGLSDAEYQAAGAELVASAAEIFARAEMIVKVKEPQPEECELLRPGQLLFTYLHLAPDPVQTQLLVHSGAVAIAYETVTDDRGGLPLLAPMSEVAGRMAIQAGAHSLEKAQGGNGTLLGGVPAVAPAKVVVLGGGVVGVNSARMALGLGADVTILARSLPRLRELDSLYGPALKTLYSTRTNIEACIAQADLVIGAVLIPGAAAPKLLTRDMLKLMRKGSVLVDVAIDQGGCFETSHPTTHEEPTYVVDGIIHYCVANMPGGVARTSTFALTNATLPFVLALADKGYVRALQEDRHLRAGLNVFKGKITQAAVAQALGYEYVSAEVVLD

SEQ ID NO: 46 - protein, 372 aa, Artificial  Sequence
  Modified Alanine Dehydrogenase
MRVGCPKEIKNHEYRVGLTPASVREYVAHGHEVRVETKAGAGIGADDAAYVAAGAKIAASAKEIFEKCDMIVKVKEPQPSEWAQLRDGQILYTYLHLAPDPEQTKGLLASGVTAVAYETVTDDRGGLPLLAPMSEVAGRLSIQAGATALQKANGGLGILLGGVPGVLPAKVTVIGGGVVGLHAARMAAGLGADVSILAKSLPRLRQLDDIFGGRVHTRYSSIQALEEEVFSADLVIGAVLIPGAAAPKLVTREMLSGMKRGSVIVDVAIDQGGCFETSHATTHSEPTYVVDDVVHYCVANMPGAVPVTSAHALNNATLVYGLALADRGLRAIAEDRHLRNGLNVHKGRVTNKPVAEALGYESYTPESVLNVA

SEQ ID NO: 47 - protein, 373 aa, Artificial Sequence
  Modified Alanine Dehydrogenase
MRIGVPKEIKNHEYRVGLTPQSVAELTTLGHEVWIETLAGAAIGFADEDYRKAGAQIAPCAGEVFQQAQLIVKVKEPLAVERAKLREQHTLFTYLHLAPDRPQTDELMASGATCIAYETVTDVQGRLPLLAPMSEVAGRMSIQAGAGCLEKARGGRGVLLGGVPGVAPGKVVILGGGVVGSHALAMAVGLGADVTVLAKSVDALRRLDAQYGNRITTLYSTRAAVQEQVLAADLVIGGVLIPGAAAPKLISADMVRQMKAGAVLVDVAIDQGGCAETSRATTHAEPTYVVDDVVHYCVANMPGAVARTSTLALNNATLPFVVALAQKGTRRALEDDPHLLAGLNVARGAITCASVAEAHGLPFQPPASVLERL

SEQ ID NO: 48 - protein, 371 aa, Artificial Sequence
  Modified Alanine Dehydrogenase
MRVGVPKEIKMQEYRVGLTPGAVREYVAAGHQVTVETGAGSGIGASDEVYQRAGAAIAENARDIFDSSDMIVKVKEPQKSEWAQLRESQILFTYLHLAPDPEQAKGLLTSGCTAVAYETVTDAAGHLPLLAPMSEVAGRLAIEAAGAALKRSAGGRGVLLGGVPGVQPARVVVLGGGVVGTQAARMAAGLGAEVTVIARSVPRLRELDDVFLGRVRTRFSTIEAVEDEVFAADVVIGAVLVPGASAPKLVTRGMLKSMRPGAVLVDVAIDQGGCFETSHPTTHADPTYQVDGVVHYCVANMPGAVPVTSSQGLNNATLPFGLMLANKGFAAVLENPHLRNGLNVHRGRITNKAVAESLGLEFTPVESGLAA

SEQ ID NO: 49 - protein, 370 aa, Artificial Sequence
  Modified Alanine Dehydrogenase
MKVGVPKEIKAHEYRVGLTPGAAREYVAAGHRVMIETNAGAGIGATDGDYRNAGATILTSAAEVFASSEMIVKVKEPQPAEWSQLREDQILFTYLHLAPDPEQAAGLLKSGCIAIAYETVTDAHGGLPLLAPMSEVAGRLSIEAAGSALKRSTGGRGLLIGGVPGVQPARIVVIGGGVVGTHAARMAAGLGAEVTIIARSIIRLRELDELFEGRVRTRFSTIESVEEEVFAADVVIGAVLVPGASAPKLVRRSMLSSMRKRAVLVDVAIDQGGCFETSRPTTHADPTYEVDGIIHYCVANMPGAVPLTSSQALNNATLPFGLALANKGFSAVLENPHLRAGLNVHRGRLTYKAVAESLGLPFSPIEQAAA

SEQ ID NO: 50 - protein, 371 aa, Artificial Sequence
  Modified Alanine Dehydrogenase
MKVGIPREVKNNEFRVAITPAGVHELVRHGHQVVIEHGAGVGSSIPDEEYVAAGARILDTADEVWATADLLLKVKEPIAQEYHRLRKDQTLFTYLHLAASKECTDALVESGTTAIAYETVELPGRALPLLAPMSEVAGRIAPQVGAYHLMAPNGGRGVLPGGVPGVTPAKAVVIGGGVSGWNATQIAVGMGFDVTLLARDINKLREADRIFGTKVKAVMSNSFELEKAVVDADLVIGAVLIPGAKAPKLVTNELVSRMKPGSVLVDIAIDQGGCFEDSHPTTHAEPTFRVHNSVFYCVANMPGAVPNTSTNALTNATLPYIVELADRGWAEALRRDPALARGLNTHDGKVVYREVAEAHGLEYVDPATLLA

SEQ ID NO: 51 - protein, 363 aa, Artificial Sequence
  Modified Alanine Dehydrogenase
MEIGVPKETKDQEFRVGLSPSSVRVLRENGHSIFVQTQAGTGAGFTDEDYISAGAEIVSTLEAAWNRELVIKVKEPLVSEYNLLQKGQLLFTYLHLAADRKLTEHLLDCGISAIAYETVEQAGVNRLPLLTPMSIIAGRLAVQFGARFLERQQGGKGVLLGGVPGVKAGKVVILGGGVVGTEAAKIAVGMGAIVQILAVNVERLSYLETLFGSRVELLYSNSAHIETAVKEANLLIGAVLIPGRRAPILVSRDLVKQMHPGSVIVDVAVDQGGCVETLHPTSHTSPVYIDEGVVHYGVPNMPGAVPWTSTQALNNSTLPYAVQLANLGIKALDVNPALAKGLNVQNHRLIHPAVQEVFPDLVS

SEQ ID NO: 52 - protein, 378 aa, Artificial Sequence
  Modified Alanine Dehydrogenase
MIIGVPKEIKNNENRVALTPGGVSQLISNGHRVLVETGAGLGSGFENEAYESAGAEIIADPKQVWDAEMVMKVKEPLPEEYVYFRKGLVLFTYLHLAAEPELAQALKDKGVTAIAYETVSEGRTLPLLTPMSEVAGRMAAQIGAQFLEKPKGGKGILLAGVPGVSRGKVTIIGGGVVGTNAAKMAVGLGADVTIIALNADRLRQLDDIFGHQIKTLISNPVNIADAVAEADLLICAVLIPGAKAPTLVTEEMVKQMKPGSVIVDVAIDQGGIVETVDHITTHDQPTYEKHGVVHYAVANMPGAVPRTSTIALTNVTVPYALQIANKGAVKALADNTALRAGLNTANGHVTYEAVARDLGYEYVPAEKALQDESSVAGA

SEQ ID NO: 53 - DNA, 1038 bp, Saccharomyces eubayanus
atgtccgtcactgaattgagaaacgatatccataagttagataccggttatggtctgatgagtttgacctggagagctgagcccattcctcaatcgcaagctttcgaagcgatgcacagagctgttgagttggccagagaacgtggacacaaggccttcttcaacgttggtgagttttacggcccggacttcgttaacttaatatttgttcgcgacttctttgcaaagtaccctgatttgagaaagcatgtagtcatcagttgtaaaggtggtatggaggttagcactctgacccccaatggtagccatgatgcagtcattaaaagtgtcaagaactcggtcagcgccattggtggctacattgacatctttgaggttgccaggcttgatacctctctatgtgccaaaggcgaagtctatccatacgaatcgttcgaagcccttgctgaaatgatctccgaaggcgttattggcggtatttctttgagtgaagttactgaagagcaaatcagagctatttacaaggactggggcaagttcttgacctgtgttgagctcgaagtttctttgttcagtaccgatattttccataacggaattgccaagacctgtgccgaattgggattgactgtcatctgctactctcccctaggtagagggctattgacaggccaattgaagtctaacgctgacatcccagaaggcgacttcagaaaggcacttaagaggtttagtgatgaatctttgaagaaaaacctggatttggtcaggttcctacaggaagaaatcgtcggcaagcgttccaaggacaactctattactctgcctcaattggctttgggctggatcaaacactggaactatgttccggaatacaaaggcgccaaattcatccccattcccagtggttcttctatctctaaggtcaatgaaaactttgatgaaaagaaaacacaacttaccgacaaagagttcaaagccattaacgacttcttggctactttccacactgttggcgataggtacgaattcaattga

SEQ ID NO: 54 - DNA, 1050 bp, Torulaspora delbrueckii
atggtcgaacaatctgcaatcaacaagctaaagcaagatctctctgcgattgacaccggatatgggctgatgtcgcttacgtggagggctgagcccattccagagacccaggctcacgaaactatgaaaagagtcgttgaattggctcaatctaaaggccataaggcatttttcaactgtggtgagttttatggacccaatcggatcaatctgacctacatcagggatttttttgtcaagaatccagaattgaggaagtatgtgatcatcagttgtaagggtggttgcgactgtgaaacccttactcccaagggaaaacatgacgatgttatcaaaagtgtggaggcatcagttgcagccattggtggttttattgacatctttgaagtcgctagattggatttgagtctatgcactaatggcgaaacatacccttatgagtcctttgaagcattggccgagatggttgacaatggtgccatcggtgccattagtttgagtgaggtcactgctgagcagatccgtggaatttccaaggactggcacaagtatttggtcagtgtggaggttgaactatcgatgttcaccaaagcgattttgaccaatggtgtcgttaaagcaaataatgacctaaacttggtcactattttgtactctccattgggtagaggattgctgactggtgcagttactagtagtaaagatataccagctggtgatttcagaaacctactgaagagatttgatgatgaaagtttgagacaaaacctgacattacttgacttcttaagggacgaaatcatcgctaagagacctgcaaataacgagattacactaccacaagtagcccttggttgggacaagtattggaacaagacaggcgagtaccccaacactcactttttaccaatcccaagtggttccagtgtaaaaaaactcgaggagaactttgatgaagccaagactcaaatcaccgctgaggagtttagaaaaatcaatgatttcttgaagaacttcaagactgtaggtgacgcttacgagatgagatga

SEQ ID NO: 55 - DNA, 1026 bp, Zygosaccharomyces bailii
atgtcaaagacgggtgaattaaaaagacaattgaaccaattgcataccggttatgggttgatgagtttgacttggagggctgatccagttccccaagaacaagcctttgaagccatggagcgagtagtcagtcttgcgcaggctagcggcaacaaggctttcttcaatgttggtgagttctacggtcccgactattccaatttggagcttgtaaaagcattcttcgaggccaagccagagttacgtgagcacgttctgatcagttgtaagggtggcgtcgataataccaagcttttgcctaagggaaaatacgcagacgttctttcgagtattgagcaatgcgtgaaacatttgggcacgcacttagacatttttgaagtggcccggttggacaagtctcttggtggcgaatatcctcgtgaatcatttgaggccatggcatcgatggtagacaaaggtatcatcgatggtatatctctaagcgaagtaacagcggatgagattcgagcaataggacatgattgggcgaaataccttgtctgtgtagaagtcgaattttcaatgttcagccctcagatcctgcacaatggggtattggatgcatgcaatgacctgggattaatcgtcattgcctattctcccttgggcagaggcttacttaccggtaccatcacgaaggattcagcctttaaagatttccgttccagtttgaagcgtttccagaaagattccctgcaaaagaacttggcgttgaccgaatttctgcaggatcagatcgtggaccaaagaagttcaacaaacccgatttccttgccacaggtggcacttggctgggtcaaaagtctgaataatgggaagtatccccacactcacattgttcccatccccagcggatctgacaagagaaaagtggaggagaactttgacgaatccagggctaagctcaccgtttctgagattgaaaaaatcaacgatttcttgaagtcattcaacgttgtcggcgacagatacgagtgggtttcttag

SEQ ID NO: 56 - DNA, 963 bp, Aspergillus oryzae
atgccttctcttgttggacgggatgttggccatactggctatgggttgatgagaatgacctgggtgcctcaaccacccccgcaagaacaatgcttcgaggctctgaacaccgccctcgcccatggctccaatttctggaacgccggtgaactctacggcacacccgaatataactctttgcatctgctccatagttacttcgcccagcacccagagaatgcggacaaggttgttcttagcattaagggtgggttgaagccgggccagctggtgcctgatggttccgaggccaatattcggcgcagtgtggatgagtgtcttcgtgtgctggatgggaagaagaagattgatatctttgagtgtgcgagacaggatcccaagactactgtagagcagacggttactgttcttgcgcagttggttaaggaggggaagattgggggcattgggttgagtgaggttgatgcggagactattcggagagcacacaaggtgcacccaattgcagctgtagaggttgagatgtctctgttcgacctgaccatcctccagaacgacgtcgcgaaagtatgcgcagaactcaacatccccattgtggcatattcgccattgggacggggtgtcttggccggcgctttcaccacagccgccgacatccccgatggagatttccgcaagacgttgcccaagttccaagatgaggcaatgaagcagaacatcaagctagtcaatgaggtcaatgacctcgccgctcggaagggcgttgctcccgttcagatcgcactcgcatgggtcctaactcttagcggcaagcctggcatgcctacgattattcctatccctggtggtaccacgagcgccaaggtggcgcagaacttgcaagcgcctcgtctgactgatgcagagatggcagagattgatgcaattttgaagcggaatgagattgttgggactcgatactga

SEQ ID NO: 57 - DNA, 1032 bp, Kluyveromyces marxianus
atgccttccccatcagattatcgtaaggaattgcttgacgtcgaagttggttatggattgatgagtttgacatggaggaaagatccacttcctgctgaaaaggtgtttcctaccatgaagaaggtggtagaaaaggcgggcaataagaaggcattgttcaatgtgggtgaattctatggtcctcatttggctaattacaagttgctacagtcgttcttcgataagtaccctgaggatcgcaagaaagttattatcagtgctaagggaggtgtgaatgtcgaaacattacaacccaccggagatgccgacagcatttctgcttcaattgagaatgcattgaaggtgtttggtggatatctagacatttacgagccagctagaattgatcttgcactagcggagaagaacggtgagaagttgtttcctcgtgagacttttgacactatcgtcaagtacatcaaggatggtaaagttggcggtttctcgttaagtgaagttacaaacgagcaaatcagagctatttacaaggaatacggcgagtatctggcttgtgttgaagttgaactctctatagcttcgccagaaattatatccaacgggattttggacacatgcaatgaatttggaattcctgtcgttgcatatgccccactaggaagaggtttactaacgggtagcttgaaaagcactgctgatatccctgaaggtgatttcaggggccaattgaaaaggttccaggatgatgccattgcacataacctgactttggtaaaattccttcaagacgaaattgctgcaaagagatccgacaacccttctctccctcaagtggccattggctgggtccgtggactcagaagggagtatccaaaaacaaatattataccaattccatcagggtcttcggttgaaaaggtaagtgtcaactttgatcacattgatttatcagacgatgagatgagcaagataaaagactttttaaagtcatttaaaatcgctggtgatagatatgagttcgcgcaagcacattaa

SEQ ID NO: 58 - DNA, 1050 bp, Candida albicans
atgtcttttaaacctgtcgaaatatcgggaaagtttggatttgggacaatgtccatgacttggacaccaacgccaccaccagcacaacaatccattgataccttgaaatttgtgacatcacatccaaaatttggaactaaattgatcaatggtggtgaattttatggtccagattttgctaatttgaaattattaaaacaatttttagaagaaaatgatcctgaagaaaataaacaattgattatatctattaaaggaggtgctgataatgaaacattaaaaccaaatggtaccaaagagtttgtttcaaaatcaattgaaaacattgtatcatttttccctaaacaaaaacaaaacagaccaaaattattatttgagatggccagagttgatccttctgttccctatggagaaactattggttatatcagtgaatatgtgaaactgggtgtgattgatggtatttctttatctgaagttggtaaagaatccattcaggctgctttgaaagtatttccaatatcatgtgttgaattagaattgtcattgttttcacaagaagttattaccactggtatattggaagaattgtcaaagcacaacttgccactcattgcttattcaccattgtgtcgtggtcttttaacagattatgcagttgagaattcagatacatttttggcatcaatcccacaaggtgatattagacatcatttggacaaatttcaaccagacacttttaacaaaaacttgcccgctttaaaggaattatacaagtttgctcatgaagttaaaaacacaactttggaatctttggcattatcatggattgttacagtttcggaagcaagaaatttccgaggcattgaaaaagtgaccagaattttaccaattccttctggatcaactaaaaagagagttgagtcaaattttggaagtttaatagagttgactgatgatgatttacaatcaatcgatgaaatttttgccaaatatccaattagtgggttgagatacaatcaatttctcgaaggaactcttttccaatag

SEQ ID NO: 59 - DNA, 990 bp, Yarrowia lipolytica
atgacattcaacaaaatcacacccacttccaagggtttcggactcatgggcttcacatggagagaaaccgagacccccgatgagattgccttccccaccatgaagcgagccattgaaaagggcattcttgtgtggaatagcgccgagttctatggtaccaaggacccactaggcaacctcaagttgattcgacgatactttgagaagtaccccgaagacgccgacaaggtgtgtctgatggtcaagggaggtctgggacccaacttggtgcccaatggctccaaggagtttgtgcagaagtccattgacactgttctggagactctgggaggctctgttcctgtgagcatcttccagtgtgcccgtgtggaccccaatacccccattgaggagacggtcgctgccatcaaggagtatgttgatgctggcaaaattggagctctgggtttatctgaggtcggggctgccaccattgagaaggtccactctctcagtcccattgctgctgttgagaatgagctatctctgtgggccaccgatatcttcaccaacggtgttgctgaagtctgtgccaagtacaacattcccatcactgcctatgctcctctgggctctggcgtgcttactggccggttcaagagcacccaggatgtgcctgagggagacgcccgaaagcacatggtcaagtaccaggaggaaaacttgaaacagaatctcgttctcgttgaccgacttaaggaggtggccaccaaggccggtattcctcttcctcagatggccattgcatgggtcaaggctcaatctaaccacaatggccatggaaccatcatccctatcccaggtaacaccactgttgctcggctcgaggagaaccacaatgatctcactctgtcggccgagactctcaaggagattgatgatgttctgaagagtactaagattgttggagatcgaggatttactcttggtggagagtttatgaatggttaa

SEQ ID NO: 60 - DNA, 1179 bp, Mesorhizobium sp. YR577
atgcgatatcccgaccacactgagccggtgatcacactcaccgccggtcccgtcaacgcctatcccgatgtgctacgggggctcgggcgcacggtgctttacgactacgacccggcgttccagctcttctacgagaaggtcatcggcaaggcgcagaaggccatgcgtctgtcgaataagccggtcgtcctgcatggcgagccggtgatgggtctggaggccgccgcggcgtcgctcatctctagggatgatgtggtgctcaatctggcatcgggtgtctacggcaaggggttcggctattgggcgaagcgttattcgccgaacctgctggagatcgaggttccttacaatgaggcgatcgatccgcagtccgtcgcggacatgctgaaatcgcatcccgaaatcaccgtcgtttcggtctgccatcacgacacgccgtcaggcacgatcaacccgatcgatgagatcggagcgatcgtttcggcgcatggcggctatctgatcgtcgacgccgtttcgtccttcggcggcatgaaaacgcatcccgaggattgcaaggccgacatctatgtgaccgggccgaacaagtgccttggcgcacctcccggactgacgctgatgagcgtgagcgaccgtgcctggaccaggatgaaagcgaactctgcagcacctcgcgcatcggttctgagcattctcgattgggagcatgcatggtccaaggaaaagccgtttccgtttacgccgtccgtcgccgaaatgaacggcctggatgtcgcgctcgacctttatctcaacgaggggccggaagcggtgtgggcgcggcacgccctgaccgccaaggcgacacgcgccggggctcaggcgatgggcctgtcgatctgggctgcaaatgaaaagatcgcctcaccgacaacaaccgccatccgcacgccggacggggtcgatgaaaaggcgctgcgtcaggccgcgcgtgagcgttacggcgtggtgttttcttccggccggggcgaaacgctcggcaaactgacgcgcattggccacatggggccaacggcccagccgatctacgcgatcgcagcgctgacggctttgggcggtgcgctgaatgcggccggccggaagcttcaggttggcaagggtatcgaggcagcactcgccgtgatcgatgcggatgcctga

SEQ ID NO: 61 - DNA, 1179 bp, Pseudaminobacter salicylatoxidans
atgcgatataaagacaatgccgacccggtgatcacgctcacctcggggcccgtcaacgcctatgcggatgtgctgcgcgggctgagccgcactgtgctctacgactatgatccggcgttccagctcttctatgaaaacgtggtggccaaggcgcagaaggccatgcgtctttccacccggccggtgctgttgcagggtgagccggttatgggactggaggcagcggcagcttcactgatttccagcgaggatgtcgtcctgaacctcgcctctggcgtctatggcaagggcttcggctactgggcaaagcgctattcgccgaacctgctcgagatcgaaaccccctataacgaatccatcgacccgcaggcggttgccgatatgcttaagaagcatccggaaatcaccgttgtctcggtgtgccaccacgacacaccgtcgggcacgatcaacccgatcgacgaaattggcgcgatcgtatcggcgcacggggcctatttcatcgtcgatgccgtatcttcattcggaggcatgaagacccatcccgaagactgcaaggccgacctctatgtcactggcccgaacaagtgcctgggcgcgcctccgggactgaccatgatgggcgtcagcgacagggcctgggccaggatgaaagccaatccatccgcaccgcgcgcctcggtgctgagcattctcgactgggaggatgcctggtcgcacgaaaagccgtttccgttcacaccctctgtggcggagatcaacggcctcgacgtcgcgctggacctctatctgaacgaggggccggaagtggtgtgggcgcgccatgcgctgacggcaaaagccacgcgtgcgggcgcattggccatggggttgtcgctctgggcggctagcgagaagattgcctcgcccaccacgacgtcgatccgcacgccgcagggggtggacgaagccgcgcttcggcaggcggtgcgagagcgctatggcgtagtgttttcatccggccgtggcgagacgttgggcaagcttacccgcatcggccacatgggccccacggcgcagccgatttacgcagtcgcggcgctgaccgcgctgggtggcgcgttgaacgcgatgggccagaagctggccgtcggcagaggcatcgacgccgccctcgcggtgatcgacgccgatgcatga

SEQ ID NO: 62 - DNA, 1179 bp, Bauldia litoralis
atgcgctatgcactcgatgccgatcccgtcatcacgctgacctcggggccggtcaacgcctatccggatgtcctgcgggcgttggcgcgcacggttctttacgactacgacccggccttccagctccattacgagcaggtgatcggcaagatgcagacggcgatgcggacgtcgaaccgtccggtcatcctgcatggcgagccggtgctcggactcgaggcggcggcggcctcgctgatcaccgccgacgatactgtgctcaacctggcgtcgggcgtctacggcaaggggttcggctactgggcagcgcgctattcacccaaccagctcgagatcgaggttccctacaaccaggcgatcgacccgcagcaggtcgccgacatgctgaaggccaatccggccatttcggttgtcgccgtctgccatcacgacacgccgtcggggaccatcaacccgatcgacgagataggcgccatcgtcgccgaccatggcgcctacctgatcgtcgacgcggtgtcctccttcggcgggatgaagacccacccggaggattgcaaggccgacatctacgtcaccggcccgaacaaatgcctcggcgcgccgccggccctgaccatgctcggcgtcagcgaacgggcgtggcagaagatgaaggacaatccggccgcgccgcgcgcctcgatgctcagcatcctcgactgggagaatgcctggtcgcgcgacgaggcctttcccttcacgccgtcggtggccgaggtcaacggtctcgacgtcgcgctcgacctctaccttgccgagggaccggaggccgtctgggcgcgccacgcgctgaccgccgcggcgacgcgggccggcatcctggcgatgggcctcgacctgtgggcggcggacgcggccatcgcctcgccgacgacgaccgcggtgcggacgccggagggcatcgatgaagcgaagctgcgcgcgacggtccgcgaacgctatggcgtggtgatgtcgtccgggcgcggcgaaacgctcggcaagctcacgcggatcggccacatgggcccgacggcgcagccgatctatgcgatcgccgcgctgaccgcctttggcggcgctctccaggcgctcggccatcccgcgccggtcggcaaggcgatcgacgcggcgctcgcggtcatcgacgccgccgcctga

SEQ ID NO: 63 - DNA, 1176 bp, Skermanella stibiiresistens
atgacagtcgagcgtatcgtcgagcctgctttcactctcagttccggtcccgtcgacgcgtatcccgccgtgctcagggcactgtcgcggacggtgctctacgacttcgatcccgtgttccagcacttctacgaggcggtgacggataaggccaaggtggcactccgcaccgcggcgccacccgtgatcctccatggcgagccggtgctggggctggaggccgcggccgcctcgctgatcgcccgcgacgatgtcgtgctcaacctagcgtccggcgtctatggcaagggcttcggtttctgggccaaacggtactgcgccgagttggtcgagatcgaggtgccttatgacgacgcgatcgacccggcggcggtcgaggccgccttcagggaacgccccgacatccgtgtggtctcggtctgccatcatgacacgccgtctggcacgctgaacccaatcgacgagatcggccgtatcgtggcggcccatggtgcctatctgatcgtggacgccgtttcctcgttcggggggatggacgtccatcccgaggcctgccaagccgacatcttcgtgacagggccgaacaagtgcctgggctgcccgcctggtctgacgctgctggcggtcagtggccgcgcctgggacaagatgaaggccaatccggacgcgccgcgcgcctcgatcctcagcatcctggattgggagaacgcgtggcggcgtgaccagccgtttcccttcaccccatcgatttccgaagtgaacgcgctggacgcggctctggacctgtacctggaggaggggccggaagccgtctggcgccgtcacgcgctcacatccaaggcctgccgcgacggcatccgcgccatgggcatgacgatatggcccgcgcgggaggcgatcgcctctccgaccaccacggcggtgcgggtgcccgacgggatcgacgcggatgccctgttggcgacggcgcgggatggcttcggcgtcagcttctcgtccgggcggggcgagaccaagggcaagctgatccgcatcggacacatgggccgcaccgcccaaccactctacgccgtcgtcgggctggcggcgctgggcggtgcgttgcggcggctgggagccaatgtcgacgccggcgccggcatggaggcggctctcgccgtgatctcaaaccaataa

SEQ ID NO: 64 - DNA, 1185 bp, Rhizobium sp. AC44/96
atgaaccagacgaaaattgccgatccaatctttaccttgaccacggggccggtcgatgcctatccagcggtgctgcgcgccctgtcccgcccggtgctctacgactacgatccggcgttccttgcgttctacgaagccgtcaatgccaaggtacagcgcgccttccacacgaaaaccccacccgtgatcctccagggcgagcccgtgctggggctcgaagcggctgcggcttcgctgatcgccaagaaagacgtggtgcttaacctcgtttccggcgtctacggcaaggggttcggcttttgggcagcgcgctatgcgcgggagctggtcgaattggaagttccctataacgatgtcctgacggccgatgcggtggccgacatgttgaaaaagcgtccggatatcgccgtggtcgcgctctgccaccatgataccccgtcaggcaccgtcaatcccgtggccgaaatcggtgcagtggtgagagcggcgggaaagctcttcatcgtcgattccgtatcggcctttggcggcatggacgtgcttccggagacggcttgcgccgatattttcgtaaccggccccaataaatgtctcggctgcccgcccggtttgtcgcttctgcacgtcagcgaggctgcctgggagaagatcgccgccaatcccgatgccccgacagcttcgatgctttcgatcagtgattggaaacgagctcacgaggctggccaaccgttcccgtttaccccctcggtttccgaaatcaacgccctcgacgccgccatggatctctatctggaggaaggcgagcaggcagtatgggcgcgccatgcgctgacagccaaggcatgccgggcaggcgtgcaagcagccggcctgaagctctgggcggcacgggaagacatcgcctcgccaacctgcactgccgtggcaatccccgatgggatcgacgaagcaaagctgcgggcgtcgatccgtgatcgctacggcgttgtcttttcttctggccgtgccgagacgctgggcaagcttacgcgcatcgggcatatgggcccgactgcacgtccgacctattcactcgtctcggttgcagccatcgttggcgggctcaaggccgccggcgtcaaggaccttgatgtggaggctggagttgcggcggcaatgaaggtcatagacagtgcagaaaagtga

SEQ ID NO: 65 - DNA, 1173 bp, Erwinia toletana
atgatgcgttccgcctttgcccagccgttatttaccctgactaccggtccggttgatgtctatccggcggtgtcgcgggcattatcgatgccggtctggtacgactacgacccggcttttcagaactgctttgaacgcgtcagcctgaaggccgcacaggcgctggaaagcgccacgccgccgctgatcttacagggtgaaccggtactggcgctggaagccgcggcggcatcgctgcttagcgctgaggatatggtgctgaatctggtttccggcgtatatgccgccggtttctctgagtgggcgcggcgctattcgcgtcaggttgaggagctgcgcgttgaatttaatcaggtgattgatcctgtcgcggttgatgcatggcttagtcgtcatccgcagattaccgtggtggtggtttgccaccatgacacgccgtccggcaccctaaacccactggatgaaattgggcgggtggtgaaaaaacatggcaaattgctgctggtggatgcggtttcttcgtttgccggtgtggcggtgacggcggccagttgccaggcggacctgtttattaccggccccaataaatgtctcggctgtccgccgggcttaagcctggtggcggtatctgacgctgcctggcggaaaatcgaggccaatgacgctgcgccgcgtgcgtcggtactcagcctgctcgactggcgtgatgcctggtcagcggaaaaaccttttccgtataccccatcagtggcggagatcaacgggctggaggcggcgctggacggttatttgcaggaggggccagcgcaggtctgggcgcgccacaggctaaccgcacaggcatttcgcgccgggattcaggcgatgggacttagcctgtgggcggcggatgaaagcattgcttcaccaacggcaactgcgatacgcgtgcccgacggggtaaatgaagcgcaatggcgactgcgcgcgcgtgagtgttatggcgtgatgttttcgtcagggcgcggtgaaacgctgggcaaggtattgcgtgtcggccatatggggccgactgcgcaaccgatgtttgcgattattgcgctgaccgcactggcgggggcacttaatcagctacgcgagccgcagctggatattggcgctggcgtggcggcggcaatggcggtgattggctaa

SEQ ID NO: 66 - DNA, 1161 bp, Herbiconiux ginsengi
atggcacaccccgacttcacgctctcggccgggccggtcaccgtcaccgcccgcacgctcgcgggcctcggctcgccgatcctctaccactacgaccccgaattcctcgcgacgttccggcgcacgcagggcaaggtggggcagatgttccagaccgacaacgacatcatcctcatgcagggtgaggccatcgtcggtctcgagggcgccttgcgctcgctgatcacgcccgggatgcacgtgctgaacctcgtgcagggcgtgttcggcaagggcaccggctactggatcgccgacttcggcgccgtgctgcacgagatcgaggtcggctacgacgacgcggtctcgccggcacaggtcgaggagtacctcgacgcgcatccggagatccagatggtgtgcctggtggcctccgagaccccgtcgggcacggtgacggatgtcgcggcgatcggcccgatgtgccgggaccgcggcatcctgacctacatcgacactgtctcgggcgtgctcggcatgccctggaagaccgacgagtggggcctggacctctgcgtcgccggcgctcagaaatgcctcgggggccctcctggcgtggcgctcatctcggtgagccagaaggcgtgggacgtgatgtacgcgaacccggccgccccgcgtgactcctatctctcgctgatcgactggaaggagaagtggctcggtgagggccgctttccgtacacgccgtcggtgagcgacatgaacgggctcgaagcggcgctcgaccaggcgctggaggaggggatcgacgcggtcgtcgcgcgtcacgaggccgccgccgccgtgacccgcgccggcgcccgagccatgggtctcgagctctgggcgaagagcgaggagatcgcgtcggcctgcgtcacctccatccgcctgcccgacgacatcgacaacgccgtggtgcgcgaccacgcccgcgaggtctacggggtgatgctctcgcacgggcagggtgccggcaacctcgtgcggctctcgcacatgggcccgacagccggtggcctccactcggtggtgggcctcgccgcgctcggccgcaccctcgccgatctgggcatgtccgtcgacatcggagcgggactggaggcggcgctcgcgctgctcagtacccagcggcggtga

SEQ ID NO: 67 - DNA, 1116 bp, Artificial Sequence
  Modified Nucleotide Sequence for Alanine Dehydrogenase
atgattattggcgtacccaaggaaatcaaaaaccatgaataccgggtcggcatgactcccgccggagtgcaggagctgacggcccggggacacaaggtgctggtgcagcagcagggaggcgaggcgatcgggctgtccgacgccgagtaccaggcagccggtgccgaactggtggccagcgcggcggagatcttcgcccgggccgagatgatagtgaaggtgaaggagccccagccggaggagtgcgagctgctcagaccgggtcagctgctgttcacttacctgcacctggcgccggatccggtgcagacccagctgctggtgcactcgggggcggtggccatcgcctacgagacggtaacagacgatcgcggcggcctgccgctgctggcgcccatgtccgaggtggcggggcggatggcgatccaggccggcgcccacagcctggagaaggcgcaggggggcaacggcaccctgctgggcggggtgccggcagtggcgcccgccaaggtggtggtgctgggcggtggcgtggtcggcgtcaactcggcgcgcatggcgctgggactgggggccgacgtcaccatcctggctcgctccctgccgcgactgcgggaactggacagcctgtacggcccggccctcaagaccctctactccactcgcaccaacatagaggcgtgcatcgcccaggctgacctggtgatcggtgccgtgctgatcccgggggcggcggcgcccaagctgctgacccgcgacatgctcaagctgatgcgcaagggctcggtgctggtggacgtggccatcgatcagggcggctgtttcgagacctcccacccgaccacccacgaggagcccacctacgtggtggacggcatcatccactactgtgtggccaacatgccgggcggggtggcgcgcacctccaccttcgcgctgaccaacgccaccctgccgttcgtgctggcgctggccgacaagggctatgtcagggcgctgcaggaggatcgccacctgcgggccggtctgaacgtgttcaagggcaagatcacccaggcggcggtggcgcaggcgctcggctacgagtacgtcagcgccgaggtggttctggactga

SEQ ID NO: 68 - DNA, 1119 bp, Artificial Sequence
  Modified Nucleotide Sequence for Alanine Dehydrogenase
atgcgtgtcggttgcccgaaggaaatcaagaatcatgaatatcgtgtcggcctgacacctgcgtccgttcgtgagtacgttgcccatggccatgaggtccgggtcgaaaccaaggcaggcgccggcattggcgccgatgatgccgcctatgtggccgctggcgcaaagattgccgcctcggccaaggagatcttcgagaagtgcgacatgatcgtaaaggtcaaggagccgcagccctccgagtgggcgcagcttcgcgatggccagattctctatacctatcttcacctcgcgcccgatccggaacagaccaagggcctgcttgcctccggcgtcaccgctgttgcctatgagactgtgacggatgatcgcggcggcctgccgctccttgctccgatgtccgaggtcgccggacgtctgtcgatccaggcgggggcgaccgcattgcagaaggccaatggcggccttggcatcttgctcggaggcgtgcctggcgtgttgccggccaaggtcacggtgatcggcggcggtgtggtcggcttgcacgcggccaggatggcggcaggcctcggtgccgacgtcagcatcctcgcaaagtctctaccgcgcctgcgccagctcgatgatatcttcggcggtcgtgtccatacgcgctattccagcattcaggccttggaagaggaggtattctcggccgacctcgtcatcggcgctgtgctcattccgggcgctgccgcaccgaagcttgtcacccgtgaaatgctgtcgggcatgaagcgaggctctgtcatcgtcgacgtcgccatcgaccaaggtggctgctttgagacgtcgcatgcaacgacgcattccgagccgacctacgttgtcgatgacgtggtgcattattgcgttgccaacatgcctggcgctgtgccggtcacttcggcgcatgccctgaacaatgcgacgctcgtttatggcctggcgcttgcagaccgcggactgcgcgcgatcgcggaagatcgccacctgaggaacggcctcaacgtccataagggccgcgtcaccaacaagccggtggccgaagcgctgggctatgaatcctacactccggaaagcgtactgaacgttgcgtaa

SEQ ID NO: 69 - DNA, 1122 bp, Artificial Sequence
  Modified Nucleotide Sequence for Alanine Dehydrogenase
atgcgaatcggcgtacccaaggaaatcaagaatcacgagtaccgtgttggccttacgccgcagtccgtggccgaactgaccacgctggggcatgaggtgtggatcgagaccctggccggcgccgccatcggttttgccgatgaggattaccgtaaggccggggcgcagatcgctccgtgcgccggcgaggtgttccagcaggcgcagctgatcgtcaaggtcaaggagcctctggcggtggaacgggccaagctgcgcgagcagcatacgctgttcacctacctgcaccttgcgccggaccggccgcagaccgacgagctgatggccagcggcgccacctgtatcgcctacgagacggtgaccgatgtgcagggccgcctgccgttgttggcgccgatgtcggaagtggctgggcgcatgtcgatccaggccggtgccggttgcctggaaaaggcgcgtggcggtcgtggcgtactgctcggcggcgtgcctggggtggcgccgggcaaggtggtgatccttggcggtggtgtggttggcagtcatgccctggccatggccgtgggcctgggcgccgatgtcacggtgctggccaagagtgtcgatgccctgcgccggctcgatgcgcagtacggcaaccgcatcaccacgctctattccacacgcgccgcagtgcaggagcaggtgttggcggccgacttggtcatcggcggcgtgttgattcccggggcggcagcgcccaagttgatcagcgcggacatggtgcgccagatgaaggccggcgcggtgttggtggatgtggccatcgaccagggcggctgcgctgaaacctcgcgggccaccactcatgccgagcccacctacgtggtcgacgatgtggtgcattactgcgtggccaatatgcccggtgcggtggcccgtacctcgacgttggcgctgaacaacgccactttgccattcgtcgtggcgctggcgcagaagggcacgcggcgagcgctggaagacgatccgcacctgctggccggtctcaatgtcgcgcgcggcgccatcacctgcgccagtgtcgccgaggctcacgggctacccttccagccgccggccagcgtgctcgagcggctctag

SEQ ID NO: 70 - DNA, 1116 bp, Artificial Sequence
  Modified Nucleotide Sequence for Alanine Dehydrogenase
atgcgcgtcggtgtgcccaaggagatcaagatgcaggaatatcgcgtcgggctcaccccaggcgccgtccgcgaatatgtcgcggccgggcaccaggtgacggtcgagaccggcgccggcagcggcatcggcgcgtctgacgaggtgtaccagcgggcaggggccgcgatcgcggagaacgcccgcgacatcttcgacagctccgacatgatcgtgaaggtgaaggagccgcagaaaagcgaatgggcccagcttcgcgaaagtcagatcctctttacctacctccatcttgcgccggatcccgaacaggccaagggcctgctcacctcgggttgcactgcggtcgcctatgagaccgtcaccgacgcggccggtcacctccccctgcttgcgccgatgagcgaagtcgccggccgcctcgccatcgaagccgccggcgccgcgctcaagcgatcagccggcggccgcggggtgctgctcggcggtgtgcccggcgtgcagccggcgcgcgtcgtcgtgctcggcggcggcgtggtgggaacgcaggccgcgcgcatggcggcgggtctcggtgcggaagtcactgtgatcgcacgctcggttccccgtctgcgcgagctggacgatgtctttctcgggcgggtgcgcacccgcttctcgaccatcgaggcagtcgaggacgaggtgttcgccgccgacgtcgtgatcggggcagtgctggtgccgggcgccagtgcaccaaagctcgtcacgcgcgggatgctcaagtcgatgcggccgggcgccgtgctggtcgacgtcgcgatcgaccagggcggctgcttcgagacctcgcatccgacaacgcacgccgatccgacctatcaggtcgacggcgtcgtgcattattgcgtcgccaacatgccgggcgcggtgcccgtgacgtcgagccagggactgaacaacgcgacgctgccgttcggcctgatgctcgcgaacaagggctttgccgcggtcctggaaaatccgcatttgcgcaacggactgaacgtgcatcgtggccgcataaccaacaaggcggttgccgagagccttgggctggaatttaccccggtcgagagcggcctcgccgcgtaa

SEQ ID NO: 71 - DNA, 1113 bp, Artificial Sequence
  Modified Nucleotide Sequence for Alanine Dehydrogenase
atgaaggtcggagttccaaaggaaatcaaggcgcacgaatatcgcgtgggccttaccccaggagctgcccgtgaatacgtggcagcgggccaccgcgtgatgatcgagaccaatgccggcgctggcattggtgcaaccgatggcgattatcgcaatgccggcgcaacgatcttaacttcggcagctgaggtattcgcatcgagcgagatgatcgtaaaggtcaaggagcctcagccggccgaatggtcccagctgcgtgaggatcaaatcctgttcacctatttgcacttggccccggacccggaacaggctgccggcctcttgaaatctggatgcattgcaatcgcctatgagaccgtaactgatgctcatggtggtcttccactgctggcgccgatgagcgaggtcgcgggcaggctttcgatcgaagcggcgggtagcgctctaaagcggagtacaggggggcgagggctgctgatcggtggggtgcccggggttcagcctgcccgaatcgtggtgattggaggtggggtcgtcggtacgcacgccgcgcgcatggcggctggtctgggcgcagaggtgaccatcatcgcacgttcgatcatccggctccgtgagctggacgagctattcgaaggacgggttcgcactaggttttcgaccatcgaatccgtggaggaagaggtatttgcggcggacgtggtgattggtgcggtgctcgtcccaggggcaagcgctccgaagctggtccgccggagcatgctgagctcaatgcgcaagagagccgtactcgtggacgtcgctatcgatcagggcggctgctttgagacatcgcgcccaaccactcacgctgatccaacatacgaagtagatggcatcattcattattgtgtggccaatatgccgggtgccgtgccgctaacctcgagccaggcactgaataacgcgaccctgccgtttggtttggctctggccaacaagggtttttccgccgtactcgagaatccacatctgcgcgcaggcctcaacgtccatcggggccggctaacatacaaggcggtggccgagagcctcgggctgcccttctccccgatcgaacaggctgcggcctga

SEQ ID NO: 72 - DNA, 1116 bp, Artificial Sequence
  Modified Nucleotide Sequence for Alanine Dehydrogenase
atgaaggtcggcatcccccgcgaggtcaagaaccacgagtaccgcgtggccatcacgcccgccggcgtgcatgagctggtccgcaacggacacgaggtctacatcgaggacaacgccggtctcggctcctcgatccccaacgaggagtacgtggccgccggcgccaccatcctccccaccgccgacgaggtgtgggccaccgccgacctgctgctgaaggtcaaggagccgatctcctccgagtaccaccggctgcggaagggccagaccctcttcacgtacctgcacctggcggccgaccgggccggtaccgacgcgttgatcgcctcgggcaccaccgcgatcgcgtacgagaccgtgcagctgggcaacggcgcgctgccgctgctcgccccgatgtccgaggtcgcgggccgcctggccccgcaggtcggctcgtaccacctgatgcgtccggccggcggccgcggcgtgctgcccggcggcgtgcccggcacccacccggccaaggccgtcgtcatcggcggtggcgtctccggctggcacgcggccaccatcgccatcggcatgggctacgacgtgaccctgctggcacgcgacatcaacaagctgcgcgaggccgaccggatcttcggcacccagatcaaggccatcatgtccaactcgttcgagctggagaaggccgtcctggacgccgacctggtcatcggcgccgtgctgatcccgggcgccaaggccccgaagctggtcaccaacgagctggtctcccgcatgaagccgggctccgtgctcgtcgacatcgccatcgaccagggcggctgcttcgaggactcgcacccgaccacgcacgacgacccgaccttccaggtccacaactcggtcttctactgcgtggccaacatgccgggcgccgtcccgaacacctccacctacgcgctgaccaacgccaccctgccgtacgtgctggagctggccaaccgtggctggaaggacgcgctgcgccgcgacccggcgctcgcgaagggcctgaacgtccacgagggtcagatcaccttcccggccgtggccgacgccttcggcctggagtcggtcacgctggacagcgtgctcgcctga

SEQ ID NO: 73 - DNA, 1092 bp, Artificial Sequence
  Modified Nucleotide Sequence for Alanine Dehydrogenase
atggaaatcggcgttcctaaggaaactaaggatcaagagtttcgtgtagggttaagtccttctagtgtgcgggtactgcgagaaaatggtcatagtatcttcgtccaaacacaggcgggtactggtgctggatttacagatgaagactatatcagtgcgggggcagaaattgtctctacactggaagccgcttggaatcgggagttagtgatcaaggttaaagaaccgttggtaagcgagtataatcttctgcaaaagggacagttattatttacttatttacatttggcagccgatcgcaaattaacagaacatttactagattgtggcattagtgcgatcgcttacgaaacagtagaacaagctggagtgaatagattacccttgcttaccccgatgagcattatcgctggtcggttagcggtacaatttggcgctagatttctagaacgtcaacaaggtggtaaaggcgtacttctcggcggtgtacccggtgtcaaagctggcaaagtagtaattttaggtggtggtgtcgtcggcacagaagccgctaaaattgcggtgggtatgggtgcgatcgtacaaattttagcagtcaacgttgaacgtctttcttacttagaaactctttttggttctagagttgaactgctgtatagcaactctgctcatattgaaaccgcagttaaagaagccaatttactcattggtgcggttttaattcctggcaggagagcgccaattttagtatcccgtgatttggttaaacaaatgcatcccggttcagtgattgtggatgttgcagttgatcaaggtggatgtgtagaaacattacaccctacatctcacaccagtccggtatatattgatgagggtgtggtacattatggtgttcctaatatgccaggggctgtaccctggacatccactcaagctctcaacaacagcactttgccctatgctgtgcagttggctaatttgggaattaaggctttggatgttaacccagctttggctaagggtttgaatgtgcagaatcatcgtttaatacatccggctgtacaagaggttttccctgatttggtgagttaa

SEQ ID NO: 74 - DNA, 1137 bp, Artificial Sequence
  Modified Nucleotide Sequence for Alanine Dehydrogenase
atgatcataggggttcctaaagagataaaaaacaatgaaaaccgtgtcgcattaacacccgggggcgtttctcagctcatttcaaacggccaccgggtgctggttgaaacaggcgcgggccttggaagcggatttgaaaatgaagcctatgagtcagcaggagcggaaatcattgctgatccgaagcaggtctgggacgccgaaatggtcatgaaagtaaaagaaccgctgccggaagaatatgtttattttcgcaaaggacttgtgctgtttacgtaccttcatttagcagctgagcctgagcttgcacaggccttgaaggataaaggagtaactgccatcgcatatgaaacggtcagtgaaggccggacattgcctcttctgacgccaatgtcagaggttgcgggcagaatggcagcgcaaatcggcgctcaattcttagaaaagcctaaaggcggaaaaggcattctgcttgccggggtgcctggcgtttcccgcggaaaagtaacaattatcggaggaggcgttgtcgggacaaacgcggcgaaaatggctgtcggcctcggtgcagatgtgacgatcattgccttaaacgcagaccgcttgcgccagcttgatgacatcttcggccatcagattaaaacgttaatttctaatccggtcaatattgctgatgctgtggcggaagcggatctcctcatttgcgcggtattaattccgggtgctaaagctccgactcttgtcactgaggaaatggtaaaacaaatgaaacccggttcagttattgttgatgtagcgatcgaccaaggcggcatcgtcgaaactgtcgaccatatcacaacacatgatcagccaacatatgaaaaacacggggttgtgcattatgctgtagcgaacatgccaggcgcagtccctcgtacatcaacaatcgccctgactaacgttactgttccatacgcgctgcaaatcgcgaacaaaggggcagtaaaagcgctcgcagacaatacggcactgagagcgggtttaaacaccgcaaacggacacgtgacctatgaagctgtagcaagagatctaggctatgagtatgttcctgccgagaaagctttacaggatgaatcatctgtggcgggtgcttaa

SEQ ID NO: 75 - DNA, 1055 bp, Artificial Sequence
  Artificial Fragment for Pyridoxine Dehydrogenase
acaaaaaggataaaacaatgtccgtgactgaactgcgtaatgatatccataaattagataccggttatggtctgatgagcctgacctggcgtgcagagccgattcctcagtcgcaggcatttgaagcgatgcaccgtgctgttgagctggcccgcgaacgtggacacaaagcctttttcaacgttggtgagttttatggcccggattttgtgaatttaatatttgttcgcgacttctttgcaaagtaccctgatttgcgtaaacatgtagtgatcagttgtaaaggtggtatggaggttagcacgctgaccccgaatggaagccatgatgcagtcattaaaagtgtgaagaactcggtcagcgcgattggtggctatattgacatatttgaggtggcccgccttgatacatctctgtgtgcgaaaggggaagtatatccatacgaatcgttcgaagcccttgctgaaatgatctccgaaggcgttattggcggtatttcactgagtgaagttactgaagagcaaatcagagcaatttataaagattgggggaagtttttaacctgcgtggagctggaagtttctttattcagcacggatatttttcataatggaattgcgaaaacctgtgccgaattgggactgacggtcatatgctactcaccgctgggtcgcgggctgttaacaggccagcttaagtctaacgctgacatcccagaaggcgacttcagaaaagcacttaagcgctttagtgatgaatcattgaagaaaaatctggatctggtacggtttctgcaggaagagatcgtcggaaaacgttccaaagataactctattactctgcctcagttagctttaggctggataaaacactggaattatgttccggaatataaaggcgcgaaattcatcccaattcccagtggttcttcaatatcaaaagtaaatgaaaactttgatgaaaagaaaacacaacttacggacaaagagtttaaagccattaacgacttcttggctacatttcatactgtgggggatcggtacgagttcaattaa

SEQ ID NO: 76 - DNA, 1067 bp, Artificial Sequence
  Artificial Fragment for Pyridoxine Dehydrogenase
acaaaaaggataaaacaatggtggaacagtctgcaattaataaactgaaacaagatctgtctgcgattgataccggctatgggctgatgtcgcttacgtggcgtgcggaaccgattcctgagacccaggctcatgaaacaatgaaacgtgtcgttgaactggcgcagtctaaaggccataaggcatttttcaactgtggtgaattttatggaccgaatcggatcaatctgacctatattcgtgatttttttgtgaaaaatccagaattacgcaagtatgtgatcataagctgtaaaggtggctgcgattgtgaaacccttactcccaagggaaaacatgacgatgttatcaaaagcgtggaggcatcagttgcagccattggtggctttattgatatatttgaagtcgctcgcttggatctgtcactgtgcacgaatggcgaaacatacccttatgaatcctttgaagcattagccgagatggttgacaatggtgccatcggcgccattagtttgtcagaggtgactgcggagcagatacgtggaatttccaaagactggcacaagtatcttgtcagtgtggaggttgaactgtcgatgttcaccaaagcgattttaaccaatggggtagttaaagcaaataatgatctgaacttggtcacgattctgtactctccgttaggtcgcggattgctgactggtgcagttacgagctcaaaagatataccagctggggatttccgtaatctgctgaaacgttttgatgatgaaagtttaagacaaaacctgacattacttgacttcttacgcgacgaaattatcgcgaagcgccctgcaaataacgaaattacactgccgcaggtagcccttggttgggacaaatattggaacaagacaggcgagtaccccaacactcactttttaccaatcccgagcggttccagtgtaaaaaaactggaagagaactttgatgaagccaaaacgcaaataaccgctgaagagtttcggaaaatcaatgatttcttgaagaacttcaaaactgtaggggacgcttacgagatgagataa

SEQ ID NO: 77 - DNA, 1043 bp, Artificial Sequence
  Artificial Fragment for Pyridoxine Dehydrogenase
acaaaaaggataaaacaatgtcaaaaacgggtgaattaaaacgccagctgaatcagctgcataccggttatgggttgatgagcctgacttggcgcgctgatccagttccgcaagaacaagcatttgaagccatggagcgtgtggtcagtttagcgcaggctagcggcaacaaggcttttttcaatgttggtgaattttatggtccggattattccaatctggaacttgtaaaagcatttttcgaggccaaaccagaattacgtgagcatgttctgattagctgtaagggtggcgtggataataccaaattattgcctaagggaaaatacgcagatgttctttcgagtattgaacaatgcgtgaaacatctgggcacgcacttagacatttttgaagtggcgcggctggataaatctcttggtggcgaatatcctcgtgaatcatttgaggccatggcatcgatggtagacaaaggtattatcgatggtatatctctcagcgaagtaacagcggatgagattcgtgcaataggacatgattgggcgaaataccttgtgtgtgtagaagtcgaattttcaatgttcagccctcagatcctgcacaatggggtattggatgcatgcaatgacctgggattaatcgtcattgcctattctccgctgggccgcggcttactcaccggtacaatcacgaaggattcagcctttaaagatttccgttccagtctgaaacgttttcagaaagattccctgcaaaagaacctggcgctgaccgaatttttgcaggatcagatagtggaccaacgcagttcaacaaacccgatttcgctgccacaggtggcacttggctgggtcaaaagtctgaataatgggaaatatccccatactcacattgttccgatccccagcggatctgacaaaagaaaagtggaggagaactttgacgaatcccgggctaagctcaccgtttctgagattgaaaaaatcaacgatttcttgaagtcattcaacgttgtcggcgaccgctacgagtgggtttcttaa

SEQ ID NO: 78 - DNA, 980 bp, Artificial Sequence
  Artificial Fragment for Pyridoxine Dehydrogenase
acaaaaaggataaaacaatgccgtctcttgttggacgtgatgttggccataccggctatggtctgatgcgcatgacctgggtgcctcaaccaccaccgcaagaacaatgctttgaagctctgaataccgccctggcccatggctcaaatttttggaacgctggtgaactgtacggcacaccggaatataactctttgcatctgttacatagttatttcgcccagcacccagagaatgcggacaaagttgtgcttagcattaaaggtgggctgaaaccgggccagctggtgcctgatggttccgaagccaatattcgtcgctcagtggatgagtgtcttcgtgtgctggatggtaaaaagaaaattgatatctttgaatgtgcgagacaggaccccaaaactacagtagagcagacggttactgtacttgcgcagttggttaaagaagggaaaataggtggaattgggctgagtgaggttgatgcggaaacaattcgtagagcacacaaggtgcacccaattgcagctgtagaggttgaaatgtctctgtttgacctgaccatcttacagaatgacgtcgcgaaagtatgcgcagaactcaacatcccgatagtggcatattcgccattgggacggggtgtcctggccggcgctttcaccacagccgctgacatccccgatggagattttcgcaaaacgttgccgaaattccaagatgaagcaatgaaacagaacatcaagttagtcaatgaggtcaatgacttagccgctcgcaaaggcgtagctcccgttcagatagcactcgcgtgggtcttaactcttagcggaaagccgggtatgcctacgattattccgatccctggtggcaccacgagcgccaaagtggcgcagaacttacaagcgcctcgtctgacagatgcagaaatggcagagattgatgcaatactgaagcggaatgaaattgttgggactcgctactaa

SEQ ID NO: 79 - DNA, 1049 bp, Artificial Sequence
  Artificial Fragment for Pyridoxine Dehydrogenase
acaaaaaggataaaacaatgccgtccccgtcagattatcgtaaagaactgcttgatgtggaagttggttatggactgatgagtctgacatggcgcaaagatcctcttccagcagaaaaagtgtttccgaccatgaaaaaggtggtagaaaaggcgggcaataaaaaggcactgtttaatgtgggtgaattttatggtcctcatctggcgaattacaaactgttgcagtcttttttcgataaatatccggaggatcgcaagaaagttattatcagtgctaaaggaggcgtgaatgtcgaaacattacagcccaccggagatgccgacagcatctctgcgtcaattgaaaatgcactgaaagtgtttggtgggtatctggacatttacgagccggcccgtattgatcttgcactggcggaaaagaatggtgagaaattgtttcctcgtgagacctttgacactatcgtaaagtacatcaaagatggcaaagttggcggtttcagcttaagtgaagttacgaacgagcaaatccgtgctatttacaaggaatacggcgagtatctggcttgtgttgaagttgaactttctatagcgtcgccagaaattatatccaacgggattctggacacatgcaatgaatttggaataccggtcgttgcctatgcacccctggggcggggtttactgacgggcagcttgaaaagcaccgctgatatccctgaaggtgatttccgcggccagttaaaacgcttccaggatgatgccattgcacataacctgactttggtaaaattccttcaagacgaaattgccgcaaaacgttccgacaacccgtctctccctcaggtggccattggctgggtccgtggactccggcgcgagtatccaaaaacgaatatcatacccattccatcagggtcttcggttgaaaaggtaagtgtcaactttgatcacattgatttatcagacgatgagatgagcaaaataaaagactttttaaagtcatttaaaatcgctggtgatagatatgagttcgcgcaagcacattaa

SEQ ID NO: 80 - DNA, 1067 bp, Artificial Sequence
  Artificial Fragment for Pyridoxine Dehydrogenase
acaaaaaggataaaacaatgtcttttaaacctgttgaaatatcgggcaagtttggctttgggacaatgtccatgacctggacaccgacgccgccaccggcacagcaatccattgataccctgaaatttgtgacgagccatccgaaattcggcactaaactgatcaatggtggtgaattttatggcccagattttgccaatctgaaactgttaaagcagttcctggaagagaatgatcctgaagagaataaacagttgattatatctatcaaaggaggtgcggacaatgaaacattaaagccgaatggtaccaaagagtttgtttcaaaaagcattgaaaacatcgtaagttttttccctaaacaaaaacagaaccgtccgaaattactgtttgagatggcccgcgttgatccttctgtgccctatggagaaaccattggctatatcagtgaatatgtgaaactgggtgtgattgatggtattagcctgtctgaagtcggcaaggaatccattcaggccgctctgaaagtattcccaatatcatgtgttgaattagagctgagcctttttagtcaagaagttatcaccactggtatattggaagagctgtcaaagcacaacctgccgctgattgcttatagcccgctttgccgtgggcttttaacggattatgcagttgagaatagcgatacatttttggcatcaatcccacagggtgatatacgtcatcatctggacaaatttcaaccggacaccttcaacaaaaaccttcccgcgttaaaggaactgtacaagtttgctcacgaagtcaaaaacacgactctggaatctttggcacttagctggattgttacagtctcggaggcgcgcaatttccggggcattgaaaaagtgacccgtatcttacccattccttccggaagtacgaaaaagcgcgtagagtcaaattttgggagtttaatagagctgactgatgacgatttacagtcgatcgacgaaattttcgccaaatacccaattagtgggttgagatacaatcagtttctggaaggaactcttttccaataa

SEQ ID NO: 81 - DNA, 1007 bp, Artificial Sequence
  Artificial Fragment for Pyridoxine Dehydrogenase
acaaaaaggataaaacaatgacatttaataaaatcacaccgacgagtaaaggttttggactgatgggcttcacatggagagaaaccgaaaccccggatgaaattgcatttccgaccatgaaacgtgccattgaaaagggcattttagtgtggaatagcgcagagttctatggcaccaaagatccattaggtaacctgaaattgattcgtcgctattttgaaaagtacccggaagacgccgacaaagtgtgtctgatggtcaaaggaggtctggggccgaacttggtgccgaatggctccaaggagtttgtgcagaaatccattgatactgttctggaaacgctgggtggctctgtacctgtgagcatcttccagtgtgcacgtgtggacccgaatacacccatagaagagacggtcgcggccatcaaagagtatgttgatgctggcaaaattggagcgctgggtttatcagaagtcggggctgcaaccattgagaaggtacattctctgagtccgatagcggctgttgaaaatgagttatcactgtgggccaccgatatctttacaaacggtgttgcggaagtatgcgcaaaatataatattcccatcactgcctatgctcctctgggttctggcgtacttacgggccgtttcaaaagcacccaggatgtgccagaaggggacgcacgcaagcacatggtcaaataccaagaggaaaacttgaaacagaatctggttctcgtagaccgccttaaagaagtggccaccaaggcgggtattcctcttccacagatggccattgcatgggtcaaagctcaatcaaaccacaatggccatgggaccataatccctatcccaggtaacacaactgttgcgcggctggaagagaaccataatgatttaacgctgtcggcagaaactctcaaagagatagatgatgttctgaagagtacgaaaattgttggagatcgcgggtttactcttggtggagaatttatgaatggttaa

SEQ ID NO: 82 - DNA, 1196 bp, Artificial Sequence
  Artificial Fragment for Pyridoxamine Pyruvate Transaminase
acaaaaaggataaaacaatgcgttatcctgatcacactgaaccggtgattacattaaccgcaggtccagttaatgcctatcccgatgtgttacgcggtttgggtcgcaccgtgctttatgattacgacccggcttttcagttattttatgagaaagtcattggtaaagcgcagaaggccatgcgtctgagcaataaaccggttgtcctgcatggcgaaccggtgatgggtctggaggccgcagctgcgtcattgatctctagagatgatgtggtacttaatctggcaagcggtgtttacggtaaagggttcggctattgggctaagcgttattcgcctaacctgttagaaattgaggttccttacaatgaagcgatcgatccgcagtccgtcgctgacatgctgaaaagtcatcccgaaattaccgtagttagtgtctgtcatcacgacactccatcaggtacgatcaatccgattgatgagatcggagcgattgtttcggctcatggcggatatctgatcgtagatgccgttagttcattcggtggcatgaaaacacatcccgaagattgcaaagccgacatatatgtgaccgggccaaacaaatgtcttggcgcacctcccggactgacgctgatgagcgtgagcgaccgtgcctggaccagaatgaaagcgaactctgcagcacctcgcgcaagtgttctgagcattttggattgggagcatgcatggtccaaggaaaaaccgtttccatttactccgtctgtcgccgaaatgaacggactggatgttgctttagacctttatttgaacgaagggcctgaagcggtgtgggctcggcacgcactgaccgccaaagcgacacgcgccggggctcaagcaatgggcttatcgatctgggctgcaaatgaaaagattgcctcaccgacaacaaccgcaatacgcacgccagacggggtcgatgaaaaagcgctgcgtcaggccgctcgtgagcgttacggagtggtattttcttccggccggggagaaactttaggcaaactgacgcgcattggtcacatggggccaactgcgcagccgatctacgcgatagcagcgctgacggctttgggcggtgcgctgaatgccgctggacggaaacttcaagttggcaagggtattgaggcagcattagccgtaatagatgcggatgcctaa

SEQ ID NO: 83 - DNA, 1196 bp, Artificial Sequence
  Artificial Fragment for Pyridoxamine Pyruvate Transaminase
acaaaaaggataaaacaatgcgttataaagataatgccgatccggtgattacactgaccagtggtcctgttaatgcatatgcggatgtgctgcgtggtctgagccgcactgttttatacgactatgatccagcatttcagttattctatgaaaatgtggtggccaaagcacagaaagctatgcgtctttctacccggccggtactgttgcagggtgaacctgttatgggactggaagcagcggcagcttcactgatttccagcgaagatgtcgttctgaacttagcctctggtgtctatggtaagggctttggttactgggcaaaacgctatagtccgaatctgttagagattgaaaccccctataacgaatctatcgacccacaagctgttgccgatatgcttaagaaacatccggaaattaccgttgtctcggtgtgtcatcacgatacacctagtggcacgattaatccgatcgacgaaattggtgcgatagtatcagcacacggggcctattttatcgttgatgctgtatcttcattcggaggcatgaaaacccatcccgaagattgtaaggccgacctttacgttactggtccaaacaaatgcctgggcgcgcctccgggactgacaatgatgggggtcagcgatcgggcctgggccagaatgaaagctaatccatccgcacctcgtgcctcggtgctgagcattttagactgggaggatgcttggagtcatgaaaaaccgtttccattcacaccttctgtagcggagataaatggcttagatgtcgcgctggacctttacctgaacgaggggccggaagtggtgtgggcacgccatgcgctgactgcaaaagccacgcgtgcaggtgcattggctatggggttgtcattatgggcggctagcgagaagattgccagtcccaccacaacgtcgatacgcactccacagggggttgatgaagctgcacttcggcaagcggtgcgtgagcgctatggcgtagtgttttcatccggtcgtggcgagacgttgggtaaacttacccgcatcggacacatgggccccacggcgcagccgatttacgcagttgcggctctgactgcgctgggcggggctttgaacgcgatgggacaaaagctggccgtcggcagaggaatagacgccgctttagcggtaatcgacgccgatgcataa

SEQ ID NO: 84 - DNA, 1196 bp, Artificial Sequence
  Artificial Fragment for Pyridoxamine Pyruvate Transaminase
acaaaaaggataaaacaatgcgttatgcactggatgccgatcctgttattacactgaccagcggtccggttaatgcatatccggatgtgctgcgtgctttggcgcgcacagttctttatgattacgaccctgcttttcagttacattatgaacaggtgatcggtaaaatgcaaacggcaatgcggacaagcaaccgtccggtcattctgcatggcgaaccagtgctgggattagaggcggctgcggccagtctgattaccgcagacgatactgtgttaaatctggcatctggtgtttacggcaaagggtttggttattgggcagcgcgttattcacccaaccagctggaaatcgaggttccctacaaccaggctattgatccgcagcaagtcgccgatatgctgaaggctaatcctgccatttctgttgttgcagtatgtcatcacgacactccgtcagggaccattaatccaatcgatgaaataggtgccattgtcgccgaccatggagcctatctgattgttgatgcggtgagttccttcggcgggatgaaaacccacccggaggattgcaaagcagacatctacgtaacaggtcctaacaaatgtttaggcgcaccgccagccctgaccatgttgggtgtcagcgaaagagcgtggcagaagatgaaagataatccggcagctcctcgcgcctcgatgttaagcattcttgactgggaaaatgcctggtctcgtgatgaggcctttcccttcacgccgtcagtggctgaagttaacgggttagacgtagcgctggatttataccttgccgagggaccagaagcagtctgggcacgccatgcgctgaccgccgctgcgactcgggctggcatactggcaatgggtttggacctgtgggcggctgatgcggccatcgcaagtccgacaacgaccgcagtgagaactcctgagggcattgatgaagcgaaactgcgtgctacagtacgcgaacgttatggagtggtgatgtcgtccgggcgcggcgaaacgttgggtaaacttactcggataggccacatgggaccaacggcgcagcctatctatgcaattgccgcgctgaccgcttttggtggcgctttacaagcgttgggtcatcccgctccagtaggaaaggcaatcgacgcggctcttgcggtcatagatgccgcagcctaa

SEQ ID NO: 85 - DNA, 1193 bp, Artificial Sequence
  Artificial Fragment for Pyridoxamine Pyruvate Transaminase
acaaaaaggataaaacaatgacagttgaacgtattgttgaacctgcttttactttaagtagcggtcctgtcgatgcgtatccagccgtgttacgcgcactgtcacgtacggttttatacgattttgatcctgtgttccagcacttttatgaagcagttactgataaagctaaagtggcacttcgtaccgcggcaccacctgtaatcttgcatggcgaaccggtgctgggtttagaggccgcggctgccagcctgattgcacgcgacgatgttgtacttaatttagcgtctggtgtctatggcaagggtttcggtttttgggccaaacgttactgtgctgaattggttgagattgaagtgccttatgatgacgcgatcgatccggcggcggtcgaggccgcattcagagaacgccccgacattcgtgtagttagtgtctgccatcatgatacgccgtctggcactctgaacccaattgatgaaatcggacgtatagtggcagcccatggtgcttatctgattgtagacgccgtttcttcgtttggtgggatggatgttcatccagaggcttgtcaagccgacatctttgtgacagggccgaataaatgcctgggctgtccgcctggtttgacgctgctggcggtcagtggccgcgcttgggataaaatgaaggccaatccggacgcgccgagagcatcaattcttagcatactggattgggaaaacgcgtggcggcgtgatcagccgtttcccttcaccccaagtatttccgaagtgaatgcactggatgcggctttagacctgtatctggaagaggggccggaagccgtatggcgccgtcacgcacttacatcaaaagcctgccgcgatggcatccgcgctatgggaatgactatatggcccgcgcgtgaggcaattgcttctccgaccaccacggcggtgcgggtacccgacgggattgatgcagatgctctgttggcgacagcacgtgatggctttggtgttagcttctcgtccgggcggggcgaaaccaaaggaaagctgatccgcataggacacatgggtcgcaccgcccagccattatacgccgttgtcgggttagcagcgctgggcggtgcgttgagacggctgggagctaatgtggacgccggcgcaggaatggaggcagctcttgccgtcatctcaaaccaataa

SEQ ID NO: 86 - DNA, 1202 bp, Artificial Sequence
  Artificial Fragment for Pyridoxamine Pyruvate Transaminase
acaaaaaggataaaacaatgaatcagacgaaaattgcagatccaatttttaccttgaccacaggtccggttgatgcctatccagcggtgctgcgtgcactgtctcgcccggtgttatatgactacgatccggcgtttcttgcgttctatgaagccgtcaatgctaaagtacagcgtgcctttcatacgaaaaccccacctgtgatcctgcagggtgaacctgtgctggggttagaagcggctgcggctagcctgattgccaagaaagatgtggtgcttaacctggtttctggcgtttacggtaaagggttcggcttttgggcagcgcgctatgcgcgtgagctggtcgaattggaagttccctataacgatgtcctgacagccgatgcggtagctgacatgttgaaaaagcgtccagatatcgccgtggttgcgttatgtcaccatgataccccgtcaggtaccgtcaatcctgtagccgaaataggtgcagtggtgagagcggcgggaaaactgtttattgttgattccgtaagtgcctttggtggcatggacgtgttaccggaaacggcttgcgctgatattttcgtaactggtcccaataaatgtttaggctgcccgcctggtttgtctcttctgcacgtcagcgaagctgcctgggagaagattgcagccaatcccgatgcaccaacagcttcaatgcttagcatcagtgattggaaaagagctcacgaggctggtcaaccgttcccgtttactccttcggtttccgaaataaacgccctggacgctgccatggatttatatctggaggaaggcgagcaggcagtatgggcgcgccatgcgctgacagccaaagcatgtcgggcaggagtgcaagcagccggcctgaagttatgggcggcacgggaagacatcgcttcaccaacctgcactgccgtggcaattcccgatgggattgacgaagcaaaactgcgggcgagtatacgtgatcgctacggagttgtcttttctagcggccgtgctgagacgctgggtaagttgacacgcatcgggcatatgggcccgactgcacgtcctacctattcattagtctcggttgcagccattgttggagggctgaaagcagccggcgttaaagaccttgatgtagaggctggagttgcggcggcaatgaaggtcatagacagtgcagaaaaataa

SEQ ID NO: 87 - DNA, 1190 bp, Artificial Sequence
  Artificial Fragment for Pyridoxamine Pyruvate Transaminase
acaaaaaggataaaacaatgatgcgttccgcctttgcccagccgttatttaccctgactacaggtcctgttgatgtctatccggcggtgagtcgtgcattatctatgcctgtctggtatgattacgacccggcttttcagaactgctttgaacgcgtcagcctgaaggccgcacaggctctggaaagtgccacgccgcctctgatcttacagggtgaacccgtactggcactggaagccgcagcggcatcactgcttagcgctgaagatatggtgctgaatctggtttccggcgtatatgccgccggtttctctgagtgggcgcggcgttactctcgtcaagttgaagagctgcgcgttgaatttaatcaggtgattgatcctgtcgctgttgatgcatggcttagtcgtcatccgcagattaccgtggttgtggtttgccaccatgatacgccttccggcaccttaaacccactggatgaaattgggcgggttgtgaaaaaacatggtaaattgctgcttgtggatgcggtttcttcgtttgccggtgtggcagtcacagcggccagttgccaagctgacctgttcataaccggccccaataaatgtttaggctgtccgccaggattaagcctggtggcggtatctgatgctgcctggcggaaaatcgaggccaatgacgctgcaccgcgtgcgagcgtattatcgttgttagactggcgtgatgcctggtcagctgaaaaaccttttccctatacaccatcagtagcggagatcaacggactggaagcagcgctggacggttacttgcaggaggggccagctcaggtctgggcgcgccacagattaaccgcacaagcattccgcgccgggattcaggcaatgggacttagcttgtgggcggctgatgaaagtattgcttcaccaacggcaactgcgatacgcgttcccgacggagtaaatgaagcccaatggagactgcgcgcgcgtgagtgttatggcgtgatgttttcgtcagggcgcggtgaaacgctgggcaaggtattgcgtgtcggtcatatgggaccgactgcccaaccaatgttcgcgattatcgctcttacagcactggcgggggcacttaaccagttaagagagccgcaactggatattggcgctggcgttgcagccgcaatggctgtgataggttaa

SEQ ID NO: 88 - DNA, 1178 bp, Artificial Sequence
  Artificial Fragment for Pyridoxamine Pyruvate Transaminase
acaaaaaggataaaacaatggcacatcctgattttacactgagcgccggtccggttaccgttaccgcacgcacgctggcgggtttaggcagtccgattctgtatcattatgaccctgaatttttagcaacatttcgtcgcacgcagggtaaagtggggcagatgttccaaaccgataatgacattatcctgatgcagggtgaagccattgttggtcttgaaggcgctttgcgttctctgataacgcccgggatgcatgttctgaacttagtgcagggagtatttggcaaaggtaccggctactggattgccgatttcggagctgtgctgcacgagatcgaagttggctatgatgacgcggtctcaccggcacaagttgaagagtatcttgatgcacatccagaaattcagatggtttgtctggtggcctccgagaccccgagtggtacagtaacggatgtcgcggcaattggcccaatgtgtcgggatcgtggaatcctgacttatatagatactgtttctggcgtgttgggtatgccctggaaaacagacgaatggggcctggatttatgcgtcgcaggagctcagaaatgtctggggggccctcctggtgttgcgcttatttcagtgagccaaaaggcatgggatgtaatgtacgcgaatccggccgctccacgtgactcatatttaagtctgatcgattggaaagagaaatggctgggtgaaggccgctttccgtatacgccatctgtgagcgacatgaacgggcttgaagcagcgttagatcaggcactggaagaggggattgacgcggttgtcgcacgtcacgaagccgctgccgctgtgacccgcgccggagctagagccatgggtctggaactttgggcgaagagcgaagagatagcatcggcttgcgttacttccattcgtctgcccgatgacatcgataatgccgtggtacgcgatcatgctcgtgaagtctacggggtgatgttatcacacgggcagggtgccggcaacttggtacggttatctcacatgggtccgacagctggtggcctgcattcggtggtaggacttgccgcgttaggccgcaccttggctgacctgggaatgtccgtcgatataggtgcgggactggaggcggcattagcgctgttgagtactcaaagacggtaa

SEQ ID NO: 89 - DNA, 1133 bp, Artificial Sequence
  Artificial Fragment for Alanine Dehydrogenase
acaaaaaggataaaacaatgattattggtgtacctaaagaaattaaaaatcatgaatatcgtgtcggcatgactccagcaggagttcaggaactgacggcccgtggacataaagtgctggttcagcaacagggaggtgaagcgattggactgagtgatgccgagtatcaagcagccggtgctgaattagtggccagcgcagcggaaatctttgctcgtgccgagatgatagtaaaggttaaagaaccccagccggaagagtgcgaactgttgagaccgggtcaattactgtttacttaccttcatctggcacctgatccggtgcagacccagctgttagttcactcaggggcggtggctattgcctatgagacggtaacagacgatcgcggcggtttgcctctgttagcaccaatgtctgaagtcgcggggcggatggcaattcaggctggcgcccatagcctggaaaaagcgcaagggggtaacggcacactgttaggtggggttccggcagtggcgcccgctaaggttgtggtacttggcggtggcgttgtcggcgtcaattcagcgcgtatggcactgggattaggggccgatgtcaccatcctggctcgctcccttcctcgtctgcgggaattagacagcctgtacggtccggctcttaaaacgttgtatagtactcgtaccaacatagaggcgtgcattgcccaggctgatctggtgattggtgccgttcttatccctggggcagcggcaccaaaactgttaacacgcgacatgttgaagctgatgcgtaaaggctcggtgcttgtagatgttgccatagatcaaggtggctgtttcgaaacctctcacccgacgacccatgaggaacccacatatgtggttgacggtattatccattactgtgtggctaatatgccaggcggggtagcgcgcacctccacttttgcactgaccaatgccacattgccgtttgttttagcgctggctgataaaggatatgtcagagcacttcaggaggatcgccacctgcgggccggtctgaacgtgttcaaaggcaagatcacgcaagcggcagttgcgcaggcattgggatatgaatacgtcagcgctgaggtagttttagactaa

SEQ ID NO: 90 - DNA, 1136 bp, Artificial Sequence
  Artificial Fragment for Alanine Dehydrogenase
acaaaaaggataaaacaatgcgtgttggttgtccgaaagaaattaaaaatcatgaatatcgtgttggcctgacacctgcgagcgttcgtgaatacgttgcccatggtcatgaggtccgggttgaaaccaaggcaggcgcaggtattggagccgatgatgccgcttatgtggccgctggcgcaaaaattgccgcttctgccaaagagatctttgaaaagtgcgacatgattgtaaaagttaaagagccgcagcccagcgagtgggcgcagttacgcgatggtcagattctgtatacctatcttcacttagcgcccgatccggaacagaccaagggcctgcttgcatcaggtgtcaccgctgttgcctatgaaactgtgacggatgatcgtggaggcctgcctctgcttgctccgatgtcagaggtcgcaggacgtctgagtatccaggcaggggcgaccgcattgcaaaaagccaatggtggccttggtattttgttaggaggggtgcctggcgtgttgccagctaaagttacagtgatcggtggcggtgtggtcgggttgcacgcggccagaatggcggcaggactgggtgcagacgttagcattttagcaaagtctttaccgcgcctgcggcagctggatgatatctttggcggtcgtgtacatacgcgctattccagcattcaagccttggaagaggaagtattcagtgcagacttagtaataggtgctgtgttgattccaggcgctgccgcaccgaaacttgtcactcgtgaaatgctgtcgggtatgaaaagagggtctgtgatcgtagatgtcgctatagaccaaggtgggtgttttgagacaagtcatgcaacaacgcattcagagccaacctacgttgtagatgacgtggtgcattattgcgttgccaatatgcctggcgctgtgccggtcacttcggcgcatgccctgaacaatgcgacgttagtttatgggctggcgcttgcagaccgcggactgcgggcgatagcggaagatcgccacctgagaaatggcttaaacgtccacaagggacgcgtaacaaacaaaccagtggctgaagcgctggggtacgaatcctacactcccgaaagcgtactgaacgttgcgtaa

SEQ ID NO: 91 - DNA, 1139 bp, Artificial Sequence
  Artificial Fragment for Alanine Dehydrogenase
acaaaaaggataaaacaatgcgtatcggtgtacctaaagaaattaaaaatcatgaatatcgtgttggccttacgccgcagtctgtggcagaactgaccacgctggggcatgaggtttggattgaaaccctggccggtgcagccattggttttgcagatgaggattaccgtaaagccggggcgcagattgctccgtgtgcaggcgaagtgtttcagcaagcgcagttaatcgtcaaagtaaaggagcctctggcggttgaacgtgccaaactgcgcgaacaacatacgctgttcacatatctgcaccttgctccggatcggccgcagaccgacgagctgatggcaagcggagccacatgtattgcatatgaaactgtgaccgatgttcagggccgtctgccattattggcgccgatgtcagaagtggctgggcgcatgtctatacaagccggtgcaggttgcctggaaaaagcgcgtggtggtcgtggcgtactgttaggaggcgttcctggggtggcgccgggaaaggttgtgattcttggcggtggtgtagttggaagccatgccctggcaatggccgtgggcctgggtgctgatgtcacggtactggccaaaagtgtcgatgcactgcgcagacttgatgctcagtacggcaatcgcatcaccactttatattccacacgcgccgcagtgcaggagcaagttttagcggctgacttggtcataggtggcgtgttaattcccggggcggcagctccaaaattgattagcgcggacatggttcgccagatgaaagccggagcggtgttagttgatgtggcaatcgaccaaggcggatgtgctgaaacctcgcgggccacaactcatgcagaacccacctatgttgtcgatgacgttgtacattactgcgtagccaatatgcccggtgctgtggcacgtacatcaacgttggcgctgaacaacgccactttgccatttgtcgttgcgctggctcagaagggcactcggagagcgctggaagacgatcctcacctgctggctggtcttaatgtcgcgcgcggagcaataacatgcgccagtgtcgcagaggctcacgggttaccattccaaccgcctgctagcgtattagagagactttaa

SEQ ID NO: 92 - DNA, 1133 bp, Artificial Sequence
  Artificial Fragment for Alanine Dehydrogenase
acaaaaaggataaaacaatgcgtgttggtgtgcctaaagaaattaaaatgcaggaatatcgtgttggtctgaccccaggtgcagtccgcgaatatgttgcagccgggcaccaggtgacggttgagaccggcgcaggtagcggcattggagcgtctgatgaagtgtaccaacgggcaggggccgcgattgctgaaaacgcacgtgacatctttgatagctccgacatgattgtgaaagtgaaagagccgcagaaaagcgaatgggcccagcttcgcgaaagtcaaatcctgtttacatacttacatcttgcgcctgatcccgaacaggcaaaaggcctgttaacctctggttgtactgcggtcgcctatgagacagttaccgatgcagctggtcacttacctctgcttgcgccgatgagcgaagttgccggtcgtctggcaattgaagccgcaggcgccgcgttaaagcgttcagctggaggccgcggggtgctgttaggtggtgtgcccggcgtacagccggctcgtgtcgttgtgttaggaggcggtgtagtgggaacgcaagccgctcgcatggcggcgggtcttggtgctgaagttactgtaatcgcacgttcggttcctcgtctgcgcgaactggacgatgtctttttagggcgggtgagaacacgcttctctaccattgaggcagttgaagatgaggtatttgctgccgacgttgtgatcggggcagtgctggtaccgggcgctagtgcaccaaaattggtcacgagagggatgttaaagtcaatgcggcctggtgccgtgctggttgatgttgcgatagaccagggcggatgtttcgagacttcgcatccgacaacgcacgctgatccgacctatcaggtcgatggcgttgtacattattgcgtcgccaacatgccaggtgcggtgcccgtaacgtcaagccaaggactgaataatgcgacactgccgttcggcctgatgttggcgaataaaggatttgccgctgttctggaaaatccgcatttgcgcaatggactgaacgtacatcgtggcagaataactaacaaggcagttgctgagagtcttgggctggaatttaccccagtcgagtccggtttggccgcgtaa

SEQ ID NO: 93 - DNA, 1130 bp, Artificial Sequence
  Artificial Fragment for Alanine Dehydrogenase
acaaaaaggataaaacaatgaaagttggagttcctaaagaaattaaagcacatgaatatcgtgttggtcttaccccaggagctgcccgtgaatacgtggcagcgggccatcgcgtgatgatcgaaaccaatgcaggtgctggcattggtgcaaccgatggcgattatcgcaatgccggcgcaacgattttaacttcagcagctgaggtatttgcatctagcgaaatgatcgttaaggtcaaagagcctcagccggcagaatggtcccagctgcgtgaggatcaaattctgttcacctatttgcacttggcaccggacccggaacaggctgccggcctgttgaaatctggatgtattgcaatcgcctatgagacagtaactgatgctcatggtggtcttccactgctggcgccgatgagcgaagtcgcgggcagactttcaatagaagcggcgggtagcgctttaaaacgtagtacaggagggcgtggactgctgatcggtggggttcccggggttcagcctgcccgtattgtggtgattggaggtggggttgtcggtacgcacgccgcgcgcatggcggctggtctgggcgcagaggttaccatcatagcacgttctatcattcggttacgtgaactggacgaattattcgaaggacgtgttcgcactagattttcgacgatcgaatccgtggaggaagaggtatttgcggcggacgtggttattggtgcggtgcttgtccctggggcaagtgctccgaagctggttcgccggagcatgctgagttcaatgcgcaaaagagccgtattggtggacgtcgctatagatcaaggcggctgctttgaaacatctcgcccaaccactcacgctgatcctacatacgaagtagatggcattattcattattgtgttgccaatatgccgggtgccgtgcccttaacgtcgagccaggcactgaataacgcgaccctgccgtttggtttggctctggccaacaaaggtttttccgccgtacttgagaatccacatctgcgcgcaggcttaaacgtccatcgtggccggttaacatacaaggcggtggccgagagtcttgggctgcccttctcaccgatagaacaagctgcggcctaa

SEQ ID NO: 94 - DNA, 1133 bp, Artificial Sequence
  Artificial Fragment for Alanine Dehydrogenase
acaaaaaggataaaacaatgaaagttggtattcctcgtgaagttaaaaatcatgaatatcgcgtggcaatcacgccagccggcgtgcatgagctggtccgtaatggacatgaagtttatattgaagataatgcgggtttaggaagcagtatccctaacgaagagtacgtggcagccggtgctaccattttacccaccgcagatgaagtatgggccacagctgacttactgctgaaagtcaaggagccgatcagctctgaatatcaccgtcttcggaaagggcagactttatttacgtatctgcatctggcggcagatcgtgccggtacagatgcgttgattgcttcaggcaccaccgcgatagcgtacgaaacagtgcagttaggtaatggagcgctgcctcttttagcaccgatgagtgaggttgcgggccgcctggccccacaagttggttcttatcatctgatgcgtccggctggcgggcgtggtgtacttcccggaggcgtgcccgggacacaccctgcaaaagccgttgtaattggcggtggtgtctccggatggcatgcggctactatcgcaattggtatgggctatgacgtgaccctgttggcacgcgatataaacaaactgagagaagccgatcggattttcgggacccagatcaaggctattatgtcaaatagctttgaactggagaaagcagttcttgacgccgatctggttataggagctgtgttgattccgggcgcaaaagccccaaaactggtcacaaacgaactggtttctcgcatgaagccgggttcagtgttagtagatatcgctattgaccagggcgggtgttttgaagattcgcatccgactacgcacgatgacccaacgttccaggttcataatagtgttttttactgcgtagcaaacatgccgggagccgtccctaatacctccacctatgcgcttactaacgctacactgccgtacgtgttggagctggcaaaccgtgggtggaaagatgcgctgcgcagagatccagcgttagcgaagggccttaatgttcacgaaggtcaaataacttttccggccgtagctgatgcattcggcctggagtcggtcacgttggacagcgtgttagcctaa

SEQ ID NO: 95 - DNA, 1109 bp, Artificial Sequence
  Artificial Fragment for Alanine Dehydrogenase
acaaaaaggataaaacaatggaaatcggcgttccgaaagaaaccaaagatcaggagtttcgtgttgggctgagtccttctagcgtgcgcgtactgcgtgaaaatggtcatagtatcttcgtccaaacacaggcgggtactggtgcaggatttacggatgaagactatatcagcgcgggggcagagattgtctcaacactggaagccgcttggaatcgggagttagtgatcaaggttaaagaaccgttggtgagcgagtataaccttctgcagaagggacagctgttatttacctatttacatctggcagccgatcgcaaattaacggaacacttactggattgtggcattagtgcgatcgcatacgaaacagtagaacaagccggagtgaatcgcttaccattgcttaccccgatgagcattatcgccggtcggctggcggttcagtttggcgctagattccttgagcgtcagcagggtggtaaaggcgtacttctgggcggtgttcccggcgtcaaagcaggcaaagtggtaattttaggtggtggtgtcgtcggcacggaagccgctaaaattgcggtgggcatgggtgcgatcgtacaaattctggctgtcaacgttgagcgtctttcttacttagaaactctttttggatcacgcgttgaactgctgtatagcaactctgcacatattgaaaccgcagttaaagaagccaatctgctgataggtgcggttttaattcctggccgcagagcgccaatactggtctcccgtgatctggttaaacaaatgcatccggggtcagtgattgtggacgttgcagttgatcagggtggatgcgtagaaacattacaccctacgtctcacacctcaccggtgtatatagacgagggcgtggtacattatggtgttccgaatatgccaggggctgtcccctggacatccacccaagccctgaacaacagcactttgccgtacgctgtgcagctggcaaatttgggaattaaggccttggatgttaacccagctttggcaaaggggcttaatgtgcagaatcatcgtctgatacacccggccgtacaagaggttttccctgacttggtgagttaa

SEQ ID NO: 96 - DNA, 1154 bp, Artificial Sequence
  Artificial Fragment for Alanine Dehydrogenase
acaaaaaggataaaacaatgattataggggttccgaaagagataaaaaacaatgaaaatcgtgtggcattaacccccgggggtgtttctcagctgattagcaatggccaccgggtgctggttgaaacaggtgccggccttggtagcggatttgaaaatgaagcctatgagagcgcaggtgcggaaatcattgccgatccgaagcaggtgtgggacgccgaaatggtcatgaaagtaaaagaaccgctgccggaagaatatgtttattttcgtaaaggactggtgctgtttacctaccttcatttagcagctgagcctgagctggcacaggccttgaaggataaaggagtaactgccatcgcatatgaaacggtgagtgaaggtcggacattgcctcttctgacgccaatgtcagaggttgcgggccgtatggcagcgcagataggtgctcaattcttagaaaagcctaaaggcggaaaaggtattctgttggccggggtgcctggcgtttcccgcggaaaagtaacaattatcggaggaggtgttgtcgggaccaacgccgcgaaaatggctgtcggcctgggtgcagatgtgacgatcattgccttaaatgcagaccgcttgcgccagcttgatgacatattcggccatcagattaaaacgttaatttctaatccggtcaatattgctgatgctgtggcggaagcggatctgctgatttgcgccgtattaattccgggtgctaaagctccgacccttgtcactgaggaaatggtaaaacaaatgaagcccggttcagttattgttgatgtcgcgatcgaccaaggcggcatcgtcgagactgtggaccatataacaacccatgatcagccaacatatgaaaagcacggggttgtgcattacgctgtagcgaacatgccaggcgcagtccctcgtacctcgacaatcgccctgactaacgttactgttccatacgcgctgcaaatcgccaacaaaggggcagtaaaggcgctggcagacaatacggcactgagagcgggtttaaataccgcaaacgggcacgtgacctatgaagctgtagcacgtgatctgggctatgagtacgttcccgccgagaaagcgttacaggatgaaagtagcgtggctggtgcttaa

SEQ ID NO: 97 - protein, 7 aa, Artificial Sequence
  Specific Amino Acid Residues
  misc_feature (2)..(2): Xaa is Val, Cys, Ile, Ala, Met, Ser, Gly or Leu
  misc_feature (3)..(3): Xaa is Gly or Ala
  misc_feature (5)..(5): Xaa is Phe or Leu
NXXEXYG

SEQ ID NO: 98 - protein, 6 aa, Artificial Sequence
  Specific Amino Acid Residues
  misc_feature (1)..(1): Xaa is Ile, Val, Phe or Leu
  misc_feature (2)..(2): Xaa is Ser, Thr, Asn, Cys or Met
  misc_feature (3)..(3): Xaa is Cys, Val, Ala, Ile, Trp or Phe
  misc_feature (6)..(6): Xaa is Gly, Ala, Ser or Cys
XXXKGX

SEQ ID NO: 99 - protein, 6 aa, Artificial Sequence
  Specific Amino Acid Residues
  misc_feature (1)..(1): Xaa is Leu, Met, Ile or Val
  misc_feature (2)..(2): Xaa is His or Gln
  misc_feature (3)..(3): Xaa is Gly, Cys or Ala
  misc_feature (4)..(4): Xaa is Glu or Asp
  misc_feature (5)..(5): Xaa is Pro or Ala
  misc_feature (6)..(6): Xaa is Val, Ile, Leu or Ala
XXXXXX

SEQ ID NO: 100 - protein, 9 aa, Artificial Sequence
  Specific Amino Acid Residues
  misc_feature (1)..(1): Xaa is His or Ser
  misc_feature (2)..(2): Xaa is Glu or Asp
  misc_feature (8)..(8): Xaa is Ile, Val or Leu
  misc_feature (9)..(9): Xaa is Asn or Thr
XXTPSGTXX

SEQ ID NO: 101 - protein, 7 aa, Artificial Sequence
  Specific Amino Acid Residues
  misc_feature (1)..(1): Xaa is Val, Ile or Ala
  misc_feature (3)..(3): Xaa is Ala, Thr or Ser
  misc_feature (6)..(6): Xaa is Ser, Ala or Gly
  misc_feature (7)..(7): Xaa is Phe, Trp or Val
XDXVSXX

SEQ ID NO: 102 - protein, 10 aa, Artificial Sequence
  Specific Amino Acid Residues
  misc_feature (1)..(1): Xaa is Gly or Ser
  misc_feature (2)..(2): Xaa is Pro, Ser or Ala
  misc_feature (3)..(3): Xaa is Asn, Gly, Ser, Ala or Gln
  misc_feature (6)..(6): Xaa is Leu or Met
  misc_feature (8)..(8): Xaa is Ala, Ser, Cys or Gly
  misc_feature (9)..(9): Xaa is Pro, Thr, Ser or Ala
XXXKCXGXXP

SEQ ID NO: 103 - protein, 9 aa, Artificial Sequence
  Specific Amino Acid Residues
  misc_feature (1)..(1): Xaa is Gly or Asp
  misc_feature (2)..(2): Xaa is Val or Ile
  misc_feature (3)..(3): Xaa is Val, Thr, Ala, Ser, Met, Ile or Leu
  misc_feature (4)..(4): Xaa is Phe, Met, Leu, Ile or Val
  misc_feature (6)..(6): Xaa is Ser, Gly, Ala, Thr, Ile, Leu or His
  misc_feature (8)..(8): Xaa is Arg, Met or Gln
  misc_feature (9)..(9): Xaa is Gly, Arg, Ala, Asp, His or Lys
XXXXSXGXX

SEQ ID NO: 104 - protein, 11 aa, Artificial Sequence
  Specific Amino Acid Residues
  misc_feature (1)..(1): Xaa is Leu or Val
  misc_feature (2)..(2): Xaa is Thr, Ile, Val or Leu
  misc_feature (4)..(4): Xaa is Ile, Val or Leu
  misc_feature (5)..(5): Xaa is Gly or Ser
  misc_feature (9)..(9): Xaa is Pro, Ala or Arg
  misc_feature (10)..(10): Xaa is Thr, Val or Ser
XXRXXHMGXXA

SEQ ID NO: 105 - protein, 10 aa, Artificial Sequence
  Specific Amino Acid Residues
  misc_feature (2)..(2): Xaa is Ile, Thr, Ser, Phe, Asn or Val
  misc_feature (4)..(4): Xaa is Asn, Met, Ala, Val, Leu, Thr or Asp
  misc_feature (5)..(5): Xaa is His, Asn, Leu or Gln
  misc_feature (7)..(7): Xaa is Tyr, Asn or Phe
  misc_feature (9)..(9): Xaa is Val or Ile
  misc_feature (10)..(10): Xaa is Gly or Ala
EXKXXEXRXX

SEQ ID NO: 106 - protein, 9 aa, Artificial Sequence
  Specific Amino Acid Residues
  misc_feature (1)..(1): Xaa is Met or Leu
  misc_feature (2)..(2): Xaa is Ile, Leu or Val
  misc_feature (3)..(3): Xaa is Val, Leu, Ile or Met
  misc_feature (9)..(9): Xaa is Gln, Leu, Val, Asn or Ile
XXXKVKEPX

SEQ ID NO: 107 - protein, 8 aa, Artificial Sequence
  Specific Amino Acid Residues
  misc_feature (2)..(2): Xaa is Phe or Tyr
LXTYLHLA

SEQ ID NO: 108 - protein, 9 aa, Artificial Sequence
  Specific Amino Acid Residues
  misc_feature (1)..(1): Xaa is Val or Ala
  misc_feature (3)..(3): Xaa is Val or Ile
  misc_feature (5)..(5): Xaa is Ile or Val
XDXAXDQGG

SEQ ID NO: 109 - protein, 11 aa, Artificial Sequence
  Specific Amino Acid Residues
  misc_feature (1)..(1): Xaa is Phe, Leu, Ile, Met, Tyr or Val
  misc_feature (2)..(2): Xaa is Phe, Ile, Tyr, Leu, Trp or Val
  misc_feature (4)..(4): Xaa is Val, Cys, Ile, Ala, Met, Ser, Gly or Leu
  misc_feature (5)..(5): Xaa is Gly or Ala
  misc_feature (7)..(7): Xaa is Phe or Leu
  misc_feature (10)..(10): Xaa is Pro, Lys, Arg, Gly, Glu, Thr, Ala, Asn or Ser
  misc_feature (11)..(11): Xaa is Asp, Asn, His, Lys, Glu, Pro, Leu or Ile
XXNXXEXYGXX

SEQ ID NO: 110 - protein, 11 aa, Artificial Sequence
  Specific Amino Acid Residues
  misc_feature (1)..(1): Xaa is Arg, Lys, Ala, Ser or Asn
  misc_feature (2)..(2): Xaa is Lys, Ser, Glu, Gln, Asp or Ala
  misc_feature (3)..(3): Xaa is Asp, His, Asn, Tyr, Glu, Lys, Cys, Gln or Arg
  misc_feature (4)..(4): Xaa is Val, Thr, Ile, Met or Leu
  misc_feature (5)..(5): Xaa is Val, Ile, Leu, Phe, Met or Thr
  misc_feature (6)..(6): Xaa is Ile, Val, Phe or Leu
  misc_feature (7)..(7): Xaa is Ser, Thr, Asn, Cys or Met
  misc_feature (8)..(8): Xaa is Cys, Val, Ala, Ile, Trp or Phe
  misc_feature (11)..(11): Xaa is Gly, Ala, Ser or Cys
XXXXXXXXKGX

SEQ ID NO: 111 - protein, 19 aa, Artificial Sequence
  Specific Amino Acid Residues
  misc_feature (1)..(1): Xaa is Val, Leu, Ile or Met
  misc_feature (2)..(2): Xaa is Ile, Leu or Val
  misc_feature (3)..(3): Xaa is Leu, Met, Ile or Val
  misc_feature (4)..(4): Xaa is His or Gln
  misc_feature (5)..(5): Xaa is Gly, Cys or Ala
  misc_feature (6)..(6): Xaa is Glu or Asp
  misc_feature (7)..(7): Xaa is Pro or Ala
  misc_feature (8)..(8): Xaa is Val, Ile, Ala or Leu
  misc_feature (9)..(9): Xaa is Leu Met, Pro or Val
  misc_feature (10)..(10): Xaa is Gly or Ala
  misc_feature (11)..(11): Xaa is Leu or Ile
  misc_feature (12)..(12): Xaa is Glu or Gln
  misc_feature (13)..(13): Xaa is Ala or Gly
  misc_feature (14)..(14): Xaa is Ala or Val
  misc_feature (15)..(15): Xaa is Ala or Leu
  misc_feature (16)..(16): Xaa is Ala, Leu, His or Tyr
  misc_feature (17)..(17): Xaa is Ser, Gly or Ala
  misc_feature (18)..(18): Xaa is Leu, Phe, Val or Ala
  misc_feature (19)..(19): Xaa is Ile, Phe, Val or Leu
XXXXXXXXXXXXXXXXXXX

SEQ ID NO: 112 - protein, 21 aa, Artificial Sequence
  Specific Amino Acid Residues
  misc_feature (1)..(1): Xaa is Val, Ile, Leu or Met
  misc_feature (2)..(2): Xaa is Val or Ile
  misc_feature (3)..(3): Xaa is Ser, Ala, Val, Cys or Phe
  misc_feature (4)..(4): Xaa is Val, Ile, Ala, Leu or Thr
  misc_feature (5)..(5): Xaa is Cys or Val
  misc_feature (6)..(6): Xaa is His, Asn or Ala
  misc_feature (7)..(7): Xaa is His or Ser
  misc_feature (8)..(8): Xaa is Asp or Glu
  misc_feature (14)..(14): Xaa is Ile, Val or Leu
  misc_feature (15)..(15): Xaa is Asn or Thr
  misc_feature (16)..(16): Xaa is Pro or Asp
  misc_feature (17)..(17): Xaa is Ile, Val, Leu or Ala
  misc_feature (18)..(18): Xaa is Asp, Asn, Glu, Ala, Gly, Gln, Val, Arg or Pro
  misc_feature (19)..(19): Xaa is Ala, Glu, Gln or Asp
  misc_feature (20)..(20): Xaa is Ile or Leu
  misc_feature (21)..(21): Xaa is Gly or Ala
XXXXXXXXTPSGTXXXXXXXX

SEQ ID NO: 113 - protein, 15 aa, Artificial Sequence
  Specific Amino Acid Residues
  misc_feature (1)..(1): Xaa is Gly, Asp or Ala
  misc_feature (2)..(2): Xaa is Ala, Gly, Lys, Thr, Gln, Arg or Glu
  misc_feature (3)..(3): Xaa is Tyr, Asn, Leu or Phe
  misc_feature (4)..(4): Xaa is Leu, Phe, Met or Val
  misc_feature (5)..(5): Xaa is Ile, Leu or Tyr
  misc_feature (6)..(6): Xaa is Val, Ala or Ile
  misc_feature (8)..(8): Xaa is Ala, Ser or Thr
  misc_feature (11)..(11): Xaa is Ser, Ala or Gly
  misc_feature (12)..(12): Xaa is Phe, Trp or Val
  misc_feature (13)..(13): Xaa is Gly, Ala or Leu
  misc_feature (14)..(14): Xaa is Gly or Ser
  misc_feature (15)..(15): Xaa is Met, Val or Leu
XXXXXXDXVSXXXXX

SEQ ID NO: 114 - protein, 24 aa, Artificial Sequence
  Specific Amino Acid Residues
  misc_feature (1)..(1): Xaa is Ala, Ser, Val or Ile
  misc_feature (2)..(2): Xaa is Asp, Gly or Ala
  misc_feature (3)..(3): Xaa is Ile, Leu, Phe, Val or Met
  misc_feature (4)..(4): Xaa is Tyr, Phe, Leu or Cys
  misc_feature (5)..(5): Xaa is Val or Ile
  misc_feature (6)..(6): Xaa is Thr or Ala
  misc_feature (7)..(7): Xaa is Gly or Ser
  misc_feature (8)..(8): Xaa is Pro, Ser or Ala
  misc_feature (9)..(9): Xaa is Asn, Gly, Ser, Gln or Ala
  misc_feature (12)..(12): Xaa is Leu or Met
  misc_feature (14)..(14): Xaa is Ala, Ser, Cys or Gly
  misc_feature (15)..(15): Xaa is Pro, Thr, Ser or Ala
  misc_feature (17)..(17): Xaa is Gly, Ala or Ser
  misc_feature (18)..(18): Xaa is Leu or Val
  misc_feature (19)..(19): Xaa is Thr, Ser or Ala
  misc_feature (20)..(20): Xaa is Met, Ile, Leu, Val or Phe
  misc_feature (21)..(21): Xaa is Met, Leu, Val, Ala or Ile
  misc_feature (22)..(22): Xaa is Gly, Ala, His or Ser
  misc_feature (23)..(23): Xaa is Val, Ile or Ala
XXXXXXXXXKCXGXXPXXXXXXXS

SEQ ID NO: 115 - protein, 10 aa, Artificial Sequence
  Specific Amino Acid Residues
  misc_feature (1)..(1): Xaa is Tyr, Phe, His or Ser
  misc_feature (2)..(2): Xaa is Gly or Asp
  misc_feature (3)..(3): Xaa is Val or Ile
  misc_feature (4)..(4): Xaa is Val, Thr, Ala, Ser, Met, Ile or Leu
  misc_feature (5)..(5): Xaa is Phe, Met, Leu, Ile or Val
  misc_feature (7)..(7): Xaa is Ser, Gly, Ala, Thr, Ile, Leu or His
  misc_feature (9)..(9): Xaa is Arg, Met or Gln
  misc_feature (10)..(10): Xaa is Gly, Arg, Ala, Asp, His or Lys
XXXXXSXGXX

SEQ ID NO: 116 - protein, 16 aa, Artificial Sequence
  Specific Amino Acid Residues
  misc_feature (1)..(1): Xaa is Leu, Gln, Lys, Ala, Phe, Tyr or Trp
  misc_feature (2)..(2): Xaa is Gly, Asn, His or Asp
  misc_feature (3)..(3): Xaa is Lys, Arg or Asn
  misc_feature (4)..(4): Xaa is Leu or Val
  misc_feature (5)..(5): Xaa is Thr, Ile, Val or Leu
  misc_feature (7)..(7): Xaa is Ile, Val or Leu
  misc_feature (8)..(8): Xaa is Gly or Ser
  misc_feature (12)..(12): Xaa is Pro, Ala or Arg
  misc_feature (13)..(13): Xaa is Thr, Val or Ser
  misc_feature (15)..(15): Xaa is Gln, Arg, Glu, Lys, His, Tyr or Gly
  misc_feature (16)..(16): Xaa is Pro or Gly
XXXXXRXXHMGXXAXX

SEQ ID NO: 117 - protein, 23 aa, Artificial Sequence
  Specific Amino Acid Residues
  misc_feature (2)..(2): Xaa is Val, Ile, Leu or Cys
  misc_feature (4)..(4): Xaa is Thr, Lys or Arg
  misc_feature (6)..(6): Xaa is Ile, Val, Thr, Ser, Phe or Asn
  misc_feature (8)..(8): Xaa is Asn, Met, Ala, Asp, Val, Leu or Thr
  misc_feature (9)..(9): Xaa is His, Asn, Gln or Leu
  misc_feature (11)..(11): Xaa is Tyr, Asn or Phe
  misc_feature (13)..(13): Xaa is Val or Ile
  misc_feature (14)..(14): Xaa is Gly or Ala
  misc_feature (15)..(15): Xaa is Met, Leu or Ile
  misc_feature (16)..(16): Xaa is Val, Thr, Ile or Ser
  misc_feature (18)..(18): Xaa is Ser, Ala, Thr, Gln, His, Asn, Leu or Gly
  misc_feature (19)..(19): Xaa is Ser, Ala, Asn, Gly or Val
  misc_feature (20)..(20): Xaa is Val or Ala
  misc_feature (21)..(21): Xaa is Arg, Lys, Asn, Gln, Ser, Ala, Leu or His
  misc_feature (22)..(22): Xaa is Glu, Gln, Asp, Val or Ala
  misc_feature (23)..(23): Xaa is Leu, Ala, Val, Phe or Tyr
GXPXEXKXXEXRXXXXPXXXXXX

SEQ ID NO: 118 - protein, 16 aa, Artificial Sequence
  Specific Amino Acid Residues
  misc_feature (1)..(1): Xaa is Asp, Glu, Gln or Lys
  misc_feature (2)..(2): Xaa is Met or Leu
  misc_feature (3)..(3): Xaa is Ile, Leu or Val
  misc_feature (4)..(4): Xaa is Val, Leu, Ile or Met
  misc_feature (10)..(10): Xaa is Gln, Leu, Ile, Val or Gln
  misc_feature (11)..(11): Xaa is Ala, Thr, Ser, Pro, Met, Lys, Arg, Gln, Val, Ile or Glu
  misc_feature (12)..(12): Xaa is Val, Ile, Glu, Asn, Gln, Thr, Ala, Asp, His, Met, Asn, Val, Ala, Ser, Ile, Asp, Gly, Trp or Lys
  misc_feature (14)..(14): Xaa is Arg, Cys, Tyr or Trp
  misc_feature (15)..(15): Xaa is Ala, Glu, Arg, Gln, Lys, Thr, Ser, Met, Asn, Val, Pro, Gly, Cys or His
  misc_feature (16)..(16): Xaa is Met, Leu, Lys, Arg, Gln, Glu, Trp, Phe or Tyr
XXXXKVKEPXXXEXXX

SEQ ID NO: 119 - protein, 27 aa, Artificial Sequence
  Specific Amino Acid Residues
  misc_feature (1)..(1): Xaa is Leu or Phe
  misc_feature (2)..(2): Xaa is Arg, Lys, Cys, Ser, His, Gly or Gln
  misc_feature (3)..(3): Xaa is His, Glu, Pro, Ser, Asp, Arg, Lys, Gln or Ala
  misc_feature (4)..(4): Xaa is Asp, Gly, Gln, His, Glu, Ser, Asn or Met
  misc_feature (5)..(5): Xaa is Gln or His
  misc_feature (6)..(6): Xaa is Ile, Leu, Val, Thr, Cys or Ala
  misc_feature (8)..(8): Xaa is Phe or Tyr
  misc_feature (15)..(15): Xaa is Pro or Ala
  misc_feature (16)..(16): Xaa is Asp, Ser, Asn, His or Glu
  misc_feature (17)..(17): Xaa is Leu, Met, Pro, Arg, Val, Gln, Glu or Lys
  misc_feature (18)..(18): Xaa is Pro, Ala, Val, Gln, Lys, Glu, Asp, Thr, Asn, Arg, Ser or Met
  misc_feature (19)..(19): Xaa is Gln, Cys or Leu
  misc_feature (20)..(20): Xaa is Thr, Ala or Val
  misc_feature (21)..(21): Xaa is Glu, Ile, Gln, Ala, Arg, Lys, Thr, Asp or Asn
  misc_feature (22)..(22): Xaa is Glu, Asp, Leu, Ala, Gly, His, Tyr or Ser
  misc_feature (24)..(24): Xaa is Ile, Met, Val, Leu, Thr or Lys
  misc_feature (25)..(25): Xaa is Thr, Lys, Ser, Asp, His, Glu, Ala, Asn, Arg, Gly or Gln
  misc_feature (26)..(26): Xaa is Ser, Gly, Cys, Ala or Lys
  misc_feature (27)..(27): Xaa is Gly, Lys, Arg, Gln or Asp
XXXXXXLXTYLHLAXXXXXXXXLXXXX

SEQ ID NO: 120 - protein, 17 aa, Artificial Sequence
  Specific Amino Acid Residues
  misc_feature (1)..(1): Xaa is Gly, Arg or Ser
  misc_feature (2)..(2): Xaa is Ser, Ala or Gly
  misc_feature (3)..(3): Xaa is Ala or Val
  misc_feature (4)..(4): Xaa is Ile, Leu, Met or Val
  misc_feature (5)..(5): Xaa is Val or Ala
  misc_feature (7)..(7): Xaa is Val or Ile
  misc_feature (9)..(9): Xaa is Ile or Val
  misc_feature (14)..(14): Xaa is Cys or Ile
  misc_feature (15)..(15): Xaa is Val, Ile, Ala, Phe, Ser or Cys
  misc_feature (16)..(16): Xaa is Glu or Ala
  misc_feature (17)..(17): Xaa is Thr or Asp
XXXXXDXAXDQGGXXXX

(19) United States
(12) Patent Application Publication
(10) Pub. No.: US 2020/0123581 A1
(43) Pub. Date: Apr. 23, 2020

(54) RECOMBINANT MICROORGANISM, METHOD OF PRODUCING PYRIDOXAMINE OR SALT THEREOF USING RECOMBINANT MICROORGANISM, AND METHOD OF PRODUCING PYRIDOXAL OR SALT THEREOF USING RECOMBINANT MICROORGANISM

(71) Applicants: Mitsui Chemicals, Inc., Minato-ku, Tokyo (JP); RENASCIENCE CO., LTD., Chuo-ku, Tokyo (JP)

(72) Inventors: Toshihiro TATENO, Mobara-shi, Chiba (JP); Motoi YOKOTA, Chiba-shi, Chiba (JP); Tomonori HIDESAKI, Kawasaki-shi, Kanagawa (JP); Tadashi ARAKI, Chosei-gun, Chiba (JP); Atsunori SHINDO, Kawasaki-shi, Kanagawa (JP); Yoshiko MATSUMOTO, Mobara-shi, Chiba (JP); Toshio MIYATA, Nagoya-shi, Aichi (JP); Masaru HONJO, Mobara-shi, Chiba (JP)

(73) Assignees: Mitsui Chemicals, Inc., Minato-ku, Tokyo (JP); RENASCIENCE CO., LTD, Chuo-ku, Tokyo (JP)

(21) Appl. No.: 16/611,053

(22) PCT Filed: May 11, 2018
(86) PCT No.: PCT/JP2018/018413

§ 371 Date: Nov. 5, 2019

(30) Foreign Application Priority Data
May 12, 2017 (JP) 2017-095571
May 12, 2017 (JP) 2017-095573

(51) Int. Cl. C12P 17/12; C12N 9/04; C12N 9/10; C12N 9/06

(52) U.S. Cl. CPC C12P 17/12; C12N 9/0006; C12N 9/1096; C12Y 101/01065; C12Y 104/01002; C12N 9/0016; C12Y 104/01001; C12Y 206/01031; C12Y 206/01001; C12Y 206/0103

(57) ABSTRACT

A recombinant microorganism includes a gene encoding a pyridoxine dehydrogenase, a gene encoding a pyridoxamine synthetase having an enzymatic activity of synthesizing pyridoxamine from pyridoxal, and a gene encoding an amino acid regeneration enzyme having an enzymatic activity of regenerating an amino acid consumed by the pyridoxamine synthetase, in which at least two of the gene encoding the pyridoxine dehydrogenase, the gene encoding the pyridoxamine synthetase, or the gene encoding the amino acid regeneration enzyme, are introduced from outside of a bacterial cell, or are endogenous to the bacterial cell and have an enhanced expression. In addition, a recombinant microorganism into which a gene encoding a pyridoxine dehydrogenase is introduced is provided.

### TECHNICAL FIELD

The present disclosure relates to a recombinant microorganism that can produce pyridoxamine or a salt thereof, a method of producing pyridoxamine or a salt thereof using the recombinant microorganism, a recombinant microorganism that can produce pyridoxal or a salt thereof, and a method of producing pyridoxal or a salt thereof using the recombinant microorganism.

Pyridoxamine and a salt thereof belong to vitamin B₆ family, and are known to have a glycation reaction inhibitory action. Pyridoxamine and a salt thereof, for example, inhibit accumulation of advanced glycation end products (AGEs) in the body which is involved in various aging processes. AGE is a generic term for substances produced by glycation of proteins, and AGE accumulation is believed to aggravate a disease such as diabetes mellitus, atherosclerosis, chronic renal failure, or Alzheimer's dementia. For this reason, pyridoxamine and a salt thereof are promising substances that are expected to make it possible to prevent or treat such a disease by preventing AGE accumulation.

Pyridoxamine and a salt thereof are also known to have activity as a schizophrenia drug, and various studies for their practical use have been made. In addition, development of health foods and cosmetics utilizing various physiological activities of pyridoxamine and a salt thereof is also in progress.

Pyridoxamine and a salt thereof can be synthesized chemically. For example, International Publication No. WO 2006/066806 discloses a method of chemically synthesizing pyridoxamine dihydrochloride using alanine and formic acid as starting materials. WO 2005/077902 discloses a method of chemically synthesizing pyridoxamine from pyridoxine.

On the other hand, biological synthesis of pyridoxamine is also being studied. WO 2007/142222 discloses a method of obtaining pyridoxamine from pyridoxal using a specific microorganism such as Achromobacter. WO 2007/142222 also discloses an experiment in which, by culturing Acremonium fusidioides in the presence of pyridoxine, a certain amount of the pyridoxine was converted to pyridoxal.

Japanese Patent Application Laid-Open (JP-A) No. H09-107985 discloses a method of producing vitamin B₆, in which a microorganism belonging to the genus Rhizobium and capable of producing vitamin B₆ is cultured under aerobic conditions in a culture medium, and produced vitamin B₆ is obtained from a culture solution.

As a study on vitamin B₆ synthesis, WO 2004/035010 discloses a method of producing vitamin B₆ by culturing an organism in which at least one of the activity of yaaD or yaaE of Bacillus subtilis is enhanced compared to the parent organism. Japanese Patent Application Laid-Open (JP-A) No. 2000-23690 describes a production of a vitamin B₆ mixture by using cell-free extract derived from Rhizobium meliloti IFO 14782. Journal of Molecular Catalysis B: Enzymatic, 2010, vol. 67, p. 104-110 discloses a pyridoxamine-pyruvate aminotransferase (PPAT) that is enabled to use L-glutamate by modifying its sequence. It is described that pyridoxamine was able to be produced by incubating E. coli expressing PPAT having such an modified sequence in the presence of a saturated amount of pyridoxal.

Pyridoxal is also one form of vitamin B₆, and a nutrient rcred for the survival of organisms. Vitamin B₆ is not only used in food and food additives but also has wide applications such as raw materials for pharmaceutical products and cosmetics. Pyridoxal has a useful application as a single component. For example, pyridoxal phosphate is used as an active vitamin B₆ agent, and pyridoxal is a material that can be an intermediate product for producing pyridoxal phosphate. Pyridoxal is useful for pharmaceutical products, cosmetics, food, or intermediate products for producing the pharmaceutical products, the cosmetics, and the food. Japanese Patent Publication (JP-B) No. S43-5712 discloses a method of producing a pyridoxal-carbonyl derivative, the method including adding a carbonyl reagent into a reaction liquid in a converting enzyme reaction from pyridoxine to pyridoxal by pyridoxine oxidase. The Journal of Biological Chemistry, 1999, Vol. 274, No. 33, Issue of August 13, pp. 23185-23190 also describes such a converting enzyme.

### SUMMARY OF INVENTION

### Technical Problem

However, high reaction yields have not been achieved by pyridoxamine synthesis by chemical synthesis. For example, in a method described in WO 2006/066806, pyridoxamine dihydrochloride is synthesized through many chemical reactions, which results in lower reaction yields. In a method described in WO 2005/077902, a dimer and a trimer of pyridoxamine are produced, and the reaction yield is not significantly high.

On the other hand, in a method described in WO 2007/142222, in which a specific kind of microorganism is used, the conversion efficiency from pyridoxal to pyridoxamine is varied widely among microbial species and is not significantly high in all. In a culture experiment of Acremonium fusidioides in the presence of pyridoxine described in WO 2007/142222, most of a product is pyridoxal rather than pyridoxamine. JP-A No. H09-107985 describes that most of produced vitamin B₆ was pyridoxol (pyridoxine), and production of pyridoxamine was slight.

WO 2004/035010 describes that vitamin B₆ is obtained by culturing in a culture medium a microorganism highly expressing the genes for yaaD and yaaE; however, the product is a mixture of pyridoxine, pyridoxal, pyridoxamine, pyridoxamine phosphate, and the like and pyridoxamine is not selectively obtained.

As described above, chemical synthesis of pyridoxamine or a salt thereof (such as WO 2006/066806 or WO 2005/077902) involves many reaction and purification steps, and high yields have not been achieved. Although some microorganisms have pyridoxamine-synthesizing ability (for example, WO 2007/142222 and JP-A No. H09-107985), it takes time and effort to newly discover such microorganisms. In addition, selective synthesis of pyridoxamine or a salt thereof among the vitamin B₆ family has not been realized and high pyridoxamine production efficiency has not been achieved. The approach of screening specific microbial species from naturally occurring microbial species as described above does not provide molecular biological information as to which enzyme or gene is important for a high production of vitamin B₆.

For example, a method of producing vitamin B₆ using recombinant microorganisms produced by genetic modification technology is described (WO 2004/035010 and Journal of Molecular Catalysis B: Enzymatic, 2010, vol. 67, p. 104-110). However, in production of substances, vitamin B₆ is non-selectively obtained using a common culture medium (WO 2004/035010), or alternatively, a saturated amount of pyridoxal, which is an expensive raw material, is used to produce pyridoxamine (Journal of Molecular Catalysis B: Enzymatic, 2010, vol. 67, p. 104-110). Therefore, inexpensive and selective production of pyridoxamine or a salt thereof has not been achieved.

In view of the above, according to a first aspect of the disclosure, a recombinant microorganism capable of inexpensively producing pyridoxamine or a salt thereof from pyridoxine or a salt thereof at high production efficiency, and a method of inexpensively producing pyridoxamine or a salt thereof from pyridoxine or a salt thereof at high production efficiency using the recombinant microorganism are provided.

An efficient enzymatic production method that can be used in industrial production has not yet been put to practical use in the production of pyridoxal. In the method described in WO 2004/035010, and the method described in JP-A No. 2000-23690, a mixture of pyridoxine, pyridoxal, and pyridoxamine is obtained by an enzymatic method; however, these methods are not methods in which pyridoxal can be selectively obtained, i.e., can be obtained as pyridoxal with high purity rather than as a mixture of pyridoxine and pyridoxamine.

It is essential to add a carbonyl reagent to a reaction system in the method described in JP-B No. S43-5712. The carbonyl reagent converts produced pyridoxal into a carbonyl derivative. However, a method in which a carbonyl derivative is produced is not often suitable for subjecting pyridoxal as an intermediate product to a subsequent reaction.

From the viewpoint of industrial production of pyridoxal, the present inventors attempted production of an industrially available recombinant microorganism into which a gene encoding a pyridoxine dehydrogenase is introduced. A host cell for such a genetically modified microorganism is preferably a prokaryote from the viewpoint of a cost and the easiness of production. However, for example, in a case in which the introduced gene is a eukaryotic gene, there are many difficulties in introducing, into a prokaryote, a gene encoding a protein that is expressed in a eukaryote, and in expressing the gene, and it is often impossible to express the protein maintaining activity. Thus, a second aspect of the disclosure is to provide a recombinant microorganism that includes and expresses an introduced pyridoxine dehydrogenase.

### Solution to Problem

A first aspect of the disclosure includes the following.

<1>

A recombinant microorganism, including:

a gene encoding a pyridoxine dehydrogenase, a gene encoding a pyridoxamine synthetase having an enzymatic activity of synthesizing pyridoxamine from pyridoxal, and a gene encoding an amino acid regeneration enzyme having an enzymatic activity of regenerating an amino acid consumed by the pyridoxamine synthetase,

wherein at least two of the gene encoding the pyridoxine dehydrogenase, the gene encoding the pyridoxamine synthetase, or the gene encoding the amino acid regeneration enzyme, are introduced from outside of a bacterial cell, or are endogenous to the bacterial cell and have an enhanced expression.

<2>

The recombinant microorganism according to <1>, wherein the pyridoxamine synthetase is a pyridoxamine-pyruvate transaminase, a pyridoxamine-oxaloacetate transaminase, an aspartate transaminase, or a pyridoxamine phosphate transaminase.

<3>

The recombinant microorganism according to <1> or <2>, wherein the pyridoxine dehydrogenase includes at least one of the following partial amino acid sequence (a) or partial amino acid sequence (b), and has pyridoxine dehydrogenase activity:

(a)
(SEQ ID NO: 97)
NX₁X₂EX₃YG

wherein X₁ represents V, C, I, A, M, S, or L,

X₂ represents G or A, and

X₃ represents F or L; and

(b)
(SEQ ID NO: 98)
X₄X₅X₆KGX₇

wherein X₄ represents I, V, F, or L,

X₅ represents S, T, N, C, or M,

X₆ represents C, V, A, I, W, or F, and

X₇ represents G, A, S, or C.

<4>

The recombinant microorganism according to any one of <1> to <3>, wherein the pyridoxine dehydrogenase is represented by enzyme number EC1.1.1.65.

<5>

The recombinant microorganism according to any one of <1> to <4>, wherein the gene encoding the pyridoxine dehydrogenase is derived from Saccharomyces cerevisiae.

<6>

The recombinant microorganism according to any one of <1> to <5>, wherein the gene encoding the pyridoxine dehydrogenase has:

a nucleotide sequence of any one of SEQ ID NO:7 or SEQ ID NO:53 to SEQ ID NO:59,

a region between an 18th nucleotide and a 3′ end in a nucleotide sequence of any one of SEQ ID NO:13 or SEQ ID NO:75 to SEQ ID NO:81, or

a nucleotide sequence that hybridizes with DNA having a nucleotide sequence complementary to a nucleotide sequence of any one of SEQ ID NO:7 or SEQ ID NO:53 to SEQ ID NO:59, or with DNA having a nucleotide sequence complementary to a region between an 18th nucleotide and a 3′ end in a nucleotide sequence of any one of SEQ ID NO:13 or SEQ ID NO:75 to SEQ ID NO:81 under a stringent condition, and that encodes a protein having pyridoxine dehydrogenase activity.

<7>

The recombinant microorganism according to any one of <1> to <6>, wherein the gene encoding the pyridoxine dehydrogenase has a nucleotide sequence of SEQ ID NO:7, or has a nucleotide sequence that hybridizes with DNA having a nucleotide sequence complementary to the nucleotide sequence of SEQ ID NO:7 under a stringent condition, and that encodes a protein having pyridoxine dehydrogenase activity.

<8>

The recombinant microorganism according to any one of <1> to <4>, wherein the gene encoding the pyridoxine dehydrogenase is derived from Schizosaccharomyces pombe

<9>

The recombinant microorganism according to any one of <1> to <4> and <8>, wherein the gene encoding the pyridoxine dehydrogenase has a nucleotide sequence of SEQ ID NO:8, or has a nucleotide sequence that hybridizes with DNA having a nucleotide sequence complementary to the nucleotide sequence of SEQ ID NO:8 under a stringent condition, and that encodes a protein having pyridoxine dehydrogenase activity.

<10>

The recombinant microorganism according to any one of <1> to <9>, wherein the gene encoding the pyridoxine dehydrogenase has a nucleotide sequence encoding an amino acid sequence of any one of SEQ ID NO:1, SEQ ID NO:2, or SEQ ID NO:22 to SEQ ID NO:28, or an amino acid sequence that has 80% or more sequence identity with at least one amino acid sequence selected from the group consisting of SEQ ID NO:1, SEQ ID NO:2, and SEQ ID NO:22 to SEQ ID NO:28, and that has pyridoxine dehydrogenase activity.

<11>

The recombinant microorganism according to any one of <1> to <10>, wherein the gene encoding the pyridoxine dehydrogenase has a nucleotide sequence encoding an amino acid sequence of SEQ ID NO:1 or SEQ ID NO:2, or an amino acid sequence that has 80% or more sequence identity with at least one of the amino acid sequence of SEQ ID NO:1 or the amino acid sequence of SEQ ID NO:2, and that has pyridoxine dehydrogenase activity.

<12>

The recombinant microorganism according to any one of <1> to <11>, wherein the pyridoxamine synthetase includes at least one of the following partial amino acid sequence (c), partial amino acid sequence (d), partial amino acid sequence (e), partial amino acid sequence (f), partial amino acid sequence (g), or partial amino acid sequence (h), and has an enzymatic activity of synthesizing pyridoxamine from pyridoxal:

(c)
(SEQ ID NO: 99)
X₈X₉X₁₀X₁₁X₁₂X₁₃

wherein X₈ represents L, M, I, or V,

X₉ represents H or Q,

X₁₀ represents G, C, or A,

X₁₁ represents E or D,

X₁₂ represents P or A, and

X₁₃ represents V, I, L, or A;

(d)
(SEQ ID NO: 100)
X₁₄X₁₅TPSGTX₁₆X₁₇

wherein X₁₄ represents H or S,

X₁₅ represents D or E,

X₁₆ represents I, V, or L, and

X₁₇ represents N or T;

(e)
(SEQ ID NO: 101)
X₁₈DX₁₉VSX₂₀X₂₁

wherein X₁₈ represents V, I, or A,

X₁₉ represents A, T, or S,

X₂₀ represents S, A, or and

X₂₁ represents F, W, or V;

(f)
(SEQ ID NO: 102)
X₂₂X₂₃X₂₄KCX₂₅GX₂₆X₂₇P

wherein X₂₂ represents G or S,

X₂₃ represents P, S, or A,

X₂₄ represents N, S, A, or Q,

X₂₅ represents L or M,

X₂₆ represents A, S, C, or and

X₂₇ represents P, T, S, or A;

(g)
(SEQ ID NO: 103)
X₂₈X₂₉X₃₀X₃₁SX₃₂GX₃₃X₃₄

wherein X₂₈ represents G or D,

X₂₉ represents V or I,

X₃₀ represents V, T, A, S, M, I, or L,

X₃₁ represents F, M, L, I, or V,

X₃₂ represents S, A, T, I, L, or H,

X₃₃ represents R, M, or Q, and

X₃₄ represents R, A, D, H, or K; and

(h)
(SEQ ID NO: 104)
X₃₅X₃₆RX₃₇X₃₈HMGX₃₉X₄₀A

wherein X₃₅ represents L or V,

X₃₆ represents T, I, V, or L,

X₃₇ represents I, V, or L,

X₃₈ represents G or S,

X₃₉ represents P, A, or R, and

X₄₀ represents T, V, or S.

<13>

The recombinant microorganism according to any one of <1> to <12>, wherein the pyridoxamine synthetase is represented by enzyme number EC2.6.1.30.

<14>

The recombinant microorganism according to any one of <1> to <13>, wherein the gene encoding the pyridoxamine synthetase is derived from Mesorhizobium loti.

<15>

The recombinant microorganism according to any one of <1> to <14>, wherein the gene encoding the pyridoxamine synthetase has:

a nucleotide sequence of any one of SEQ ID NO:9 or SEQ ID NO:60 to SEQ ID NO:66,

a region between an 18th nucleotide and a 3′ end in a nucleotide sequence of SEQ ID NO:14, or a region between an 18th nucleotide and a 3′ end in a nucleotide sequence of any one of SEQ ID NO:82 to SEQ ID NO:88, or

a nucleotide sequence that hybridizes with DNA having a nucleotide sequence complementary to a nucleotide sequence of any one of SEQ ID NO:9 or SEQ ID NO:60 to SEQ ID NO:66, or with DNA having a nucleotide sequence complementary to the region between an 18th nucleotide and a 3′ end in the nucleotide sequence of SEQ ID NO:14, or a region between an 18th nucleotide and a 3′ end in a nucleotide sequence of any one of SEQ ID NO:82 to SEQ ID NO:88, under a stringent condition, and that encodes a protein having an enzymatic activity of synthesizing pyridoxamine from pyridoxal.

<16>

The recombinant microorganism according to any one of <1> to <15>, wherein the gene encoding the pyridoxamine synthetase has a nucleotide sequence of SEQ ID NO:9, or has a nucleotide sequence that hybridizes with DNA having a nucleotide sequence complementary to the nucleotide sequence of SEQ ID NO:9 under a stringent condition, and that encodes a protein having an enzymatic activity of synthesizing pyridoxamine from pyridoxal.

<17>

The recombinant microorganism according to any one of <1> to <16>, wherein the gene encoding the pyridoxamine synthetase has a nucleotide sequence encoding an amino acid sequence of any one of SEQ ID NO:3 or SEQ ID NO:29 to SEQ ID NO:35, or an amino acid sequence that has 80% or more sequence identity with at least one amino acid sequence selected from the group consisting of SEQ ID NO:3 and SEQ ID NO:29 to SEQ ID NO:35, and that has an enzymatic activity of synthesizing pyridoxamine from pyridoxal.

<18>

The recombinant microorganism according to any one of <1> to <17>, wherein the gene encoding the pyridoxamine synthetase has a nucleotide sequence encoding an amino acid sequence of SEQ ID NO:3, or an amino acid sequence that has 80% or more sequence identity with the amino acid sequence of SEQ ID NO:3, and that has an enzymatic activity of synthesizing pyridoxamine from pyridoxal.

<19>

The recombinant microorganism according to any one of <12> to <18>, wherein the amino acid regeneration enzyme is an alanine dehydrogenase.

<20>

The recombinant microorganism according to <19>, wherein the alanine dehydrogenase is enabled to use NADPH as a coenzyme in a reaction in which L-alanine is produced from pyruvic acid and NH₃.

<21>

The recombinant microorganism according to <19> or <20>, wherein the alanine dehydrogenase includes at least one of the following partial amino acid sequence (i), partial amino acid sequence (j), partial amino acid sequence (k), or partial amino acid sequence (1), and has an activity of producing L-alanine from pyruvic acid and NH₃:

(i)
(SEQ ID NO: 105)
EX₄₁KX₄₂X₄₃EX₄₄RX₄₅X₄₆

wherein X₄₁ represents I, T, S, F, N, or V,

X₄₂ represents N, M, A, V, L, T, or D,

X₄₃ represents H, N, L, or Q,

X₄₄ represents Y, N, or F,

X₄₅ represents V or I, and

X₄₆ represents G or A;

(j)
(SEQ ID NO: 106)
X₄₇X₄₈X₄₉KVKEPX₅₀

wherein X₄₇ represents M or L,

X₄₈ represents I, L, or V,

X₄₉ represents V, L, I, or M, and

X₅₀ represents Q, L, V, N, or I;

(k)
(SEQ ID NO: 107)
LX₅₁TYLHLA

wherein X₅₁ represents F or Y; and

(l)
(SEQ ID NO: 108)
X₅₂DX₅₃AX₅₄DQGG

wherein X₅₂ represents V or A,

X₅₃ represents V or I, and

X₅₄ represents I or V.

<22>

The recombinant microorganism according to any one of <19> to <21>, wherein the gene encoding the amino acid regeneration enzyme is derived from Shewanella sp. AC10.

<23>

The recombinant microorganism according to any one of <19> to <22>, wherein the gene encoding the amino acid regeneration enzyme has:

a nucleotide sequence of any one of SEQ ID NO:11 or SEQ ID NO:67 to SEQ ID NO:74,

a region between an 18th nucleotide and a 3′ end in a nucleotide sequence of any one of SEQ ID NO:15 or SEQ ID NO:89 to SEQ ID NO:96, or a nucleotide sequence that hybridizes with DNA having a nucleotide sequence complementary to a nucleotide sequence of any one of SEQ ID NO:11 or SEQ ID NO:67 to SEQ ID NO:74, or with DNA having a nucleotide sequence complementary to a region between an 18th nucleotide and a 3′ end in a nucleotide sequence of any one of SEQ ID NO:15 or SEQ ID NO:89 to SEQ ID NO:96 under a stringent condition, and that encodes a protein having alanine regeneration enzymatic activity.

<24>

The recombinant microorganism according to any one of <19> to <23>, wherein the gene encoding the amino acid regeneration enzyme has a nucleotide sequence of SEQ ID NO:11, or has a nucleotide sequence that hybridizes with DNA having a nucleotide sequence complementary to the nucleotide sequence of SEQ ID NO:11 under a stringent condition, and that encodes a protein having alanine regeneration enzymatic activity.

<25>

The recombinant microorganism according to any one of <19> to <24>, wherein the gene encoding the amino acid regeneration enzyme has a nucleotide sequence encoding an amino acid sequence of any one of SEQ ID: NO 5 or SEQ ID NO:45 to SEQ ID NO:52, or an amino acid sequence that has 80% or more sequence identity with at least one amino acid sequence selected from the group consisting of SEQ ID NO:5 and SEQ ID NO:45 to SEQ ID NO:52, and that has alanine regeneration enzymatic activity.

<26>

The recombinant microorganism according to any one of <19> to <25>, wherein the gene encoding the amino acid regeneration enzyme has a nucleotide sequence encoding an amino acid sequence of SEQ ID NO:5, or an amino acid sequence that has 80% or more sequence identity with the amino acid sequence of SEQ ID NO:5, and that has alanine regeneration enzymatic activity.

<27>

The recombinant microorganism according to any one of <1> to <11>, wherein the pyridoxamine synthetase is represented by enzyme number EC2.6.1.31 or EC2.6.1.1.

<28>

The recombinant microorganism according to any one of <1> to <11> or <27>, wherein the gene encoding the pyridoxamine synthetase is derived from Escherichia coli.

<29>

The recombinant microorganism according to any one of <1> to <11>, <27>, or <28>, wherein the gene encoding the pyridoxamine synthetase has a nucleotide sequence of SEQ ID NO:10, or has a nucleotide sequence that hybridizes with DNA having a nucleotide sequence complementary to the nucleotide sequence of SEQ ID NO:10 under a stringent condition, and that encodes a protein having an enzymatic activity of synthesizing pyridoxamine from pyridoxal.

<30>

The recombinant microorganism according to any one of <1> to <11>, or <27> to <29>, wherein the gene encoding the pyridoxamine synthetase has a nucleotide sequence encoding an amino acid sequence of SEQ ID NO:4, or an amino acid sequence that has 80% or more sequence identity with the amino acid sequence of SEQ ID NO:4, and that has an enzymatic activity of synthesizing pyridoxamine from pyridoxal.

<31>

The recombinant microorganism according to any one of <27> to <30>, wherein the amino acid regeneration enzyme is a glutamate dehydrogenase.

<32>

The recombinant microorganism according to <31>, wherein the gene encoding the amino acid regeneration enzyme is derived from Escherichia coli.

<33>

The recombinant microorganism according to <31> or <32>, wherein the gene encoding the amino acid regeneration enzyme has a nucleotide sequence of SEQ ID NO:12, or has a nucleotide sequence that hybridizes with DNA having a nucleotide sequence complementary to the nucleotide sequence of SEQ ID NO:12 under a stringent condition, and that encodes a protein having glutamate regeneration enzymatic activity.

<34>

The recombinant microorganism according to any one of <31> to <33>, wherein the gene encoding the amino acid regeneration enzyme has a nucleotide sequence encoding an amino acid sequence of SEQ ID NO:6, or an amino acid sequence that has 80% or more sequence identity with the amino acid sequence of SEQ ID NO:6, and that has glutamate regeneration enzymatic activity.

<35>

The recombinant microorganism according to any one of <1> to <34>, the recombinant microorganism being recombinant E. coli.

<36>

A method of producing pyridoxamine or a salt thereof, the method including bringing the recombinant microorganism according to any one of <1> to <35>, a culture of the recombinant microorganism, or a treated product of the recombinant microorganism or the culture, into contact with pyridoxine or a salt thereof to produce pyridoxamine or a salt thereof.

<37>

The production method according to <36>, wherein the recombinant microorganism, the culture of the recombinant microorganism, or the treated product of the recombinant microorganism or the culture, includes the pyridoxine dehydrogenase, the pyridoxamine synthetase, and the amino acid regeneration enzyme.

<38>

The production method according to <36> or <37>, wherein the treated product of the recombinant microorganism or the treated product of the culture of the recombinant microorganism is treated by treatment including one or more selected from the group consisting of heat treatment, cooling treatment, mechanical destruction of a cell, ultrasonic treatment, freeze-thaw treatment, drying treatment, pressurized or reduced pressure treatment, osmotic pressure treatment, cell autolysis, surfactant treatment, enzyme treatment, cell separation treatment, purification treatment, and extraction treatment.

A second aspect of the disclosure includes the following.

<39>

A recombinant microorganism, into which at least one polynucleotide selected from the group consisting of the following (1) to (7) is introduced:

(1) a polynucleotide that encodes a protein having an amino acid sequence of SEQ ID NO:1;

(2) a polynucleotide that encodes a protein having an amino acid sequence in which from 1 to 50 amino acids are deleted, added, or substituted in the amino acid sequence of SEQ ID NO:1, the protein having an activity of synthesizing pyridoxine or a salt thereof, or pyridoxal or a salt thereof;

(3) a polynucleotide that encodes a protein having an amino acid sequence having 60% or more sequence identity with the amino acid sequence of SEQ ID NO:1, the protein having an activity of synthesizing pyridoxine or a salt thereof, or pyridoxal or a salt thereof;

(4) a polynucleotide having a nucleotide sequence of SEQ ID NO:13;

(5) a polynucleotide having a nucleotide sequence in which from 1 to 150 nucleotides are deleted, added, or substituted in the nucleotide sequence of SEQ ID NO:13, the polynucleotide encoding a protein having an activity of synthesizing pyridoxine or a salt thereof, or pyridoxal or a salt thereof;

(6) a polynucleotide having a nucleotide sequence having 60% or more sequence identity with the nucleotide sequence of SEQ ID NO:13, the polynucleotide encoding a protein having an activity of synthesizing pyridoxine or a salt thereof, or pyridoxal or a salt thereof; and

(7) a polynucleotide that hybridizes with a polynucleotide having a nucleotide sequence complementary to the nucleotide sequence of SEQ ID NO:13 under a stringent condition, the polynucleotide encoding a protein having an activity of synthesizing pyridoxine or a salt thereof, or pyridoxal or a salt thereof.

<40>

The recombinant microorganism according to <39>, wherein the recombinant microorganism is a recombinant bacterium.

<41>

The recombinant microorganism according to <40>, wherein the recombinant microorganism is recombinant E. coli.

<42>

The recombinant microorganism according to any one of <39> to <41>, wherein the protein is an enzyme classified under EC number 1.1.1.65.

<43>

The recombinant microorganism according to any one of <39> to <42>, wherein the protein is a pyridoxine dehydrogenase derived from Saccharomyces cerevisiae.

<44>

A method of producing pyridoxal or a salt thereof, the method including bringing the recombinant microorganism according to any one of <39> to <43> into contact with pyridoxine or a salt thereof.

<45>

A method of producing pyridoxal or a salt thereof, the method including bringing a culture obtained by culturing the recombinant microorganism according to any one of <39> to <43>, or a treated product of the culture, into contact with pyridoxine or a salt thereof.

### Advantageous Effects of Invention

In accordance with the first aspect of the disclosure, a recombinant microorganism capable of inexpensively producing pyridoxamine or a salt thereof from pyridoxine or a salt thereof at high production efficiency, and a method of inexpensively producing pyridoxamine or a salt thereof from pyridoxine or a salt thereof at high production efficiency using the recombinant microorganism are provided.

In accordance with the second aspect of the disclosure, a recombinant microorganism that includes and expresses an introduced pyridoxine dehydrogenase is provided.

### BRIEF DESCRIPTION OF DRAWINGS

FIG. 1-1 illustrates the alignment of the sequences of SEQ ID NO:1 and SEQ ID NO:22 to SEQ ID NO:28.

FIG. 1-2 illustrates the alignment of the sequences of SEQ ID NO:1 and SEQ ID NO:22 to SEQ ID NO:28.

FIG. 2-1 illustrates the alignment of the sequences of SEQ ID NO:3 and SEQ ID NO:29 to SEQ ID NO:35.

FIG. 2-2 illustrates the alignment of the sequences of SEQ ID NO:3 and SEQ ID NO:29 to SEQ ID NO:35.

FIG. 3-1 illustrates the alignment of the sequences of SEQ ID NO:36 to SEQ ID NO:44.

FIG. 3-2 illustrates the alignment of the sequences of SEQ ID NO:36 to SEQ ID NO:44.

### DESCRIPTION OF EMBODIMENTS

A first aspect of the disclosure (hereinafter, simply referred to as “first aspect”) is to provide a recombinant microorganism (hereinafter referred to as “recombinant microorganism according to first aspect”) including a gene encoding a pyridoxine dehydrogenase, a gene encoding a pyridoxamine synthetase that has an enzymatic activity of synthesizing pyridoxamine from pyridoxal, and a gene encoding an amino acid regeneration enzyme that has an enzymatic activity of regenerating an amino acid consumed by the pyridoxamine synthetase,

wherein at least two of the gene encoding the pyridoxine dehydrogenase, the gene encoding the pyridoxamine synthetase, or the gene encoding the amino acid regeneration enzyme, are introduced from outside of a bacterial cell, or are originally endogenous to the bacterial cell and have an enhanced expression. A second aspect of the disclosure (hereinafter, simply referred to as “second aspect”) is to provide a recombinant microorganism into which (1) a polynucleotide that encodes a protein having an amino acid sequence of SEQ ID NO:1, (2) a polynucleotide that encodes a protein having an amino acid sequence in which from 1 to 50 amino acids are deleted, added, or substituted in the amino acid sequence of SEQ ID NO:1, (3) a polynucleotide that encodes a protein having an amino acid sequence having 60% or more sequence identity with the amino acid sequence of SEQ ID NO:1, (4) a polynucleotide having the nucleotide sequence of SEQ ID NO:13, (5) a polynucleotide having a nucleotide sequence in which from 1 to 150 nucleotides are deleted, added, or substituted in the nucleotide sequence of SEQ ID NO:13, (6) a polynucleotide having a nucleotide sequence having 60% or more sequence identity with the nucleotide sequence of SEQ ID NO:13, or (7) a polynucleotide that hybridizes with a polynucleotide having a nucleotide sequence complementary to the nucleotide sequence of SEQ ID NO:13 under a stringent condition is introduced. A description of “the disclosure” is used so as to be intended to encompass the first aspect and the second aspect. In the disclosure, “introduction” refers to introduction that allows expression in a bacterial cell. The term “recombinant” is used for the same meaning as “gene recombination”.

<First Aspect>

Until now, it has not been known a method for inexpensively producing pyridoxamine or a salt thereof from pyridoxine or a salt thereof at high production efficiency either by a chemical method or a biological method. The structure of pyridoxamine is shown below.

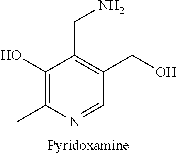

However, the present inventors have surprisingly found that pyridoxamine or a salt thereof can be inexpensively produced from pyridoxine or a salt thereof at high production efficiency by using a recombinant microorganism having above described constitution or a culture of the recombinant microorganism, or a treated product of the recombinant microorganism or the culture. Although the reason for this is not necessarily clear, it is assumed that in a case in which the above three kinds of enzymes exist in combination, due to the cooperative action of the above three kinds of enzymes, the equilibrium of each reaction in a process of producing pyridoxamine or a salt thereof from pyridoxine or a salt thereof and the amount of a by-product or the like produced in this process or the amount of a raw material or the like consumed in this process advantageously affect the production of pyridoxamine or a salt thereof from pyridoxine or a salt thereof. Further, it is assumed that since each of the at least two of the genes encoding the above three kinds of enzymes is introduced from outside of a bacterial cell, or is endogenous to a bacterial cell and has an enhanced expression, the enzymes can be expressed at a high expression level from the introduced or enhanced gene and the production efficiency of pyridoxamine or a salt thereof can be further improved.

Such a cooperative action of the above-described three kinds of enzymes, which has not been found so far, allows the production of pyridoxamine or a salt thereof with high production efficiency by using pyridoxine or a salt thereof, without the need to carry out a multi-step reaction as in a chemical synthesis method. Moreover, by using the recombinant microorganism according to the first aspect, it is possible to avoid by-production of large amounts of other substances included in the vitamin B₆ complex in. Pyridoxine is a raw material which is industrially available at a low cost compared to pyridoxal. By using pyridoxine as a starting material, pyridoxamine or a salt thereof can be inexpensively produced.

<Pyridoxine Dehydrogenase>

The pyridoxine dehydrogenase is an enzyme also called pyridoxal reductase or pyridoxine-4-dehydrogenase. The pyridoxine dehydrogenase may be an enzyme represented by the enzyme number EC1.1.1.65 or may be an enzyme represented by other enzyme number. Pyridoxine dehydrogenase is an enzyme having an enzymatic activity that catalyzes a reaction of converting pyridoxal into pyridoxine by adding hydrogen, and a reaction reverse thereof. Although pyridoxal or pyridoxine may also exist as a salt depending on a surrounding environment; however, in the disclosure, a description of enzymatic activity is given with the omission of the mention of a salt in order to simplify an expression. Pyridoxine dehydrogenase consumes NADH (reduced nicotinamide adenine dinucleotide) or NADPH (reduced nicotinamide adenine dinucleotide phosphate) as coenzymes when hydrogen is added to pyridoxal; and consumes NAD+(oxidized nicotinamide adenine dinucleotide) or NADP+(oxidized nicotinamide adenine dinucleotide phosphate) as a coenzyme when hydrogen is abstracted from pyridoxine. Under general conditions, since the equilibrium of a reaction catalyzed by this enzyme is greatly shifted in favor of the pyridoxine generation, it has been considered that it is impossible to achieve a high yield when pyridoxine dehydrogenase is used in a reaction in which pyridoxine is consumed. In the first aspect of the disclosure, an unexpected effect of achieving inexpensive production of pyridoxamine or a salt thereof from pyridoxine or a salt thereof at high production efficiency can be obtained when pyridoxine dehydrogenase is used in combination with a specific enzyme(s) as described above.

The pyridoxine dehydrogenase is preferably derived from, for example, a microorganism belonging to the division Ascomycota, and may be derived from a microorganism belonging to the subdivision Saccharomycotina or the subdivision Pezizomycotina. More specifically, the pyridoxine dehydrogenase may be, for example, a pyridoxine dehydrogenase (having the amino acid sequence of SEQ ID NO:1) derived from Saccharomyces cerevisiae, a pyridoxine dehydrogenase (having the amino acid sequence of SEQ ID NO:22) derived from Saccharomyces eubayanus, a pyridoxine dehydrogenase (having the amino acid sequence of SEQ ID NO:23) derived from Torulaspora delbrueckii, a pyridoxine dehydrogenase (having the amino acid sequence of SEQ ID NO:24) derived from Zygosaccharomyces bailii, a pyridoxine dehydrogenase (having the amino acid sequence of SEQ ID NO:26) derived from Kluyveromyces marxianus, a pyridoxine dehydrogenase (having the amino acid sequence of SEQ ID NO:25) derived from Aspergillus oryzae, a pyridoxine dehydrogenase (having the amino acid sequence of SEQ ID NO:27) derived from Candida albicans, a pyridoxine dehydrogenase (having the amino acid sequence of SEQ ID NO:28) derived from Yarrowia lipolytica, or the like. Among these microorganisms, Aspergillus oryzae belongs to the subphylum Pezizomycotina, and the others belong to the subphylum Saccharomycotina.

The pyridoxine dehydrogenase of EC1.1.1.65 may be a pyridoxine dehydrogenase derived from, for example, Saccharomyces cerevisiae, Schizosaccharomyces pombe, or the like. Here, the pyridoxine dehydrogenase from Saccharomyces cerevisiae has the amino acid sequence of SEQ ID NO:1, and the pyridoxine dehydrogenase from Schizosaccharomyces pombe has the amino acid sequence of SEQ ID NO:2.

<Pyridoxamine Synthetase>

The pyridoxamine synthetase used in the first aspect of the disclosure refers to any enzyme having an enzymatic activity of synthesizing pyridoxamine from pyridoxal. Pyridoxal or pyridoxamine may also exist as a salt depending on a surrounding environment; however, in the disclosure, a description of enzymatic activity is given with the omission of the mention of a salt in order to simplify an expression. Examples of the pyridoxamine synthetase include, for example, a pyridoxamine-pyruvate transaminase (such as an enzyme represented by the enzyme number EC 2.6.1.30), an aspartate transaminase (such as an enzyme represented by EC 2.6.1.1), a pyridoxamine-oxaloacetate transaminase (such as an enzyme represented by EC 2.6.1.31), and a pyridoxamine phosphate transaminase (such as an enzyme represented by EC 2.6.1.54).

The aspartate transaminase represented by EC 2.6.1.1 is a holoenzyme using pyridoxal phosphate as a coenzyme, and has an enzymatic activity of produce glutamic acid and oxaloacetic acid by transferring the amino group of aspartic acid to 2-oxoglutaric acid. The aspartate transaminase in a state of an apoenzyme not bound to pyridoxal phosphate is known synthesize pyridoxamine by transferring the amino group of glutamic acid or aspartic acid to pyridoxal (Journal of Biological Chemistry, January 1962, Vol. 237, No. 1, p. 127-132). In other words, an apoenzyme form of the aspartate transaminase represented by EC 2.6.1.1 is the pyridoxamine-oxaloacetate transaminase of EC 2.6.1.31. For this reason, aspartate transaminase exists as an apoenzyme when a coenzyme pyridoxal phosphate is absent or present in a small amount, and synthesizes pyridoxamine.

The pyridoxamine synthetase has an enzymatic activity of producing pyridoxamine or a salt thereof by oxidizing an amino group moiety of a specific amino acid to (═O) and transferring the amino group in the case of synthesizing pyridoxamine or a salt thereof from pyridoxal or a salt thereof. For example, the pyridoxamine-pyruvate transaminase can use both L-alanine and D-alanine, each of the pyridoxamine-oxaloacetate transaminase and the aspartate transaminase in an apoenzyme state can use D-aspartic acid, L-aspartic acid, D-glutamic acid, and L-glutamic acid, and the pyridoxamine phosphate transaminase can use D-glutamic acid.

The pyridoxamine-pyruvate transaminase may be derived from, for example, a microorganism belonging to the phylum Proteobacteria, a microorganism belonging to the phylum Actinobacteria, a microorganism belonging to the phylum Spirocheta, or a microorganism belonging to the phylum Filmictes. The pyridoxamine-pyruvate transaminase may be a pyridoxamine-pyruvate transaminase derived from, for example, Mesorhizobium loti, Ochrobactrum anthropi, or the genus Pseudomonas (such as Pseudomonas sp. MA-1). Here, the pyridoxamine-pyruvate transaminase from Mesorhizobium loti has the amino acid sequence of SEQ ID NO:3, for example

The pyridoxamine-pyruvate transaminase may also be, for example, a pyridoxamine-pyruvate transaminase (having the amino acid sequence of SEQ ID NO:29) derived from Mesorhizobium sp. YR577, a pyridoxamine-pyruvate transaminase (having the amino acid sequence of SEQ ID NO:30) derived from Pseudaminobacter salicylatoxidans, a pyridoxamine-pyruvate transaminase (having the amino acid sequence of SEQ ID NO:31) derived from Bauldia litoralis, a pyridoxamine-pyruvate transaminase (having the amino acid sequence of SEQ ID NO:32) derived from Skermanella stibiiresistens, a pyridoxamine-pyruvate transaminase (having the amino acid sequence of SEQ ID NO:33) derived from Rhizobium sp. AC44/96, a pyridoxamine-pyruvate transaminase (having the amino acid sequence of SEQ ID NO:34) derived from Erwinia toletana, or a pyridoxamine-pyruvate transaminase (having the amino acid sequence of SEQ ID NO:35) derived from Herbiconiux ginsengi.

The pyridoxamine-oxaloacetate transaminase may be, for example, a pyridoxamine-oxaloacetate transaminase derived from Escherichia coli, Oryctolagus cuniculus, or Rattus norvegicus. For example, the pyridoxamine-oxaloacetate transaminase from Escherichia coli has the amino acid sequence of SEQ ID NO:4. The aspartate transaminase may be an aspartate transaminase derived from, for example, Escherichia coli, Trichoderma viride, or the like. The pyridoxamine phosphate transaminase may be a pyridoxamine phosphate transaminase derived from, for example, Clostridium butyricum.

<Amino Acid Regeneration Enzyme>

The amino acid regeneration enzyme used in the first aspect refers to any enzyme having an enzymatic activity of regenerating an amino acid consumed by the pyridoxamine synthetase. Pyridoxamine has an amino group, and an amino acid is considered to be consumed as a source providing an amino group in the case of synthesizing pyridoxamine. An amino acid may also exist as a salt depending on a surrounding environment; however, in the disclosure, a description of enzymatic activity is given with the omission of the mention of a salt in order to simplify an expression.

For example, in a case in which the pyridoxamine synthetase consumes L-alanine, an enzyme capable of regenerating L-alanine can be used as the amino acid regeneration enzyme. In a case in which the pyridoxamine synthetase consumes L-glutamic acid or L-aspartic acid, an enzyme capable of regenerating L-glutamic acid or L-aspartic acid can be used as the amino acid regeneration enzyme.

Examples of the amino acid regeneration enzymes include an alanine dehydrogenase (for example, an enzyme represented by enzyme number 1.4.1.1), a glutamate dehydrogenase (for example, an enzyme represented by enzyme number 1.4.1.2, 1.4.1.3, or 1.4.1.4), and a modified alanine dehydrogenase that is enabled to use NADP⁺/NADPH as a coenzyme by modifying an amino acid sequence. In the first aspect, an enzymatic activity of producing L-alanine from pyruvic acid and NH₃ using NADH or NADPH as a coenzyme may be referred to as “alanine regeneration enzymatic activity”, and an enzymatic activity of producing L-glutamic acid from 2-oxoglutaric acid and NH₃ using NADH or NADPH as a coenzyme may be referred to as “glutamic acid regeneration enzymatic activity”.

Examples of preferred combinations of the pyridoxamine synthetase and the amino acid regeneration enzyme include a combination of a pyridoxamine-pyruvate transaminase as the pyridoxamine synthetase and an alanine dehydrogenase as the amino acid regeneration enzyme, and a combination of a pyridoxamine-oxaloacetate transaminase or an aspartate transaminase as the pyridoxamine synthetase and a glutamate dehydrogenase as the amino acid regeneration enzyme. The amino acid regeneration enzyme preferably has an enzymatic activity of regenerating an amino acid consumed by the pyridoxamine synthetase encoded by a gene that is introduced from outside of a bacterial cell, or is endogenous to a bacterial cell and has an enhanced expression.

The amino acid regeneration enzyme may be, for example, an alanine dehydrogenase (having the amino acid sequence of SEQ ID NO:36) derived from Shewanella sp. Ac10, an alanine dehydrogenase (having the amino acid sequence of SEQ ID NO:37) derived from Aeromonas hydrophila, an alanine dehydrogenase (having the amino acid sequence of SEQ ID NO:38) derived from Rhizobium sp. LPU83, an alanine dehydrogenase (having the amino acid sequence of SEQ ID NO:39) derived from Pseudomonas mendocina, an alanine dehydrogenase (having the amino acid sequence of SEQ ID NO:40, or SEQ ID NO:41) derived from Bradyrhizobium japonicum, an alanine dehydrogenase (having the amino acid sequence of SEQ ID NO:42) derived from Streptomyces aureofaciens, an alanine dehydrogenase (having the amino acid sequence of SEQ ID NO:43) derived from Anabaena cylindrica, an alanine dehydrogenase (having the amino acid sequence of SEQ ID NO:44) derived from Bacillus subtilis, or the like. Alternatively, the amino acid regeneration enzyme may be, for example, a glutamate dehydrogenase derived from Escherichia coli or the like. It is preferable that the amino acid regeneration enzyme (for example, an alanine dehydrogenase or a glutamate dehydrogenase) can use NADPH (reduced nicotinamide adenine dinucleotide phosphate) as a coenzyme in a reaction in which an amino acid is regenerated.

For example, the alanine dehydrogenase is preferably an alanine dehydrogenase that can use NADPH as a coenzyme in a reaction in which L-alanine is produced from pyruvic acid and NH₃. In the case of using an amino acid regeneration enzyme that can use NADPH as a coenzyme, a NADPH/NADP+ ratio is varied due to a consumption of NADPH, the equilibrium and rate of a reaction catalyzed by a pyridoxine dehydrogenase are influenced, and the production efficiency of pyridoxamine or a salt thereof can be further enhanced. Most alanine dehydrogenases can use NADH (reduced nicotinamide adenine dinucleotide) as a coenzyme, but are incapable of using NADPH. In the case of using an alanine dehydrogenase that can use NADPH as a coenzyme, a NADPH/NADP⁺ ratio is varied due to a consumption of NADPH, the equilibrium and rate of a reaction catalyzed by a pyridoxine dehydrogenase are influenced, and the production efficiency of pyridoxamine or a salt thereof can be further enhanced, as described above. It is also preferable that the amino acid regeneration enzyme (for example, an alanine dehydrogenase or a glutamate dehydrogenase) can use both NADH (reduced nicotinamide adenine dinucleotide) and NADPH (reduced nicotinamide adenine dinucleotide phosphate) as coenzymes in a reaction in which an amino acid is regenerated. Examples of such alanine dehydrogenases include, but are not limited to, a protein having the amino acid sequence of any one of SEQ ID NO:5 or SEQ ID NO:45 to SEQ ID NO:52 described later.

Each of the pyridoxine dehydrogenase, the pyridoxamine synthetase, and the amino acid regeneration enzyme may be a protein that has a known amino acid sequence (such as an amino acid sequence encoded by a gene naturally occurring in an organism, or an amino acid sequence encoded by a gene possessed by a naturally occurring microorganism such as the above-described microorganism) having the enzymatic activity and that is unmodified, or may be a protein that has an amino acid sequence obtained by modifying such an amino acid sequence as long as the activity of the enzymatic activity (such as the above-described enzymatic activity) is not lost. Examples of the modification include insertion, deletion, substitution of an amino acid residue, and addition of an additional amino acid residue to either or both of the N-terminus and the C-terminus of an amino acid sequence. In a case in which there is one or more of the insertion, deletion, and substitution of the amino acid residue, the number of each of insertion, deletion, and substitution, if present, may be, for example, from 1 to 30 amino acid residues, from 1 to 20 amino acid residues, from 1 to 10 amino acid residues, or from 1 to 5 amino acid residues; and the total number of insertion, deletion, and substitution of the amino acid residue may be, for example, from 1 to 50 amino acid residues, from 1 to 30 amino acid residues, from 1 to 10 amino acid residues, or from 1 to 5 amino acid residues. The number of amino acid residue added to the terminus, if present, may be, for example, from 1 to 50 amino acid residues, from 1 to 30 amino acid residues, from 1 to 10 amino acid residues, or from 1 to 5 amino acid residues per one terminus. The additional amino acid residue may form a signal sequence for extracellular secretion or the like. Examples of the signal sequence include an E. coli OmpA signal sequence.

Alternatively, each of the enzymes may be a protein having a known amino acid sequence (such as an amino acid sequence encoded by a gene naturally occurring in an organism) having the enzymatic activity per se, or a protein having an amino acid sequence that has 80% or more, 85% or more, 90% or more, or 95% or more sequence identity with a known amino acid sequence (such as an amino acid sequence encoded by a gene naturally occurring in an organism) having the enzymatic activity and that has a desired enzymatic activity (such as the above-mentioned enzymatic activity). Here, the sequence identity can be evaluated using, for example, a BLAST (registered trademark, National Library of Medicine) program with default parameters.

For example, the pyridoxine dehydrogenase may be, for example, a protein having the amino acid sequence of SEQ ID NO:1 or SEQ ID NO:2, or may be a protein having an amino acid sequence in which one or more of substitution, deletion, or insertion of an amino acid residue, or addition of an additional amino acid residue to either or both of the N-terminus and C-terminus of the amino acid sequence are performed in the amino acid sequence of SEQ ID NO:1 or SEQ ID NO:2. Examples of the degree of the substitution, deletion, and insertion of an amino acid residue, and the addition of an additional amino acid residue to either or both of the N-terminus and the C-terminus of the amino acid sequence are as described above.

Alternatively, the pyridoxine dehydrogenase may be a protein having the amino acid sequence of SEQ ID NO:1 or SEQ ID NO:2, or may be a protein having an amino acid sequence that has, for example, 80% or more, 85% or more, 90% or more, or 95% or more sequence identity with the amino acid sequence of SEQ ID NO:1 or SEQ ID NO:2.

In a case of using such a protein having an amino acid sequence similar to at least one of the amino acid sequence of SEQ ID NO:1 or SEQ ID NO:2 as described above, it is necessary that the protein has an activity for a pyridoxine dehydrogenase. The pyridoxine dehydrogenase activity can be measured by, for example, adding a protein to be tested to an aqueous solution including pyridoxine as a substrate and a necessary NAD⁺ or NADP⁺, and quantifying the amount of produced pyridoxal by high performance liquid chromatography, or forming a Schiff base between produced pyridoxal and an amine such as trishydroxymethylaminomethane and quantifying the amount of the Schiff base with absorbance measurement at 415 nm or the like.

Alternatively, the pyridoxine dehydrogenase may be a protein having the amino acid sequence of any one of SEQ ID NO:1, SEQ ID NO:2 or SEQ ID NO:22 to SEQ ID NO:28, or a protein having an amino acid sequence that has, for example, 80% or more, 85% or more, 90% or more, or 95% or more sequence identity with at least one of the amino acid sequence of SEQ ID NO:1, the amino acid sequence of SEQ ID NO:2, the amino acid sequence of SEQ ID NO:22, the amino acid sequence of SEQ ID NO:23, the amino acid sequence of SEQ ID NO:24, the amino acid sequence of SEQ ID NO:25, the amino acid sequence of SEQ ID NO:26, the amino acid sequence of SEQ ID NO:27, or the amino acid sequence of SEQ ID NO:28. It is necessary that the protein has an activity for a pyridoxine dehydrogenase.

Alternatively, the pyridoxine dehydrogenase may be a pyridoxine dehydrogenase including at least one of the following partial amino acid sequence (a) or partial amino acid sequence (b), and having pyridoxine dehydrogenase activity:

(a)
(SEQ ID NO: 97)
NX₁X₂EX₃YG

wherein X₁ represents V, C, I, A, M, S, or L,

X₂ represents G or A, and

X₃ represents F or L; and

(b)
(SEQ ID NO: 98)
X₄X₅X₆KGX₇

wherein X₄ represents I, V, F, or L,

X₅ represents S, T, N, C, or M,

X₆ represents C, V, A, I, W, or F, and

X₇ represents G, A, S, or C.

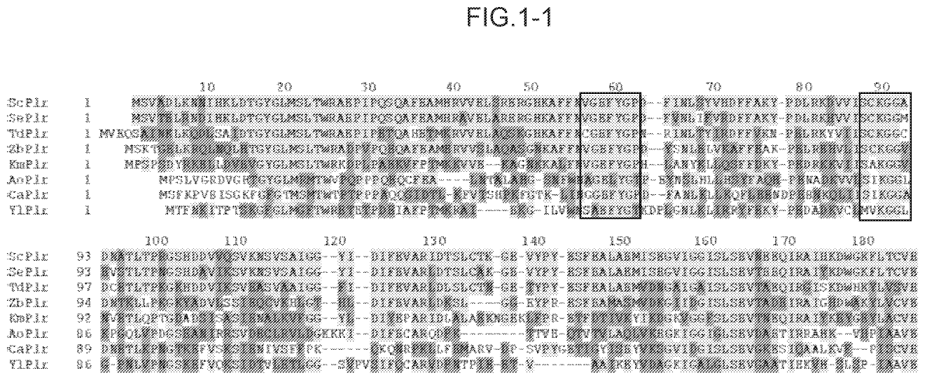

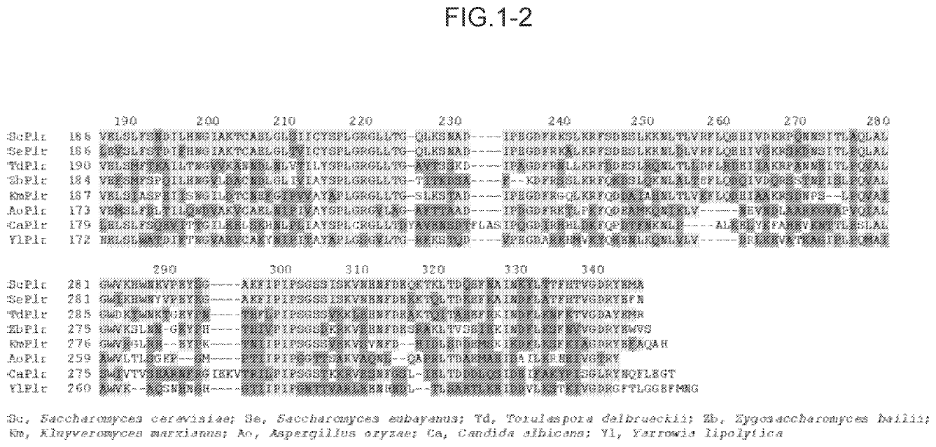

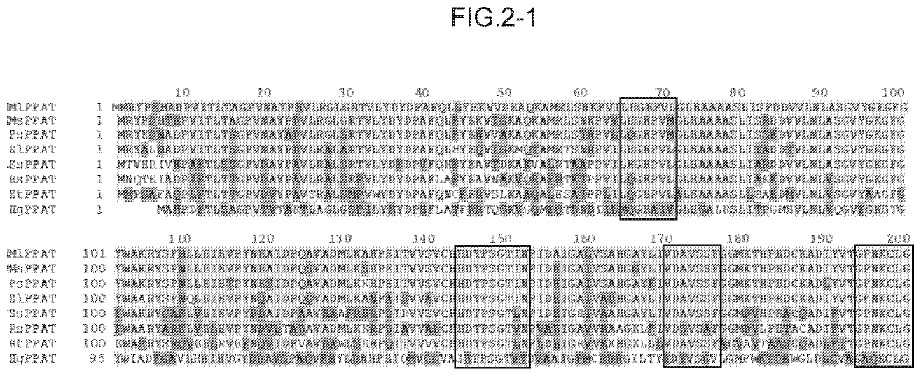

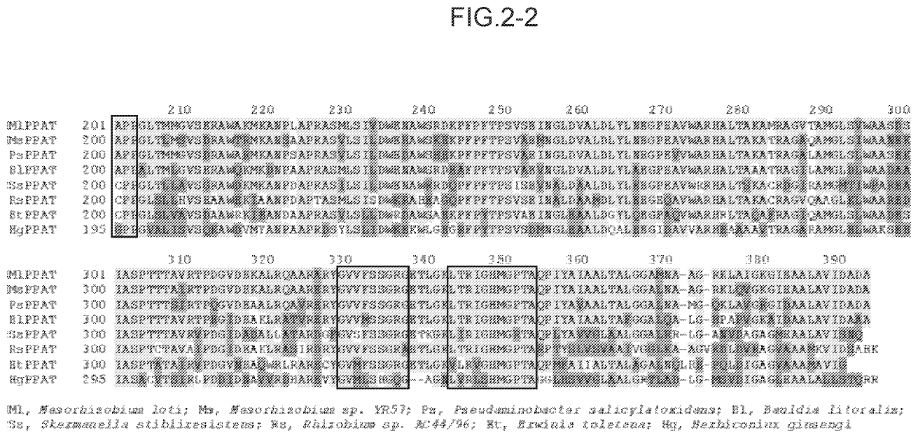

FIG. 1-1 and FIG. 1-2 illustrate the alignment of the sequences of SEQ ID NO:1 and SEQ ID NO:22 to SEQ ID NO:28. In FIG. 1-1 and FIG. 1-2, ScPlr represents a pyridoxine dehydrogenase from Saccharomyces cerevisiae, SePlr represents a pyridoxine dehydrogenase from Saccharomyces eubayanus, TdPlr represents a pyridoxine dehydrogenase from Torulaspora delbrueckii, ZbPlr represents a pyridoxine dehydrogenase from Zygosaccharomyces bailii, KmPlr represents a pyridoxine dehydrogenase from Kluyveromyces marxianus, AoPlr represents a pyridoxine dehydrogenase from Aspergillus oryzae, CaPlr represents a pyridoxine dehydrogenase from Candida albicans, and YlPlr represents a pyridoxine dehydrogenase from Yarrowia lipolytica. The partial amino acid sequence (a) corresponds to amino acid residues corresponding to the 55th to 61st amino acid residues from the N-terminus of the pyridoxine dehydrogenase from Saccharomyces cerevisiae on the alignment, and the partial amino acid sequence (b) corresponds to amino acid residues corresponding to the 86th to 91st amino acid residues from the N-terminus of the pyridoxine dehydrogenase from Saccharomyces cerevisiae on the alignment. In the disclosure, the alignment of sequences can be performed using, for example, a BLAST (registered trademark, National Library of Medicine) program with default parameters. The partial amino acid sequence (a) preferably exists in the region of the 45th to 71st amino acid residues from the N-terminus of the protein, and more preferably exists in the region of the 46th to 66th amino acid residues from the N-terminus. The partial amino acid sequence (b) preferably exists in the region of the 76th to 101st amino acid residues from the N-terminus of the protein, and more preferably exists in the region of the 79th to 96th amino acid residues from the N-terminus.

In the disclosure, “amino acid residue corresponding to Xth amino acid residue from N-terminus of enzyme A” in the amino acid sequence of enzyme B refers to an amino acid residue on the amino acid sequence of the enzyme B, corresponding to the Xth amino acid residue from the N-terminus of the amino acid sequence of the enzyme A, in a case in which the amino acid sequence of the enzyme A is aligned with the amino acid sequence of the enzyme B.

As is clear from FIG. 1-1 and FIG. 1-2, the partial amino acid sequence (a) and the partial amino acid sequence (b) are regions highly conserved in a group of pyridoxine dehydrogenases. Accordingly, a variation of the pyridoxine dehydrogenase including at least one of the partial amino acid sequence (a) or the partial amino acid sequence (b) is considered to be highly probable to achieve functioning as the pyridoxine dehydrogenase in the disclosure. Both an amino acid residue corresponding to the 60th tyrosyl residue from the N-terminus of the pyridoxine dehydrogenase from Saccharomyces cerevisiae on the alignment and an amino acid residue corresponding to the 89th lysine residue from the N-terminus of the pyridoxine dehydrogenase from Saccharomyces cerevisiae on the alignment are considered to be NADPH-binding residues (Journal of Biological Chemistry 1990, 274(33), 23185-23190), and are important residues in view of the function of the pyridoxine dehydrogenase. In a case in which these residues are conserved, it is considered to be highly probable to achieve functioning as the pyridoxine dehydrogenase in the disclosure.

Examples of other expressions of a region including the amino acid residue corresponding to the 60th tyrosyl residue from the N-terminus of the pyridoxine dehydrogenase from Saccharomyces cerevisiae on the alignment include the following partial amino acid sequence (a-1). Instead of the partial amino acid sequence (a), the pyridoxine dehydrogenase may include the partial amino acid sequence (a-1):

(a-1)
(SEQ ID NO: 109)
X₁X₂NX₃X₄EX₅YGX₆X₇

wherein X₁ represents F, L, I, M, Y, or V,

X₂ represents F, I, Y, L, W, or V,

X₃ represents V, C, I, A, M, S, or L,

X₄ represents G or A,

X₅ represents F or L,

X₆ represents P, K, R, E, T, A, N, or S, and

X₇ represents D, N, H, K, E, P, L, or I.

The partial amino acid sequence (a-1) corresponds to amino acid residues corresponding to the 53rd to 63rd amino acid residues from the N-terminus of the pyridoxine dehydrogenase from Saccharomyces cerevisiae on the alignment. The partial amino acid sequence (a-1) preferably exists in the region of the 43rd to 73rd amino acid residues from the N-terminus of the protein, and more preferably exists in the region of the 45th to 68th amino acid residues from the N-terminus.

Examples of other expressions of a region including the amino acid residue corresponding to the 89th lysine residue from the N-terminus of the pyridoxine dehydrogenase from Saccharomyces cerevisiae on the alignment include the following partial amino acid sequence (b-1). Instead of the partial amino acid sequence (b), the pyridoxine dehydrogenase may include the partial amino acid sequence (b-1):

(b-1)
(SEQ ID NO: 110)
X₁X₂X₃X₄X₅X₆X₇X₈KGX₉

wherein X₁ represents R, K, A, S, or N,

X₂ represents K, S, E, Q, D, or A,

X₃ represents D, H, N, Y, E, K, C, Q, or R,

X₄ represents V, T, I, M, or L,

X₅ represents V, I, L, F, M, or T,

X₆ represents I, V, F, or L,

X₇ represents S, T, N, C, or M,

X₈ represents C, V, A, I, W, or F, and

X₉ represents G, A, S, or C.

The partial amino acid sequence (b-1) corresponds to amino acid residues corresponding to the 81st to 91st amino acid residues from the N-terminus of the pyridoxine dehydrogenase from Saccharomyces cerevisiae on the alignment. The partial amino acid sequence (b-1) preferably exists in the region of the 71st to 101st amino acid residues from the N-terminus of the protein, and more preferably exists in the region of the 74th to 96th amino acid residues from the N-terminus.

The pyridoxamine synthetase may be, for example, a protein having the amino acid sequence of SEQ ID NO:3, or a protein having an amino acid sequence in which one or more of substitution, deletion, or insertion of an amino acid residue, or addition of an additional amino acid residue to either or both of the N-terminus and C-terminus of the amino acid sequence are performed in the amino acid sequence of SEQ ID NO:3. Examples of the degrees of the substitution, deletion, and insertion of the amino acid residue, and the addition of an additional amino acid residue to either or both of the N-terminus and C-terminus of the amino acid sequence are as described above.

Alternatively, the pyridoxamine synthetase may be a protein having the amino acid sequence of SEQ ID NO:3, or may be a protein having an amino acid sequence having, for example, 80% or more, 85% or more, 90% or more, or 95% or more sequence identity with the amino acid sequence of SEQ ID NO:3.

In the case of using such a protein having an amino acid sequence similar to the amino acid sequence of SEQ ID NO:3 as described above, it is necessary that the protein has an activity for a pyridoxamine synthetase (an enzymatic activity of synthesizing pyridoxamine from pyridoxal, also referred to as pyridoxamine synthetase activity in the first aspect). The enzymatic activity of synthesizing pyridoxamine from pyridoxal can be measured by, for example, adding a protein to be tested to an aqueous solution including pyridoxal as a substrate, and a necessary amino acid (for example, L-alanine in the case of a protein having an amino acid sequence similar to the amino acid sequence of SEQ ID NO:3), and quantifying the amount of produced pyridoxamine by high performance liquid chromatography or the like.

Alternatively, the pyridoxamine synthetase may be a protein having an amino acid sequence of any one of SEQ ID NO:3 or SEQ ID NO:29 to SEQ ID NO:35, or may be a protein having an amino acid sequence having, for example, 80% or more, 85% or more, 90% or more, or 95% or more sequence identity with at least one of the amino acid sequence of SEQ ID NO:3, the amino acid sequence of SEQ ID NO:29, the amino acid sequence of SEQ ID NO:30, the amino acid sequence of SEQ ID NO:31, the amino acid sequence of SEQ ID NO:32, the amino acid sequence of SEQ ID NO:33, the amino acid sequence of SEQ ID NO:34, or the among amino acid sequence of SEQ ID NO:35. It is necessary that the protein also has an activity for a pyridoxamine synthetase. In this case, the enzymatic activity of synthesizing pyridoxamine from pyridoxal can be measured by, for example, adding a protein to be tested to an aqueous solution including pyridoxal as a substrate, and L-alanine, and quantifying the amount of produced pyridoxamine by high performance liquid chromatography or the like.

Alternatively, the pyridoxamine synthetase may be a pyridoxamine synthetase including at least one of the following partial amino acid sequence (c), partial amino acid sequence (d), partial amino acid sequence (e), partial amino acid sequence (f), partial amino acid sequence (g), or partial amino acid sequence (h), and having an enzymatic activity of synthesizing pyridoxamine from pyridoxal. In this case, the enzymatic activity of synthesizing pyridoxamine from pyridoxal can be measured by, for example, adding a protein to be tested to an aqueous solution including pyridoxal as a substrate, and L-alanine, and quantifying the amount of produced pyridoxamine by high performance liquid chromatography or the like.

(c)
(SEQ ID NO: 99)
X₈X₉X₁₀X₁₁X₁₂X₁₃

wherein X₈ represents L, M, I, or V,

X₉ represents H or Q,

X₁₀ represents G, C, or A,

X₁₁ represents E or D,

X₁₂ represents P or A, and

X₁₃ represents V, I, L, or A;

(d)
(SEQ ID NO: 100)
X₁₄X₁₅TPSGTX₁₆X₁₇

wherein X₁₄ represents H or S,

X₁₅ represents D or E,

X₁₆ represents I, V, or L, and

X₁₇ represents N or T;

(e)
(SEQ ID NO: 101)
X₁₈DX₁₉VSX₂₀X₂₁

wherein X₁₈ represents V, I, or A,

X₁₉ represents A, T, or S,

X₂₀ represents S, A, or and

X₂₁ represents F, W, or V;

(f)
(SEQ ID NO: 102)
X₂₂X₂₃X₂₄KCX₂₅GX₂₆X₂₇P

wherein X₂₂ represents G or S,

X₂₃ represents P, S, or A,

X₂₄ represents N, S, A, or Q,

X₂₅ represents L or M,

X₂₆ represents A, S, C, or and

X₂₇ represents P, T, S, or A;

(g)
(SEQ ID NO: 103)
X₂₈X₂₉X₃₀X₃₁SX₃₂GX₃₃X₃₄

wherein X₂₈ represents G or D,

X₂₉ represents V or I,

X₃₀ represents V, T, A, S, M, I or L,

X₃₁ represents F, M, L, I, or V,

X₃₂ represents S, A, T, I, L, or H,

X₃₃ represents R, M, or Q, and

X₃₄ represents G, R, A, D, H, or K;

(h)
(SEQ ID NO: 104)
X₃₅X₃₆RX₃₇X₃₈HMGX₃₉X₄₀A

wherein X₃₅ represents L or V,

X₃₆ represents T, I, V, or L,

X₃₇ represents I, V, or L,

X₃₈ represents G or S,

X₃₉ represents P, A, or R, and

X₄₀ represents T, V, or S.

FIG. 2-1 and FIG. 2-2 illustrate the alignment of the sequences of SEQ ID NO:3 and SEQ ID NO:29 to SEQ ID NO:35. In FIG. 2-1 and FIG. 2-2, M1PPAT represents a pyridoxamine-pyruvate transaminase from Mesorhizobium loti, MsPPAT represents a pyridoxamine-pyruvate transaminase from Mesorhizobium sp. YR577, PsPPAT represents a pyridoxamine-pyruvate transaminase from Pseudaminobacter salicylatoxidans, BlPPAT represents a pyridoxamine-pyruvate transaminase from Bauldia litoralis, SsPPAT represents a pyridoxamine-pyruvate transaminase from Skermanella stibiiresistens, RsPPAT represents a pyridoxamine-pyruvate transaminase from Rhizobium sp. AC44/96, EtPPAT represents a pyridoxamine-pyruvate transaminase from Envinia toletana, and HgPPAT represents a pyridoxamine-pyruvate transaminase from Herbiconiux ginsengi. The partial amino acid sequence (c) corresponds to amino acid residues corresponding to the 65th to 70th amino acid residues from the N-terminus of the pyridoxamine-pyruvate transaminase from Mesorhizobium loti on the alignment, the partial amino acid sequence (d) corresponds to amino acid residues corresponding to the 144th to 152nd amino acid residues from the N-terminus of the pyridoxamine-pyruvate transaminase from Mesorhizobium loti on the alignment, the partial amino acid sequence (e) corresponds to amino acid residues corresponding to the 170th to 176th amino acid residues from the N-terminus of the pyridoxamine-pyruvate transaminase from Mesorhizobium loti on the alignment, the partial amino acid sequence (f) corresponds to amino acid residues corresponding to the 194th to 203rd amino acid residues from the N-terminus of the pyridoxamine-pyruvate transaminase from Mesorhizobium loti on the alignment, the partial amino acid sequence (g) corresponds to amino acid residues corresponding to the 329th to 337th amino acid residues from the N-terminus of the pyridoxamine-pyruvate transaminase from Mesorhizobium loti on the alignment, and the partial amino acid sequence (h) corresponds to amino acid residues corresponding to the 343rd to 353rd amino acid residues from the N-terminus of the pyridoxamine-pyruvate transaminase from Mesorhizobium loti on the alignment. The partial amino acid sequence (c) preferably exists in the region of the 55th to 80th amino acid residues from the N-terminus of the protein, and more preferably exists in the region of the 56th to 75th amino acid residues from the N-terminus. The partial amino acid sequence (d) preferably exists in the region of the 134th to 162nd amino acid residues from the N-terminus of the protein, and more preferably exists in the region of the 139th to 157th amino acid residues from the N-terminus. The partial amino acid sequence (e) preferably exists in the region of the 160th to 186th amino acid residues from the N-terminus of the protein, and more preferably exists in the region of the 165th to 181st amino acid residues from the N-terminus. The partial amino acid sequence (f) preferably exists in the region of the 184th to 213th amino acid residues from the N-terminus of the protein, and more preferably exists in the region of the 189th to 208th amino acid residues from the N-terminus. The partial amino acid sequence (g) preferably exists in the region of the 319th to 347th amino acid residues from the N-terminus of the protein, and more preferably exists in the region of the 324th to 342nd amino acid residues from the N-terminus. The partial amino acid sequence (h) preferably exists in the region of the 333rd to 363rd amino acid residues from the N-terminus of the protein, and more preferably exists in the region of the 338th to 358th amino acid residues from the N-terminus.

As is clear from FIG. 2-1 and FIG. 2-2, the partial amino acid sequence (c), the partial amino acid sequence (d), the partial amino acid sequence (e), the partial amino acid sequence (f), the partial amino acid sequence (g), and the partial amino acid sequence (h) are regions highly conserved in a group of pyridoxamine-pyruvate transaminases. Accordingly, a variation of the pyridoxamine-pyruvate transaminase including at least one of the partial amino acid sequence (c), the partial amino acid sequence (d), the partial amino acid sequence (e), the partial amino acid sequence (f), the partial amino acid sequence (g), or the partial amino acid sequence (h) is considered to have a high probability of functioning as the pyridoxamine synthetase in the disclosure. It is considered that an amino acid residue corresponding to the 197th lysine residue from the N-terminus of the pyridoxamine-pyruvate transaminase from Mesorhizobium loti on the alignment is important for binding to pyridoxal, an amino acid residue corresponding to the 68th glutamate residue from the N-terminus on the alignment is important for catalytic activity, an amino acid residue corresponding to the 171st aspartate residue from the N-terminus on the alignment and an amino acid residue corresponding to the 146th threonine residue from the N-terminus on the alignment assist binding to pyridoxal, and an amino acid residue corresponding to the 336th arginine residue from the N-terminus on the alignment and an amino acid residue corresponding to the 345th arginine residue from the N-terminus on the alignment are important for recognizing an amino acid (Journal of Biological Chemistry, 2008, vol. 283, No. 2 pp 1120-112′7), and the amino acid residues are important residues in view of the function of the pyridoxine synthetase. In a case in which these residues are conserved, it is considered to be highly probable to achieve functioning as the pyridoxine synthetase in the disclosure. However, an amino acid residue corresponding to the 68th glutamate residue from the N-terminus of the pyridoxamine-pyruvate transaminase from Mesorhizobium loti on the alignment may be not only glutamate but also aspartate. An amino acid residue corresponding to the 336th arginine residue from the N-terminus of the pyridoxamine-pyruvate transaminase from Mesorhizobium loti on the alignment may be not only arginine but also methionine or glutamine.

Examples of other expressions of a region including the amino acid residue corresponding to the 68th glutamate residue from the N-terminus of the pyridoxamine-pyruvate transaminase from Mesorhizobium loti on the alignment include the following partial amino acid sequence (c-1). Instead of the partial amino acid sequence (c), the pyridoxamine synthetase may include the partial amino acid sequence (c-1).

(c-1)
(SEQ ID NO: 111)
X₁X₂X₃X₄X₅X₆X₇X₈X₉X₁₀X₁₁X₁₂X₁₃X₁₄X₁₅X₁₆X₁₇X₁₈X₁₉

wherein X₁ represents V, L, I, or M,

X₂ represents I, L, or V,

X₃ represents L, M, I, or V,

X₄ represents H or Q,

X₅ represents G, C, or A,

X₆ represents E or D,

X₇ represents P or A,

X₈ represents V, I, A, or L,

X₉ represents L, M, P, or V,

X₁₀ represents G or A,

X₁₁ represents L or I,

X₁₂ represents E or Q,

X₁₃ represents A or

X₁₄ represents A or V,

X₁₅ represents A or L,

X₁₆ represents A, L, H, or Y,

X₁₇ represents S, or A,

X₁₈ represents L, F, V, or A, and

X₁₉ represents I, F, V, or L.

The partial amino acid sequence (c-1) corresponds to amino acid residues corresponding to the 63rd to 81st amino acid residues from the N-terminus of the pyridoxamine-pyruvate transaminase from Mesorhizobium loti on the alignment. The partial amino acid sequence (c-1) preferably exists in the region of the 53rd to 91st amino acid residues from the N-terminus of a protein, and more preferably exists in the region of the 58th to 86th amino acid residues from the N-terminus.

Examples of other expressions of a region including the amino acid residue corresponding to the 146th threonine residue from the N-terminus of a pyridoxamine-pyruvate transaminase from Mesorhizobium loti on the alignment include the following partial amino acid sequence (d-1). Instead of the partial amino acid sequence (d), the pyridoxamine synthetase may include the partial amino acid sequence (d-1).

(d-1)
(SEQ ID NO: 112)
X₁X₂X₃X₄X₅X₆X₇X₈TPSGTX₉X₁₀X₁₁X₁₂X₁₃X₁₄X₁₅X₁₆

wherein X₁ represents V, I, L, or M,

X₂ represents V or I,

X₃ represents S, A, V, C, or F,

X₄ represents V, I, A, L, or T,

X₅ represents C or V,

X₆ represents H, N, or A,

X₇ represents H or S,

X₈ represents D or E,

X₉ represents I, V, or L,

X₁₀ represents N or T,

X_{II} represents P or D,

X₁₂ represents I, V, L, or A,

X₁₃ represents D, N, E, A, Q, V, R, or P,

X₁₄ represents A, E, Q, or D,

X₁₅ represents I or L, and

X₁₆ represents G or A.

The partial amino acid sequence (d-1) corresponds to the amino acid residues corresponding to the 138th to 158th amino acid residues from the N-terminus of the pyridoxamine-pyruvate transaminase from Mesorhizobium loti on the alignment. The partial amino acid sequence (d-1) preferably exists in the region of the 128th to 168th amino acid residues from the N-terminus of a protein, and more preferably exists in the region of the 133rd to 163rd amino acid residues from the N-terminus.

Examples of other expressions of a region including the amino acid residue corresponding to the 171st aspartate residue from the N-terminus of a pyridoxamine-pyruvate transaminase from Mesorhizobium loti include the following partial amino acid sequence (e-1). Instead of the partial amino acid sequence (e), the pyridoxamine synthetase may include the partial amino acid sequence (e-1).

(e-1)
(SEQ ID NO: 113)
X₁X₂X₃X₄X₅X₆DX₇VSX₈X₉X₁₀X₁₁X₁₂

wherein X₁ represents D, or A,

X₂ represents A, K, T, Q, R, or E,

X₃ represents Y, N, L, or F,

X₄ represents L, F, M, or V,

X₅ represents I, L, or Y,

X₆ represents V, A, or I,

X₇ represents A, S, or T,

X₈ represents S, A, or

X₉ represents F, W, or V,

X₁₀ represents G, A, or L,

X₁₁ represents G or S, and

X₁₂ represents M, V, or L.

The partial amino acid sequence (e-1) corresponds to the amino acid residues corresponding to the 165th to 179th amino acid residues from the N-terminus of the pyridoxamine-pyruvate transaminase from Mesorhizobium loti on the alignment. The partial amino acid sequence (e-1) preferably exists in the region of the 155th to 189th amino acid residues from the N-terminus of the protein, and more preferably exists in the region of the 160th to 184th amino acid residues from the N-terminus.

Examples of other expressions of a region including the amino acid residue corresponding to the 197th lysine residue from the N-terminus of a pyridoxamine-pyruvate transaminase from Mesorhizobium loti on the alignment include the following partial amino acid sequence (f-1). Instead of the partial amino acid sequence (f), the pyridoxamine synthetase may include the partial amino acid sequence (f-1).

(f-1)
(SEQ ID NO: 114)
X₁X₂X₃X₄X₅X₆X₇X₈X₉KCX₁₀GX₁₁X₁₂PX₁₃X₁₄X₁₅X₁₆X₁₇X₁₈X₁₉S

wherein X₁ represents A, S, V, or I,

X₂ represents D, or A,

X₃ represents I, L, F, V, or M,

X₄ represents Y, F, L, or C,

X₅ represents V or I,

X₆ represents T or A,

X₇ represents G or S,

X₈ represents P, S, or A,

X₉ represents N, S, Q, or A,

X₁₀ represents L or M,

X_{II} represents A, S, C, or

X₁₂ represents P, T, S, or A,

X₁₃ represents G, A, or S,

X₁₄ represents L or V,

X₁₅ represents T, S, or A,

X₁₆ represents M, I, L, V, or F,

X₁₇ represents M, L, V, A, or I,

X₁₈ represents G, A, H, or S, and

X₁₉ represents V, I, or A.

The partial amino acid sequence (f-1) corresponds to the amino acid residues corresponding to the 188th to 211st amino acid residues from the N-terminus of the pyridoxamine-pyruvate transaminase of Mesorhizobium loti on the alignment. The partial amino acid sequence (f-1) preferably exists in the region of from the 178th to 221st amino acid residues from the N-terminus of the protein, and more preferably exists in the region of from the 183rd to 216th amino acid residues from the N-terminus.

Examples of other expressions of a region including the amino acid residue corresponding to the 336th arginine residue from the N-terminus of the pyridoxamine-pyruvate transaminase from Mesorhizobium loti on the alignment include the following partial amino acid sequence (g-1). Instead of the partial amino acid sequence (g), the pyridoxamine synthetase may include the partial amino acid sequence (g-1).

(g-1)
(SEQ ID NO: 115)
X₁X₂X₃X₄X₅SX₆GX₇X₈

wherein X₁ represents Y, F, H, or S,

X₂ represents G or D,

X₃ represents V or I,

X₄ represents V, T, A, S, M, I, or L,

X₅ represents F, M, L, I, or V,

X₆ represents S, A, T, I, L, or H,

X₇ represents R, M, or Q, and

X₈ represents G, R, A, D, H, or K.

The partial amino acid sequence (g-1) corresponds to the amino acid residues corresponding to the 328th to 337th amino acid residues from the N-terminus of the pyridoxamine-pyruvate transaminase from Mesorhizobium loti on the alignment. The partial amino acid sequence (g-1) preferably exists in the region of the 318th to 347th amino acid residues from the N-terminus of the protein, and more preferably exists in the region of the 323rd to 342nd amino acid residues from the N-terminus.

Examples of other expressions of a region including the amino acid residue corresponding to the 345th arginine residue from the N-terminus of the pyridoxamine-pyruvate transaminase from Mesorhizobium loti on the alignment include the following partial amino acid sequence (h-1). Instead of the partial amino acid sequence (h), the pyridoxamine synthetase may include the partial amino acid sequence (h-1).

(h-1)
(SEQ ID NO: 116)
X₁X₂X₃X₄X₅RX₆X₇HMGX₈X₉AX₁₀X₁₁

wherein X₁ represents L, Q, K, A, F, Y, or W,

X₂ represents G, N, H, or D,

X₃ represents K, R, or N,

X₄ represents L or V,

X₅ represents T, I, V, or L,

X₆ represents I, V, or L,

X₇ represents G or S,

X₈ represents P, A, or R,

X₉ represents T, V, or S,

X₁₀ represents Q, R, E, K, H, Y, or and

X₁₁ represents P or G

The partial amino acid sequence (h-1) corresponds to the amino acid residues corresponding to the 340th to 355th amino acid residues from the N-terminus of the pyridoxamine-pyruvate transaminase from Mesorhizobium loti on the alignment. The partial amino acid sequence (h-1) preferably exists in the region of the 330th to 365th amino acid residues from the N-terminus of a protein, and more preferably exists in the region of the 335th to 360th amino acid residues from the N-terminus.

The pyridoxamine synthetase may be, for example, a protein having the amino acid sequence of SEQ ID NO:4, or may be a protein having an amino acid sequence in which one or more of substitution, deletion, or insertion of an amino acid residue, or addition of an additional amino acid residue to either or both of the N-terminus and C-terminus of the amino acid sequence are performed in the amino acid sequence of SEQ ID NO:4. Examples of the degrees of the substitution, deletion, and insertion of the amino acid residue, and the addition of an additional amino acid residue to either or both of the N-terminus and the C-terminus of the amino acid sequence are as described above.

Alternatively, the pyridoxamine synthetase may be a protein having the amino acid sequence of SEQ ID NO:4, or may be a protein having an amino acid sequence having, for example, 80% or more, 85% or more, 90% or more, or 95% or more sequence identity with the amino acid sequence of SEQ ID NO:4.

In the case of using such a protein having an amino acid sequence similar to the amino acid sequence of SEQ ID NO:4 as described above, it is necessary that the protein has an activity for pyridoxamine synthetase (an enzymatic activity of synthesizing pyridoxamine from pyridoxal). The enzymatic activity of synthesizing pyridoxamine from pyridoxal can be measured by, for example, adding a protein to be tested to an aqueous solution including pyridoxal as a substrate, and necessary amino acid (for example, L-glutamic acid, L-aspartic acid, or a salt thereof in the case of a protein having an amino acid sequence similar to the amino acid sequence of SEQ ID NO:4), and quantifying the amount of produced pyridoxamine by high performance liquid chromatography or the like.

As the amino acid regeneration enzyme, for example, a protein having an amino acid sequence (SEQ ID NO:5) in which an amino acid of D198A is substituted in the amino acid sequence (SEQ ID NO:36) of an amino acid regeneration enzyme derived from Shewanella sp. AC10 can be used. The protein is an alanine dehydrogenase represented by enzyme number 1.4.1.1. Not only such an alanine dehydrogenase but also an alanine dehydrogenase in which an amino acid substitution to use NADP⁺ is introduced in the alanine dehydrogenase represented by the enzyme number 1.4.1.1 can be used. In most alanine dehydrogenases, it is possible to use NAD⁺ (oxidized nicotinamide adenine dinucleotide) as a coenzyme, but it is impossible to use NADP⁺ (oxidized nicotinamide adenine dinucleotide phosphate), as described in Journal of Molecular Catalysis B: Enzymatic 30 (2004) 173-176. However, both NAD⁺ and NADP⁺ can be used as coenzymes in the protein having the amino acid sequence of SEQ ID NO:5, in which the amino acid of D198A is substituted (see the document described above). Here, the protein having the amino acid sequence of SEQ ID NO:5 can use both NADH and NADPH in reverse reaction (regeneration of L-alanine from pyruvic acid and NH₃).

Similar amino acid substitution can be performed in the alanine dehydrogenases other than the alanine dehydrogenase derived from Shewanella sp. AC10. Herein, substitution of an alanine residue for a residue corresponding to the 198th aspartate residue from the N-terminus of the amino acid sequence of SEQ ID NO:36 on alignment in the amino acid sequences of an alanine dehydrogenase is referred to as “amino acid substitution to use NADP⁺”. The alanine dehydrogenase as the amino acid regeneration enzyme may be a protein having an amino acid sequence (SEQ ID NO:45) obtained by amino acid substitution to use NADP⁺ (D198A in this case) of an alanine dehydrogenase derived from Aeromonas hydrophila, a protein having an amino acid sequence (SEQ ID NO:46) obtained by amino acid substitution to use NADP⁺ (D198A in this case) of an alanine dehydrogenase derived from Rhizobium sp. LPU83, a protein having an amino acid sequence (SEQ ID NO:47) obtained by amino acid substitution to use NADP⁺ (D198A in this case) of an alanine dehydrogenase derived from Pseudomonas mendocina, a protein having an amino acid sequence (SEQ ID NO:48 or SEQ ID NO:49) obtained by amino acid substitution to use NADP⁺ (D198A in this case) of an alanine dehydrogenase derived from Bradyrhizobium japonicum, a protein having an amino acid sequence (SEQ ID NO:50) obtained by amino acid substitution to use NADP⁺ (D198A in this case) of an alanine dehydrogenase derived from Streptomyces aureofaciens, a protein having an amino acid sequence (SEQ ID NO:51) obtained by amino acid substitution to use NADP⁺ (D198A in this case) of an alanine dehydrogenase derived from Anabaena cylindrica, a protein having an amino acid sequence (SEQ ID NO:52) obtained by amino acid substitution to use NADP⁺ (D196A in this case) of an alanine dehydrogenase derived from Bacillus subtilis, or the like.

Under reaction conditions in which NAD⁺ is available, the amino acid regeneration enzyme can be used without amino acid substitution to use NADP⁺, and, in addition, the alanine dehydrogenase represented by the enzyme number 1.4.1.1 can be commonly used.

The amino acid regeneration enzyme may be, for example, a glutamate dehydrogenase having the amino acid sequence of SEQ ID NO:6 and derived from E. coli. The glutamate dehydrogenase has an enzymatic activity of producing L-glutamic acid from 2-oxoglutaric acid and NH₃ using NADH or NADPH as a coenzyme.

The amino acid regeneration enzyme may be, for example, a protein having the amino acid sequence of SEQ ID NO:5, or a protein having an amino acid sequence in which one or more of substitution, deletion, or insertion of an amino acid residue, or addition of an additional amino acid residue to either or both of the N-terminus and C-terminus of the amino acid sequence are performed in the amino acid sequence of SEQ ID NO:5 Examples of the degrees of the substitution, deletion, and insertion of the amino acid residue, and the addition of an additional amino acid residue to either or both of the N-terminus and C-terminus of the amino acid sequence are as described above.

Alternatively, the amino acid regeneration enzyme may be a protein having the amino acid sequence of SEQ ID NO:5, or may be a protein having an amino acid sequence having, for example, 80% or more, 85% or more, 90% or more, 95% or more sequence identity with the amino acid sequence of SEQ ID NO:5.

In the case of using such a protein having an amino acid sequence similar to the amino acid sequence of SEQ ID NO:5 as described above, it is necessary that the protein has alanine regeneration enzymatic activity. It is preferable to have the 198th Ala residue from the N-terminus of the amino acid sequence of SEQ ID NO:5 or an Ala residue corresponding thereto on sequence alignment.

Alternatively, the amino acid regeneration enzyme may be a protein having an amino acid sequence of any one of SEQ ID NO:5 or SEQ ID NO:45 to SEQ ID NO:52, or may be a protein having an amino acid sequence having, for example, 80% or more, 85% or more, 90% or more, or 95% or more sequence identity with the amino acid sequence of SEQ ID NO:5, SEQ ID NO:45, SEQ ID NO:46, SEQ ID NO:47, SEQ ID NO:48, SEQ ID NO:49, SEQ ID NO:50, SEQ ID NO:51, or SEQ ID NO:52.

In the case of using such a protein having an amino acid sequence similar to the amino acid sequence of SEQ ID NO:5, SEQ ID NO:45, SEQ ID NO:46, SEQ ID NO:47, SEQ ID NO:48, SEQ ID NO:49, SEQ ID NO:50, SEQ ID NO:51, or SEQ ID NO:52 as described above, it is necessary that the protein has alanine regeneration enzymatic activity. It is preferable to have the 198th Ala residue from the N-terminus of the amino acid sequence of SEQ ID NO:5 or an Ala residue corresponding thereto on sequence alignment.

Alternatively, the amino acid regeneration enzyme may be, for example, a protein having the amino acid sequence of SEQ ID NO:6, or a protein having an amino acid sequence in which one or more of substitution, deletion, or insertion of an amino acid residue in the amino acid sequence of SEQ ID NO:6, or addition of an additional amino acid residue to either or both of the N-terminus and C-terminus of the amino acid sequence are performed. Examples of the degrees of the substitution, deletion, and insertion of the amino acid residue, and the addition of an additional amino acid residue to either or both of the N-terminus and C-terminus of the amino acid sequence are as described above.

Alternatively, the amino acid regeneration enzyme may be a protein having the amino acid sequence of SEQ ID NO:6, or may be a protein having an amino acid sequence having, for example, 80% or more, 85% or more, 90% or more, or 95% or more sequence identity with the amino acid sequence of SEQ ID NO:6.

In the case of using such a protein having an amino acid sequence similar to the amino acid sequence of SEQ ID NO:6 as described above, it is necessary that the protein has glutamic acid regeneration enzymatic activity.

An amino acid regeneration enzymatic activity can be measured by, for example, adding a protein to be tested to an aqueous solution including a necessary substrate and NADP or NADPH, if necessary, and quantifying the amount of desired amino acid as a reaction product by high performance liquid chromatography or the like. For example, the alanine regeneration enzymatic activity can be measured by, for example, adding a protein to be tested to an aqueous solution including pyruvic acid, NH₃, and NADH or NADPH, and quantifying the amount of L-alanine as a reaction product by high performance liquid chromatography or the like. The glutamic acid regeneration enzymatic activity can be measured by, for example, adding a protein to be tested to an aqueous solution including 2-oxoglutaric acid, NH₃, and NADH or NADPH, and quantifying the amount of L-glutamic acid as a reaction product by high performance liquid chromatography or the like.

Alternatively, the alanine dehydrogenase may be an enzyme including at least one of a partial amino acid sequence (i), a partial amino acid sequence (j), a partial amino acid sequence (k), or a partial amino acid sequence (1), and having an activity of producing L-alanine from pyruvic acid and NH₃. In such an alanine dehydrogenase, substitution of an alanine residue for an amino acid residue corresponding to the 198th aspartate residue from the N-terminus of Shewanella sp. AC10 on alignment can enable both of NAD⁺ and NADP⁺ to be used as coenzymes.

(i)
(SEQ ID NO: 105)
EX₄₁KX₄₂X₄₃EX₄₄RX₄₅X₄₆

wherein X₄₁ represents I, T, S, F, N, or V,

X₄₂ represents N, M, A, V, L, T, or D,

X₄₃ represents H, N, L, or Q,

X₄₄ represents Y, N, or F,

X₄₅ represents V or I, and

X₄₆ represents G or A;

(j)
(SEQ ID NO: 106)
X₄₇X₄₈X₄₉KVKEPX₅₀

wherein X₄₇ represents M or L,

X₄₈ represents I, L, or V,

X₄₉ represents V, L, I, or M, and

X₅₀ represents Q, L, V, N, or I;

(k)
(SEQ ID NO: 107)
LX₅₁TYLHLA

wherein X₅₁ represents F or Y; and

(l)
(SEQ ID NO: 108)
X₅₂DX₅₃AX₅₄DQGG

wherein X₅₂ represents V or A,

X₅₃ represents V or I, and

X₅₄ represents I or V.

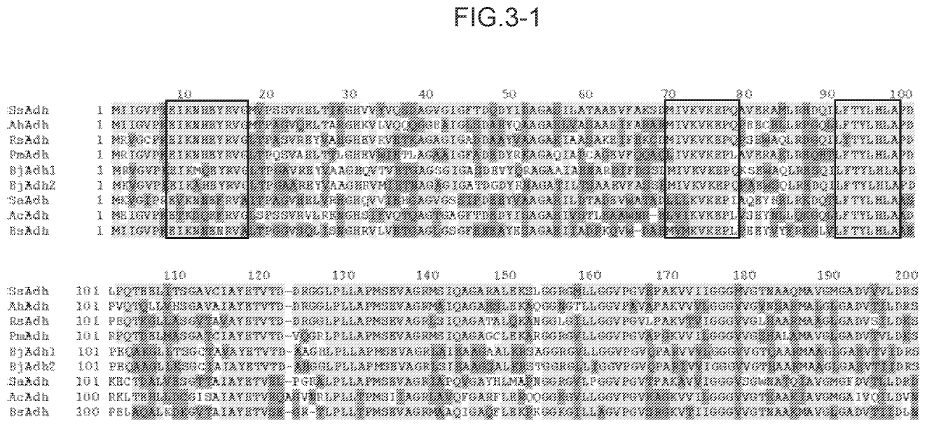

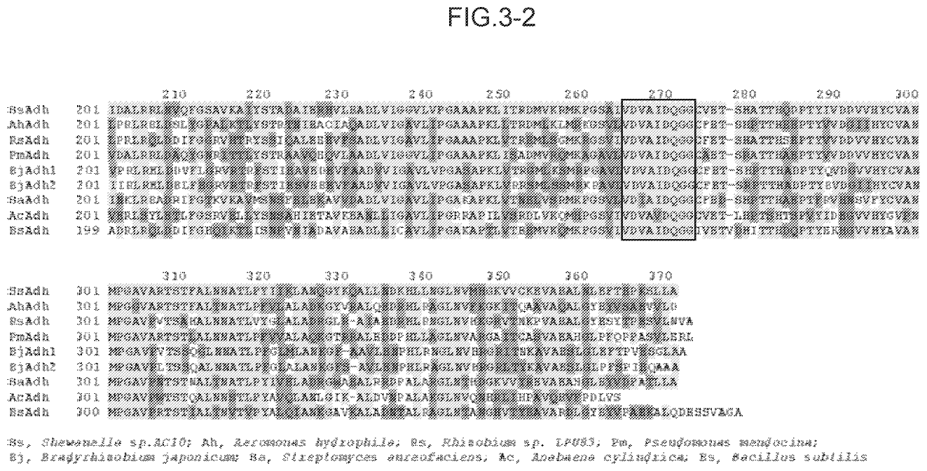

FIG. 3-1 and FIG. 3-2 illustrate the alignment of the sequences of SEQ ID NO:36 to SEQ ID NO:44. In FIG. 3-1 and FIG. 3-2, SsAdh represents an alanine dehydrogenase from Shewanella sp. AC10, AhAdh represents an alanine dehydrogenase from Aeromonas hydrophila, RsAdh represents an alanine dehydrogenase from Rhizobium sp. LPU83, PmAdh represents an alanine dehydrogenase from Pseudomonas mendocina, BjAdh1 represents one of alanine dehydrogenases from Bradyrhizobium japonicum, BjAdh2 represents another alanine dehydrogenase from Bradyrhizobium japonicum, SaAdh represents an alanine dehydrogenase from Streptomyces aureofaciens, AcAdh represents an alanine dehydrogenase from Anabaena cylindrica, and BsAdh represents an alanine dehydrogenase from Bacillus subtilis. The partial amino acid sequence (i) corresponds to amino acid residues corresponding to the 8hth to 17th amino acid residues from the N-terminus of the alanine dehydrogenase from Shewanella sp. AC10 on the alignment, the partial amino acid sequence (j) corresponds to amino acid residues corresponding to the 70th to 78th amino acid residues from the N-terminus of the alanine dehydrogenase from Shewanella sp. AC10 on the alignment, the partial amino acid sequence (k) corresponds to amino acid residues corresponding to the 91st to 98th amino acid residues from the N-terminus of the alanine dehydrogenase from Shewanella sp. AC10 on the alignment, and the partial amino acid sequence (1) corresponds to amino acid residues corresponding to the 265th to 273rd amino acid residues from the N-terminus of the alanine dehydrogenase from Shewanella sp. AC10 on the alignment. The partial amino acid sequence (i) preferably exists in the region of the 2nd to 27th amino acid residues from the N-terminus of the protein, and more preferably exists in the region of the 3rd to 22nd amino acid residues from the N-terminus. The partial amino acid sequence (j) preferably exists in the region of the 60th to 88th amino acid residues from the N-terminus of the protein, and more preferably exists in the region of the 65th to 83rd amino acid residues from the N-terminus. The partial amino acid sequence (k) preferably exists in the region of the 81st to 108th amino acid residues from the N-terminus of the protein, and more preferably exists in the region in the 86th to 103rd amino acid residues from the N-terminus. The partial amino acid sequence (1) preferably exists in the region of the 255th to 283rd amino acid residues from the N-terminus of the protein, and more preferably exists in the region of the 260th to 278th amino acid residues from the N-terminus.

As is clear from FIG. 3-1 and FIG. 3-2, the partial amino acid sequence (i), the partial amino acid sequence (j), the partial amino acid sequence (k), and the partial amino acid sequence (1) are regions highly conserved in a group of alanine dehydrogenases. Accordingly, a variation of the alanine dehydrogenase including at least one of the partial amino acid sequence (i), the partial amino acid sequence (j), the partial amino acid sequence (k), or the partial amino acid sequence (1) is considered to have a high probability of functioning as the amino acid regeneration enzyme in the disclosure. An amino acid residue corresponding to the 15th arginine residue from the N-terminus of the alanine dehydrogenase from Shewanella sp. AC10 on the alignment and an amino acid residue corresponding to the 75th lysine residue from the N-terminus on the alignment are important for binding to NADH, and amino acid residues corresponding to the 96th histidine residue from the N-terminus and the 270th aspartate residue from the N-terminus on the alignment are considered to be important for forming the structure of a protein (J. Mol. Biol., 2008, 377, 1161-1173, Enzyme and Microbial Technology, 2018, 110, 61-68), and are important residues in view of the function of the alanine dehydrogenase. In a case in which these residues are conserved, it is considered to be highly probable to achieve functioning as the amino acid regeneration enzyme in the disclosure.

Examples of other expressions of a region including an amino acid residue corresponding to the 15th arginine residue from the N-terminus of the alanine dehydrogenase from Shewanella sp. AC10 on the alignment include the following partial amino acid sequence (i-1). The alanine dehydrogenase may include the partial amino acid sequence (i-1) instead of the partial amino acid sequence (i).

(i-1)
(SEQ ID NO: 117)
GX₁PX₂EX₃KX₄X₅EX₆RX₇X₈X₉X₁₀PX₁₁X₁₃X₁₄X₁₅X₁₆

wherein X₁ represents V, I, L, or C,

X₂ represents T, K, or R,

X₃ represents I, V, T, S, F, or N,

X₄ represents N, M, A, D, V, L, or T,

X₅ represents H, N, Q, or L,

X₆ represents Y, N, or F,

X₇ represents V or I,

X₈ represents G or A,

X₉ represents M, L, or I,

X₁₀ represents V, T, I, or S,

X₁₁ represents S, A, T, Q, H, N, L, or

X₁₂ represents S, A, N, or V,

X₁₃ represents V or A,

X₁₄ represents R, K, N, Q, S, A, L, or H,

X₁₅ represents E, Q, D, V, or A, and

X₁₆ represents L, A, V, F, or Y.

The partial amino acid sequence (i-1) corresponds to amino acid residues corresponding to the 4th to 26th amino acid residues from the N-terminus of the alanine dehydrogenase from Shewanella sp. AC10 on the alignment. The partial amino acid sequence (i-1) preferably exists in the region of the 2nd to 36th amino acid residues from the N-terminus of the protein, and more preferably exists in the region of the 3d to 31st amino acid residues from the N-terminus.

Examples of other expressions of a region including an amino acid residue corresponding to the 75th lysine residue from the N-terminus of the alanine dehydrogenase from Shewanella sp. AC10 on the alignment include the following partial amino acid sequence (j-1). The alanine dehydrogenase may include the partial amino acid sequence (j-1) instead of the partial amino acid sequence (j).

(j-1)
(SEQ ID NO: 118)
X₁X₂X₃X₄KVKEPX₅X₆X₇EX₈X₉X₁₀

wherein X₁ represents D, E, Q, or K,

X₂ represents M or L,

X₃ represents I, L, or V,

X₄ represents V, L, I, or M,

X₅ represents Q, L, I, V, or N,

X₆ represents A, T, S, P, M, K, R, Q, V, I, or E,

X₇ represents V, I, E, N, Q, T, A, D, H, M, N, V, A, S, I, D, G W, or K,

X₈ represents R, C, Y, or W,

X₉ represents A, E, R, Q, K, T, S, M, N, V, P, C, or H, and

X₁₀ represents M, L, K, R, Q, E, W, F, or Y.

The partial amino acid sequence (j-1) corresponds to amino acid residues corresponding to the 69th to 84th amino acid residues from the N-terminus of the alanine dehydrogenase from Shewanella sp. AC10 on the alignment. The partial amino acid sequence (j-1) preferably exists in the region of the 59th to 94th amino acid residues from the N-terminus of the protein, and more preferably exists in the region of the 64th to 89th amino acid residues from the N-terminus.

Examples of other expressions of a region including an amino acid residue corresponding to the 96th histidine residue from the N-terminus of the alanine dehydrogenase from Shewanella sp. AC10 on the alignment include the following partial amino acid sequence (k-1). The alanine dehydrogenase may include the partial amino acid sequence (k-1) instead of the partial amino acid sequence (k).

(k-1)
(SEQ ID NO: 119)
X₁X₂X₃X₄X₅X₆LX₇TYLHLAX₈X₉X₁₀X₁₁X₁₂X₁₃X₁₄X₁₅LX₁₆X₁₇X₁₈X₁₉

wherein X₁ represents L or F,

X₂ represents R, K, C, S, H, or Q,

X₃ represents H, E, P, S, D, R, K, Q, or A,

X₄ represents D, Q, H, E, S, N, or M,

X₅ represents Q or H,

X₆ represents I, L, V, T, C, or A,

X₇ represents F or Y,

X₈ represents P or A,

X₉ represents D, S, N, H, or E,

X₁₀ represents L, M, P, R, V, Q, E, or K,

X₁₁ represents P, A, V, Q, K, E, D, T, N, R, S, or M,

X₁₂ represents Q, C, or L,

X₁₃ represents T, A, or V,

X₁₄ represents E, I, Q, A, R, K, T, D, or N,

X₁₅ represents E, D, L, A, H, Y, or S,

X₁₆ represents I, M, V, L, T, or K,

X₁₇ represents T, K, S, D, H, E, A, N, R, G, or Q,

X₁₈ represents S, C, A, or K, and

X₁₉ represents G, K, R, Q, or D.

The partial amino acid sequence (k-1) corresponds to amino acid residues corresponding to the 85th to 111th amino acid residues from the N-terminus of the alanine dehydrogenase from Shewanella sp. AC10 on the alignment. The partial amino acid sequence (k-1) preferably exists in the region of the 75th to 121st amino acid residues from the N-terminus of the protein, and more preferably exists in the region of the 80th to 116th amino acid residues from the N-terminus.

Examples of other expressions of a region including an amino acid residue corresponding to the 270th aspartate residue from the N-terminus of the alanine dehydrogenase from Shewanella sp. AC10 on the alignment include the following partial amino acid sequence (l-1). The alanine dehydrogenase may include the partial amino acid sequence (l-1) instead of the partial amino acid sequence (1).

(l-1)
(SEQ ID NO: 120)
X₁X₂X₃X₄X₅DX₆AX₇DQGGX₈X₉X₁₀X₁₁

wherein X₁ represents R, or S,

X₂ represents S, A, or

X₃ represents A or V,

X₄ represents I, L, M, or V,

X₅ represents V or A,

X₆ represents V or I,

X₇ represents I or V,

X₈ represents C or I,

X₉ represents V, I, A, F, S, or C,

X₁₀ represents E or A, and

X₁₁ represents T or D.

The partial amino acid sequence (l-1) corresponds to amino acid residues corresponding to the 261st to 277th amino acid residues from the N-terminus of the alanine dehydrogenase from Shewanella sp. AC10 on the alignment. The partial amino acid sequence (l-1) preferably exists in the region of the 251st to 287th amino acid residues from the N-terminus of the protein, and more preferably exists in the region of the 256th to 282nd amino acid residues from the N-terminus.

<Gene Encoding Pyridoxine Dehydrogenase, Gene Encoding Pyridoxamine Synthetase, and Gene Encoding Amino Acid Regeneration Enzyme>

The gene encoding the pyridoxine dehydrogenase may be any gene encoding the pyridoxine dehydrogenase described above. The gene encoding the pyridoxamine synthetase may be any gene encoding the pyridoxamine synthetase described above. The gene encoding the amino acid regeneration enzyme may be any gene encoding the amino acid regeneration enzyme described above. The enzymes encoded by these genes are not limited to known amino acid sequences having the enzymatic activities (for example, an amino acid sequence encoded by a gene naturally occurring in an organism), and may be enzymes having modified amino acid sequences different from the known amino acid sequences.

Such a gene may be a known gene such as a gene naturally possessed by the microorganism exemplified above as the microorganism (microorganism from which the enzyme is derived) having each enzyme, or a gene in which a nucleotide sequence is modified so that the nucleotide sequence encodes a modified amino acid sequence that is modified from the known amino acid sequence of the enzyme as described above as long as the desired enzymatic activity is obtained. Examples of the modified amino acid sequence include an amino acid sequence similar to any one of the amino acid sequences of SEQ ID NO:1 to SEQ ID NO:6 as described above. The nucleotide sequence of a gene encoding a particular amino acid sequence can be varied within degeneracy of a codon. In this case, it is preferable to use a codon frequently used in a microorganism used as a host of the recombinant microorganism from the viewpoint of gene expression efficiency.

It is also possible to design a nucleotide sequence of a gene based on an amino acid sequence to be encoded using the codon table. The designed nucleotide sequence may be obtained by modifying a known nucleotide sequence using genetic recombination technology, or may be obtained by chemically synthesizing the nucleotide sequence.

Examples of the method of modifying a nucleotide sequence include site-directed mutagenesis (Kramer, W. and frita, H. J., Methods in Enzymology, vol. 154, P. 350 (1987)), recombinant PCR (PCR Technology, Stockton Press (1989)), a method of chemically synthesizing a specific part of DNA, a method of treating a gene with hydroxyamine, a method of treating a strain carrying a gene with ultraviolet irradiation or a chemical agent such as nitrosoguanidine or nitrous acid, and a method of using a commercially available mutagenesis kit.

For example, each of the gene encoding the pyridoxine dehydrogenase, the gene encoding the pyridoxamine synthetase, and the gene encoding the amino acid regeneration enzyme may be DNA having an unmodified nucleotide sequence that encodes a polynucleotide having the enzymatic activity (for example, a nucleotide sequence possessed by a gene naturally occurring in an organism such as the above-described microorganism), or DNA having a nucleotide sequence in which a modification is made in the above nucleotide sequence as long as the enzymatic activity (the above-described enzymatic activity) of an enzyme encoded by the nucleotide sequence is not lost. Examples of the modification include insertion, deletion, and substitution of a nucleotide, and addition of an additional nucleotide to either or both of the 5′ end and 3′ end of the nucleotide sequence. In a case in which there is one or more of the insertion, deletion, and substitution of the nucleotide, the number of each of the insertion, deletion, and substitution, if present, may be, for example, from 1 to 90 nucleotides, from 1 to 60 nucleotides, from 1 to 30 amino acid residues, from 1 to 20 amino acid residues, from 1 to 15 nucleotides, from 1 to 10 nucleotides, or from 1 to 5 nucleotides; and the total number of the insertion, deletion, and substitution of the nucleotide may be, for example from 1 to 100 nucleotides, from 1 to 50 nucleotides, from 1 to 30 nucleotides, from 1 to 10 nucleotides, or from 1 to 5 nucleotides. Insertion or deletion of a nucleotide may occur locally, and it is preferable that the entire nucleotide sequence does not have a large frame shift. The number of nucleotide added to the end if present, may be, for example, from 1 to 150 nucleotides, from 1 to 100 nucleotides, from 1 to 50 nucleotides, from 1 to 30 nucleotides, from 1 to 10 nucleotides, or from 1 to 5 nucleotides per end. Such an additional nucleotide may encode a signal sequence for extracellular secretion or the like.

Alternatively, the gene encoding each of the enzymes may be DNA having a known nucleotide sequence (for example, a nucleotide sequence of a gene naturally occurring in an organism) that encodes a polynucleotide having the enzymatic activity per se, or DNA that has a nucleotide sequence having 80% or more, 85% or more, 90% or more, or 95% or more sequence identity with the known nucleotide sequence and that encodes an enzyme having a desired enzymatic activity (such as the above-mentioned enzymatic activity). Here, the sequence identity can be evaluated by using, for example, a BLAST (registered trademark, National Library of Medicine) program with default parameters.

Alternatively, the gene encoding each of the enzymes may be DNA having a known nucleotide sequence (for example, a nucleotide sequence of a gene naturally occurring in an organism) that encodes a polynucleotide having the enzymatic activity per se, or DNA having a nucleotide sequence that hybridizes with DNA having a nucleotide sequence complementary to the known nucleotide sequence under a stringent condition, and that encodes an enzyme having a desired enzymatic activity (such as the above-described enzymatic activity). Hybridization under a stringent condition can be performed as follows.

The hybridization is performed on DNA of interest using DNA consisting of a nucleotide sequence complementary to a reference nucleotide sequence or a partial sequence thereof as a probe. After washing the resultant under a stringent condition, the significance of the hybridization of the probe to the nucleic acid of interest is confirmed. The length of the probe can be, for example, 20 or more continuous nucleotides, preferably 50 or more nucleotides, more preferably 100 or more nucleotides, and further preferably 200 or more nucleotides. It is also preferable to use DNA as a probe that has the same nucleotide length as the reference nucleotide sequence and that is complementary to the reference nucleotide over the entire length. Examples of the conditions for hybridization may include conditions commonly used by those skilled in the art for detecting a specific hybridization signal. Such conditions preferably mean stringent hybridization conditions and stringent wash conditions. Examples thereof include conditions that a temperature of 55° C. is maintained overnight, together with a probe, in a solution including 6×SSC (saline sodium citrate) (composition of 1×SSC: 0.15 M NaCl, 0.015 M sodium citrate, pH 7.0), 0.5% SDS, 5×Denhardt, and 100 mg/mL herring sperm DNA. Examples thereof may include subsequent washing of a filter in 0.2×SSC at 42° C. Examples of the stringent conditions may include conditions of 0.1×SSC and 50° C. in the step of washing a filter, and, in addition, examples of the stringent conditions can include 0.1×SSC and 65° C. in the same step.

For example, the gene encoding the pyridoxine dehydrogenase may be, for example, DNA having the nucleotide sequence of SEQ ID NO:7 (the nucleotide sequence of a gene encoding a pyridoxine dehydrogenase from Saccharomyces cerevisiae), or DNA having a nucleotide sequence that hybridizes with DNA having a nucleotide sequence complementary to the nucleotide sequence of SEQ ID NO:7 under a stringent condition, and that encodes a protein having pyridoxine dehydrogenase activity.

Alternatively, the gene encoding the pyridoxine dehydrogenase may be, for example, DNA having the nucleotide sequence of SEQ ID NO:7, or DNA having a nucleotide sequence in which one or more of the substitution, deletion, or insertion of a nucleotide, or the addition of an additional nucleotide to either or both of the 5′ end and 3′ end of the nucleotide sequence are performed in the nucleotide sequence of SEQ ID NO:7, and which encodes a protein having pyridoxine dehydrogenase activity. Examples of the degrees of the substitution, deletion, and insertion of a nucleotide, and the addition of an additional nucleotide to either or both of the N-terminus and C-terminus of the nucleotide sequence are as described above.

Alternatively, the pyridoxine dehydrogenase may be DNA having the nucleotide sequence of SEQ ID NO:7, or may be DNA having a nucleotide sequence that has, for example, 80% or more, 85% or more, 90% or more, or 95% or more sequence identity with the nucleotide sequence of SEQ ID NO:7, and that encodes a protein having pyridoxine dehydrogenase activity.

Alternatively, the gene encoding the pyridoxine dehydrogenase may be, for example, DNA having the nucleotide sequence of SEQ ID NO:8 (the nucleotide sequence of a gene encoding a pyridoxine dehydrogenase from Schizosaccharomyces pombe), or DNA having a nucleotide sequence that hybridizes with DNA having a nucleotide sequence complementary to the nucleotide sequence of SEQ ID NO:8 under a stringent condition, and that encodes a protein having pyridoxine dehydrogenase activity.

Alternatively, the gene encoding the pyridoxine dehydrogenase may be, for example, DNA having the nucleotide sequence of SEQ ID NO:8, or DNA having a nucleotide sequence in which one or more of the substitution, deletion, or insertion of a nucleotide, or the addition of an additional nucleotide to either or both of the 5′ end and 3′ end of the nucleotide sequence are performed in the nucleotide sequence of SEQ ID NO:8, and which encodes a protein having pyridoxine dehydrogenase activity. Examples of the degrees of the substitution, deletion, and insertion of a nucleotide, and the addition of an additional nucleotide to either or both of the N-terminus and C-terminus of a nucleotide sequence are as described above.

Alternatively, the pyridoxine dehydrogenase may be DNA having the nucleotide sequence of SEQ ID NO:8, or may be DNA having a nucleotide sequence that has, for example, 80% or more, 85% or more, 90%, or 95% or more sequence identity with the nucleotide sequence of SEQ ID NO:8, and that encodes a protein having pyridoxine dehydrogenase activity.

Alternatively, the gene encoding the pyridoxine dehydrogenase may be, for example, DNA having the nucleotide sequence of any one of SEQ ID NO:7 or SEQ ID NO:53 to SEQ ID NO:59, or DNA having a nucleotide sequence that hybridizes with DNA having a nucleotide sequence complementary to the nucleotide sequence of any one of SEQ ID NO:7 or SEQ ID NO:53 to SEQ ID NO:59 under a stringent condition, and that encodes a protein having pyridoxine dehydrogenase activity.

Alternatively, the gene encoding the pyridoxine dehydrogenase may be, for example, DNA having the nucleotide sequence of any one of SEQ ID NO:7 or SEQ ID NO:53 to SEQ ID NO:59, or DNA having a nucleotide sequence in which one or more of the substitution, deletion, or insertion of a nucleotide, or the addition of an additional nucleotide to either or both of the 5′ end and 3′ end of the nucleotide sequence are performed in the nucleotide sequence of any one of SEQ ID NO:7 or SEQ ID NO:53 to SEQ ID NO:59, and which encodes a protein having pyridoxine dehydrogenase activity. Examples of the degrees of the substitution, deletion, and insertion of a nucleotide, and the addition of an additional nucleotide to either or both of the N-terminus and C-terminus of a nucleotide sequence are as described above.

Alternatively, the pyridoxine dehydrogenase may be DNA having the nucleotide sequence of any one of SEQ ID NO:7 or SEQ ID NO:53 to SEQ ID NO:59, or may be DNA having a nucleotide sequence that has, for example, 80% or more, 85% or more, 90% or more, or 95% or more sequence identity with at least one of the nucleotide sequence of SEQ ID NO:7, the nucleotide sequence of SEQ ID NO:53 (the nucleotide sequence of a gene encoding a pyridoxine dehydrogenase from Saccharomyces eubayanus), the nucleotide sequence of SEQ ID NO:54 (the nucleotide sequence of a gene encoding a pyridoxine dehydrogenase from Torulaspora delbrueckii), the nucleotide sequence of SEQ ID NO:55 (the nucleotide sequence of a gene encoding a pyridoxine dehydrogenase from Zygosaccharomyces bailii), the nucleotide sequence of SEQ ID NO:57 (the nucleotide sequence of a gene encoding a pyridoxine dehydrogenase from Kluyveromyces marxianus), the nucleotide sequence of SEQ ID NO:56 (the nucleotide sequence of a gene encoding a pyridoxine dehydrogenase from Aspergillus oryzae), the nucleotide sequence of SEQ ID NO:58 (the nucleotide sequence of a gene encoding a pyridoxine dehydrogenase from Candida albicans), or the nucleotide sequence of SEQ ID NO:59 (the nucleotide sequence of a gene encoding a pyridoxine dehydrogenase from Yarrowia lipolytica), and that encodes a protein having pyridoxine dehydrogenase activity.

In the case of expression in a recombinant microorganism in which a prokaryote such as E. coli is used as a host, a codon may be optimized to facilitate the expression. For example, a nucleotide sequence may be modified so that a codon, which is most frequently used, of codons that encode respective amino acids in a prokaryote as a host is highly frequently used as a codon for the amino acid. From such a viewpoint, for example, DNA having the region between the 18th nucleotide and the 3′ end of the nucleotide sequence of any one of SEQ ID NO:13 or SEQ ID NO:75 to SEQ ID NO:81 may be used, or DNA having a nucleotide sequence that hybridizes with DNA having a nucleotide sequence complementary to the region between the 18th nucleotide and the 3′ end of the nucleotide sequence of any one of SEQ ID NO:13 or SEQ ID NO:75 to SEQ ID NO:81 under a stringent condition, and that encodes a protein having pyridoxine dehydrogenase activity may be used, as DNA including a gene encoding the pyridoxine dehydrogenase. Seventeen nucleotides from the 5′ end of each of the nucleotide sequences of SEQ ID NO:13 and SEQ ID NO:75 to SEQ ID NO:81 are included in an upstream region, and an initiation codon is included in the 18th nucleotide to the 20th nucleotide. Therefore, the region between the 18th nucleotide and the 3′ end of each of these nucleotide sequences may be used as a gene region encoding a pyridoxine dehydrogenase.

Alternatively, the gene encoding the pyridoxine dehydrogenase may be, for example, DNA having the region between the 18th nucleotide to the 3′ end of the nucleotide sequence of any one of SEQ ID NO:13 or SEQ ID NO:75 to SEQ ID NO:81, or DNA having a nucleotide sequence in which one or more of the substitution, deletion, or insertion of a nucleotide, or the addition of an additional nucleotide to either or both of the 5′ end and 3′ end of the nucleotide sequence are performed in the region between the 18th nucleotide and 3′ end of the nucleotide sequence of any one of SEQ ID NO:13 or SEQ ID NO:75 to SEQ ID NO:81, and which encodes a protein having pyridoxine dehydrogenase activity. Examples of the degrees of the substitution, deletion, and insertion of a nucleotide, and the addition of an additional nucleotide to either or both of the N-terminus and C-terminus of a nucleotide sequence are as described above.

Alternatively, the pyridoxine dehydrogenase may be DNA having the region between the 18th nucleotide and 3′ end of the nucleotide sequence of any one of SEQ ID NO:13 or SEQ ID NO:75 to SEQ ID NO:81, or may be DNA having a nucleotide sequence that has, for example, 80% or more, 85% or more, 90% or more, or 95% or more sequence identity with at least one of the region between the 18th nucleotide and 3′ end of the nucleotide sequence of SEQ ID NO:13 (codon optimized nucleotide sequence of a gene encoding a pyridoxine dehydrogenase from Saccharomyces cerevisiae), the region between the 18th nucleotide and 3′ end of the nucleotide sequence of SEQ ID NO:75 (codon optimized nucleotide sequence of a gene encoding a pyridoxine dehydrogenase from Saccharomyces eubayanus), the region between the 18th nucleotide and 3′ end of the nucleotide sequence of SEQ ID NO:76 (codon optimized nucleotide sequence of a gene encoding a pyridoxine dehydrogenase from Torulaspora delbrueckii), the region between the 18th nucleotide and 3′ end of the nucleotide sequence of SEQ ID NO:77 (codon optimized nucleotide sequence of a gene encoding a pyridoxine dehydrogenase from Zygosaccharomyces bailii), the region between the 18th nucleotide and 3′ end of the nucleotide sequence of SEQ ID NO:79 (codon optimized nucleotide sequence of a gene encoding a pyridoxine dehydrogenase from Kluyveromyces marxianus), the region between the 18th nucleotide and 3′ end of the nucleotide sequence of SEQ ID NO:78 (codon optimized nucleotide sequence of a gene encoding a pyridoxine dehydrogenase from Aspergillus oryzae), the region between the 18th nucleotide and 3′ end of the nucleotide sequence of SEQ ID NO:80 (codon optimized nucleotide sequence of a gene encoding a pyridoxine dehydrogenase from Candida albicans), or the region between the 18th nucleotide and 3′ end of the nucleotide sequence of SEQ ID NO:81 (codon optimized nucleotide sequence of a gene encoding a pyridoxine dehydrogenase from Yarrowia lipolytica), and that encodes a protein having pyridoxine dehydrogenase activity.

The gene encoding the pyridoxamine synthetase may be, for example, DNA having the nucleotide sequence of SEQ ID NO:9 (nucleotide sequence of pyridoxamine-pyruvate transaminase gene from Mesorhizobium loti), or may be DNA having a nucleotide sequence that hybridizes with DNA having a nucleotide sequence complementary to the nucleotide sequence of SEQ ID NO:9 under a stringent condition, and that encodes a protein having pyridoxamine synthetase activity.

Alternatively, the gene encoding the pyridoxamine synthetase may be, for example, DNA having the nucleotide sequence of SEQ ID NO:9, or DNA having a nucleotide sequence in which one or more of the substitution, deletion, or insertion of a nucleotide, or the addition of an additional nucleotide to either or both of the 5′ end and the 3′ end of the nucleotide sequence are performed in the nucleotide sequence of SEQ ID NO:9, and which encodes a protein having pyridoxamine synthetase activity. Examples of the degrees of the substitution, deletion, and insertion of the nucleotide, and the addition of an additional nucleotide to either or both of the N-terminus and C-terminus of the nucleotide sequence are as described above.

Alternatively, the pyridoxamine synthetase may be DNA having the nucleotide sequence of SEQ ID NO:9, or may be DNA having a nucleotide sequence that has, for example, 80% or more, 85% or more, 90% or more, or 95% or more sequence identity with the nucleotide sequence of SEQ ID NO:9, and that encodes a protein having pyridoxamine synthetase activity.

The gene encoding the pyridoxamine synthetase may be, for example, DNA having the nucleotide sequence of SEQ ID NO:10 (nucleotide sequence of pyridoxamine-oxaloacetate transaminase gene from Escherichia coli), or may be DNA having a nucleotide sequence that hybridizes with DNA having a nucleotide sequence complementary to the nucleotide sequence of SEQ ID NO:10 under a stringent condition, and that encodes a protein having pyridoxamine synthetase activity.

Alternatively, the gene encoding the pyridoxamine synthetase may be, for example, DNA having the nucleotide sequence of SEQ ID NO:10, or DNA having a nucleotide sequence in which one or more of the substitution, deletion, or insertion of a nucleotide, or the addition of an additional nucleotide to either or both of the 5′ end and the 3′ end of the nucleotide sequence are performed in the nucleotide sequence of SEQ ID NO:10, and which encodes a protein having pyridoxamine synthetase activity. Examples of the degrees of the substitution, deletion, and insertion of the nucleotide, and the addition of an additional nucleotide to either or both of the N-terminus and C-terminus of the nucleotide sequence are as described above.

Alternatively, the pyridoxamine synthetase may be DNA having the nucleotide sequence of SEQ ID NO:9, or may be DNA having a nucleotide sequence that has, for example, 80% or more, 85% or more, 90% or more, or 95% or more sequence identity with the nucleotide sequence of SEQ ID NO:9, and that encodes a protein having pyridoxamine synthetase activity.

Alternatively, the gene encoding the pyridoxamine synthetase may be, for example, DNA having the nucleotide sequence of any one of SEQ ID NO:9 or SEQ ID NO:60 to SEQ ID NO:66, or may be DNA having a nucleotide sequence that hybridizes with DNA having a nucleotide sequence complementary to the nucleotide sequence of any one of SEQ ID NO:9 or SEQ ID NO:60 to SEQ ID NO:66 under a stringent condition, and that encodes a protein having pyridoxamine synthetase activity.

Alternatively, the gene encoding the pyridoxamine synthetase may be, for example, DNA having the nucleotide sequence of any one of SEQ ID NO:9 or SEQ ID NO:60 to SEQ ID NO:66, or DNA having a nucleotide sequence in which one or more of the substitution, deletion, or insertion of a nucleotide, or the addition of an additional nucleotide to either or both of the 5′ end and the 3′ end of the nucleotide sequence are performed in the nucleotide sequence of any one of SEQ ID NO:9 or SEQ ID NO:60 to SEQ ID NO:66, and which encodes a protein having pyridoxamine synthetase activity. Examples of the degrees of the substitution, deletion, and insertion of a nucleotide, and the addition of an additional nucleotide either or both of the N-terminus and C-terminus of a nucleotide sequence are as described above.

Alternatively, the pyridoxamine synthetase may be DNA having the nucleotide sequence of any one of SEQ ID NO:9 or SEQ ID NO:60 to SEQ ID NO:66, or may DNA having a nucleotide sequence that has, for example, 80% or more, 85% or more, 90% or more, or 95% or more sequence identity with at least one of the nucleotide sequence of SEQ ID NO:9, the nucleotide sequence of SEQ ID NO:60 (nucleotide sequence of a gene encoding pyridoxamine-pyruvate transaminase from Mesorhizobium sp. YR577), the nucleotide sequence of SEQ ID NO:61 (nucleotide sequence of a gene encoding pyridoxamine-pyruvate transaminase from Pseudaminobacter salicylatoxidans), the nucleotide sequence of SEQ ID NO:62 (nucleotide sequence of a gene encoding pyridoxamine-pyruvate transaminase from Bauldia litoralis), the nucleotide sequence of SEQ ID NO:63 (nucleotide sequence of a gene encoding pyridoxamine-pyruvate transaminase from Skermanella stibiiresistens), the nucleotide sequence of SEQ ID NO:64 (nucleotide sequence of gene encoding pyridoxamine-pyruvate transaminase from Rhizobium sp. AC44/96), the nucleotide sequence of SEQ ID NO:65 (nucleotide sequence of a gene encoding pyridoxamine-pyruvate transaminase from Erwinia toletana), or the nucleotide sequence of SEQ ID NO:66 (nucleotide sequence of a gene encoding pyridoxamine-pyruvate transaminase from Herbiconiux ginsengi), and that encodes a protein having pyridoxamine synthetase activity.

In the case of expression in a recombinant microorganism in which prokaryote such as E. coli is used as a host, a codon may be optimized, as described above, to facilitate expression. From such a viewpoint, as DNA including a gene encoding the pyridoxamine synthetase, for example, DNA having the region between the 18th nucleotide and the 3′ end of the nucleotide sequence of SEQ ID NO:14 or the region between the 18th nucleotide and the 3′ end of the nucleotide sequence of any one of SEQ ID NO:82 to SEQ ID NO:88 may be used, or DNA having a nucleotide sequence that hybridizes with DNA having a nucleotide sequence complementary to the region between 18th nucleotide and the 3′ end in the nucleotide sequence of SEQ ID NO:14 or the region between the 18th nucleotide and the 3′ end in the nucleotide sequence of any one of SEQ ID NO:82 to SEQ ID NO:88 under a stringent condition, and that encodes a protein having pyridoxamine synthetase activity may be used. Seventeen nucleotides from the 5′ end of the nucleotide sequence of SEQ ID NO:14 are included in an upstream region, and an initiation codon is included in the 18th nucleotide to the 20th nucleotide. Therefore, the region between the 18th nucleotide and the 3′ end of this nucleotide sequence may be used as a gene region encoding a pyridoxamine synthetase. Similarly, seventeen nucleotides from the 5′ end of each of the nucleotide sequences of SEQ ID NO:82 to SEQ ID NO:88 are included in an upstream, and an initiation codon is included in the 18th nucleotide to the 20th nucleotide. Therefore, the region between the 18th nucleotide and the 3′ end of these nucleotide sequences may be used as a gene region encoding a pyridoxamine synthetase.

Alternatively, the gene encoding the pyridoxamine synthetase may be, for example, DNA having the region between the 18th nucleotide and the 3′ end of the nucleotide sequence of SEQ ID NO:14 or the region between the 18th nucleotide and the 3′ end of the nucleotide sequence of any one of SEQ ID NO:82 to SEQ ID NO:88, or DNA having a nucleotide sequence in which one or more of the substitution, deletion, or insertion of a nucleotide, or the addition of an additional nucleotide to either or both of the 5′ end and the 3′ end of the are performed in the region between the 18th nucleotide and the 3′ end of the nucleotide sequence of the nucleotide sequence of SEQ ID NO:14 or the region between the 18th nucleotide and the 3′ end of the nucleotide sequence of any one of SEQ ID NO:82 to SEQ ID NO:88, and which encodes a protein having pyridoxamine synthetase activity. Examples of the degrees of the substitution, deletion, and insertion of a nucleotide, and the addition of an additional nucleotide to either or both of the N-terminus and C-terminus of the nucleotide sequence are as described above.

Alternatively, the pyridoxamine synthetase may be DNA having the region between the 18th nucleotide and the 3′ end of the nucleotide sequence of SEQ ID NO:14 or the region between the 18th nucleotide and the 3′ end of any one of nucleotide sequences of SEQ ID NO:82 to SEQ ID NO:88, or may be DNA having a nucleotide sequence that has, for example, 80% or more, 85% or more, 90% or more, or 95% or more sequence identity with at least one of the region between the 18th nucleotide and the 3′ end of the nucleotide sequence of SEQ ID NO:14 (codon-optimized nucleotide sequence of a gene encoding pyridoxamine-pyruvate transaminase from Mesorhizobium loti), the region between the 18th nucleotide and the 3′ end of the nucleotide sequence of SEQ ID NO:82 (codon-optimized nucleotide sequence of a gene encoding pyridoxamine-pyruvate transaminase from Mesorhizobium sp. YR577), the region between the 18th nucleotide and the 3′ end of the nucleotide sequence of SEQ ID NO:83 (codon-optimized nucleotide sequence of a gene encoding pyridoxamine-pyruvate transaminase from Pseudaminobacter salicylatoxidans), the region between the 18th nucleotide and the 3′ end of the nucleotide sequence of SEQ ID NO:84 (codon-optimized nucleotide sequence of a gene encoding pyridoxamine-pyruvate transaminase from Bauldia litoralis), the region between the 18th nucleotide and the 3′ end of the nucleotide sequence of SEQ ID NO:85 (codon-optimized nucleotide sequence of a gene encoding pyridoxamine-pyruvate transaminase from Skermanella stibiiresistens), the region between the 18th nucleotide and the 3′ end of the nucleotide sequence of SEQ ID NO:86 (codon-optimized nucleotide sequence of gene encoding pyridoxamine-pyruvate transaminase from Rhizobium sp. AC44/96), the region between the 18th nucleotide and the 3′ end of the nucleotide sequence of SEQ ID NO:87 (codon-optimized nucleotide sequence of a gene encoding pyridoxamine-pyruvate transaminase from Erwinia toletana), or the region between the 18th nucleotide and the 3′ end of the nucleotide sequence of SEQ ID NO:88 (codon-optimized nucleotide sequence of a gene encoding pyridoxamine-pyruvate transaminase from Herbiconiux ginsengi), and that encodes a protein having pyridoxamine synthetase activity.

The gene encoding the amino acid regeneration enzyme may be, for example, DNA having the nucleotide sequence of SEQ ID NO:11 (the nucleotide sequence of a gene encoding an alanine dehydrogenase from Shewanella sp. Ac10), or DNA having a nucleotide sequence that hybridizes with DNA having a nucleotide sequence complementary to the nucleotide sequence of SEQ ID NO:11 under a stringent condition, and that encodes a protein having alanine regeneration enzymatic activity.

Alternatively, the gene encoding the amino acid regeneration enzyme may be, for example, DNA having the nucleotide sequence of SEQ ID NO:11, or DNA having a nucleotide sequence in which one or more of the substitution, deletion, or insertion of a nucleotide, or the addition of an additional nucleotide to either or both of the 5′ end and 3′ end of the nucleotide sequence are performed in the nucleotide sequence of SEQ ID NO:11, and which encodes a protein having alanine regeneration enzymatic activity. Examples of the degrees of the substitution, deletion, and insertion of a nucleotide, and the addition of an additional nucleotide to either or both of the N-terminus and C-terminus of a nucleotide sequence are as described above.

Alternatively, the amino acid regeneration enzyme may be DNA having the nucleotide sequence of SEQ ID NO:11, or DNA having a nucleotide sequence that has, for example, 80% or more, 85% or more, 90% or more, or 95% or more sequence identity with the nucleotide sequence of SEQ ID NO:11, and that encodes a protein having alanine regeneration enzymatic activity.

Alternatively, the gene encoding the amino acid regeneration enzyme may be, for example, DNA having the nucleotide sequence of SEQ ID NO:12 (the nucleotide sequence of a gene encoding a glutamate dehydrogenase from Escherichia coli), or DNA having a nucleotide sequence that hybridizes with DNA having a nucleotide sequence complementary to the nucleotide sequence of SEQ ID NO:12 under a stringent condition, and that encodes a protein having glutamic acid regeneration enzymatic activity.

Alternatively, the gene encoding the amino acid regeneration enzyme may be, for example, DNA having the nucleotide sequence of SEQ ID NO:12, or DNA having a nucleotide sequence in which one or more of the substitution, deletion, or insertion of a nucleotide, or the addition of an additional nucleotide to either or both of the 5′ end and 3′ end of the nucleotide sequence are performed in the nucleotide sequence of SEQ ID NO:12, and which encodes a protein having glutamic acid regeneration enzymatic activity. Examples of the degrees of the substitution, deletion, and insertion of a nucleotide, and the addition of an additional nucleotide to either or both of the N-terminus and C-terminus of a nucleotide sequence are as described above.

Alternatively, the amino acid regeneration enzyme may be DNA having the nucleotide sequence of SEQ ID NO:12, or DNA having a nucleotide sequence that has, for example, 80% or more, 85% or more, 90% or more, or 95% or more a sequence identity with the nucleotide sequence of SEQ ID NO:12, and that encodes a protein having glutamic acid regeneration enzymatic activity.

The gene encoding the amino acid regeneration enzyme may be, for example, DNA having the nucleotide sequence of any one of SEQ ID NO:11 or SEQ ID NO:67 to SEQ ID NO:74, or DNA having a nucleotide sequence that hybridizes with DNA having a nucleotide sequence complementary to the nucleotide sequence of any one of SEQ ID NO:11 or SEQ ID NO:67 to SEQ ID NO:74 under s stringent condition, and that encodes a protein having alanine regeneration enzymatic activity.

Alternatively, the gene encoding the amino acid regeneration enzyme may be, for example, DNA having the nucleotide sequence of any one of SEQ ID NO:11 or SEQ ID NO:67 to SEQ ID NO:74, or DNA having a nucleotide sequence in which one or more of the substitution, deletion, or insertion of a nucleotide, or the addition of an additional nucleotide to either or both of the 5′ end and 3′ end of the nucleotide sequence are performed in the nucleotide sequence of any one of SEQ ID NO:11 or SEQ ID NO:67 to SEQ ID NO:74, and which encodes a protein having alanine regeneration enzymatic activity. Examples of the degrees of the substitution, deletion, and insertion of a nucleotide, and the addition of an additional nucleotide to either or both of the N-terminus and C-terminus of a nucleotide sequence are as described above.

Alternatively, the amino acid regeneration enzyme may be DNA having the nucleotide sequence of any one of SEQ ID NO:11 or SEQ ID NO:67 to SEQ ID NO:74, or may be DNA having a nucleotide sequence that has, for example, 80% or more, 85% or more, 90% or more, or 95% or more sequence identity with at least one of the nucleotide sequence of SEQ ID NO:11, the nucleotide sequence of SEQ ID NO:67 (the nucleotide sequence of a gene encoding an alanine dehydrogenase from Aeromonas hydrophila), the nucleotide sequence of SEQ ID NO:68 (the nucleotide sequence of a gene encoding an alanine dehydrogenase from Rhizobium sp. LPU83), the nucleotide sequence of SEQ ID NO:69 (the nucleotide sequence of a gene encoding an alanine dehydrogenase from Pseudomonas mendocina), the nucleotide sequence of SEQ ID NO:70 (the nucleotide sequence of a gene encoding one of alanine dehydrogenases from Bradyrhizobium japonicum), the nucleotide sequence of SEQ ID NO:71 (the nucleotide sequence of a gene encoding another alanine dehydrogenase from Bradyrhizobium japonicum), the nucleotide sequence of SEQ ID NO:72 (the nucleotide sequence of a gene encoding an alanine dehydrogenase from Streptomyces aureofaciens), the nucleotide sequence of SEQ ID NO:73 (the nucleotide sequence of a gene encoding an alanine dehydrogenase from Anabaena cylindrica), or the nucleotide sequence of SEQ ID NO:74 (the nucleotide sequence of a gene encoding an alanine dehydrogenase from Bacillus subtilis), and that encodes a protein having alanine regeneration enzymatic activity.

In the case of expression in a recombinant microorganism in which a prokaryote such as E. coli is used as a host, a codon may be optimized, as described above, to facilitate the expression. From such a viewpoint, as DNA including a gene encoding the amino acid regeneration enzyme, for example, DNA having the region between the 18th nucleotide and 3′ end of the nucleotide sequence of any one of SEQ ID NO:15 or SEQ ID NO:89 to SEQ ID NO:96 may be used, or DNA having a nucleotide sequence that hybridizes with DNA having a nucleotide sequence complementary to the region between the 18th nucleotide and 3′ end of the nucleotide sequence of any one of SEQ ID NO:15 or SEQ ID NO:89 to SEQ ID NO:96 under a stringent condition, and that encodes a protein having alanine regeneration enzymatic activity may be used. Seventeen nucleotides from the 5′ end of each of the nucleotide sequences of SEQ ID NO:15 and SEQ ID NO:89 to SEQ ID NO:96 are included in an upstream region, and an initiation codon is included in the 18th nucleotide to the 20th nucleotide. Therefore, the region between the 18th nucleotide and 3′ end of each of these nucleotide sequences may be used as a gene region encoding an alanine regeneration enzyme.

Alternatively, the gene encoding the amino acid regeneration enzyme may be, for example, DNA having the region between the 18th nucleotide to 3′ end of the nucleotide sequence of any one of SEQ ID NO:15 or SEQ ID NO:89 to SEQ ID NO:96, or DNA having a nucleotide sequence in which one or more of the substitution, deletion, or insertion of a nucleotide, or the addition of an additional nucleotide to either or both of the 5′ end and 3′ end of the nucleotide sequence are performed in the region between the 18th nucleotide and 3′ end of the nucleotide sequence of any one of SEQ ID NO:15 or SEQ ID NO:89 to SEQ ID NO:96, and which encodes a protein having alanine regeneration enzymatic activity. Examples of the degrees of the substitution, deletion, and insertion of a nucleotide, and the addition of an additional nucleotide to either or both of the N-terminus and C-terminus of a nucleotide sequence are as described above.

Alternatively, the amino acid regeneration enzyme may be DNA having the region between the 18th nucleotide and 3′ end of the nucleotide sequence of any one of SEQ ID NO:15 or SEQ ID NO:89 to SEQ ID NO:96, or may be DNA having a nucleotide sequence that has, for example, 80% or more, 85% or more, 90% or more, or 95% or more sequence identity with at least one of the region between the 18th nucleotide and 3′ end of the nucleotide sequence of SEQ ID NO:15 (codon optimized nucleotide sequence encoding an amino acid sequence in which the amino acid substitution of D198A is performed in the amino acid sequence of an alanine dehydrogenase from Shewanella sp. AC10), the region between the 18th nucleotide and 3′ end of the nucleotide sequence of SEQ ID NO:89 (codon optimized nucleotide sequence of a gene encoding an amino acid sequence in which amino acid substitution to use NADP⁺ is performed in an alanine dehydrogenase from Aeromonas hydrophila), the region between the 18th nucleotide and 3′ end of the nucleotide sequence of SEQ ID NO:90 (codon optimized nucleotide sequence of a gene encoding an amino acid sequence in which amino acid substitution to use NADP⁺ is performed in an alanine dehydrogenase from Rhizobium sp. LPU83), the region between the 18th nucleotide and 3′ end of the nucleotide sequence of SEQ ID NO:91 (codon optimized nucleotide sequence of a gene encoding an amino acid sequence in which amino acid substitution to use NADP⁺ is performed in an alanine dehydrogenase from Pseudomonas mendocina), the region between the 18th nucleotide and 3′ end of the nucleotide sequence of SEQ ID NO:92 (codon optimized nucleotide sequence of a gene encoding an amino acid sequence in which amino acid substitution to use NADP⁺ is performed in one of alanine dehydrogenases from Bradyrhizobium japonicum), the region between the 18th nucleotide and 3′ end of the nucleotide sequence of SEQ ID NO:93 (codon optimized nucleotide sequence of a gene encoding an amino acid sequence in which amino acid substitution to use NADP⁺ is performed in another alanine dehydrogenase from Bradyrhizobium japonicum), the region between the 18th nucleotide and 3′ end of the nucleotide sequence of SEQ ID NO:94 (codon optimized nucleotide sequence of a gene encoding an amino acid sequence in which amino acid substitution to use NADP⁺ is performed in an alanine dehydrogenase from Streptomyces aureofaciens), the region between the 18th nucleotide and 3′ end of the nucleotide sequence of SEQ ID NO:95 (codon optimized nucleotide sequence of a gene encoding an amino acid sequence in which amino acid substitution to use NADP⁺ is performed in an alanine dehydrogenase from Anabaena cylindrica), or the region between the 18th nucleotide and 3′ end of the nucleotide sequence of SEQ ID NO:96 (codon optimized nucleotide sequence of a gene encoding an amino acid sequence in which amino acid substitution to use NADP⁺ is performed in an alanine dehydrogenase from Bacillus subtilis), and that encodes a protein having alanine regeneration enzymatic activity.

<Recombinant Microorganism Having Gene Encoding Pyridoxine Dehydrogenase, Gene Encoding Pyridoxamine Synthetase, and Gene Encoding Amino Acid Regeneration Enzyme>

In the recombinant microorganism according to the disclosure, each of a gene encoding a pyridoxine dehydrogenase, a gene encoding a pyridoxamine synthetase, and a gene encoding an amino acid regeneration enzyme having an enzymatic activity of regenerating an amino acid consumed by the pyridoxamine synthetase may be endogenous to a bacterial cell, endogenous to a bacterial cell and has an enhanced expression, or introduced from outside of a bacterial cell into the bacterial cell. However, at least two of the gene encoding a pyridoxine dehydrogenase, the gene encoding a pyridoxamine synthetase, or the gene encoding an amino acid regeneration enzyme having an enzymatic activity of regenerating an amino acid consumed by the pyridoxamine synthetase, are introduced from outside of a bacterial cell, or are endogenous to a bacterial cell and have an enhanced expression.

In other words, each of the gene encoding a pyridoxine dehydrogenase, the gene encoding a pyridoxamine synthetase, and the gene encoding an amino acid regeneration enzyme having an enzymatic activity of regenerating an amino acid consumed by the pyridoxamine synthetase may be endogenous to the genome of a host microorganism prior to recombination, endogenous to the genome of a host microorganism and has an enhanced expression by an operation such as the substitution of a promoter, or introduced from outside of a bacterial cell into the bacterial cell using a vector such as a plasmid. In the first aspect, a pyridoxamine production capacity due to a combination of the three enzymes is exhibited, and each of at least two of the gene encoding a pyridoxine dehydrogenase, the gene encoding a pyridoxamine synthetase, or the gene encoding an amino acid regeneration enzyme having an enzymatic activity of regenerating an amino acid consumed by the pyridoxamine synthetase, is introduced from outside of a cell in the host microorganism, or has an enhanced expression of the gene endogenous to the cell in the host microorganism by the substitution of a promoter, or the like, whereby the expression of the gene is increased, and the pyridoxamine production capability due to the combination of the three enzymes is further enhanced. In the case of performing the introduction or expression enhancement of each of the gene encoding a pyridoxine dehydrogenase, the gene encoding a pyridoxamine synthetase, and a gene encoding an amino acid regeneration enzyme having an enzymatic activity of regenerating an amino acid consumed by the pyridoxamine synthetase, either or both of the introduction from outside of a cell in a host microorganism and the enhancement of the gene expression by the substitution of a promoter, or the like may be performed.

It is impossible to obtain a sufficient production capability for highly producing pyridoxamine or a salt thereof unless such increase in gene expression as described above is performed in at least two of the gene encoding a pyridoxine dehydrogenase, the gene encoding a pyridoxamine synthetase, or the gene encoding an amino acid regeneration enzyme having an enzymatic activity of regenerating an amino acid consumed by the pyridoxamine synthetase. Since the amino acid regeneration enzyme described herein refers to an amino acid regeneration enzyme having an enzymatic activity of regenerating a specific kind of an amino acid consumed by the pyridoxamine synthetase, the pyridoxamine synthetase and the amino acid regeneration enzyme can also be considered to be in a pair. Since such gene expression as described above is increased in at least two of the gene encoding a pyridoxine dehydrogenase, the gene encoding a pyridoxamine synthetase, or the gene encoding an amino acid regeneration enzyme having an enzymatic activity of regenerating an amino acid consumed by the pyridoxamine synthetase, the pair exists in a bacterial cell in a recombinant microorganism, and the gene expression is increased in at least either of the pyridoxamine synthetase and the amino acid regeneration enzyme in the pair.

Targets in which at least one of the introduction from outside of a bacterial cell or the enhancement of the expression of a gene endogenous to the bacterial cell is performed, may be only two of the gene encoding a pyridoxine dehydrogenase, the gene encoding a pyridoxamine synthetase, or the gene encoding an amino acid regeneration enzyme having an enzymatic activity of regenerating an amino acid consumed by the pyridoxamine synthetase. In a case in which at least one of the introduction from outside of a bacterial cell or the enhancement of the expression of a gene endogenous to the bacterial cell is performed in each of two of the gene encoding a pyridoxine dehydrogenase, the gene encoding a pyridoxamine synthetase, or the gene encoding an amino acid regeneration enzyme having an enzymatic activity of regenerating an amino acid consumed by the pyridoxamine synthetase, at least one of the introduction from outside of a bacterial cell or the enhancement of the expression of a gene endogenous to the bacterial cell may be performed in each of the gene encoding a pyridoxine dehydrogenase and the gene encoding a pyridoxamine synthetase, at least one of the introduction from outside of a bacterial cell or the enhancement of the expression of a gene endogenous to the bacterial cell may be performed in each of the gene encoding a pyridoxine dehydrogenase and the gene encoding the amino acid regeneration enzyme, and at least one of the introduction from outside of a bacterial cell or the enhancement of the expression of a gene endogenous to the bacterial cell may be performed in each of the gene encoding a pyridoxamine synthetase and the gene encoding the amino acid regeneration enzyme.

From the view point of increasing the production efficiency in the production of pyridoxamine or a salt thereof, it is preferable that at least one of the introduction from outside of a bacterial cell or the enhancement of the expression of a gene endogenous to the bacterial cell is performed in all of the gene encoding a pyridoxine dehydrogenase, the gene encoding a pyridoxamine synthetase, and the gene encoding an amino acid regeneration enzyme. From the view point of increasing the production efficiency in the production of pyridoxamine or a salt thereof, it is preferable that at least one of the introduction from outside of a bacterial cell or the enhancement of the expression of a gene endogenous to the bacterial cell is performed in the gene encoding an amino acid regeneration enzyme. The introduction of an enzyme gene from outside of a bacterial cell is not necessarily limited to the introduction for compensating an enzyme gene which is not present in a host microorganism, and the introduction may be performed for the purpose of increasing the expression of an enzyme gene endogenous to a host microorganism. An enzyme gene which is not present in a host microorganism can be easily confirmed using an enzyme database such as KEGG or BRENDA.

In a case in which the expression of a gene endogenous to a host microorganism is enhanced by the substitution of a promoter of the gene with another promoter, the another promoter (newly introduced promoter) is not particularly limited as long as the promoter can enhance the gene expression (more than before promoter substitution) in the host microorganism, and may be a constitutive promoter or an inducible promoter. Substitution of a promoter can be performed using a general genetic modification technology. The sequence of a promoter endogenous to a host microorganism may be partially or completely left unless the sequence adversely affects expression by a newly introduced promoter in practical use.

When the host microorganism is, for example, a prokaryote, examples of the promoter that can be used as the newly introduced promoter include a trp promoter, a lac promoter, and a GAPDH promoter from E. coli, a PL promoter and a PR promoter from lambda phage, and a gluconate synthetase promoter (gnt), an alkaline protease promoter (apr), a neutral protease promoter (npr), and an α-amylase promoter (amy) from Bacillus subtilis. An originally modified or designed promoter such as a tac promoter can also be used.

When the host microorganism is, for example, a filamentous fungus, examples of the promoter that can be used as the newly introduced promoter include a cellobiohydrolase (cbh) promoter, an endoglucanase (egl) promoter, a xylanase III (xyn3) promoter, a U6 promoter, and an α-amylase (amy) promoter.

When the host microorganism is, for example, yeast, examples of the promoter that can be used as the newly introduced promoter include an alcohol dehydrogenase (ADH1) promoter, a phosphoglycerate kinase (PGK1) promoter, a peptide chain elongation factor (TEF) promoter, a glycerol 3-phosphate dehydrogenase (GPD) promoter, a galactokinase (GAL1) promoter, a metallothionein (CUP1) promoter, a repressive acid phosphatase (PHO5) promoter, and a glyceraldehyde 3-phosphate dehydrogenase (GAPDH) promoter. The sequences of the above-described promoters are not limited to those derived from yeast used as a host microorganism. An exogenous promoter such as a cytomegalovirus (CMV) promoter may also be used.

In the case of introducing a gene (heterogenous gene) from outside of a host microorganism, the method is not particularly limited as long as a gene can be introduced into the inside of a bacterial cell (inside a cell) and an enzyme encoded by the gene can be expressed, and examples thereof include transformation with a plasmid carrying an enzyme gene, introduction of an enzyme gene into a genome, and a combination thereof. When introducing a gene, an expression vector into which the gene is integrated may be introduced into a bacterial cell. The expression vector is not particularly limited as long as the vector is a vector into which the nucleotide sequence of the gene is integrated, and the expression vector is more preferably a plasmid vector or a phage vector having the following constitution, from the viewpoint of improving transformation efficiency or translation efficiency.

The expression vector is not particularly limited as long as the vector includes the nucleotide sequence of the gene and can transform the host microorganism. If necessary, in addition to such a nucleotide sequence, a nucleotide sequence (hereinafter, also simply referred to as “another region”) constituting another region may be included. Examples of such a region include a control region required to the production of a desired enzyme by a recombinant microorganism obtained by transformation, and a region required for autonomous replication.

From the viewpoint of facilitating selection of the recombinant microorganism, the vector may further include a nucleotide sequence encoding a selection gene which can be used as a selection marker.

Examples of a control region required to the production of a desired enzyme include a promoter sequence (including an operator sequence for controlling transcription), a ribosome-binding sequence (SD sequence), and a transcription termination sequence.

In the case of using yeast as a host microorganism, from the viewpoint of expression efficiency of the gene, an expression vector that can be used preferably includes a promoter sequence in addition to the nucleotide sequence of the above gene. Any promoter sequence may be used as long as the sequence can express the gene in a transformant obtained using yeast as a host microorganism. Examples of the promoter sequence include the promoters described above as examples of the promoter that can be used as the newly introduced promoter in the case of using yeast as a host microorganism.

The expression vector may include a secretion signal. This allows to, when a recombinant microorganism produces a desired enzyme, extracellular secretion of the enzyme.

The secretion signal is not particularly limited as long as a desired enzyme can be secreted from yeast as a host microorganism. From the viewpoint of secretion efficiency, it is preferable to use an α factor signal sequence, an invertase signal sequence, an acid phosphatase signal sequence, a glucoamylase signal sequence, or the like.

Specific examples of the expression vector having the promoter sequence and the secretion signal as described above include pRS423, pRS424, and YEplac195.

From the viewpoint of expression efficiency of the gene, an expression vector that can be used when a filamentous fungus is used as a host microorganism preferably includes a promoter sequence in addition to the nucleotide sequence of the above gene. Any promoter sequence may be used as long as the sequence can express the gene in a transformant obtained by using a filamentous fungus as a host microorganism. Examples of the promoter include the promoters described above as examples of the promoter that can be used as the newly introduced promoter in the case of using a filamentous fungus as the host microorganism.

Suitable expression vectors for filamentous fungi are described in van den Hondel, C. A. M. J. J. et al. (1991) In: Bennett, J. W. and Lasure, L. L. (eds.) More Gene Manipulations in Fungi. Academic Press, pp. 396-428.

Another commonly used expression vector such as pUC18, pBR322, pUC100, pSL1180 (manufactured by Pharmacia Inc.), pFB6 and Aspergillus pRAX, or Trichoderma pTEX can also be used.

From the viewpoint of the expression efficiency of the gene, an expression vector which can be used when a prokaryote such as E. coli, Bacillus subtilis, or Actinomycetes is used as a host microorganism preferably includes a promoter sequence in addition to the nucleotide sequence of the above gene. The expression vector may include a ribosome-binding sequence, a transcription termination sequence, or the like other than a promoter sequence.

Examples of the promoter sequence include the promoters described above as examples of the promoter that can be used as the newly introduced promoter in the case of using a prokaryote as the host microorganism.

Examples of the ribosome-binding sequence include a sequence derived from E. coli or B. subtilis, and the ribosome-binding sequence is not particularly limited thereto as long as the sequence is a sequence that functions in a desired host microorganism such as E. coli or B. subtilis.

Examples of the ribosome-binding sequence include a sequence in which a consensus sequence having four or more consecutive nucleotides, of sequences complementary to the 3′ end region of 16S ribosomal RNA is produced by DNA synthesis.

The transcription termination sequence is not necessarily required, and a p-factor independent transcription termination, for example, as a lipoprotein terminator, a trp operon terminator, or the like can be used.

The order of these sequences on the expression vector of the control region is not particularly limited, and the promoter sequence, the ribosome-binding sequence, the gene encoding a target enzyme, and the transcription termination sequence are desirably arranged in this order from the side upstream of the 5′-end in consideration of transcription efficiency.

Specific examples of the expression vector that can be used when a host microorganism is a prokaryote include pBR322, pUC18, Bluescript II SK (+), pKK223-3, or pSC101 including a region that can be autonomously replicated in E. coli, or pUB110, pTZ4, pC194, ρ11, φ1, or φ105 including a region that can be autonomously replicated in Bacillus subtilis.

Examples of the vector that can be autonomously replicated in two or more kinds of host microorganisms include pHV14, TRp7, YEp7, and pBS7.

In the case of introducing a gene from outside of a cell in the host microorganism, the gene may be a gene having a nucleotide sequence that is not present in the genome of the host microorganism, and may be a gene having a nucleotide sequence that is present in the genome of the host microorganism. Even when the same gene originally exists in the genome of a host microorganism, the introduction of a gene from outside the bacterial cell causes stronger gene expression, and the enzymatic activity is enhanced and the production efficiency of pyridoxamine or a salt thereof can be further enhanced.

Conventional methods well known to those skilled in the art can be used as methods necessary for introducing a gene from outside of a bacterial cell (extracellular) into the bacterial cell (intracellular), such as preparation of a genomic DNA, cleavage and ligation of DNA, transformation, PCR (Polymerase Chain Reaction), and design and synthesis of an oligonucleotide used as a primer. These methods are described in Sambrook, J., et al., “Molecular Cloning A Laboratory Manual, Second Edition”, Cold Spring Harbor Laboratory Press, (1989) and the like. For example, a method using competent cells and a method using electroporation are described.

The host microorganism is not particularly limited as long as the microorganism is a microorganism capable of expressing, if present, a gene encoding the pyridoxine dehydrogenase, a gene encoding the pyridoxamine synthetase, and a gene encoding the amino acid regeneration enzyme. Examples of the host microorganism include yeasts, filamentous fungi, and prokaryotes. Examples of the yeasts include a yeast belonging to the genus Saccharomyces, such as Saccharomyces cerevisiae, a yeast belonging to the genus Schizosaccharomyces, such as Schizosaccharomyces pombe, a yeast belonging tothe genus Hansenula, and a yeast belonging to the genus Pichia. Examples of the filamentous fungi include a filamentous fungus belonging to the genus Trichoderma, such as Trichoderma reesei or viride, a filamentous fungus belonging to the genus Aspergillus, such as Aspergillus niger or oryzae, a filamentous fungus belonging to the genus Humicola, such as Humicola insolens, and a filamentous fungus belonging to the genus Acremonium, such as Acremonium cellulolyticus or fusidioides. Examples of the prokaryotes include a bacterium belonging to the genus Escherichia, such as Escherichia coli, a bacterium belonging to the genus Schewanella, such as Schewanella sp. AC10, a bacterium belonging to the genus Mesorhizobium, such as Mesorhizobium loti, a bacterium belonging to the genus Rhizobium, such as Rhizobium meliloti, a Bacillus bacterium belonging to the genus Bacillus, such as Bacillus subtilis, and an actinomycete such as an actinomycete belonging to the genus Streptomyces, such as Streptomyces lividans.

A desired gene can be introduced into a host microorganism by, for example, introducing (transforming) into these host microorganisms an expression vector including the desired gene. The introduced gene can be highly expressed in the obtained recombinant microorganism, for example, by the activity of a promoter included in the expression vector.

In a case in which the host microorganism is E. coli, for example, a competent cell method by calcium treatment, an electroporation method, or the like can be used as a method of introducing the recombinant DNA such as an expression vector into a cell of the host microorganism. The enzyme encoded by the introduced gene can be stably produced at a high expression level by culturing a recombinant microorganism obtained in such a manner.

The DNA encoding the enzyme can be isolated from the recombinant microorganism, and can be transferred into another microorganism. Using this DNA as a template, the DNA encoding the enzyme can be easily introduced into another host microorganism, by amplifying a DNA fragment encoding an enzyme by PCR, treating the resultant with a restriction enzyme or the like, and then the obtained fragment is ligated with another vector DNA fragment.

<Method of Producing Pyridoxamine or Salt Thereof>

In one embodiment, a method of producing pyridoxamine or a salt thereof includes bringing the recombinant microorganism, the culture of the recombinant microorganism, or the treated product of the recombinant microorganism or the culture according to the first aspect, into contact with pyridoxine or a salt thereof to produce pyridoxamine or a salt thereof.

Examples of a salt of pyridoxine include a salt of pyridoxine and an acid. Examples of the acid include hydrochloric acid, sulfuric acid, nitric acid, and phosphoric acid. Examples of the salt of pyridoxine include pyridoxine hydrochloride.

Examples of the salt of pyridoxamine include a salt of pyridoxamine with an acid. Examples of the acid include hydrochloric acid, sulfuric acid, nitric acid, acetic acid, and phosphoric acid. From the viewpoint of use as a medicament, the salt of pyridoxamine is preferably pyridoxamine dihydrochloride, which is most advanced for medical applications.

Such a method of producing pyridoxamine or a salt thereof using the recombinant microorganism, the culture of the recombinant microorganism, or the treated product of the recombinant microorganism or the culture is also simply referred to as “method of producing pyridoxamine or a salt thereof using a recombinant microorganism”.

The culture of the recombinant microorganism refers to a product obtained by culturing the recombinant microorganism, which is composed of a bacterial cell, a surrounding medium, and the like. The use of the culture is not necessarily required. For example, a dried or frozen recombinant microorganism cell prepared in advance may be added directly to a reaction system.

When culturing a recombinant microorganism, any of a synthetic medium or a natural medium can be used as a culture medium, as long as the culture medium is a culture medium including adequate amounts of a carbon source, a nitrogen source, an inorganic substance, and another nutrient. The culture can be performed using a general culture method, such as shaking culture, aeration stirring culture, continuous culture, or fed-batch culture in a liquid medium including the culture components.

More specifically, the culture condition for the recombinant microorganism is the same as the culture condition for the original host microorganism, and known conditions can be used.

Known components can be used as the components used in the culture medium. For example, organic nutrient sources such as a meat extract, a yeast extract, a malt extract, peptone, NZ amine, and potato, carbon sources such as glucose, maltose, sucrose, starch, and an organic acid, nitrogen sources such as ammonium sulfate, urea, and ammonium chloride, inorganic nutrient sourced such as phosphate, magnesium, potassium, and iron, and vitamins can be used in combination, if appropriate.

In culture of a recombinant microorganism transformed by the expression vector including the selection marker, for example, a culture medium including an agent against the drug resistance is used in a case in which the selection marker has drug resistance, and a culture medium that does not include a nutrient is used in a case in which the selection marker has a requirement for the nutrient.

The culture conditions may be selected, if appropriate, depending on the kinds of the recombinant microorganism, a culture medium, and a culture method, and are not particularly limited as long as the culture conditions are conditions under which the recombinant microorganism grows and can produce a pyridoxine dehydrogenase, a pyridoxamine synthetase, and an amino acid regeneration enzyme.

The pH of the culture medium may be selected, for example, in a range of from pH 4 to pH 8, and may be in a range of from pH 5 to pH 8.

The culture temperature is, for example, from 20° C. to 45° C., and preferably from 24° C. to 37° C. The culture may be performed aerobically or anaerobically depending on the kinds of a microorganism.

The culture period is, for example, from 1 day to 7 days. The culture period may be set to maximize the production amount of the target enzyme.

The treated product of the recombinant microorganism refers to a product obtained by any treatment of the recombinant microorganism as long as the activities of the pyridoxine dehydrogenase, the pyridoxamine synthetase, and the amino acid regeneration enzyme produced by the recombinant microorganism are not lost. Examples of such treatment include one or more selected from the group consisting of heat treatment, cooling treatment, mechanical destruction, ultrasonic treatment, freeze-thaw treatment, drying treatment, pressurized or reduced pressure treatment, osmotic pressure treatment, autolysis, surfactant treatment, and enzyme treatment (for example, cell lysis treatment). Even in a case in which the recombinant microorganism per se is killed by such treatment, such a treated product can be used for a reaction as long as the activity of the enzyme produced by the microorganism remains.

The treated product of the culture refers to a product obtained by any treatment of the culture of the recombinant microorganism as long as the activities of the pyridoxine dehydrogenase, the pyridoxamine synthetase, and the amino acid regeneration enzyme produced by the recombinant microorganism are not lost. Examples of such treatment include one or more selected from the group consisting of heat treatment, cooling treatment, mechanical destruction of a cell, ultrasonic treatment, freeze-thaw treatment, drying treatment, pressurized or reduced pressure treatment, osmotic pressure treatment, cell autolysis, surfactant treatment, enzyme treatment (for example, cell destruction treatment), cell separation treatment, purification treatment, and extraction treatment. For example, a cell of the recombinant microorganism may be separated from a culture medium or the like, and the separated cell may be added to a reaction system. Means such as filtration or centrifugation can be used for such separation. Alternatively, purification treatment for separating, from a contaminant, the pyridoxine dehydrogenase, the pyridoxamine synthetase, and the amino acid regeneration enzyme may be performed, and a solution including an enzyme obtained by the purification treatment may be added to the reaction system. Alternatively, an extract obtained by extracting the culture using an organic solvent such as methanol or acetonitrile, or a mixed solvent of an organic solvent and water may be added to the reaction system. It is also acceptable that such a purified product or extract does not include a cell of a recombinant microorganism. Even in a case in which the cell of the microorganism does not exist, such a purified product or extract can be used for a reaction as long as the enzymatic activity remains.

Such disruption or lysis treatment of a cell as described above can be performed by destructing the cell membrane of the recombinant microorganism according to a known method such as lysozyme treatment, freeze-thawing, or ultrasonic disruption.

It is preferable that the recombinant microorganism, the culture of the recombinant microorganism, or the treated product of the recombinant microorganism or the culture according to the first aspect, is brought into contact with pyridoxine or a salt thereof under the following conditions.

The contact is preferably performed in a solution including pyridoxine or a salt thereof as a substrate. The pH of the solution is not particularly limited as long as the enzymatic activities of the pyridoxine dehydrogenase, the pyridoxamine synthetase, and the amino acid regeneration enzyme are maintained, and is preferably from pH 6.0 to pH 9.0, and more preferably from pH 7.0 to pH 8.5. The temperature of the solution is also not particularly limited as long as the enzymatic activities of the pyridoxine dehydrogenase, the pyridoxamine synthetase, and the amino acid regeneration enzyme are maintained, and is preferably from 20° C. to 70° C., and more preferably from 25° C. to 50° C.

As a medium for the solution, water or an aqueous medium, an organic solvent, or a mixed solvent of an organic solvent and water or an aqueous medium may be used. Examples of the aqueous medium include buffers such as a phosphate buffer, a HEPES (N-2-hydroxyethylpiperazine-N-ethanesulfonic acid) buffer, and a tris[tris(hydroxymethyl)aminomethane]hydrochloride buffer. Any organic solvent may be used as long as the solvent does not inhibit a reaction, and examples thereof include acetone, ethyl acetate, dimethyl sulfoxide, xylene, methanol, ethanol, and butanol.

It is preferable that the recombinant microorganism, the culture of the recombinant microorganism, or the treated product of the recombinant microorganism or the culture according to the first aspect, is brought into contact with pyridoxine or a salt thereof under a shaking or stirring condition. For example, such contact can be performed in a solution. For example, pyridoxine or a salt thereof in the form of a substrate solution or in the form of a solid may be added to a solution including the recombinant microorganism, the culture of the recombinant microorganism, or the treated product of the recombinant microorganism or the culture. An amino acid to be consumed by a pyridoxamine synthetase may be further added to a solution including the recombinant microorganism, the culture of the recombinant microorganism, or the treated product of the recombinant microorganism or the culture. The amino acid may be included in a substrate solution including pyridoxine or a salt thereof, and may be added to a solution including the recombinant microorganism, the culture of the recombinant microorganism, or the treated product of the recombinant microorganism or the culture together with the pyridoxine or a salt thereof. Alternatively, pyridoxine or a salt thereof included in a substrate solution other than the substrate solution, or included in the form of a solid may be added to a solution including the recombinant microorganism, the culture of the recombinant microorganism, or the treated product of the recombinant microorganism or the culture.

An acid or an alkali may be added to maintain the pH of a reaction liquid in an appropriate range at the initiation of the reaction or during the reaction. Examples of the alkali that can be added to the reaction liquid include an alkali metal hydroxide such as lithium hydroxide, sodium hydroxide, or potassium hydroxide; and one such as ammonium hydroxide, calcium hydroxide, dipotassium phosphate, disodium phosphate, potassium pyrophosphate, or ammonia which is dissolved in water to make the liquid basic. Examples of the acid that can be added to a reaction liquid include hydrochloric acid, sulfuric acid, nitric acid, acetic acid, and phosphoric acid.

The contact may be performed, for example, under an air atmosphere or under a partial deoxygenation atmosphere. The deoxidizing atmosphere can be achieved by substitution with an inert gas, pressure reduction, boiling, or a combination thereof. It is preferable at least to replace with an inert gas, or to use an inert gas atmosphere. Examples of the inert gas include nitrogen gas, helium gas, argon gas, and carbon dioxide, and nitrogen gas is preferable.

In a preferable embodiment, the recombinant microorganism, the culture of the recombinant microorganism, or the treated product of the recombinant microorganism or the culture to be used includes the pyridoxine dehydrogenase, the pyridoxamine synthetase, and the amino acid regeneration enzyme having an enzymatic activity of regenerating an amino acid consumed by the pyridoxamine synthetase. For this reason, the pyridoxine dehydrogenase, the pyridoxamine synthetase, and the amino acid regeneration enzyme having an enzymatic activity of regenerating an amino acid consumed by the pyridoxamine synthetase, which are present together in the reaction liquid, act cooperatively when contacting, and pyridoxamine or a salt thereof is produced with high production efficiency. Although it is not essential that the treated product of a recombinant microorganism or the treated product of a culture includes the recombinant microorganism in a living state, from the viewpoint of continuously supplying the substance involved in the reaction by metabolism, it is preferable to include the recombinant microorganism in a living state.

Regarding the timing of addition, the recombinant microorganism, the culture of the recombinant microorganism, or the treated product of the recombinant microorganism or the culture, may be added all at once at the initiation of a reaction, or may be added in batches or continuously during a reaction. Similarly, pyridoxamine, which is a raw material, may be added all at once at the initiation of a reaction, or may be added in batches or continuously during a reaction.

The reaction liquid may include an amino acid to be consumed in the production of pyridoxamine or a salt thereof by a pyridoxamine synthetase. This amino acid may also be added all at once at the initiation of a reaction, or may be added in batches or continuously during a reaction. For example, in a case in which a pyridoxamine-pyruvate transaminase is used as a pyridoxamine synthetase, the reaction liquid may include one or more of L-alanine and D-alanine. In a case in which a pyridoxamine-oxaloacetate transaminase or an aspartate transaminase is used as a pyridoxamine synthetase, the reaction liquid may include at least one of L-aspartic acid, D-aspartic acid, L-glutamic acid, or D-glutamic acid. An amino acid may also exist as a salt depending on a surrounding environment; however, in the disclosure, a description of amino acid is given with inclusion of the mention of a salt. For example, the description of L-glutamic acid includes not only L-glutamic acid which has not formed salt, but also L-glutamic acid which has formed salt (for example, monosodium L-glutamate monohydrate). Examples of counter ions in the case of forming a salt include cations such as sodium ion and potassium ion, and anions such as chloride ion, acetate ion, and nitrate ion.

The concentration of pyridoxine or a salt thereof in the reaction liquid may be, for example, from 10 mM to 1 M, or may be from 50 mM to 800 mM, from 70 mM to 500 mM, or from 0.8 mM to 50 mM. The concentration of the amino acid (the amino acid to be consumed in producing pyridoxamine or a salt thereof by a pyridoxamine synthetase) may be, for example, from 0.1 mM to 2 M, or may be from 1 mM to 1 M, from 2 mM to 500 mM, from 2 mM to 400 mM, from 5 mM to 150 mM, or from 10 mM to 100 mM. Alternatively, the concentration of the amino acid may be from 5 mM to 800 mM, from 10 mM to 400 mM, or from 10 mM to 200 mM.

In the method of producing pyridoxamine or a salt thereof according to the first aspect, pyridoxamine or a salt thereof can be produced with high production efficiency even when the concentration of the amino acid in the reaction liquid is relatively low. It is a surprising effect that pyridoxamine or a salt thereof can be produced with high production efficiency even when relatively low concentration of the amino acid in used. Until now, for the production of pyridoxamine from pyridoxal, a relatively high concentration of glutamic acid was used in Journal of Molecular Catalysis B: Enzymatic, 2010, vol. 67, p. 104-110, and a relatively high concentration of alanine and glutamic acid was used in the wording example of WO 2007/142222. Pyridoxamine has an amino group, and an amino acid is considered to be consumed as a source providing the amino group. Therefore, it is considered that, in Journal of Molecular Catalysis B: Enzymatic, 2010, vol. 67, p. 104-110 and WO 2007/142222, a high concentration of amino acid was included in a reaction liquid in consideration of amino acid consumption. Although the reason that the above effect can be obtained in the method of producing pyridoxamine or a salt thereof according to the first aspect is not necessarily clear, it is assumed that an amino acid can be regenerated due to the presence of the amino acid regeneration enzyme in the recombinant microorganism according to the first aspect, whereby the production of pyridoxamine or a salt thereof is not arrested even when the amino acid concentration is not high, and pyridoxamine or a salt thereof can be produced at high production efficiency. As a result, according to the method of producing pyridoxamine or a salt thereof according to the first aspect, a cost of adding a large amount of amino acid can be saved, and time for performing an operation to maintain a high amino acid concentration by monitoring the concentration of an amino acid during a reaction can be save.

Examples of the method of bringing the recombinant microorganism, the culture of the recombinant microorganism, or the treated product of the recombinant microorganism or the culture, into contact with pyridoxine or a salt thereof include: a method of adding the recombinant microorganism, the culture of the recombinant microorganism, or the treated product of the recombinant microorganism or the culture, to a solution including pyridoxine or a salt thereof and allowing a reaction to proceed while stirring; a method of adding the recombinant microorganism, the culture of the recombinant microorganism, or the treated product of the recombinant microorganism or the culture, to a solution including pyridoxine or a salt thereof and allowing a reaction to proceed while shaking; and a method of mixing the recombinant microorganism, the culture of the recombinant microorganism, or the treated product of the recombinant microorganism or the culture and pyridoxine or a salt thereof thoroughly in a solution, then allowing the mixture to stand still for allowing a reaction to proceed. From the viewpoint of reaction efficiency, preferable examples thereof include a method of adding the recombinant microorganism, the culture of the recombinant microorganism, or the treated product of the recombinant microorganism or the culture, to a solution including pyridoxine or a salt thereof and allowing a reaction to proceed while stirring.

A reaction vessel that can be used for a reaction is not particularly limited. The reaction vessel is preferably a vessel in which the recombinant microorganism, the culture of the recombinant microorganism, or the treated product of the recombinant microorganism or the culture, and the solution including pyridoxine or a salt thereof added thereto can be stirred enough to mix them sufficiently, and which has a temperature control function of keeping the temperature within an optimum temperature range for a pyridoxine dehydrogenase, a pyridoxamine synthetase, and an amino acid regeneration enzyme.

The contact time (reaction time) of the recombinant microorganism, the culture of the recombinant microorganism, or the treated product of the recombinant microorganism or the culture, with pyridoxine or a salt thereof is not particularly limited as long as the enzymatic activities of the pyridoxine dehydrogenase, the pyridoxamine synthetase, and the amino acid regeneration enzyme are maintained. For example, the contact time may be from 30 minutes to 100 hours, and may be from 2 hours to 50 hours. The reaction may be performed with a batch method, may be performed with a semi-batch method in which either or both of a substrate, and the microorganism, the culture or the treated product are added in batches during the reaction, or may be performed with a continuous method. In the case of the semi-batch method or the continuous method, the upper limit of the reaction time is not particularly limited since an operation such as supplying either or both of a new raw material, and the recombinant microorganism, the culture, or the treated product is conducted.

In the above method, the use of the recombinant microorganism, the culture of the recombinant microorganism, or the treated product of the recombinant microorganism or the culture according to the first aspect, allows the production of pyridoxamine or a salt thereof inexpensively with high production efficiency by using pyridoxine or a salt thereof as a raw material, without the need to carry out a complicated step as in a chemical synthesis method. Pyridoxamine or a salt thereof obtained by the above-described method can be used, for example, for producing a product that utilizes its physiological activity. For example, the product can be used for an application such as prevention or treatment of a disease or schizophrenia caused by AGE accumulation such as diabetes mellitus, atherosclerosis, chronic renal failure, or Alzheimer's dementia, a health food, or a cosmetic.

<Second Aspect>

A recombinant microorganism selectively producing pyridoxal with high efficiency using pyridoxine as a substrate has not been known until now.

The structures of pyridoxine and pyridoxal are illustrated below. In the disclosure, the words “pyridoxine” and “pyridoxal” are used so as to be intended to encompass each salt thereof unless it is clearly described that the salt is particularly excluded. Examples of salts of pyridoxine include pyridoxine hydrochloride, pyridoxine sulfate, pyridoxine nitrate, and pyridoxine phosphate. Examples of salts of pyridoxal include pyridoxal hydrochloride, pyridoxal sulfate, pyridoxal nitrate, and pyridoxal phosphate.

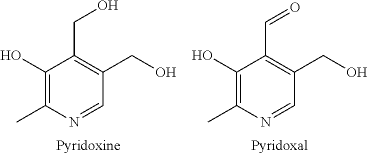

<Recombinant Microorganism According to Second Aspect>

[Amino Acid Sequence of SEQ ID NO:1]

The amino acid sequence of SEQ ID NO:1 is a pyridoxine dehydrogenase derived from Saccharomyces cerevisiae. The pyridoxine dehydrogenase is an oxidoreductase that catalyzes a reaction of producing pyridoxal, NADPH, and H⁺ from pyridoxine and NADP⁺ and a reaction reverse thereto, as described below. The pyridoxine dehydrogenase derived from Saccharomyces cerevisiae is classified under enzyme number EC1.1.1.65 according to the report of the enzyme commission of the International Union of Biochemistry (I.U.B.).

Pyridoxine+NADP ⇄Pyridoxal+NADPH+H⁺

In the recombinant microorganism according to the second aspect, a polynucleotide introduced into a host microorganism is at least one polynucleotide selected from the group consisting of the following (1) to (7).

(1) Polynucleotide Encoding Protein Having Amino Acid Sequence of SEQ ID NO:1

In the recombinant microorganism according to the second aspect, the polynucleotide introduced into a host microorganism is preferably a polynucleotide encoding a protein having the amino acid sequence of SEQ ID NO:1. In the case of the polynucleotide that encodes a protein including the amino acid sequence of SEQ ID NO:1, any codon may be used. Preferably, a polynucleotide having the nucleotide sequence of SEQ ID NO:13 is used.

(2) Polynucleotide Encoding Protein Having Amino Acid Sequence in Which from 1 to 50 Amino Acids Are Deleted, Added, or Substituted in Amino Acid Sequence of SEQ ID NO:1

In the recombinant microorganism according to the second aspect, the polynucleotide introduced into a host microorganism may be a polynucleotide that encodes a protein having an amino acid sequence in which from 1 to 50 amino acids are deleted, added, or substituted in the amino acid sequence of SEQ ID NO:1. The protein has an activity of synthesizing pyridoxine or pyridoxal. The protein preferably has an enzymatic activity classified under EC1.1.1.65.

In the second aspect, the activity of synthesizing pyridoxine refers to a capability of synthesizing pyridoxine using pyridoxal as a substrate, and the activity of synthesizing pyridoxal refers to a capability of synthesizing pyridoxal using pyridoxine as a substrate.

The number of amino acid residues deleted, added, or substituted in the amino acid sequence of SEQ ID NO:1 is preferably from 1 to 40, or from 1 to 30, still more preferably from 1 to 20, or from 1 to 15, and particularly preferably from 1 to 10, or from 1 to 5.

The activity of synthesizing pyridoxine can be confirmed by, for example, bringing a protein encoded by the polynucleotide of any one of the above (1) to (7) into contact with pyridoxal and NADPH under an appropriate environment such as phosphate buffered saline, and detecting the production of pyridoxine. The activity of synthesizing pyridoxal can be confirmed by bringing a protein encoded by the polynucleotide of any one of the above (1) to (7) into contact with pyridoxine and NADP⁺ under an appropriate environment such as phosphate buffered saline, and detecting the production of pyridoxal.

The detection of pyridoxal or pyridoxine can be performed by a known method, for example, high performance liquid chromatography (HPLC).

(3) Polynucleotide Encoding Protein Having Amino Acid Sequence Having 60% or More Sequence Identity with Amino Acid Sequence of SEQ ID NO:1

In the recombinant microorganism according to the second aspect, the polynucleotide introduced into a host microorganism may be a polynucleotide that encodes a protein having at least 60% sequence identity with the amino acid sequence of SEQ ID NO 1, and having an activity of synthesizing pyridoxine or pyridoxal. The protein preferably has an enzymatic activity classified under EC1.1.1.65.

The sequence identity is preferably at least 70%, more preferably at least 80%, or at least 85% or more, and still more preferably at least 90%, at least 91%, at least 92%, at least 93%, at least 94%, at least 95%, at least 96%, at least 97%, at least 98%, at least 99%, or 100%.

(4) Polynucleotide Having Nucleotide Sequence of SEQ ID NO:13

In the recombinant microorganism according to the second aspect, the polynucleotide introduced into a host microorganism is preferably a polynucleotide having the nucleotide sequence of SEQ ID NO:13.

The polynucleotide having the nucleotide sequence of SEQ ID NO:13 encodes a pyridoxine dehydrogenase from Saccharomyces cerevisiae, which is a protein having the amino acid sequence of SEQ ID NO:1.

(5) Polynucleotide Having Nucleotide Sequence in Which from 1 to 150 Nucleotides Are Deleted, Added, or Substituted in Nucleotide Sequence of SEQ ID NO:13

In the recombinant microorganism according to the second aspect, the polynucleotide introduced into a host microorganism may be a polynucleotide having a nucleotide sequence in which from 1 to 150 nucleotides are deleted, added, or substituted in the nucleotide sequence of SEQ ID NO:13. The protein encoded by the polynucleotide has an activity of synthesizing pyridoxine or pyridoxal, and preferably has an enzymatic activity classified under EC1.1.1.65.

The number of nucleotides deleted, added, or substituted in the nucleotide sequence of SEQ ID NO:13 is preferably from 1 to 140, from 1 to 120, from 1 to 100, from 1 to 90, from 1 to 80, from 1 to 70, from 1 to 60, or from 1 to 50, more preferably from 1 to 40, from 1 to 30, from 1 to 20, or from 1 to 15, and still more preferably from 1 to 10, or from 1 to 5.

(6) Polynucleotide Having Nucleotide Sequence Having 60% or More Sequence Identity with Nucleotide Sequence of SEQ ID NO:13

In the recombinant microorganism according to the second aspect, the polynucleotide introduced into a host microorganism may be a polynucleotide including a nucleotide sequence having 60% or more sequence identity with the nucleotide sequence of SEQ ID NO:13. A protein encoded by the polynucleotide has an activity of synthesizing pyridoxine or pyridoxal, and the protein preferably has an enzymatic activity classified under EC1.1.1.65.

The sequence identity is preferably at least 70%, more preferably at least 80%, or at least 85% or more, and still more preferably at least 90%, at least 91%, at least 92%, at least 93%, at least 94%, at least 95%, at least 96%, at least 97%, at least 98%, at least 99%, or 100%.

(7) Polynucleotide that Hybridizes with Polynucleotide Having Nucleotide Sequence Complementary to Nucleotide Sequence of SEQ ID NO:13 under Stringent Condition

In the recombinant microorganism according to the second aspect, the polynucleotide introduced into a host microorganism may be a polynucleotide that hybridizes with a polynucleotide having a nucleotide sequence complementary to the nucleotide sequence of SEQ ID NO:13 under a stringent condition. A protein encoded by the polynucleotide has an activity of synthesizing pyridoxine or pyridoxal, and the protein preferably has an enzymatic activity classified under EC1.1.1.65.

The stringent conditions refer to conditions under which a specific hybrid is formed, and a nonspecific hybrid is not formed. As the stringent conditions, conditions according to a sequence to be compared can be set, if appropriate, on the basis of the description of Molecular Cloning 3rd (J. Sambrook et al., Cold Spring Harbor Lab. Press, 2001). This document is incorporated herein by reference.

Typical stringent conditions mean, for example, stringent hybridization conditions and stringent washing conditions. Examples thereof include conditions that a temperature of 55° C. is maintained overnight, together with a probe, in a solution including 6×SSC (composition of 1×SSC: 0.15 M NaCl, 0.015 M sodium citrate, pH 7.0), 0.5% SDS, 5×Denhardt, and 100 mg/mL herring sperm DNA. Examples thereof may include subsequent washing of a filter in 0.2×SSC at 42° C. Examples of the stringent conditions may include conditions of 0.1×SSC and 50° C. in a step of washing a filter, and, in addition, examples of the stringent conditions can include conditions of 0.1×SSC and 65° C. in the same step.

The sequence identity with the amino acid sequence of SEQ ID NO:1 is expressed as a percentage obtained by multiplying, by 100, a value obtained by dividing the number of identical amino acids, in the case of performing optimal alignment to an amino acid sequence to be compared, by 345 which is the number of the amino acid residues of SEQ ID NO:1.

The sequence identity with the polynucleotide sequence of SEQ ID NO:13 is expressed as a percentage obtained by multiplying, by 100, a value obtained by dividing the number of identical nucleotides, in the case of performing optimal alignment to a polynucleotide sequence to be compared, by 1055 which is the number of the nucleotides of the polynucleotide of SEQ ID NO:13.

The optimal alignment refers to alignment in which the number of amino acids or nucleotides matching between two sequences is the greatest. The optimal alignment between sequences can be performed by using a known method, for example, a BLAST (registered trademark, National Library of Medicine) program while turning off mask filtering in the default parameters of the program.

The less number of amino acid residues or nucleotides deleted, added, or substituted in the amino acid sequence of SEQ ID NO:1 or the nucleotide sequence of SEQ ID NO:13 in the polynucleotide described in any one of the (2) (3), (5), or (6) described above is preferred, and a higher identity with the amino acid sequence of SEQ ID NO:1 or the nucleotide sequence of SEQ ID NO:13 is preferred. However, in the case of a variation that has no or less influence on enzymatic activity, the effect caused by the recombinant microorganism according to the second aspect can be exhibited even in a case in which the number of deleted, added, or substituted amino acid residues or nucleotides is large. For example, it is well known to those skilled in the art that substitution between amino acids having similar properties has little influence on the function of a protein. As another example, it is well known to those skilled in the art that there are plural kinds of codons that encode one amino acid, and even in the case of polynucleotides that encode an identical amino acid sequence, differences between used codons may result in many differences from the nucleotide sequence of SEQ ID NO:13.

In the recombinant microorganism according to the second aspect, a protein having a capability of synthesizing pyridoxal using pyridoxine as a substrate, or a protein having a capability of synthesizing pyridoxine using pyridoxal as a substrate is expressed from an introduced gene, and a protein having an enzymatic activity classified under EC1.1.1.65 is preferably expressed.

The expression of the protein having a capability of synthesizing pyridoxal using pyridoxine as a substrate or the protein having a capability of synthesizing pyridoxine using pyridoxal as a substrate from an introduced gene in the recombinant microorganism according to the second aspect may be confirmed by a known method. The expression of the protein can be confirmed by bringing the recombinant microorganism, into which a gene is introduced, into contact with pyridoxine or pyridoxal as a substrate, and detecting the production of pyridoxine or pyridoxal as the resultant of enzyme reaction. In the case of a recombinant microorganism in which different reactions occur before and after the synthesis of pyridoxine or pyridoxal, the production of pyridoxine or pyridoxal as the resultant of enzyme reaction from pyridoxine or pyridoxal as a substrate can be theoretically confirmed in consideration of the reactions before and after the synthesis.

<Preparation of Recombinant Microorganism according to Second Aspect>[Host]

As the microorganism used as a host in the case of preparing the recombinant microorganism according to the second aspect, a microorganism used in the expression of a protein can be commonly used as the host. Examples of the host microorganism include yeasts, filamentous fungi, and prokaryotes. Examples of the yeasts include a yeast belonging to the genus Saccharomyces, such as Saccharomyces cerevisiae, a yeast belonging to the genus Schizosaccharomyces, such as Schizosaccharomyces pombe, a yeast belonging to the genus Hansenula, and a yeast belonging to the genus Pichia. A microorganism capable of selectively and efficiently producing pyridoxal can be obtained by introducing the polynucleotide of any one of the (1) to (7) described above as well as an endogenous pyridoxine dehydrogenase in a case in which Saccharomyces cerevisiae is used as the host.

Examples of the filamentous fungi include a filamentous fungus belonging to the genus Trichoderma, such as Trichoderma reesei or viride, a filamentous fungus belonging to the genus Aspergillus, such as Aspergillus niger or oryzae, a filamentous fungus belonging to the genus Humicola, such as Humicola insolens, and a filamentous fungus belonging to the genus Acremonium, such as Acremonium cellulolyticus or fusidioides.

Examples of bacteria include an Escherichia bacterium, a Bacillus bacterium, a bacterium belonging to the genus Corynebacterium, and the genus Rhodococcus. E. coli (Escherichia coli) which has been industrially used in many cases is particularly preferably used. Examples of hosts other than E. coli include Bacillus brevis, Bacillus megaterium, Brevibacillus choshinensis, Corynebacterium glutamicum, and Rhodococcus erythropolis.

[Introduction into Host of Polynucleotide]

A method of introducing a polynucleotide into a host is known. A target polynucleotide can be introduced into a host by, for example, a common method, described in “Molecular Cloning 3rd Edition” (J. Sambrook et al.; Cold Spring Harbor Laboratory Press, 2001) and the like, and known in the fields of molecular biology, biotechnology, and genetic engineering. For example, a recombinant microorganism can be obtained by preparing an expression vector including the polynucleotides described in (1) to (6), and transforming any host microorganism by the expression vector.

In a case in which the host is bacterium, a competent cell method by calcium treatment, an electroporation method, or the like can be used as a method of introducing the recombinant DNA of an expression vector or the like into a cell of the host bacterium. A large amount of pyridoxine dehydrogenase can be stably expressed by culturing a recombinant bacterium obtained in such a manner.

The expression vector is not particularly limited as long as the expression vector includes the polynucleotide described in any one of the (1) to (7) as above. A common expression vector known in the field of genetic engineering can be adopted as the expression vector. The expression vector may include an element, if necessary, such as a control region necessary for the expression of the polynucleotide, or a region necessary for autonomous replication, enabling a protein to be produced by a recombinant microorganism. Examples of the control region necessary for the expression include a promoter sequence (including an operator sequence that controls transcription), a ribosome-binding sequence (SD sequence), a transcription termination sequence, a restriction enzyme cleavage sequence, or other genes. The other genes may be, for example, a protein having an action of converting produced pyridoxine or pyridoxal as an intermediate product into another material.

Examples of the promoter sequence include the trp promoter of a tryptophan operon and the lac promoter of a lactose operon derived from E. coli, a PL promoter and a PR promoter derived from lambda phage, and a gluconate synthase promoter (gnt), a glyceraldehyde 3-phosphate dehydrogenase (GAPDH) promoter, a glutamate decarboxylase A (gadA) promoter, a serine hydroxymethyltransferase (glyA) promoter, an alkaline protease promoter (apr), a neutral proteinase promoter (npr), or an α-amylase promoter (amy) derived from Bacillus subtilis.

An originally modified or designed promoter sequence such as a tac promoter can also be used.

Examples of the ribosome-binding sequence include a sequence derived from E. coli or Bacillus subtilis, and the ribosome-binding sequence is not particularly limited as long as the ribosome-binding sequence is a sequence that functions in a desired host cell such as E. coli or Bacillus subtilis.

Examples of the ribosome-binding sequence include a sequence in which a consensus sequence having four or more consecutive nucleotides, of sequences complementary to the 3′ end region of a 16S ribosomal RNA, is produced by DNA synthesis.

The transcription termination sequence is not necessarily required, and a p-factor independent terminator, for example, a lipoprotein terminator, a trp operon terminator, or the like can be used.

The order of these sequences on the expression vector of the control region is not particularly limited, and the promoter sequence, the ribosome-binding sequence, a gene encoding a target protein, and the transcription termination sequence are desirably aligned in the order mentioned above from the upstream (5′-end side) in consideration of transcription efficacy.

Specific examples of the expression vector herein include pBR322, pUC18, Bluescript II SK (+), pKK223-3, or pSC101 including a region that can be autonomously replicated in E. coli, or pUB110, pTZ4, pC194, ρ11, φ1, or φ105 including a region that can be autonomously replicated in Bacillus subtilis, which can be used as the expression vector.

Examples of expression vectors that can be autonomously replicated in two or more kinds of hosts include pHV14, TRp7, YEp7, and pBS7, which can be used as the expression vectors.

<Method of Producing Pyridoxal>

A method of producing pyridoxal according to the second aspect includes bringing the recombinant microorganism according to the second aspect into contact with pyridoxine. The recombinant microorganism obtained by introducing the expression vector described above into a desired host microorganism produces a protein encoded by a polynucleotide included in the expression vector. The produced protein generates pyridoxal using pyridoxine as a substrate.

The recombinant microorganism according to the second aspect may be proliferated by culturing the recombinant microorganism. Culture conditions are similar to the conditions under which the host cell is cultured, and known conditions can be used as the culture conditions.

Any of a synthetic medium and a natural medium can be used as a culture medium as long as the culture medium is a culture medium including adequate amounts of a carbon source, a nitrogen source, an inorganic substance, and another nutrient. Known components can be used as the components used in the culture medium. For example, organic nutrient sources such as a meat extract, a yeast extract, a malt extract, peptone, NZ amine, and potato, carbon sources such as glucose, maltose, sucrose, starch, and an organic acid, nitrogen sources such as ammonium sulfate, urea, and ammonium chloride, inorganic nutrient sources such as a phosphate, magnesium, potassium, and iron, and vitamins can be used in combination, if appropriate.

In culture of a recombinant microorganism transformed by the expression vector including the selection marker, for example, a culture medium including an agent against the drug resistance is used in a case in which the selection marker has the drug resistance, and a culture medium that does not include a nutrient is used in a case in which the selection marker has a requirement for the nutrient. The pH of the culture medium may be selected in a range of from pH 4 to pH 8.

The culture of the recombinant microorganism can be performed using a usual culture method such as shaking culture, aeration stirring culture, continuous culture, or fed-batch culture in a liquid medium containing the culture medium.

The culture conditions may be selected, if appropriate, depending on the kinds of a host, a culture medium, and a culture method, and are not particularly limited as long as the culture conditions are conditions under which a recombinant microorganism grows, and the recombinant microorganism according to the second aspect can be produced.

The culture is aerobically performed at a culture temperature of from 20° C. to 45° C., preferably from 24° C. to 37° C.

For example, in a case in which the host is E. coli, an LB (Lysogeny Broth) culture medium, an M9 culture medium, or the like is commonly used as a culture medium in which recombinant E. coli is cultured, and a culture medium in which such a culture medium is allowed to contain 0.1 μg/mL or more of Fe ions and Co ions as components is more preferred. The recombinant E. coli may be inoculated into the culture medium, and then grown at an appropriate culture temperature (of commonly from 20° C. to 50° C.).

A culture obtained by culturing the recombinant microorganism according to the second aspect, or a treated product of the culture may be brought into contact with pyridoxine as a substrate in order to produce pyridoxal. A recombinant protein produced by the recombinant microorganism according to the second aspect can produce pyridoxal using pyridoxine as the substrate.

The culture obtained by culturing the recombinant microorganism is not particularly limited as long as the culture is obtained by culturing a recombinant microorganism. The culture may be a bacterial cell recovered from a culture medium, or may be a protein encoded by the polynucleotide that is recovered from a culture medium and described in any one of the (1) to (7) described above. Alternatively, a culture medium including a bacterial cell, or a culture medium that does not include a bacterial cell but includes a recombinant protein produced by a recombinant microorganism may be used as it is.

The treated product of the culture refers to a product obtained by any treatment of the culture of the recombinant microorganism according to the second aspect as long as the activity of the recombinant protein produced by the recombinant microorganism is not lost. Examples of such treatment include treatment including one or more selected from the group consisting of heat treatment, cooling treatment, mechanical destruction of a cell, ultrasonic treatment, freeze-thaw treatment, drying treatment, pressurized or reduced pressure treatment, osmotic pressure treatment, cell autolysis, surfactant treatment, enzyme treatment (for example, cell destruction treatment), cell separation treatment, purification treatment, and extraction treatment. For example, a cell of the recombinant microorganism may be separated from a culture medium or the like, and the separated cell may be added to a reaction system. Means such as filtration or centrifugation can be used for such separation. Alternatively, purification treatment for separating, from a contaminant, the recombinant protein produced by the recombinant microorganism according to the second aspect may be performed, and a solution containing an enzyme obtained by the purification treatment may be added to the reaction system. Alternatively, an extract obtained by extracting the culture using an organic solvent such as methanol or acetonitrile, or a mixed solvent of an organic solvent and water may be added to the reaction system. It is also acceptable that such a purified product or extract does not include a bacterial cell of a recombinant microorganism. Even in a case in which the bacterial cell does not exist, such a purified product or extract can be used in production of pyridoxine or pyridoxal as long as the activity of the recombinant protein produced by the recombinant microorganism according to the second aspect remains.

Such disruption or lysis treatment of a cell as described above can be performed by destructing the cell membrane of the recombinant microorganism according to a known method such as lysozyme treatment, freeze-thawing, or ultrasonic disruption.

In the second aspect, the treated product of the culture may be obtained by, for example, immobilizing a bacterial cell or a protein encoded by the polynucleotide described in any one of the (1) to (7) as above on a solid phase such as a bead or a membrane. A conventionally known method can be used as a method of immobilizing the bacterial cell or protein on the solid phase.

During culturing the recombinant microorganism according to the second aspect, the recombinant microorganism and pyridoxine as a substrate may be brought into contact with each other in order to produce pyridoxal. For example, pyridoxine may be added to a culture medium in order to bring the recombinant microorganism and pyridoxine into contact with each other. Pyridoxine may be included in the culture medium from the beginning of culture, or may be added after, for example, the proliferation period of the microorganism starts following the start of the culture.

In the method of producing pyridoxal according to the second aspect, produced pyridoxal may be recovered from the cultured recombinant microorganism per se, the culture medium, and others, or may be directly subjected to a desired subsequent step without recovering the pyridoxal. A method commonly used in this field can be used as a method of recovering pyridoxal. The subsequent step is not particularly limited as long as pyridoxal is used in the subsequent step. Examples of the subsequent step include a step of phosphorylating pyridoxal.

Pyridoxal can be selectively produced with high efficiency by the methods of producing the recombinant microorganism and pyridoxal according to the second aspect, as described above.

The term “step” as used herein includes not only a separate step but also a step that is not clearly distinguished from other steps as long as the desired purpose of the step is achieved therefrom. As used herein, the notation “to” expressing a numerical range including the numerical values before and after “to”, as the minimum value and the maximum value, respectively.

Herein, the amount of a component of a composition, when plural substances corresponding to the same component exist in the composition, the total amount of the component in the composition refers to a total amount of the plural substances in the composition, unless otherwise specified.

### EXAMPLES

The present embodiment is described more specifically with reference to the following examples. However, the disclosure is not limited at all to these examples. In addition, “%” indicating the amounts of components contained in compositions in examples is based on mass standard unless otherwise specified.

<Analysis Conditions>

Pyridoxine hydrochloride, pyridoxal hydrochloride, and pyridoxamine dihydrochloride were quantified by high performance liquid chromatography. The analysis conditions thereof are as follows.

Column: Shodex (registered trademark) Asahipak ODP-50 6E (SHOWA DENKO K.K.)

Guard column: Shodex (registered trademark) Asahipak ODP-50G 6A (SHOWA DENKO K.K.)

Column temperature: 30° C.

Pump flow rate: 1.0 mL/min

Eluent: 50 mM phosphate buffer (pH 2.0)

Detection: UV 254 nm

### Comparative Example 1: Production of ppat Expression Strain

A synthetic DNA having the nucleotide sequence of SEQ ID NO:14 was obtained by custom synthesis of the nucleotide sequence of a pyridoxamine-pyruvate aminotransferase gene derived from Mesorhizobium loti MAFF303099, codon-optimized for E. coli, from GenScript. A DNA fragment including a target gene was amplified by PCR with an oligonucleotide having the nucleotide sequence of SEQ ID NO:18 and an oligonucleotide having the nucleotide sequence of SEQ ID NO:19 as primers using the synthetic DNA as a template. The amplified DNA fragment was treated with EcoRI and BamHI, and the obtained DNA fragment and a treated product of pUC18 (manufactured by Takara Bio Inc.) with EcoRI and BamHI were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:14 according to a usual method of determining a nucleotide sequence. The obtained plasmid was named pUC18-ppat. Here, ppat is the abbreviated name of a pyridoxamine-pyruvate aminotransferase gene.

### Comparative Example 2: Production of ppat-plr Expression Strain

A synthetic DNA having the nucleotide sequence of SEQ ID NO:13 was obtained by custom synthesis of the nucleotide sequence of a pyridoxal reductase gene derived from Saccharomyces cerevisiae, codon-optimized for E. coli, from GenScript. A DNA fragment including a target gene was amplified by PCR with an oligonucleotide having the nucleotide sequence of SEQ ID NO:16 and an oligonucleotide having the nucleotide sequence of SEQ ID NO:17 as primers using the synthetic DNA as a template. The amplified DNA fragment was treated with SalI and HindIII, and the obtained DNA fragment and a treated product obtained by treating the pUC18-ppat produced in Comparative Example 1 with SalI and HindIII were ligated using Ligation High (manufactured by TOYOBO Co., Ltd.). Escherichia coli DH5α (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:13 according to a usual method of determining a nucleotide sequence. The obtained plasmid was named pUC18-ppat-plr. Here, plr is the abbreviated name of a pyridoxal reductase gene (corresponding to pyridoxine dehydrogenase). E. coli DH5a does not originally have a gene of a pyridoxine dehydrogenase. Further, E. coli DH5a does not originally have a gene of an alanine regeneration enzyme capable of regenerating L-alanine consumed by pyridoxamine-pyruvate aminotransferase. Accordingly, a ppat-plr expression strain obtained in such a case corresponds to a comparative example because the ppat-plr expression strain does not have a gene encoding an amino acid regeneration enzyme having an enzymatic activity of regenerating an amino acid consumed by an introduced pyridoxamine synthetase although two of a gene encoding a pyridoxine dehydrogenase, a gene encoding a pyridoxamine synthetase, or a gene encoding an amino acid regeneration enzyme are introduced from outside of a bacterial cell.

### Comparative Example 3: Production of ppat-adh Expression Strain

A synthetic DNA having the nucleotide sequence of SEQ ID NO:15 was obtained by custom synthesis of the nucleotide sequence of an alanine dehydrogenase gene derived from Shewanella sp. AC10, into which a mutation as D198A in an encoded amino acid sequence was introduced, and which was codon-optimized for E. coli, from GenScript. A DNA fragment including a target gene was amplified by PCR with an oligonucleotide having the nucleotide sequence of SEQ ID NO:20 and an oligonucleotide having the nucleotide sequence of SEQ ID NO:21 as primers using the synthetic DNA as a template. The amplified DNA fragment was treated with BamHI and SalI, and the obtained DNA fragment and a treated product obtained by treating the pUC18-ppat produced in Comparative Example 1 with BamHI and SalI were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:15 according to a usual method of determining a nucleotide sequence. The obtained plasmid was named pUC18-ppat-adh. Here, adh is the abbreviated name of an alanine dehydrogenase gene. As described above, E. coli DH5a does not originally has a gene of a pyridoxine dehydrogenase, and therefore, the ppat-adh expression strain obtained in such a case corresponds to a comparative example.

### Example 1: Production of ppat-adh-plr Expression Strain

A synthetic DNA having the nucleotide sequence of SEQ ID NO:15 was obtained by custom synthesis of the nucleotide sequence of an alanine dehydrogenase gene derived from Shewanella sp. AC10, into which a mutation as D198A in an encoded amino acid sequence was introduced, and which was codon-optimized for E. coli, from GenScript. A DNA fragment including a target gene was amplified by PCR with an oligonucleotide having the nucleotide sequence of SEQ ID NO:20 and an oligonucleotide having the nucleotide sequence of SEQ ID NO:21 as primers using the synthetic DNA as a template. The amplified DNA fragment was treated with BamHI and SalI, and the obtained DNA fragment and a treated product obtained by treating the pUC18-ppat-plr produced in Comparative Example 2 with BamHI and SalI were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:15 according to a usual method of determining a nucleotide sequence. The obtained plasmid was named pUC18-ppat-adh-plr.

Test 1: Production of Pyridoxamine Using Pyridoxine as Raw Material

DH5a transformed with each of pUC18 and the plasmids produced in Comparative Examples 1 to 3 and Example 1 was inoculated into 100 mL of LB medium including 100 μg/mL of ampicillin in a 500 mL baffled Erlenmeyer flask and cultured with shaking at 30° C. for 24 hours. The culture solution was centrifuged at 8,000 rpm for 20 minutes, and bacterial cells obtained as a precipitate were suspended in 1 mL of water to prepare a bacterial cell suspension.

Pyridoxine hydrochloride and L-alanine were dissolved in water to prepare a substrate solution including pyridoxine hydrochloride (600 mM) and L-alanine (600 mM). The pH of the substrate solution was adjusted to pH 8.0 with 25% aqueous ammonia, and 500 of substrate solution and 1,000 μL of bacterial cell suspension were mixed, and allowed to react at 37° C. for 24 hours. A part of the reaction liquid was collected, and analyzed under the analysis conditions described above. The results are set forth in Table 1. The yield set forth in Table 1 represents the ratio of the molar amount of obtained pyridoxamine dihydrochloride to the molar amount of pyridoxine hydrochloride in the substrate solution.

TABLE 1

| Introduced Plasmid | Yield | Remarks |
|---|---|---|
| pUC18 | 0% | |
| pUC18-ppat | 0% | Comparative Example 1 |
| pUC18-ppat-plr | 7% | Comparative Example 2 |
| pUC18-ppat-adh | 0% | Comparative Example 3 |
| pUC 18-ppat-adh-plr | 88% | Example 1 |

As set forth in Table 1, pyridoxamine dihydrochloride was produced with high production efficiency in a case in which all of the gene encoding a pyridoxine dehydrogenase, the gene encoding a pyridoxamine synthetase, and the gene encoding an amino acid regeneration enzyme having an enzymatic activity of regenerating an amino acid consumed by a pyridoxamine synthetase were included, and two or more of the genes were extracellularly introduced.

Test 2: Production of Pyridoxamine Using Pyridoxine as Raw Material (Examination of Concentration of L-alanine)

DH5a transformed with the plasmid (pUC18-ppat-adh-plr) produced in Example 1 was inoculated into 100 mL of LB medium including 100 μg/mL of ampicillin in a 500 mL baffled Erlenmeyer flask and cultured with shaking at 30° C. for 24 hours. The culture solution was centrifuged at 8,000 rpm for 20 minutes, and bacterial cells obtained as a precipitate were suspended in 0.5 mL of water to prepare a bacterial cell suspension.

Pyridoxine hydrochloride was dissolved in water, and the solution was adjusted to pH 8.0 with 25% aqueous ammonia to prepare a substrate solution (1) including pyridoxine hydrochloride (600 mM). L-alanine was dissolved in water, and the solution was adjusted to pH 8.0 with 25% aqueous ammonia to prepare a substrate solution (2) including L-alanine (1,200 mM). Mixing of 500 μL of substrate solution (1) and a predetermined amount of substrate solution (2) was performed. To each liquid mixture, 500 μL of bacterial cell suspension was added, and water was further added, to prepare 1,500 μL of reaction liquid, which was allowed to react at 37° C. for 24 hours. The amount of substrate solution (2) in such a case was changed so that the concentration of L-alanine in each reaction liquid was 0 mM, 1 mM, 5 mM, 10 mM, 50 mM, 200 mM, 400 mM, or 800 mM. A part of the reaction liquid was collected, and analyzed under the analysis conditions described above, to quantify pyridoxine hydrochloride, pyridoxal hydrochloride, and pyridoxamine dihydrochloride. The results are set forth in Table 2. The yield set forth in Table 2 represents the ratio of the molar amount of obtained pyridoxamine dihydrochloride to the molar amount of pyridoxine hydrochloride in the substrate solution.

TABLE 2

| L-alanine (mM) | Yield |
|---|---|
| 0 | 39% |
| 1 | 46% |
| 5 | 57% |
| 10 | 76% |
| 50 | 91% |
| 200 | 88% |
| 400 | 69% |
| 800 | 61% |

As set forth in Table 2, with regard to the concentration of L-alanine in the substrate liquid mixture, high yield was achieved in a wide range of the concentration of L-alanine. Pyridoxamine of which concentration was not less than the concentration of L-alanine was produced in a case in which the concentration of L-alanine was in a concentration range of from 0 mM to 50 mM. This reveals that L-alanine consumed by reaction for producing pyridoxamine was able to be regenerated by introducing alanine dehydrogenase, and pyridoxamine production reaction was allowed to proceed by the regeneration of L-alanine even in the case of the low concentration of L-alanine.

Test 3: Production of Pyridoxamine Using Pyridoxine as Raw Material (Examination of Concentration of L-alanine)

DH5a obtained by transforming the plasmid (pUC18-ppat-adh-plr) produced in Example 1 was inoculated into 100 mL of LB medium including 100 μg/mL of ampicillin in a 500 mL baffled Erlenmeyer flask and cultured with shaking at 30° C. for 24 hours. The culture solution was centrifuged at 8,000 rpm for 20 minutes, and bacterial cells obtained as a precipitate were suspended in 0.5 mL of water to prepare a bacterial cell suspension.

Pyridoxine hydrochloride was dissolved in water, the solution was adjusted to pH 8.0 with 25% aqueous ammonia to prepare a substrate solution including pyridoxine hydrochloride (600 mM). 500 μL of this substrate solution was mixed with 500 μL of bacterial cell suspension, and a residual amount of water to prepare 1500 μL of reaction liquid, an L-alanine powder was added to achieve saturation solubility, and the resultant was allowed to react at 37° C. for 24 hours. A part of the reaction liquid was collected, and analyzed under the analysis conditions described above, to quantify pyridoxine hydrochloride, pyridoxal hydrochloride, and pyridoxamine dihydrochloride. As a result, the reaction yield was 39%. In other words, higher yield was able to be achieved in the case of using L-alanine at lower concentration.

As described above, it is revealed that the method of producing pyridoxamine or a salt thereof according to the first aspect enables pyridoxamine or a salt thereof to be inexpensively produced from pyridoxine or a salt thereof with high production efficiency even in the case of using a relatively small amount of amino acid to be consumed rather than adding a large amount of amino acid to be consumed.

### Example 2: Production of ppat-adh-Seplr Expression Plasmid

A synthetic DNA having the nucleotide sequence of SEQ ID NO:75 was obtained by custom synthesis of a gene in which the nucleotide sequence of a pyridoxal reductase gene derived from Saccharomyces eubayanus was designed to be codon-optimized for E. coli, and in which SalI was introduced into the 5′ end and HindIII was introduced into the 3′ end, from eurofins. A DNA fragment obtained by treating the synthetic DNA with SalI/HindIII, and a DNA fragment obtained by treating pUC18-ppat-adh produced in Comparative Example 3 with SalI and HindIII were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:75 according to a usual method of determining a nucleotide sequence. The obtained plasmid was named pUC18-ppat-adh-Seplr.

### Example 3: Production of ppat-adh-Tdplr Expression Plasmid

A synthetic DNA having the nucleotide sequence of SEQ ID NO:76 was obtained by custom synthesis of a gene in which the nucleotide sequence of a pyridoxal reductase gene derived from Torulaspora delbrueckii was designed to be codon-optimized for E. coli, and in which SalI was introduced into the 5′ end and HindIII was introduced into the 3′ end, from eurofins. A DNA fragment obtained by treating the synthetic DNA with SalI/HindIII, and a DNA fragment obtained by treating pUC18-ppat-adh produced in Comparative Example 3 with SalI and HindIII were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:76 according to a usual method of determining a nucleotide sequence. The obtained plasmid was named pUC18-ppat-adh-Tdplr.

### Example 4: Production of ppat-adh-Zbplr Expression Plasmid

A synthetic DNA having the nucleotide sequence of SEQ ID NO:77 was obtained by custom synthesis of a gene in which the nucleotide sequence of a pyridoxal reductase gene derived from Zygosaccharomyces bailii was designed to be codon-optimized for E. coli, and in which SalI was introduced into the 5′ end and HindIII was introduced into the 3′ end, from GenScript. A DNA fragment obtained by treating the synthetic DNA with SalI/HindIII, and a DNA fragment obtained by treating pUC18-ppat-adh produced in Comparative Example 3 with SalI and HindIII were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:77 according to a usual method of determining a nucleotide sequence. The obtained plasmid was named pUC18-ppat-adh-Zbplr.

### Example 5: Production of ppat-adh-Aoplr Expression Plasmid

A synthetic DNA having the nucleotide sequence of SEQ ID NO:78 was obtained by custom synthesis of a gene in which the nucleotide sequence of a pyridoxal reductase gene derived from Aspergillus oryzae was designed to be codon-optimized for E. coli, and in which SalI was introduced into the 5′ end and HindIII was introduced into the 3′ end was synthesized, from GenScript. A DNA fragment obtained by treating the synthetic DNA with SalI/HindIII, and a DNA fragment obtained by treating pUC18-ppat-adh produced in Comparative Example 3 with SalI and HindIII were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:78 according to a usual method of determining a nucleotide sequence. The obtained plasmid was named pUC18-ppat-adh-Aoplr.

### Example 6: Production of ppat-adh-Kmplr Expression Plasmid

A synthetic DNA having the nucleotide sequence of SEQ ID NO:79 was obtained by custom synthesis of a gene in which the nucleotide sequence of a pyridoxal reductase gene derived from Kluyveromyces marxianus was designed to be codon-optimized for E. coli, and in which SalI was introduced into the 5′ end and HindIII was introduced into the 3′ end, from GenScript. A DNA fragment obtained by treating the synthetic DNA with SalI/HindIII, and a DNA fragment obtained by treating pUC18-ppat-adh produced in Comparative Example 3 with SalI and HindIII were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:79 according to a usual method of determining a nucleotide sequence. The obtained plasmid was named pUC18-ppat-adh-Kmplr.

### Example 7: Production of ppat-adh-Caplr Expression Plasmid

A synthetic DNA having the nucleotide sequence of SEQ ID NO:80 was obtained by custom synthesis of a gene in which the nucleotide sequence of a pyridoxal reductase gene derived from Candida albicans was designed to be codon-optimized for E. coli, and in which SalI was introduced into the 5′ end and HindIII was introduced into the 3′ end, from eurofins A DNA fragment obtained by treating the synthetic DNA with SalI/HindIII, and a DNA fragment obtained by treating pUC18-ppat-adh produced in Comparative Example 3 with SalI and HindIII were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:80 according to a usual method of determining a nucleotide sequence. The obtained plasmid was named pUC18-ppat-adh-Caplr.

### Example 8: Production of ppat-adh-Ylplr Expression Plasmid

A synthetic DNA having the nucleotide sequence of SEQ ID NO:81 was obtained by custom synthesis of a gene in which the nucleotide sequence of a pyridoxal reductase gene derived from Yarrowia lipolytica was designed to be codon-optimized for E. coli, and in which SalI was introduced into the 5′ end and HindIII was introduced into the 3′ end, from GenScript. A DNA fragment obtained by treating the synthetic DNA with SalI/HindIII, and a DNA fragment obtained by treating pUC18-ppat-adh produced in Comparative Example 3 with SalI and HindIII were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:81 according to a usual method of determining a nucleotide sequence. The obtained plasmid was named pUC18-ppat-adh-Ylplr.

Test 4: Production of Pyridoxamine Using Pyridoxine as Raw Material

DH5α transformed with each of the plasmids produced in Examples 2 to 8 was inoculated into 100 mL of LB medium including 100 μg/mL of ampicillin in a 500 mL baffled Erlenmeyer flask and cultured with shaking at 30° C. for 24 hours. 40 mL of culture solution was taken, and centrifuged at 5,000 rpm for 5 minutes, and bacterial cells obtained as a precipitate were suspended in 0.9 mL of water to prepare a bacterial cell suspension.

Pyridoxine hydrochloride and L-alanine were dissolved in water to prepare a substrate solution including pyridoxine hydrochloride (500 mM) and L-alanine (500 mM). The pH of the substrate solution was adjusted to pH 8.0 with 25% aqueous ammonia, and 400 of substrate solution, 500 μL of water, and 100 μL of bacterial cell suspension were mixed, and allowed to react at 37° C. for 1 hour. A part of the reaction liquid was collected, and analyzed under the analysis conditions described above. The results are set forth in Table 3. The amount of produced pyridoxamine set forth in Table 3 is expressed in a relative amount, with the molar amount of pyridoxamine dihydrochloride produced by the ppat-adh-plr expression strain produced in Example 1 set to 100.

TABLE 3

| | Amount of Produced Pyridoxamine |
|---|---|
| Introduced Plasmid | (Relative Value) |
| pUC18-ppat-adh-plr | 100 |
| pUC18-ppat-adh-Seplr | 120 |
| pUC18-ppat-adh-Tdplr | 19 |
| pUC18-ppat-adh-Zbplr | 29 |
| pUC18-ppat-adh-Aoplr | 75 |
| pUC18-ppat-adh-Kmplr | 92 |
| pUC18-ppat-adh-Caplr | 112 |
| pUC18-ppat-adh-YlpIr | 53 |

As set forth in Table 3, pyridoxamine dihydrochloride was able to be also produced from pyridoxine hydrochloride by introducing a pyridoxal reductase gene derived from Saccharomyces eubayanus, a pyridoxal reductase gene derived from Torulaspora delbrueckii, a pyridoxal reductase gene derived from Zygosaccharomyces bailii, a pyridoxal reductase gene derived from Aspergillus oryzae, a pyridoxal reductase gene derived from Kluyveromyces marxianus, a pyridoxal reductase gene derived from Candida albicans, or a pyridoxal reductase gene derived from Yarrowia lipolytica, as a gene encoding a pyridoxine dehydrogenase, instead of the pyridoxal reductase gene derived from Saccharomyces cerevisiae used in Example 1.

### Comparative Example 4: Production of adh-plr Expression Plasmid

A pyridoxal reductase gene (plr) fragment recovered by treating pUC18-ppat-plr produced in Comparative Example 2 with SalI and HindIII, an alanine dehydrogenase gene (adh) fragment recovered by treating pUC-ppat-adh produced in Comparative Example 3 with BamHI and SalI, and a treated product of pUC18 with BamHI and HindIII were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner, and the obtained plasmid was named pUC18-adh-plr.

### Example 9: Production of Msppat-adh-plr Expression Plasmid

A synthetic DNA having the nucleotide sequence of SEQ ID NO:82 was obtained by custom synthesis of a gene in which the nucleotide sequence of a pyridoxamine-pyruvate aminotransferase gene derived from Mesorhizobium sp. YR577 was designed to be codon-optimized for E. coli, and in which EcoRI was introduced into the 5′ end and BamHI was introduced into the 3′ end, from eurofins. A DNA fragment obtained by treating the synthetic DNA with EcoRI/BamHI, and a DNA fragment obtained by treating pUC18-adh-plr produced in Comparative Example 4 with EcoRI and BamHI were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:82 according to a usual method of determining a nucleotide sequence. The obtained plasmid was named pUC18-Msppat-adh-plr.

### Example 10: Production of Psppat-adh-plr Expression Plasmid

A synthetic DNA having the nucleotide sequence of SEQ ID NO:83 was obtained by custom synthesis of a gene in which the nucleotide sequence of a pyridoxamine-pyruvate aminotransferase gene derived from Pseudaminobacter salicylatoxidans was designed to be codon-optimized for E. coli, and in which EcoRI was introduced into the 5′ end and BamHI was introduced into the 3′ end, from eurofins. A DNA fragment obtained by treating the synthetic DNA with EcoRI/BamHI, and a DNA fragment obtained by treating pUC18-adh-plr produced in Comparative Example 4 with EcoRI and BamHI were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:83 according to a usual method of determining a nucleotide sequence. The obtained plasmid was named pUC18-Psppat-adh-plr.

### Example 11: Production of Blppat-adh-plr Expression Plasmid

A synthetic DNA having the nucleotide sequence of SEQ ID NO:84 was obtained by custom synthesis of a gene in which the nucleotide sequence of a pyridoxamine-pyruvate aminotransferase gene derived from Bauldia litoralis was designed to be codon-optimized for E. coli, in which EcoRI was introduced into the 5′ end and BamHI was introduced into the 3′ end, from eurofins. A DNA fragment obtained by treating the synthetic DNA with EcoRI/BamHI, and a DNA fragment obtained by treating pUC18-adh-plr produced in Comparative Example 4 with EcoRI and BamHI were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:84 according to a usual method of determining a nucleotide sequence. The obtained plasmid was named pUC18-Blppat-adh-plr.

### Example 12: Production of Ssppat-adh-plr Expression Plasmid

A synthetic DNA having the nucleotide sequence of SEQ ID NO:85 was obtained by custom synthesis of a gene in which the nucleotide sequence of a pyridoxamine-pyruvate aminotransferase gene derived from Skermanella stibiiresistens was designed to be codon-optimized for E. coli, and in which EcoRI was introduced into the 5′ end and BamHI was introduced into the 3′ end, from eurofins. A DNA fragment obtained by treating the synthetic DNA with EcoRI/BamHI, and a DNA fragment obtained by treating pUC18-adh-plr produced in Comparative Example 4 with EcoRI and BamHI were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:85 according to a usual method of determining a nucleotide sequence. The obtained plasmid was named pUC18-Ssppat-adh-plr.

### Example 13: Production of Rsppat-adh-plr Expression Plasmid

A synthetic DNA having the nucleotide sequence of SEQ ID NO:86 was obtained by custom synthesis of a gene in which the nucleotide sequence of a pyridoxamine-pyruvate aminotransferase gene derived from Rhizobium sp. AC44/96 was designed to be codon-optimized for E. coli, and in which EcoRI was introduced into the 5′ end and BamHI was introduced into the 3′ end, from eurofins. A DNA fragment obtained by treating the synthetic DNA with EcoRI/BamHI, and a DNA fragment obtained by treating pUC18-adh-plr produced in Comparative Example 4 with EcoRI and BamHI were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:86 according to a usual method of determining a nucleotide sequence. The obtained plasmid was named pUC18-Rsppat-adh-plr.

### Example 14: Production of Etppat-adh-plr Expression Plasmid

A synthetic DNA having the nucleotide sequence of SEQ ID NO:87 was obtained by custom synthesis of a gene in which the nucleotide sequence of a pyridoxamine-pyruvate aminotransferase gene derived from Erwinia toletana was designed to be codon-optimized for E. coli, and in which EcoRI was introduced into the 5′ end and BamHI was introduced into the 3′ end, from eurofins. A DNA fragment obtained by treating the synthetic DNA with EcoRI/BamHI, and a DNA fragment obtained by treating pUC18-adh-plr produced in Comparative Example 4 with EcoRI and BamHI were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:87 according to a usual method of determining a nucleotide sequence. The obtained plasmid was named pUC18-Etppat-adh-plr.

### Example 15: Production of Hgppat-adh-plr Expression Plasmid

A synthetic DNA having the nucleotide sequence of SEQ ID NO:88 was obtained by custom synthesis of a gene in which the nucleotide sequence of a pyridoxamine-pyruvate aminotransferase gene derived from Herbiconiux ginsengi was designed to be codon-optimized for E. coli, and in which EcoRI was introduced into the 5′ end and BamHI was introduced into the 3′ end, from eurofins. A DNA fragment obtained by treating the synthetic DNA with EcoRI/BamHI, and a DNA fragment obtained by treating pUC18-adh-plr produced in Comparative Example 4 with EcoRI and BamHI were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:88 according to a usual method of determining a nucleotide sequence. The obtained plasmid was named pUC18-Hgppat-adh-plr.

Test 5: Production of Pyridoxamine Using Pyridoxine as Raw Material

DH5a transformed with each of the plasmids produced in Examples 9 to 15 was inoculated into 100 mL of LB medium including 100 μg/mL of ampicillin in a 500 mL baffled Erlenmeyer flask and cultured with shaking at 30° C. for 24 hours. 40 mL of culture solution was taken, and centrifuged at 5,000 rpm for 5 minutes, and bacterial cells obtained as a precipitate were suspended in 0.9 mL of water to prepare a bacterial cell suspension.

Pyridoxine hydrochloride and L-alanine were dissolved in water to prepare a substrate solution including pyridoxine hydrochloride (500 mM) and L-alanine (500 mM). The pH of the substrate solution was adjusted to pH 8.0 with 25% aqueous ammonia. 400 of substrate solution, 500 μL of water, and 100 μL of bacterial cell suspension were mixed, and allowed to react at 37° C. for 1 hour. A part of the reaction liquid was collected, and analyzed under the analysis conditions described above. The results are set forth in Table 4. The amount of produced pyridoxamine set forth in Table 4 is expressed in a relative amount, with the molar amount of pyridoxamine dihydrochloride produced by the ppat-adh-plr expression strain produced in Example 1 set to 100.

TABLE 4

| | Amount of Produced Pyridoxamine |
|---|---|
| Introduced Plasmid | (Relative Value) |
| pUC18-ppat-adh-plr | 100 |
| pUC18-Msppat-adh-plr | 46 |
| pUC18-Psppat-adh-plr | 113 |
| pUC18-BIppat-adh-plr | 34 |
| pUC18-Ssppat-adh-plr | 77 |
| pUC18-Rsppat-adh-plr | 34 |
| pUC18-Etppat-adh-plr | 109 |
| pUC18-Hgppat-adh-plr | 75 |

As set forth in Table 4, pyridoxamine dihydrochloride was able to be also produced from pyridoxine hydrochloride by introducing a pyridoxamine-pyruvate aminotransferase gene derived from Mesorhizobium sp. YR577, a pyridoxamine-pyruvate aminotransferase gene derived from Pseudaminobacter salicylatoxidans, a pyridoxamine-pyruvate aminotransferase gene derived from Bauldia litoralis, a pyridoxamine-pyruvate aminotransferase gene derived from Skermanella stibiiresistens, a pyridoxamine-pyruvate aminotransferase gene derived from Rhizobium sp. AC44/96, a pyridoxamine-pyruvate aminotransferase gene derived from Envinia toletana, or a pyridoxamine-pyruvate aminotransferase gene derived from Herbiconiux ginsengi, as a gene encoding a pyridoxamine-pyruvate aminotransferase, instead of the pyridoxamine-pyruvate aminotransferase gene derived from Mesorhizobium loti MAFF303099 used in Example 1.

### Example 16: Production of ppat-Ahadh-plr Expression Plasmid

A synthetic DNA having the nucleotide sequence of SEQ ID NO:89 was obtained by custom synthesis of the nucleotide sequence of an alanine dehydrogenase gene derived from Aeromonas hydrophila, into which a mutation as D198A in an encoded amino acid sequence was introduced, which was designed to be codon-optimized for E. coli, and in which BamHI was introduced into the 5′ end and SalI was introduced into the 3′ end, from eurofins. A DNA fragment obtained by treating the synthetic DNA with BamHI/SalI, and a DNA fragment obtained by treating pUC18-ppat-plr produced in Comparative Example 2 with BamHI and SalI were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:89 according to a usual method of determining a nucleotide sequence. The obtained plasmid was named pUC18-ppat-Ahadh-plr.

### Example 17: Production of ppat-Rsadh-plr Expression Plasmid

A synthetic DNA having the nucleotide sequence of SEQ ID NO:90 was obtained by custom synthesis of the nucleotide sequence of an alanine dehydrogenase gene derived from Rhizobium sp. LPU83, into which a mutation as D198A in an encoded amino acid sequence was introduced, which was designed to be codon-optimized for E. coli, and in which BamHI was introduced into the 5′ end and SalI was introduced into the 3′ end, from eurofins. A DNA fragment obtained by treating the synthetic DNA with BamHI/SalI, and a DNA fragment obtained by treating pUC18-ppat-plr produced in Comparative Example 2 with BamHI and SalI were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:90 according to a usual method of determining a nucleotide sequence. The obtained plasmid was named pUC18-ppat-Rsadh-plr.

### Example 18: Production of ppat-Pmadh-plr Expression Plasmid

A synthetic DNA having the nucleotide sequence of SEQ ID NO:91 was obtained by custom synthesis of the nucleotide sequence of an alanine dehydrogenase gene derived from Pseudomonas mendocina, into which a mutation as D198A in an encoded amino acid sequence was introduced, which was designed to be codon-optimized for E. coli, and in which BamHI was introduced into the 5′ end and SalI was introduced into the 3′ end, from eurofins. A DNA fragment obtained by treating the synthetic DNA with BamHI/SalI, and a DNA fragment obtained by treating pUC18-ppat-plr produced in Comparative Example 2 with BamHI and SalI were ligated using Ligation High (manufactured by TOYOBO Co., Ltd.). Escherichia coli DH5α (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:91 according to a usual method of determining a nucleotide sequence. The obtained plasmid was named pUC18-ppat-Pmadh-plr.

### Example 19: Production of ppat-Bjadh1-plr Expression Plasmid and ppat-Bjadh2-plr Expression Plasmid

Two kinds of alanine dehydrogenase genes derived from Bradyrhizobium japonicum exist in Bradyrhizobium japonicum. Two kinds of synthetic DNAs having the nucleotide sequences of SEQ ID NO:92 and SEQ ID NO:93 were obtained by custom synthesis of the nucleotide sequences of the two kinds of the alanine dehydrogenase genes derived from Bradyrhizobium japonicum, into which a mutation as D198A in an encoded amino acid sequence was introduced, which were designed to be codon-optimized for E. coli, and in which BamHI was introduced into the 5′ end and SalI was introduced into the 3′ end, from eurofins. A DNA fragment obtained by treating the synthetic DNA including SEQ ID NO:92 with BamHI/SalI, and a DNA fragment obtained by treating pUC18-ppat-plr produced in Comparative Example 2 with BamHI and SalI were ligated using Ligation High (manufactured by TOYOBO Co., Ltd.). Escherichia coli DH5α (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:92 according to a usual method of determining a nucleotide sequence. The obtained plasmid was named pUC18-ppat-Bjadh1-plr.

Similarly, a DNA fragment obtained by treating the synthetic DNA including SEQ ID NO:93 with BamHI/SalI, and a DNA fragment obtained by treating pUC18-ppat-plr produced in Comparative Example 2 with BamHI and SalI were ligated using Ligation High (manufactured by TOYOBO Co., Ltd.). Escherichia coli DH5α (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:93 according to a usual method of determining a nucleotide sequence. The obtained plasmid was named pUC18-ppat-Bjadh2-plr.

### Example 20: Production of ppat-Saadh-plr Expression Plasmid

A synthetic DNA having the nucleotide sequence of SEQ ID NO:94 was obtained by custom synthesis of the nucleotide sequence of an alanine dehydrogenase gene derived from Streptomyces aureofaciens, into which a mutation as D198A in an encoded amino acid sequence was introduced, which was designed to be codon-optimized for E. coli, and in which BamHI was introduced into the 5′ end and SalI was introduced into the 3′ end, from eurofins.

A DNA fragment obtained by treating the synthetic DNA with BamHI/SalI, and a DNA fragment obtained by treating pUC18-ppat-plr produced in Comparative Example 2 with BamHI and SalI were ligated using Ligation High (manufactured by TOYOBO Co., Ltd.). Escherichia coli DH5α (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:94 according to a usual method of determining a nucleotide sequence. The obtained plasmid was named pUC18-ppat-Saadh-plr.

### Example 21: Production of ppat-Acadh-plr Expression Plasmid

A synthetic DNA having the nucleotide sequence of SEQ ID NO:95 was obtained by custom synthesis of the nucleotide sequence of an alanine dehydrogenase gene derived from Anabaena cylindrica, into which a mutation as D198A in an encoded amino acid sequence was introduced, which was designed to be codon-optimized for E. coli, and in which BamHI was introduced into the 5′ end and SalI was introduced into the 3′ end, from eurofins. A DNA fragment obtained by treating the synthetic DNA with BamHI/SalI, and a DNA fragment obtained by treating pUC18-ppat-plr produced in Comparative Example 2 with BamHI and SalI were ligated using Ligation High (manufactured by TOYOBO Co., Ltd.). Escherichia coli DH5α (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:95 according to a usual method of determining a nucleotide sequence. The obtained plasmid was named pUC18-ppat-Acadh-plr.

### Example 22: Production of ppat-Bsadh-plr Expression Plasmid

A synthetic DNA having the nucleotide sequence of SEQ ID NO:96 was obtained by custom synthesis of the nucleotide sequence of an alanine dehydrogenase gene derived from Bacillus subtilis, into which a mutation as D196A in an encoded amino acid sequence was introduced, which was designed to be codon-optimized for E. coli, and in which BamHI was introduced into the 5′ end and SalI was introduced into the 3′ end, from eurofins. A DNA fragment obtained by treating the synthetic DNA with BamHI/SalI, and a DNA fragment obtained by treating pUC18-ppat-plr produced in Comparative Example 2 with BamHI and SalI were ligated using Ligation High (manufactured by TOYOBO Co., Ltd.). Escherichia coli DH5α (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:96 according to a usual method of determining a nucleotide sequence. The obtained plasmid was named pUC18-ppat-Bsadh-plr.

Test 6: Production of Pyridoxamine Using Pyridoxine as Raw Material

DH5α transformed with each of the plasmids produced in Examples 16 to 22 was inoculated into 100 mL of LB medium including 100 μg/mL of ampicillin in a 500 mL baffled Erlenmeyer flask and cultured with shaking at 30° C. for 24 hours. 40 mL of culture solution was taken, and centrifuged at 5,000 rpm for 5 minutes, and bacterial cells obtained as a precipitate were suspended in 0.9 mL of water to adjust prepare a bacterial cell suspension.

Pyridoxine hydrochloride and L-alanine were dissolved in water to prepare a substrate solution including pyridoxine hydrochloride (500 mM) and L-alanine (500 mM). The pH of the substrate solution was adjusted to pH 8.0 with 25% aqueous ammonia, and 400 of substrate solution, 500 μL of water, and 100 μL of bacterial cell suspension were mixed, and allowed to react at 37° C. for 1 hour. A part of the reaction liquid was collected, and analyzed under the analysis conditions described above. The results are set forth in Table 5. The amount of produced pyridoxamine set forth in Table 5 is expressed in a relative amount, with the molar amount of pyridoxamine dihydrochloride produced by the ppat-adh-plr expression strain produced in Example 1 set to 100.

TABLE 5

| | Amount of Produced Pyridoxamine |
|---|---|
| Introduced Plasmid | (Relative Value) |
| pUC18-ppat-adh-plr | 100 |
| pUC18-ppat-Ahadh-plr | 79 |
| pUC18-ppat-Rsadh-plr | 66 |
| pUC18-ppat-Pmadh-plr | 113 |
| pUC18-ppat-Bjadh1-plr | 32 |
| pUC18-ppat-Bjadh2-plr | 22 |
| pUC18-ppat-Saadh-plr | 84 |
| pUC18-ppat-Acadh-plr | 24 |
| pUC18-ppat-Bsadh-plr | 105 |

As set forth in Table 5, pyridoxamine dihydrochloride was able to be also produced from pyridoxine hydrochloride by introducing an alanine dehydrogenase gene derived from Aeromonas hydrophila, an alanine dehydrogenase gene derived from Rhizobium sp. LPU83, an alanine dehydrogenase gene derived from Pseudomonas mendocina, an alanine dehydrogenase gene derived from Bradyrhizobium japonicum (each of two kinds of alanine dehydrogenase genes from Bradyrhizobium japonicum), an alanine dehydrogenase gene derived from Streptomyces aureofaciens, an alanine dehydrogenase gene derived from Anabaena cylindrica, or an alanine dehydrogenase gene derived from Bacillus subtilis, as a gene encoding an alanine dehydrogenase, instead of the alanine dehydrogenase gene derived from Shewanella sp. AC10 used in Example 1.

### Example 23: Production of E. coli Expressing Protein Including Amino Acid Sequence of SEQ ID NO:1

A DNA (SEQ ID NO:13) in which a nucleotide sequence encoding the pyridoxine dehydrogenase gene (SEQ ID NO:1) of Saccharomyces cerevisiae was codon-optimized for E. coli was obtained by custom synthesis from GenScript. A target gene was amplified by a polymerase chain reaction (PCR) method using, as primers, oligonucleotides shown in SEQ ID NO:18 and SEQ ID NO:17 with the obtained synthetic DNA as a template. A DNA fragment obtained by treating the amplified DNA with EcoRI/HindIII, and an EcoRI/HindIII treated product of pUC18 (manufactured by Takara Bio Inc.) were ligated using Ligation High (manufactured by TOYOBO Co., Ltd.) to obtain an expression vector. Escherichia coli DH5α (manufactured by TOYOBO Co., Ltd.) was transformed with the ligation product including the expression vector. The transformant was cultured on an LB agar medium, and a strain having a target plasmid was selected from ampicillin resistant strains.

The plasmid was extracted from the obtained transformant in order to confirm that the selected strain includes a polynucleotide including the nucleotide sequence of SEQ ID NO:13. The nucleotide sequence of the DNA fragment introduced into the plasmid was confirmed to be the nucleotide sequence of SEQ ID NO:13 according to a usual method of determining a nucleotide sequence. The obtained plasmid was named pUC18-plr.

### Example 24: Synthesis of Pyridoxal Using Pyridoxine as Substrate with Pyridoxine Dehydrogenase Expression Strain

An E. coli DH5α strain transformed with pUC18-plr was inoculated into 2 mL of LB liquid medium (containing 100 μg/mL of ampicillin), and the resultant was cultured with shaking in a test tube at 37° C. for 24 hours. 1 mL of culture solution was centrifuged at 13,000 rpm for 5 minutes, and obtained bacterial cells were suspended in 1 mL of water to obtain a bacterial cell suspension.

A substrate solution was obtained by dissolving pyridoxine hydrochloride and β-NADP⁺ in water so that each thereof was 100 mM. Mixing of 100 μL of substrate solution, 100 μL of 1 M Tris-HCl (pH 8.0), and 700 μL of water was performed, and 100 μL of bacterial cell suspension was added to the resultant to start reaction. The reaction was conducted at 37° C. for 1 hour.

After the end of the reaction, a part of the reaction liquid was collected, and analyzed by high performance liquid chromatography (HPLC).

The reaction conducted for 1 hour allowed 37.2 μg of pyridoxal to be obtained, and the rate of producing pyridoxal per OD 660 was 2.1×10⁻³ μmol/min/OD 660.

Analysis Conditions of HPLC

Column: Shodex Asahipak ODP-50 6E (trade name, manufactured by SHOWA DENKO K.K.)

Guard column: Shodex Asahipak ODP-50G 6A (trade name, manufactured by SHOWA DENKO K.K.)

Column temperature: 30° C.

Pump flow rate: 1.0 mL/min

Eluent: 50 mM phosphate buffer (pH 2.0)

Detection: UV 294 nm.

### Comparative Example 5: Synthesis of Pyridoxal Using Pyridoxine as Substrate with Yeast

Saccharomyces cerevisiae X-2180-1A (ATCC26486) was inoculated into 2 mL of YPD liquid medium, and the resultant was cultured with shaking in a test tube at 30° C. for 24 hours. 1 mL of culture solution was centrifuged at 13,000 rpm for 5 minutes, and obtained bacterial cells were suspended in 1 mL of water to obtain a bacterial cell suspension.

A substrate solution having the same composition as the composition of the substrate solution in Example 24 was prepared, and reaction was conducted under the same conditions as the reaction conditions in Example 24.

After the end of the reaction, a part of the reaction liquid was collected, and analyzed by high performance liquid chromatography (HPLC) under conditions similar to the conditions in Example 24.

The reaction conducted for 1 hour did not allow pyridoxal to be obtained.

### Example 25: Synthesis of Pyridoxine Using Pyridoxal as Substrate with Pyridoxine Dehydrogenase Expression Strain

A bacterial cell suspension was prepared in a manner similar to the manner of Example 24, and a substrate solution was obtained by dissolving pyridoxal hydrochloride and β-NADPH in water so that each thereof was 100 mM. Mixing of 100 μL of substrate solution, 100 μL of 1 M Tris-HCl (pH 8.0), and 700 μL of water was performed, and 100 μL of bacterial cell suspension was added to the resultant to start reaction.

After the end of the reaction, a part of the reaction liquid was collected, and analyzed by high performance liquid chromatography (HPLC) under conditions similar to the conditions in Example 24.

The reaction conducted for 1 hour allowed 815.5 μg of pyridoxine to be obtained, and the rate of producing pyridoxine per OD 660 was 4.5×10⁻² μmol/min/OD 660.

### Comparative Example 6: Synthesis of Pyridoxine Using Pyridoxal as Substrate with Yeast (Rate of Producing Pyridoxine per OD 660)

A bacterial cell suspension was prepared in a manner similar to the manner of Comparative Example 5, a substrate solution having the same composition as the composition of the substrate solution in Example 25 was prepared, and reaction was conducted under the same conditions as the reaction conditions in Example 25.

After the end of the reaction, a part of the reaction liquid was collected, and analyzed by high performance liquid chromatography (HPLC) under conditions similar to the conditions in Example 24.

The reaction conducted for 1 hour allowed 10.9 μg of pyridoxine to be obtained, and the rate of producing pyridoxine per OD 660 was 1.7×10⁻⁴ μmol/min/OD 660.

The results of Examples 24 to 25 and Comparative Examples 5 to 6 are set forth in Table 6. Examples 24 and 25 were prominently superior in the yield of pyridoxal or pyridoxine, and the rate of producing pyridoxal or pyridoxine to Comparative Examples 5 and 6. In particular, pyridoxal which was not more than detection sensitivity in Comparative Example 5 was able to be produced in Example 24 in the synthesis of pyridoxal.

TABLE 6

| | Synthesis of Pyridoxal | | Synthesis of Pyridoxine | |
|---|---|---|---|---|
| | | Comparative | | Comparative |
| | Example 2 | Example 1 | Example 3 | Example 2 |
| Yield (μg) | 37.2 | Not detected | 815.5 | 10.9 |
| Production Rate | 2.1 × 10⁻³ | Not detected | 4.5 × 10⁻² | 1.7 × 10⁻⁴ |
| (μmol/min/OD 660) | | | | |

### Example 26: Synthesis of Pyridoxal Using Pyridoxine as Substrate with Pyridoxine Dehydrogenase Expression Strain

An E. coli DH5α strain transformed with pUC18-plr was inoculated into 100 mL of LB liquid medium (containing 100 μg/mL of ampicillin), and the resultant was cultured with shaking in a baffled Erlenmeyer flask at 37° C. for 24 hours. 40 mL of culture solution was centrifuged at 8,000 rpm for 20 minutes, and obtained bacterial cells were suspended in 400 of water to obtain a bacterial cell suspension. A substrate solution having the same composition as the composition of the substrate solution in Example 24 was prepared, and reaction was conducted under the same conditions as the reaction conditions in Example 24. The reaction was conducted at 37° C. for 30 minutes.

After the end of the reaction, a part of the reaction liquid was collected, and analyzed by high performance liquid chromatography (HPLC) under conditions similar to the conditions in Example 24.

The reaction conducted for 30 minutes allowed 262.4 μg of pyridoxal to be obtained, and the rate of producing pyridoxal per dried bacterial cell weight was 5.8 μmol/min/g-dry cell.

### Comparative Example 7: Synthesis of Pyridoxal Using Pyridoxine as Substrate with Dry Yeast

Super Camelia Dry Yeast (trade name, Nisshin Foods Inc.) was suspended in water to achieve 100 mg/mL, and a bacterial cell suspension was obtained.

A substrate solution having the same composition as the composition of the substrate solution in Example 24 was prepared, and reaction was conducted under the same conditions as the reaction conditions in Example 24.

After the end of the reaction, a part of the reaction liquid was collected, and analyzed by high performance liquid chromatography (HPLC) under conditions similar to the conditions in Example 24.

The reaction conducted for 30 minutes allowed 34.1 μg of pyridoxal to be obtained, and the rate of producing pyridoxal per dried bacterial cell weight was 0.9 μmol/min/g-dry cell.

### Example 27: Synthesis of Pyridoxine Using Pyridoxal as Substrate with Pyridoxine Dehydrogenase Expression Strain

A bacterial cell suspension was prepared in a manner similar to the manner of Example 26, a substrate solution having the same composition as the composition of the substrate solution in Example 25 was prepared, and reaction was conducted under the same conditions as the reaction conditions in Example 25.

After the end of the reaction, a part of the reaction liquid was collected, and analyzed by high performance liquid chromatography (HPLC) under conditions similar to the conditions in Example 24.

The reaction conducted for 30 minutes allowed 1457.4 μg of pyridoxine to be obtained, and the rate of producing pyridoxine per dried bacterial cell weight was 33.5 μmol/min/g-dry cell.

### Comparative Example 8: Synthesis of Pyridoxine Using Pyridoxal as Substrate with Dry Yeast (Rate of Producing Pyridoxine Per Dried Bacterial Cell Weight)

A bacterial cell suspension was prepared in a manner similar to the manner of Comparative Example 7, a substrate solution having the same composition as the composition of the substrate solution in Example 25 was prepared, and reaction was conducted under the same conditions as the reaction conditions in Example 25.

After the end of the reaction, a part of the reaction liquid was collected, and analyzed by high performance liquid chromatography (HPLC) under conditions similar to the conditions in Example 24.

The reaction conducted for 30 minutes allowed 325.3 μg of pyridoxine to be obtained, and the rate of producing pyridoxine per dried bacterial cell weight was 13.9 μmol/min/g-dry cell.

The results of Examples 26 to 27 and Comparative Examples 7 to 8 are set forth in Table 7. Examples 26 and 27 were prominently superior in the yield of pyridoxal or pyridoxine, and the rate of producing pyridoxal or pyridoxine to Comparative Examples 7 and 8. In Example 26, the obtained yield of pyridoxal was about 8 times that in Comparative Example 7, and the production rate per gram of dried bacterial cell weight was about 6.5 times. In Example 27, the obtained yield of pyridoxine was about 4.5 times that in Comparative Example 8, and the production rate per gram of dried bacterial cell weight was about 2.5 times.

TABLE 7

| | Synthesis of Pyridoxal | | Synthesis of Pyridoxine | |
|---|---|---|---|---|
| | | Comparative | | Comparative |
| | Example 4 | Example 3 | Example 5 | Example 4 |
| Yield (μg) | 262.4 | 34.1 | 1457.4 | 325.3 |
| Production Rate | 5.8 | 0.9 | 33.5 | 13.9 |
| (μmol/min/ | | | | |
| g-dry cell) | | | | |

The disclosures of Japanese Patent Application No. 2017-095571 filed on May 12, 2017, and Japanese Patent Application No. 2017-095573 filed on May 12, 2017 are incorporated herein by reference in their entirety.

All publications, patent application, and technical standards mentioned in this specification are herein incorporated by reference to the same extent as if each individual publication, patent application, or technical standard was specifically and individually indicated to be incorporated by reference.

1. A recombinant microorganism, comprising:
a gene encoding a pyridoxine dehydrogenase, a gene encoding a pyridoxamine synthetase having an enzymatic activity of synthesizing pyridoxamine from pyridoxal, and a gene encoding an amino acid regeneration enzyme having an enzymatic activity of regenerating an amino acid consumed by the pyridoxamine synthetase,
wherein at least two of the gene encoding the pyridoxine dehydrogenase, the gene encoding the pyridoxamine synthetase, or the gene encoding the amino acid regeneration enzyme, are introduced from outside of a bacterial cell, or are endogenous to the bacterial cell and have an enhanced expression.

2. The recombinant microorganism according to claim 1, wherein the pyridoxamine synthetase is a pyridoxamine-pyruvate transaminase, a pyridoxamine-oxaloacetate transaminase, an aspartate transaminase, or a pyridoxamine phosphate transaminase.

3. The recombinant microorganism according to claim 1, wherein the pyridoxine dehydrogenase comprises at least one of the following partial amino acid sequence (a) or partial amino acid sequence (b), and has pyridoxine dehydrogenase activity:
(a)
(SEQ ID NO: 97)
NX1X2EX3YG
wherein X1 represents V, C, I, A, M, S, G, or L,
X2 represents G or A, and
X3 represents F or L; and
(b)
(SEQ ID NO: 98)
X4X5X6KGX7
wherein X4 represents I, V, F, or L,
X5 represents S, T, N, C, or M,
X6 represents C, V, A, I, W, or F, and
X7 represents G, A, S, or C.

4. The recombinant microorganism according to claim 1, wherein the pyridoxine dehydrogenase is represented by enzyme number EC1.1.1.65.

5. The recombinant microorganism according to claim 1, wherein the gene encoding the pyridoxine dehydrogenase is derived from Saccharomyces cerevisiae.

6. The recombinant microorganism according to claim 1, wherein the gene encoding the pyridoxine dehydrogenase has:
a nucleotide sequence of any one of SEQ ID NO:7 or SEQ ID NO:53 to SEQ ID NO:59,
a region between an 18th nucleotide and a 3′ end in a nucleotide sequence of any one of SEQ ID NO:13 or SEQ ID NO:75 to SEQ ID NO:81, or
a nucleotide sequence that hybridizes with DNA having a nucleotide sequence complementary to a nucleotide sequence of any one of SEQ ID NO:7 or SEQ ID NO:53 to SEQ ID NO:59, or with DNA having a nucleotide sequence complementary to a region between an 18th nucleotide and a 3′ end in a nucleotide sequence of any one of SEQ ID NO:13 or SEQ ID NO:75 to SEQ ID NO:81 under a stringent condition, and that encodes a protein having pyridoxine dehydrogenase activity.

7. The recombinant microorganism according to claim 1, wherein the gene encoding the pyridoxine dehydrogenase is derived from Schizosaccharomyces pombe.

8. The recombinant microorganism according to claim 1, wherein the gene encoding the pyridoxine dehydrogenase has a nucleotide sequence of SEQ ID NO:8, or has a nucleotide sequence that hybridizes with DNA having a nucleotide sequence complementary to the nucleotide sequence of SEQ ID NO:8 under a stringent condition, and that encodes a protein having pyridoxine dehydrogenase activity.

9. The recombinant microorganism according to claim 1, wherein the gene encoding the pyridoxine dehydrogenase has a nucleotide sequence encoding an amino acid sequence of any one of SEQ ID NO:1, SEQ ID NO:2, or SEQ ID NO:22 to SEQ ID NO:28, or an amino acid sequence that has 80% or more sequence identity with at least one amino acid sequence selected from the group consisting of SEQ ID NO:1, SEQ ID NO:2, and SEQ ID NO:22 to SEQ ID NO:28, and that has pyridoxine dehydrogenase activity.

10. The recombinant microorganism according to claim 1, wherein the pyridoxamine synthetase comprises at least one of the following partial amino acid sequence (c), partial amino acid sequence (d), partial amino acid sequence (e), partial amino acid sequence (f), partial amino acid sequence (g), or partial amino acid sequence (h), and has an enzymatic activity of synthesizing pyridoxamine from pyridoxal:
(c)
(SEQ ID NO: 99)
X8X9X10X11X12X13
wherein X8 represents L, M, I, or V,
X9 represents H or Q,
X10 represents G, C, or A,
X11 represents E or D,
X12 represents P or A, and
X13 represents V, I, L, or A;
(d)
(SEQ ID NO: 100)
X14X15TPSGTX16X17
wherein X14 represents H or S,
X15 represents D or E,
X16 represents I, V, or L, and
X17 represents N or T;
(e)
(SEQ ID NO: 101)
X18DX19VSX20X21
wherein X18 represents V, I, or A,
X19 represents A, T, or S,
X20 represents S, A, or G, and
X21 represents F, W, or V;
(f)
(SEQ ID NO: 102)
X22X23X24KCX25GX26X27P
wherein X22 represents G or S,
X23 represents P, S, or A,
X24 represents N, G, S, A, or Q,
X25 represents L or M,
X26 represents A, S, C, or G, and
X27 represents P, T, S, or A;
(g)
(SEQ ID NO: 103)
X28X29X30X31SX32GX33X34
wherein X28 represents G or D,
X29 represents V or I,
X30 represents V, T, A, S, M, I, or L,
X31 represents F, M, L, I, or V,
X32 represents S, G, A, T, I, L, or H,
X33 represents R, M, or Q, and
X34 represents G, R, A, D, H, or K; and
(h)
(SEQ ID NO: 104)
X35X36RX37X38HMGX39X40A
wherein X35 represents L or V,
X36 represents T, I, V, or L,
X37 represents I, V, or L,
X38 represents G or S,
X39 represents P, A, or R, and
X40 represents T, V, or S.

11. The recombinant microorganism according to claim 1,
wherein the pyridoxamine synthetase is represented by enzyme number EC2.6.1.30.

12. The recombinant microorganism according to claim 1, wherein the gene encoding the pyridoxamine synthetase is derived from Mesorhizobium loti.

13. The recombinant microorganism according to claim 1, wherein the gene encoding the pyridoxamine synthetase has:
a nucleotide sequence of any one of SEQ ID NO:9 or SEQ ID NO:60 to SEQ ID NO:66,
a region between an 18th nucleotide and a 3′ end in a nucleotide sequence of SEQ ID NO:14, or a region between an 18th nucleotide and a 3′ end in a nucleotide sequence of any one of SEQ ID NO:82 to SEQ ID NO:88, or
a nucleotide sequence that hybridizes with DNA having a nucleotide sequence complementary to a nucleotide sequence of any one of SEQ ID NO:9 or SEQ ID NO:60 to SEQ ID NO:66, or with DNA having a nucleotide sequence complementary to the region between a 18th nucleotide and an 3′ end in the nucleotide sequence of SEQ ID NO:14, or a region between an 18th nucleotide and a 3′ end in a nucleotide sequence of any one of SEQ ID NO:82 to SEQ ID NO:88, under a stringent condition, and that encodes a protein having an enzymatic activity of synthesizing pyridoxamine from pyridoxal.

14. The recombinant microorganism according to claim 1, wherein the gene encoding the pyridoxamine synthetase has a nucleotide sequence encoding an amino acid sequence of any one of SEQ ID NO:3 or SEQ ID NO:29 to SEQ ID NO:35, or an amino acid sequence that has 80% or more sequence identity with at least one amino acid sequence selected from the group consisting of SEQ ID NO:3 and SEQ ID NO:29 to SEQ ID NO:35, and that has an enzymatic activity of synthesizing pyridoxamine from pyridoxal.

15. The recombinant microorganism according to claim 10, wherein the amino acid regeneration enzyme is an alanine dehydrogenase.

16. The recombinant microorganism according to claim 15, wherein the alanine dehydrogenase is enabled to use NADPH as a coenzyme in a reaction in which L-alanine is produced from pyruvic acid and NH3.

17. The recombinant microorganism according to claim 15 or 17, wherein the alanine dehydrogenase comprises at least one of the following partial amino acid sequence (i), partial amino acid sequence (j), partial amino acid sequence (k), or partial amino acid sequence (I), and has an activity of producing L-alanine from pyruvic acid and NH3:
(i)
(SEQ ID NO: 105)
EX41KX42X43EX44RX45X46
wherein X41 represents I, T, S, F, N, or V,
X42 represents N, M, A, V, L, T, or D,
X43 represents H, N, L, or Q,
X44 represents Y, N, or F,
X45 represents V or I, and
X46 represents G or A;
(j)
(SEQ ID NO: 106)
X47X48X49KVKEPX50
wherein X47 represents M or L,
X48 represents I, L, or V,
X49 represents V, L, I, or M, and
X50 represents Q, L, V, N, or I;
(k)
(SEQ ID NO: 107)
LX51TYLHLA
wherein X51 represents F or Y; and
(l)
(SEQ ID NO: 108)
X52DX53AX54DQGG
wherein X52 represents V or A,
X53 represents V or I, and
X54 represents I or V.

18. The recombinant microorganism according to claim 15, wherein the gene encoding the amino acid regeneration enzyme is derived from Shewanella sp. AC10.

19. The recombinant microorganism according to claim 15, wherein the gene encoding the amino acid regeneration enzyme has:
a nucleotide sequence of any one of SEQ ID NO:11 or SEQ ID NO:67 to SEQ ID NO:74,
a region between an 18th nucleotide and a 3′ end in a nucleotide sequence of any one of SEQ ID NO:15 or SEQ ID NO:89 to SEQ ID NO:96, or
a nucleotide sequence that hybridizes with DNA having a nucleotide sequence complementary to a nucleotide sequence of any one of SEQ ID NO:11 or SEQ ID NO:67 to SEQ ID NO:74, or with DNA having a nucleotide sequence complementary to a region between an 18th nucleotide and a 3′ end in a nucleotide sequence of any one of SEQ ID NO:15 or SEQ ID NO:89 to SEQ ID NO:96 under a stringent condition, and that encodes a protein having alanine regeneration enzymatic activity.

20. The recombinant microorganism according to claim 15, wherein the gene encoding the amino acid regeneration enzyme has a nucleotide sequence encoding an amino acid sequence of any one of SEQ ID: NO 5 or SEQ ID NO:45 to SEQ ID NO:52, or an amino acid sequence that has 80% or more sequence identity with at least one amino acid sequence selected from the group consisting of SEQ ID NO:5 and SEQ ID NO:45 to SEQ ID NO:52, and that has alanine regeneration enzymatic activity.

21. The recombinant microorganism according to claim 1, wherein the pyridoxamine synthetase is represented by enzyme number EC2.6.1.31 or EC2.6.1.1.

22. The recombinant microorganism according to claim 1, wherein the gene encoding the pyridoxamine synthetase is derived from Escherichia coli.

23. The recombinant microorganism according to claim 1, wherein the gene encoding the pyridoxamine synthetase has a nucleotide sequence of SEQ ID NO:10, or has a nucleotide sequence that hybridizes with DNA having a nucleotide sequence complementary to the nucleotide sequence of SEQ ID NO:10 under a stringent condition, and that encodes a protein having an enzymatic activity of synthesizing pyridoxamine from pyridoxal.

24. The recombinant microorganism according to claim 1, wherein the gene encoding the pyridoxamine synthetase has a nucleotide sequence encoding an amino acid sequence of SEQ ID NO:4, or an amino acid sequence that has 80% or more sequence identity with the amino acid sequence of SEQ ID NO:4, and that has an enzymatic activity of synthesizing pyridoxamine from pyridoxal.

25. The recombinant microorganism according to claim 21, wherein the amino acid regeneration enzyme is a glutamate dehydrogenase.

26. The recombinant microorganism according to claim 25, wherein the gene encoding the amino acid regeneration enzyme is derived from Escherichia coli.

27. The recombinant microorganism according to claim 25, wherein the gene encoding the amino acid regeneration enzyme has a nucleotide sequence of SEQ ID NO:12, or has a nucleotide sequence that hybridizes with DNA having a nucleotide sequence complementary to the nucleotide sequence of SEQ ID NO:12 under a stringent condition, and that encodes a protein having glutamate regeneration enzymatic activity.

28. The recombinant microorganism according to claim 25, wherein the gene encoding the amino acid regeneration enzyme has a nucleotide sequence encoding an amino acid sequence of SEQ ID NO:6, or an amino acid sequence that has 80% or more sequence identity with the amino acid sequence of SEQ ID NO:6, and that has glutamate regeneration enzymatic activity.

29. The recombinant microorganism according to claim 1, the recombinant microorganism being recombinant E. coli.

30. A method of producing pyridoxamine or a salt thereof, the method comprising bringing the recombinant microorganism according to claim 1, a culture of the recombinant microorganism, or a treated product of the recombinant microorganism or the culture, into contact with pyridoxine or a salt thereof to produce pyridoxamine or a salt thereof.

31. The production method according to claim 30, wherein the recombinant microorganism, the culture of the recombinant microorganism, or the treated product of the recombinant microorganism or the culture, comprises the pyridoxine dehydrogenase, the pyridoxamine synthetase, and the amino acid regeneration enzyme.

32. The production method according to claim 30 or 31, wherein the treated product of the recombinant microorganism or the treated product of the culture of the recombinant microorganism is treated by treatment comprising one or more selected from the group consisting of heat treatment, cooling treatment, mechanical destruction of a cell, ultrasonic treatment, freeze-thaw treatment, drying treatment, pressurized or reduced pressure treatment, osmotic pressure treatment, cell autolysis, surfactant treatment, enzyme treatment, cell separation treatment, purification treatment, and extraction treatment.

33. A recombinant microorganism, into which at least one polynucleotide selected from the group consisting of the following (1) to (7) is introduced:
(1) a polynucleotide that encodes a protein having an amino acid sequence of SEQ ID NO:1;
(2) a polynucleotide that encodes a protein having an amino acid sequence in which from 1 to 50 amino acids are deleted, added, or substituted in the amino acid sequence of SEQ ID NO:1, the protein having an activity of synthesizing pyridoxine or a salt thereof, or pyridoxal or a salt thereof;
(3) a polynucleotide that encodes a protein having an amino acid sequence having 60% or more sequence identity with the amino acid sequence of SEQ ID NO:1, the protein having an activity of synthesizing pyridoxine or a salt thereof, or pyridoxal or a salt thereof;
(4) a polynucleotide having a nucleotide sequence of SEQ ID NO:13;
(5) a polynucleotide having a nucleotide sequence in which from 1 to 150 nucleotides are deleted, added, or substituted in the nucleotide sequence of SEQ ID NO:13, the polynucleotide encoding a protein having an activity of synthesizing pyridoxine or a salt thereof, or pyridoxal or a salt thereof;
(6) a polynucleotide having a nucleotide sequence having 60% or more sequence identity with the nucleotide sequence of SEQ ID NO:13, the polynucleotide encoding a protein having an activity of synthesizing pyridoxine or a salt thereof, or pyridoxal or a salt thereof; and
(7) a polynucleotide that hybridizes with a polynucleotide having a nucleotide sequence complementary to the nucleotide sequence of SEQ ID NO:13 under a stringent condition, the polynucleotide encoding a protein having an activity of synthesizing pyridoxine or a salt thereof, or pyridoxal or a salt thereof.

34. The recombinant microorganism according to claim 33, wherein the recombinant microorganism is a recombinant bacterium.

35. The recombinant microorganism according to claim 34, wherein the recombinant microorganism is recombinant E. coli.

36. The recombinant microorganism according to claim 33, wherein the protein is an enzyme classified under EC number 1.1.1.65.

37. The recombinant microorganism according to claim 33, wherein the protein is a pyridoxine dehydrogenase derived from Saccharomyces cerevisiae.

38. A method of producing pyridoxal or a salt thereof, the method comprising bringing the recombinant microorganism according to claim 33, into contact with pyridoxine or a salt thereof.

39. A method of producing pyridoxal or a salt thereof, the method comprising bringing a culture obtained by culturing the recombinant microorganism according to claim 33, or a treated product of the culture, into contact with pyridoxine or a salt thereof.
